(19) United States
(12) Patent Application Publication
(10) Pub. No.: US 2012/0309739 A1
(43) Pub. Date: Dec. 6, 2012

(54) AKT / PKB INHIBITORS

(75) Inventors: Mark Peter Bell, Craigavon (GB); Colin Roderick O'Dowd, Craigavon (GB); James Samuel Shane Rountree, Craigavon (GB); Graham Peter Trevitt, Craigavon (GB); Timothy Harrison, Craigavon (GB); Mary Melissa McFarland, Craigavon (GB)

(73) Assignee: ALMAC DISCOVERY LIMITED, Craigavon (GB)

(21) Appl. No.: 13/508,137

(22) PCT Filed: Nov. 3, 2010
(86) PCT No.: PCT/GB10/02033

§ 371 Date: Jul. 27, 2012

(30) Foreign Application Priority Data
Nov. 4, 2009 (GB) 0919380.6

(51) Int. Cl. A61K 31/4355; C07D 307/81; A61K 31/343; A61K 31/4985; A61P 35/00; A61K 31/5377; C07D 311/36; A61K 31/353; A61K 31/397; C07D 405/10; C07D 491/048; A61K 31/519

(52) U.S. Cl. 514/210.21; 546/115; 514/302; 546/116; 549/469; 514/469; 544/350; 514/249; 544/117; 544/278; 514/260.1; 544/127; 514/233.8; 549/403; 514/456; 514/210.19; 548/953; 514/252.16; 514/234.5; 514/234.2

(57) ABSTRACT

The invention relates to a series of compounds with particular activity as inhibitors of the serine-threonine kinase AKT. Also provided are pharmaceutical compositions comprising same as well as methods for treating cancer.

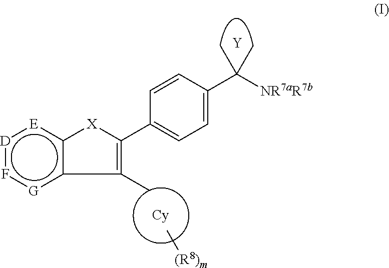

The present invention relates to compounds that are useful as inhibitors of the activity of one or more isoforms of the serine/threonine kinase, AKT. The present invention also relates to pharmaceutical compositions comprising these compounds and to methods of using these compounds in the treatment of cancer and methods of treating cancer.

### BACKGROUND TO THE INVENTION

The AKT protein family, also known as protein kinases B (PKB), are known to be involved in a wide variety of biological processes including cell proliferation, differentiation, apoptosis, tumorigenesis, as well as glycogen synthesis and glucose uptake. These enzymes are members of the serine/threonine-specific protein kinase family.

The PKB/AKT pathway has been identified as an important regulator of cell survival signalling and apoptosis in cells. Signalling is thought to occur through a range of growth factor receptors including platelet derived growth factor, insulin growth factor and nerve growth factor, resulting in activation of phosphatidylinositol 3-OH kinase (PI-3K). This activation in turn leads to the generation of phosphatidylinositol (3,4,5)triphosphate (PIP3). Activated PIP3 binds to and in turn phosphorylates the enzyme PDK-1, the main activator of AKT, through its pleckstrin homology domain. Activated PDK-1 is responsible for a phosphorylation event at Thr308 of AKT, which induces a conformational change that facilitates further phosphorylation of AKT at Ser 473 by PDK-2.

PDK-1 phosphorylation of downstream kinases is not unique to AKT, as it has been reported to activate p70 S6 kinase and protein kinase C.

The activation of AKT influences multiple events within the cell including the inhibition of apoptosis, the progression of the cell cycle, cellular survival, metabolism, angiogenesis and hormone resistance.

Presently three family members of AKT have been identified, AKT 1, AKT 2 and AKT 3 (also known as PKBα, PKBβ and PKBγ). The family members share 80% amino acid sequence homology and all retain similar regional structure. They possess a C-terminal pleckstrin homology (PH) domain, a catalytic domain, a short q helical linker region and a carboxyl terminal domain. The PH domain permits binding of proteins to the cell membrane through a phospholipid interaction. The catalytic domain of AKT family members contains two residues essential for kinase activation, namely Thr308 and Ser 473. In turn AKT can phosphorylate any protein containing the RXRXXS/T-B motif where X represents any amino acid and B represents bulky hydrophobic residues.

Turning to the cellular function of AKT, hyper activation of AKT has been linked to the inhibition of cellular apoptosis due to phosphorylation and negative regulation of the forkhead family of transcription factors which regulate various genes responsible for instigating death processes including FKHR, FKHRL1 and AFX. Conversely AKT has been reported to up-regulate genes which are known to be anti-apoptotic including IKK and CREB. It is this mixture of positive and negative regulation which highlights the importance of AKT in regulating apoptosis. AKT promotes unwanted cell survival through its' phosphorylation of several key apoptotic proteins including Bad and Pro-caspase 9, thus rendering them inactive and preventing signalling through this pathway. AKT activates and inhibits multiple mechanisms which have a major role in the progression of the cell cycle, ultimately leading to cell proliferation. The best characterised cell cycle regulator and tumour suppressor proteins p53 can be dysregulated via AKT phosphorylation and activation of the main p53 negative regulator MDM2. Phosphorylated MDM2 translocates to the nucleus where it prevents p53 transcription. The inhibition of p53 allows aberrant proliferation of the cell and progression towards a benign state.

In a similar fashion, AKT can also phosphorylate p27kip1 and p21; two main inhibitors of cell cycle progression, leading to loss of function, resulting in unchecked cell cycle progress and excessive proliferation.

AKT activation causes an increase in the rate of glycolysis by increasing the rate of glucose metabolism. It has also been reported that activated AKT stimulates the transport of amino acids and supports mTOR dependent increases in protein translation. Proangiogenic factors such as vascular endothelial growth factor (VEGF), have been reported to activate AKT, ultimately resulting in inhibition of endothelial apoptosis, as well as activating endothelial nitric oxide synthase (eNOS). The sum result of this is rapid neo-vascularisation and cell migration.

Hypoxia driven angiogenesis, primarily mediated by hypoxia inducible factor (HIF 1α) can lead to the induction of multiple proteins including VEGF. Increased activated AKT has been reported to increase HIF-1α expression leading to an increase in angiogenesis independent of a hypoxic environment. Recent data has shown that HIF-1α activity in invasive breast cancer is correlated with increased activated AKT-1 phosphorylation.

Estrogen receptor (ER) and androgen receptor (AR) inhibitors designed to inhibit cell signalling and induce apoptosis, are vital tools in cancer therapies. Incidence of resistance to these drugs arises rapidly in cancers including prostate, breast and ovarian. AKT has been reported to phosphorylate androgen receptors, leading to inhibition of AR activity and blockade of normal apoptotic signalling in prostate cancer induced by androgens.

In a similar manner, activation of AKT leads to phosphorylation of ERα resulting in an inhibition of tamoxifen mediated apoptosis or tumour regression, coupled with the creation of an estrogen independent signalling pathway. Activated AKT-2 has been identified as a promoter of ERα transcription in the presence or absence of estrogen increasing the rate of proliferation of breast cancer cells.

Hyper-activated AKT has been reported in a range of cancers compared to normal tissues including breast, lung, prostate, gastric, ovary, pancreas, thyroid, glioblastoma and haemological cancers. Phosphorylation of AKT has also been associated with clinical characteristics including increased stage and grade of tumour and increased poor prognosis. The activation of AKT can arise from a number of different genetic mutations in the AKT/PI-3K pathway.

Somatic mutations in the PI-3KCA gene have been widely reported in a large variety of tumours including breast, prostate and head and neck. A large number of these mutations will increase the copy number of the gene leading to an increase in PI-3K activity. A recent study has identified a PI-3K mutation which selectively phosphorylates AKT in colon cancer which results in increase cell proliferation and invasion.

Any mutation which increases the activity of the PI-3K pathway will ultimately result in an increased activation of AKT. Gene amplifications are common occurrences in cancer. Amplifications of AKT-2 have been reported in ovarian, pancreatic, breast and head and neck squamous cell carcinoma. No amplifications or mutations in AKT-3 have been reported to date although deletion mutations leading to hyperactivation and amplification mutations have been reported associated with AKT-1. One mutation; E17K, results in pathological localization of AKT-1 to the cell membrane, inducing its activation and resulting in down-stream signalling and cellular transformation. In vivo, this mutation has been shown to induce leukaemia in mice.

Phosphatase and tensin homolog deleted on chromosome 10 (PTEN) is a tumour suppressor gene known to negatively regulate AKT function. In cancer, loss of PTEN function results in constitutive phosphorylation of AKT and other down-stream effectors of the PI-3K pathway. Loss of PTEN, due to deletion mutations or promoter methylation, has been reported in a number of different cancers including glioblastoma, endometrial, lung, breast, prostate and thyroid. This loss is commonly associated with hyperactivation of AKT. Recent studies have shown that loss of heterozygosity (LOH) at the PTEN gene was directly correlated to increased AKT activation and chemoresistance in gastric carcinomas and decreased progesterone receptor expression in breast carcinomas.

AKT activation is commonly initiated at the cell surface through a signalling event at a receptor, usually one of the tyrosine kinase family. Two tyrosine kinase receptors commonly amplified or over-expressed in cancer are HER2 and EGFR. In HER2 over-expressing tumours there is often a hyper-activation of AKT, this has been reported in ovarian, stomach and bladder cancer. Similarly in EGFR over-expressing tumours, particularly those with the EGFRvIII activating mutation, selective activation of AKT has been reported in a range of cancers including non-small cell lung cancers, breast, ovarian and most commonly high grade gliomas.

Examples of AKT inhibitors are provided in WO 2008/070134, WO 2008/070016 and WO 2008/070041. These documents provide specific naphthyridine compounds fused to a five membered heterocycle.

### SUMMARY OF THE INVENTION

In a first aspect the present invention provides a compound according to Formula (I):

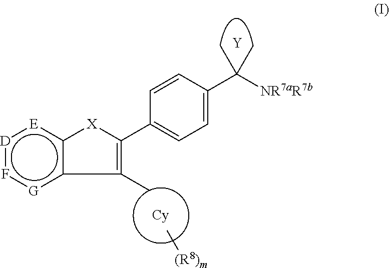

wherein:
0, 1 or 2 of D, E, F and G are independently selected from N, NH and NR¹ and the others are independently selected from CH and CR², wherein each R¹ is independently selected from aryl, C1-C10 alkyl, CONHR³, CONR^{3a}R^{3b}, COR³ and CO₂R³ and each R² is independently selected from aryl, C1-C10 alkyl, CN, CHO, CO₂H, CONH₂, CONHR³, CONR^{3a}R^{3b}, COR³, CO₂R³, NH₂, NHR³, NR^{3a}R^{3b}, NHCOR³, NHSO₂R³, NR^{3a}COR^{3b}, NR^{3a}SO₂R^{3b}, oxo, OH, OR³, SH, SR³, SOR³, SO₂R³, SO₂NHR³, SO₂NR^{3a}R^{3b}, F, Cl, Br and I, wherein each R³, R^{3a} and R^{3b} is independently selected from C1-C10 alkyl, including wherein R^{3a} and R^{3b} are joined to one another to form a heterocycle that includes the nitrogen to which they are attached;

wherein at least D or G is NH or NR¹ if E or F is CO and at least E or F is NH or NR¹ if D or G is CO;

wherein separate R¹ and/or R² groups may be joined to one another to form a heterocycle that includes the C and/or N atoms to which they are attached if the separate R¹ and/or R² groups are contained on D and E and/or F and G, or D and F and the separate R² groups are selected from OR³, SR³, SOR³, SO₂R³, SO₂NHR³, SO₂NR^{3a}R^{3b}, NHR³, NR^{3a}R^{3b}, CO₂R³, CONHR³ and CONR^{3a}R³, and/or wherein separate R¹ and/or R² groups on F and G may be joined to form the structure:

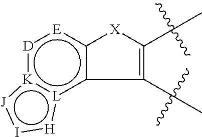

where 0 or 1 of K and L are N and the other(s) is/are C, and wherein H, I and J are independently selected from O, S, SO, SO₂, NH, NR⁴, N, CH and CR⁵, wherein each R⁴ is independently selected from aryl, C1-C10 alkyl, CONHR⁶, CONR^{6a}R^{6b}, COR⁶ and CO₂R⁶ and each R⁵ is independently selected from aryl, C1-C10 alkyl, CN, CHO, CO₂H, CONH₂, CONHR⁶, CONR^{6a}R^{6b}, COR⁶, CO₂R⁶, oxo, NH₂, NHR⁶, NR^{6a}R^{6b}, OH, OR⁶, SH, SR⁶, SOR⁶, SO₂R⁶, SO₂NHR⁶, SO₂NR^{6a}R^{6b}, F, Cl, Br and I, wherein each R⁶, R^{6a} and R^{6b} is independently selected from C1-C10 alkyl, including wherein R^{6a} and R^{6b} are joined to one another to form a heterocycle that includes the nitrogen to which they are attached,
X is selected from O, NR¹⁰, S, SO, or SO₂
R^{7a} and R^{7b} are independently selected from H and alkyl, including wherein R^{7a} and R^{7b} are joined to one another to form a heterocycle that includes the nitrogen to which they are attached; and

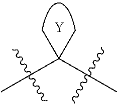

is selected from:

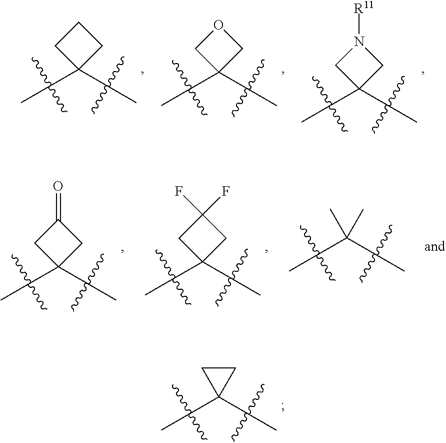

ring Cy is selected from (C₃ to C₈)cycloalkyl and aryl, wherein m is 0, 1, 2, 3, 4 or 5, and each R⁸ is independently selected from alkyl, CN, CHO CO₂H, CONH₂, CONHR⁹, CONHR^{9a}R^{9b}, COR⁹, CO₂R⁹, NH₂, NHR⁹, NR^{9a}R^{9b}, NHCOR⁹, NHSO₂R⁹, NR^{9a}COR^{9b}, NR^{9a}SO₂R^{9b}, OH, OR⁹, SH, SR⁹, F, Cl, Br and I, wherein each R⁹, R^{9a} and R^{9b} is independently selected from alkyl, including wherein R^{9a} and R^{9b} form a heterocycle that includes the nitrogen to which they are attached or Cy may be iodine;
R¹⁰ and R¹¹ are independently selected from hydrogen and C₁-C₁₀ alkyl; C₁-C₁₀ acyl, and C₁-C₁₀ sulfonyl.
and pharmaceutically acceptable salts, stereoisomers and tautomers thereof.

In preferred embodiments X is O or S. In particularly preferred embodiments X is O.

In preferred embodiments

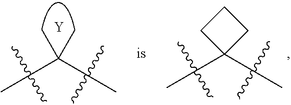

that is the ring Y is preferably cyclobutane. In further preferred embodiments the group bound to the phenyl ring in structure I is 1-aminocyclobutyl.

In preferred embodiments the ring Cy is unsubstituted C₆ aryl, that is phenyl.

In one particularly preferred embodiment of the compound of the invention the ring Cy is replaced with an iodine atom.

In another particularly preferred embodiment of the compound of the invention the phenyl ring shown in Formula (I) is replaced with a pyridine ring.

In preferred embodiments D, E, F and G together with the two carbon atoms of the 5 membered ring together form a moiety having the structure:

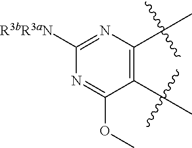

wherein R^{3a} and R^{3b} are as defined herein.

In the preferred embodiment described immediately above the preferred groups for R^{3a} and R^{3b} are either both hydrogen or are selected so as to give one of the following moieties:

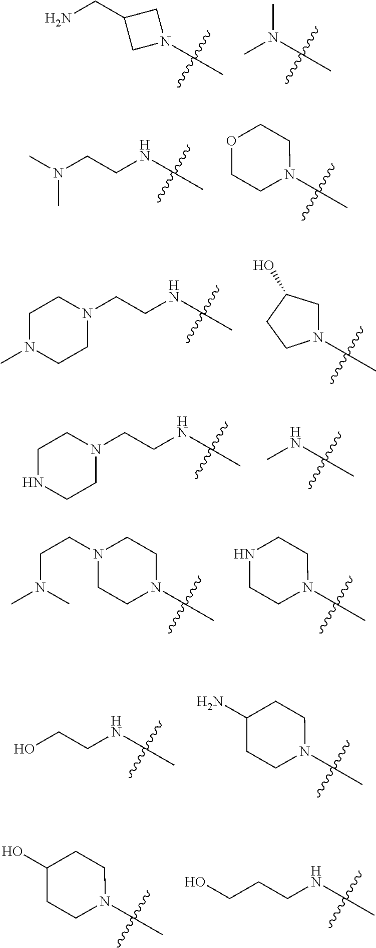

More preferred are the compounds described immediately above which have hydrogen as both R^{3a} and R^{3b} or have the following moieties:

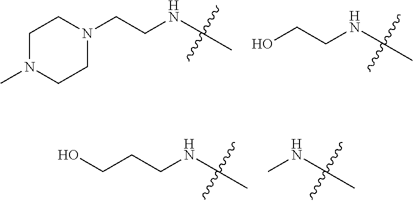

In one particularly preferred embodiment R^{3a} is hydrogen and R^{3b} is methyl.

In other preferred embodiments D, E, F and G together with the two carbon atoms on the 5 membered ring together form a moiety having either of the following structures:

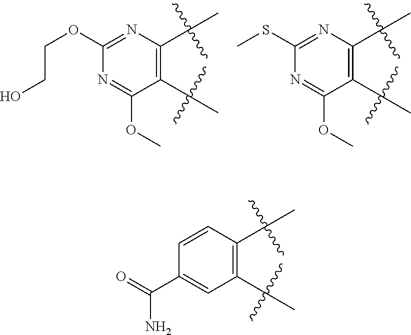

In some particularly preferred embodiments D, E, F and G together with the two carbon atoms on the 5 membered ring together form a moiety having either of the following structures:

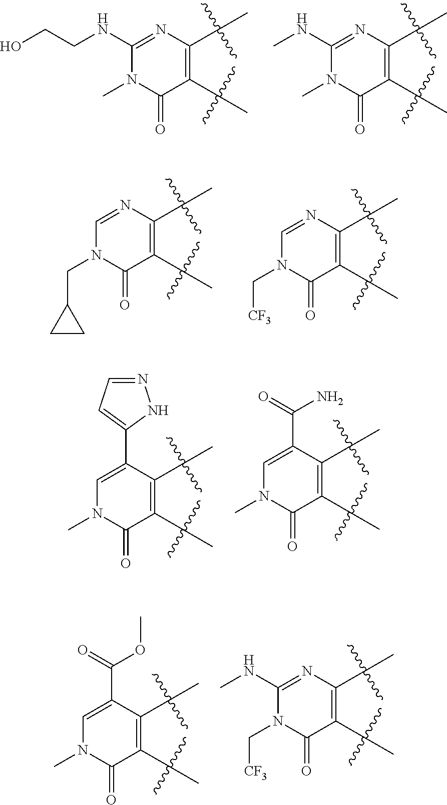

In some preferred embodiments D, E, F and G together with the two carbon atoms of the 5 membered ring together form a moiety having the structure:

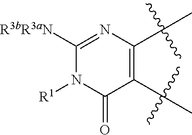

wherein R¹, R^{3a} and R^{3b} are as defined herein. In particularly preferred embodiments these compounds have R¹ as methyl or 2,2,2-trifluoroethyl.

In some preferred embodiments D, E, F and G together with the two carbon atoms of the 5 membered ring together form a moiety having the structure:

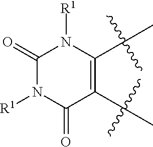

wherein each R¹ is independently as defined herein. In particularly preferred embodiments each R¹ is hydrogen or methyl, and in other preferred embodiments one is methyl and the other is hydrogen.

As can be seen from the two structures discussed immediately above, the ring formed by the grouped D, E, F, and G is not necessarily aromatic but may be partially or mostly saturated. Some unsaturated derivatives have been found to show good activity and these compounds are preferred, with or without combination with those other preferred structural motifs discussed herein.

Other preferred embodiments of the compounds according to the invention appear throughout the specification and in particular in the examples.

In a further aspect the present invention also relates to a compound of formula II:

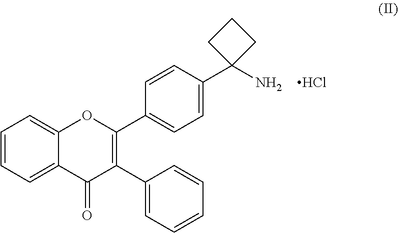

### DETAILED DESCRIPTION OF THE INVENTION

The term “alkyl group” refers to an aliphatic group containing at least carbon and hydrogen and containing 1 to 15 carbon atoms, such as 1 to 10 carbon atoms. Attachment to the alkyl group occurs through a carbon atom.

A “Cₙ alkyl” group refers to an aliphatic group containing n carbon atoms. For example, a C₁-C₁₀ alkyl group contains 1, 2, 3, 4, 5, 6, 7, 8, 9 or 10 carbon atoms.

An alkyl group may be straight chained or it may be branched.

An alkyl group may contain no ring structures or it may contain one or more rings.

For example, a “cycloalkyl” group contains at least one ring. It is understood that attachment to a cycloalkyl group is via a ring of the cycloalkyl group. Each ring may contain 3 to 10 atoms, such as 4 to 8 or 5 to 7 atoms. Each ring may be independently selected to contain just carbon atoms or to contain both carbon atoms and from 1 to 4 heteroatoms selected from O, N and S. For cyclo-heteroalkyl groups (i.e. cycloalkyl groups that contain one or more heteroatoms), attachment to the cycloalkyl group may occur either through a carbon atom or, if one or more heteroatoms are contained in a ring, attachment may also occur through a heteroatom contained in a ring.

For example, a cycloalkyl group may be mono-cyclic or bi-cyclic.

Thus, a “Cₙ cycloalkyl” group contains n carbon atoms. All n carbon atoms may be contained in the ring(s) of the cycloalkyl group or one or more of the carbons may not be contained in the ring(s) and may instead form one or more chains branching from the ring.

If a Cₙ alkyl group is joined to a separate Cₘ alkyl group containing m carbon atoms to form, for example, a heterocycle, the two alkyl groups contain a total number of m+n carbon atoms.

An alkyl group may be saturated or unsaturated. Thus, the alkyl group may be an alkenyl group (i.e. contain a carbon-carbon double bond) and/or an alkynyl group (i.e. contain a carbon-carbon triple bond). If the alkyl group is unsaturated, it may contain at least 2 carbon atoms. It is understood that any unsaturated portions of an alkyl group are non-aromatic (aromatic groups fall within the scope of the definition of “aryl”). Any part of the alkyl group may be unsaturated, for example the straight, branched or cyclic portion of an alkyl group may contain a carbon-carbon double bond or a carbon-carbon triple bond. Attachment to an unsaturated alky group may occur through the unsaturated part of the alkyl group or may occur through the unsaturated part of the group.

For example, an unsaturated alkyl group may contain 1 to 4 carbon-carbon double bonds or 1 to 3 carbon-carbon triple bonds or 1 to 4 of a combination of carbon-carbon double bonds and carbon-carbon triple bonds.

An alkyl group may be substituted with one or more heteroatoms or it may be unsubstituted (i.e. not contain any heteroatoms). If more than one hetero-substituent is present, the substituents are independently selected from one another unless they form a part of a particular functional group (e.g. an amide group).

The heteroatom substituents may in turn be substituted with further carbon-containing groups. In this case, the Cₙ or Cₘ prefix that defines the substituted alkyl group refers to the total number of carbons contained in the group, i.e. including the carbon atoms contained in any substituted heteroatomic groups, and the total alkyl group contains 1 to 15 carbon atoms as defined previously.

Accordingly, if the alkyl group is substituted, it may, for example, contain one or more of CN, CO₂H, CONH₂, CONHR, CONR^{a}R^{b}, CO₂R, NH₂, NHR, NR^{a}R^{b}, OH, OR, SH, SR, F, Cl, Br and I, wherein each R, R^{a} and R^{b} are independently selected groups (e.g. alkyl/aryl groups) attached to the atom to which the group joins through a carbon atom of each group, including wherein R^{a} and R^{b} form a heterocycle that includes the heteroatom to which they are attached. A group containing two Cₘ—Cₙ alkyl moieties that form a cycle that includes, for example, the heteroatom to which they are attached may contain from C₂ₘ to C₂ₙ carbon atoms.

Examples of unsubstituted saturated alkyl groups containing no cyclic structures include methyl, ethyl, n-propyl, sec-propyl, n-butyl, sec-butyl, tert-butyl, pentyl (branched or unbranched), hexyl (branched or unbranched), heptyl (branched or unbranched), octyl (branched or unbranched), nonyl (branched or unbranched), and decyl (branched or unbranched).

Examples of unsubstitued saturated cyclic alkyl groups include cyclopropyl, cylcobutyl, cyclopentyl and cyclohexyl.

Examples of unsaturated alkyl groups include ethenyl, propenyl, butenyl, 2-methylbutenyl and cyclohexenyl.

The term “aryl group” refers to a group containing at least one ring that is aromatic and containing 1 to 15 carbon atoms, such as 1 to 10 carbon atoms. Where an aryl group is stated as being substituted at a particular position, attachment of the position to the aryl group is onto the aromatic ring of the aryl group itself rather than the position being joined to the aryl group through any non-aromatic side-chain of the aryl group. For example, when R¹ is an aryl group in CR¹, the C is attached to the aromatic part of the aryl group.

Each ring of the aryl group has 3 to 10 atoms in the ring, such as 4 to 8 or 5 to 7 atoms. Each ring may be independently selected to contain only carbon atoms or to contain both carbon atoms and from 1 to 4 heteroatoms selected from O, N and S. For heteroaryl groups (i.e. aryl groups that contain one or more heteroatoms), attachment to the aryl group may occur either through a carbon atom or, if one or more heteroatoms are contained in a ring, attachment may also occur through a heteroatom contained in a ring.

It is noted that the heteroatoms contained in a ring of a heteroaryl group may be substituted, for example forming an N-oxide.

For example, the aromatic group may be mono-cyclic or bi-cyclic, wherein one or both of the rings of a bi-cyclic system is aromatic.

Examples of aryl groups include acridinyl, phenyl, carbazolyl, cinnolinyl, quinoxalinyl, pyrrazolyl, benzotriazolyl, furanyl, naphthyl, thienyl, thiazolyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline, benzimidazolyl and melaminyl.

It is noted that the term “heterocycle” includes within its scope both cycloalkyl groups containing one or more heteroatoms within the ring system and aryl groups containing one or more heteroatoms within the ring system.

The term “halo” refers to a group selected from chlorine, fluorine, bromine and iodine.

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the present invention provides the compound of the invention in which

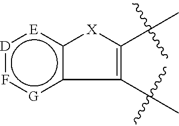

is selected from:

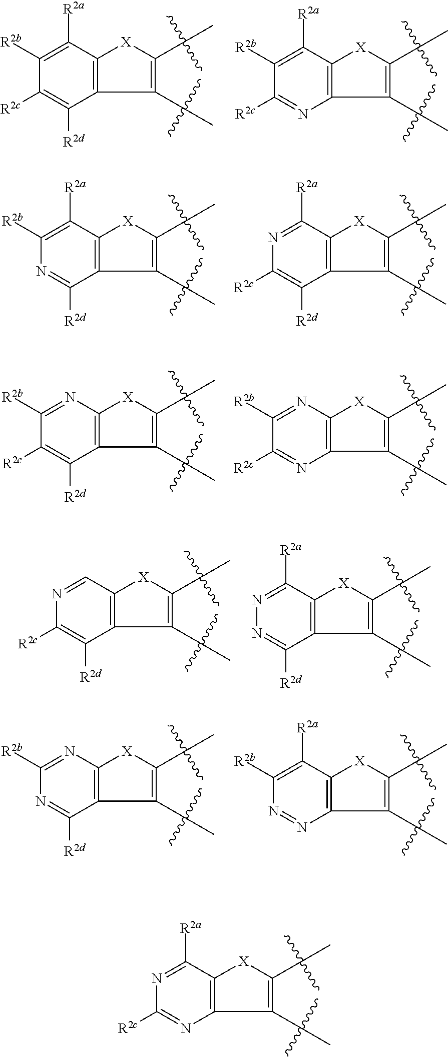

wherein R^{2a}, R^{2b}, R^{2c} and R^{2d} are independently selected from R² and H, where R² is defined herein.

For completeness, it is noted that separate R¹, R², R³, R⁴, R⁵, R⁶ and R⁷ groups may not be joined to one another unless they meet the conditions positively recited herein.

For completeness, it is also noted that certain chemical formulae used herein define delocalized systems. This definition is known in the art as a definition of aromaticity and may indicate the presence of, for example, a mono-, di- or tri-cyclic system that contains (4n+2) electrons where n is an integer. In other words, these systems may display Hückel aromaticity.

The first aspect of the invention may also provide a compound of the invention in which

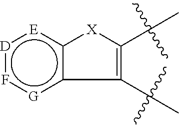

is selected from:

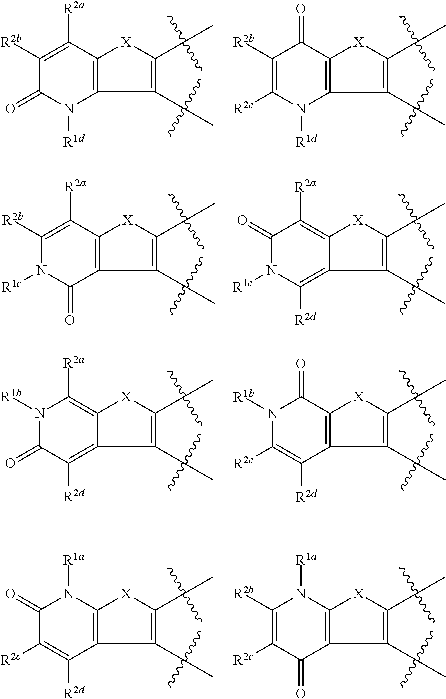

wherein R^{1a}, R^{1b}, R^{1c} and R^{1d} are independently selected from R¹ and H and R^{2a}, R^{2b}, R^{2c} and R^{2d} are independently selected from R² and H, where R¹ and R² are defined herein.

The first aspect of the invention may also provide a compound of the invention in which

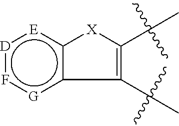

is selected from:

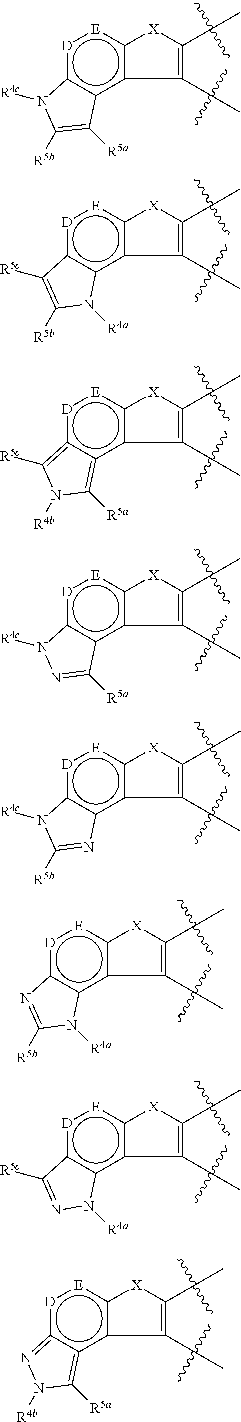

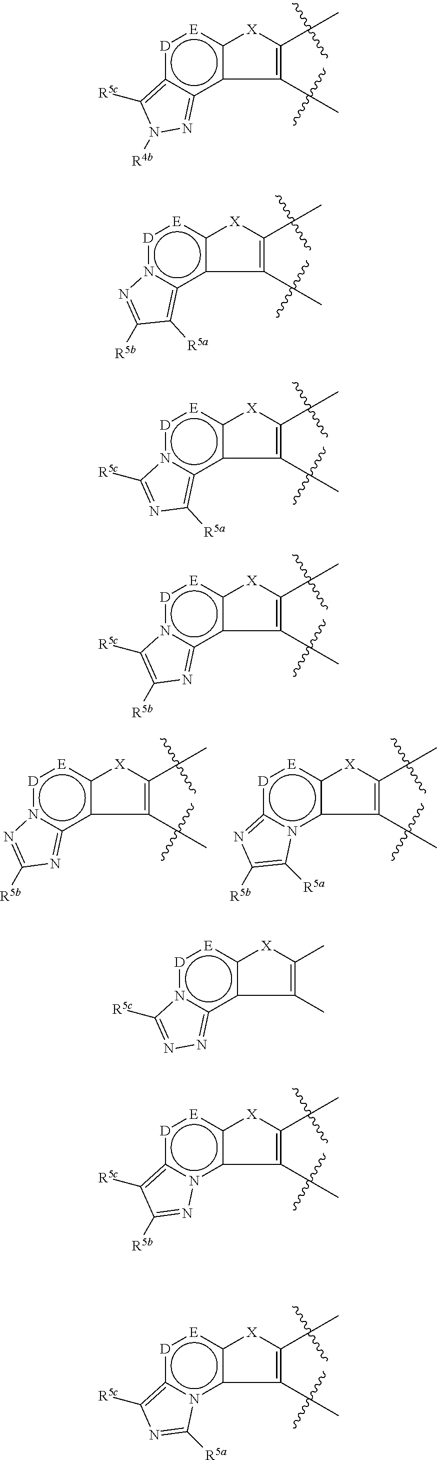

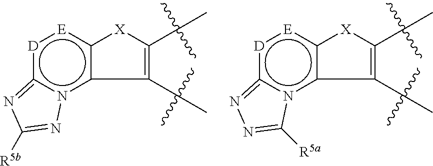

wherein R^{4a}, R^{4b} and R^{4c} are independently selected from R⁴ and H and R^{5a}, R^{5b} and R^{5c} are independently selected from R⁵ and H, wherein R⁴, R⁵, D, E and X are as defined herein.

The first aspect of the invention may also provide a compound of the invention in which

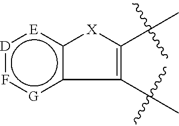

is selected from:

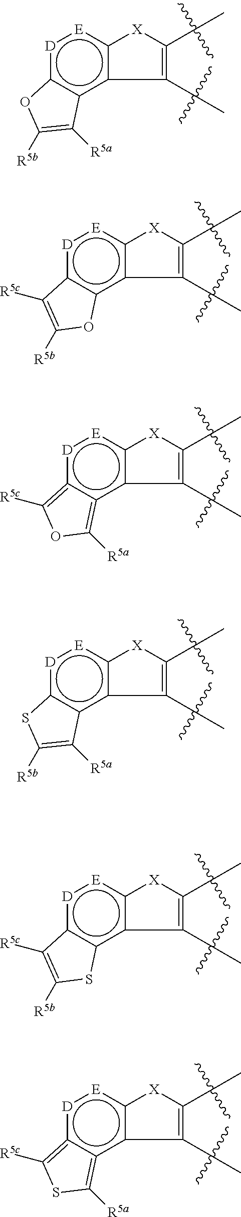

wherein R^{5a}, R^{5b} and R^{5c} are independently selected from R⁵ and H, wherein R⁵, D, E and X are as defined herein.

The first aspect of the invention may also provide a compound of the invention in which

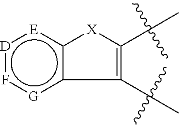

is selected from:

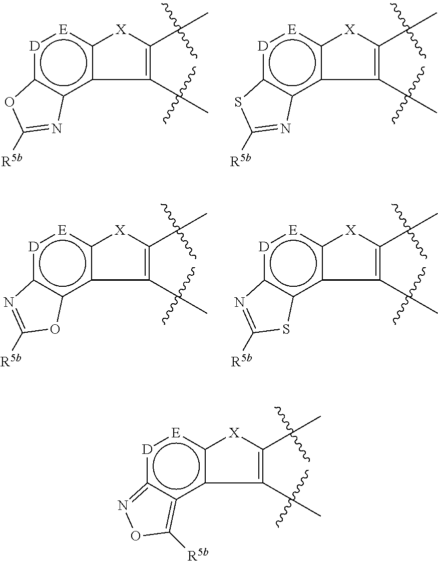

wherein R^{5b} is R⁵ or H, wherein R⁵, D, E and X are as defined herein.

The first aspect of the invention may also provide a compound of the invention in which

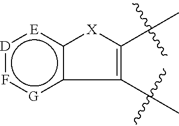

is selected from:

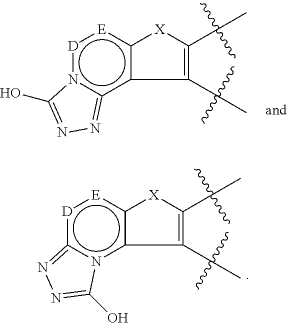

The first aspect of the invention may also provide a compound of the invention in which:

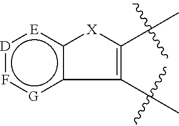

is selected from:

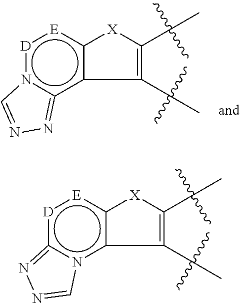

For example, X is O in the following preferred structure:

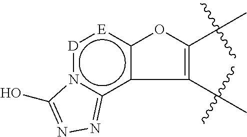

In one embodiment, D and E may be independently selected from N, CH and CR¹. For example, 0 or 1 of D and E may be selected to be N. For example, D and E may both be independently selected from CH and CR¹. For example, D and E may both be CH.

In one embodiment,

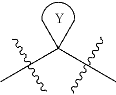

is chosen to be

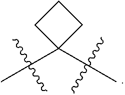

In one embodiment, ring Cy may be a six-membered aromatic ring. For example, Cy may be phenyl.

Accordingly, the first aspect may provide a compound having the formula:

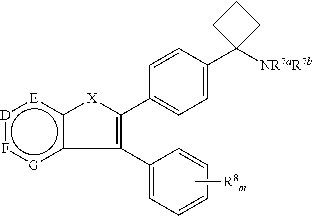

In one embodiment, m may be 0, 1 or 2. For example, m may be 0.

For example, Cy may be phenyl and m may be 0, 1 or 2, such as 0. Accordingly, the first aspect may provide a compound having the formula:

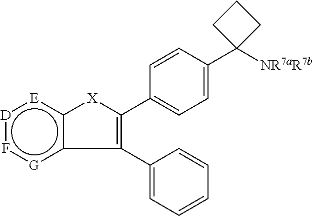

In a second aspect, R^{7a} and R^{7b} are independently chosen from a physiological hydrolyzable amide and H. For example, R^{7a} and R^{7b} may be chosen to both be H. Thus, for example, the present invention may provide a compound having the formula:

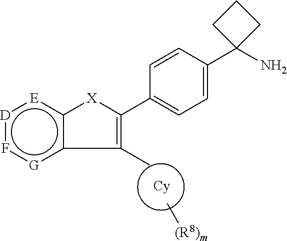

In a third aspect, the present invention provides a compound having a structure according to Formula as described herein selected from the group of compounds whose syntheses are described in the examples.

In a fourth aspect (which for the avoidance of doubt may be freely combined with Formula (I), the first aspect, the second aspect or the third aspect), the present invention provides a compound in which R¹ is selected from C1-C6 alkyl and H. For example, each R¹ may be chosen to be H.

The fourth aspect also provides a compound in which R² is selected from C2-C6 alkyl, aryl containing a five- or six-membered aromatic ring, CN, CHO, CONH₂, CHₐHal_{b}Y_{c} (wherein a is 0, 1, 2 or 3 and b is 3, 2, 1 or 0, each Hal is an independently selected halogen and c is 0 or 1 and Y is OH or NH₂ and a+b+c is 3), NH₂, NHR³, NR^{3a}R^{3b}, oxo, OH, OR³, F, Cl, Br and I, wherein each R^{3a} and R^{3b} is independently selected from C1-C6 alkyl, including wherein R^{3a} and R^{3b} join one another to form a heterocycle that includes the nitrogen to which they are attached.

The fourth aspect also provides a compound in which R⁴ is selected from H, C2-C6 alkyl CHₐHal_{b}Y_{c} (wherein a is 0, 1, 2 or 3 and b is 3, 2, 1 or 0, each Hal is an independently selected halogen and c is 0 or 1 and Y is OH or NH₂ and a+b+c is 3). For example, R⁴ may be H.

The fourth aspect also provides a compound in which R⁵ is selected from C2-C6 alkyl, CHₐHal_{b}Y_{c} (wherein a is 0, 1, 2 or 3 and b is 3, 2, 1 or 0, each Hal is an independently selected halogen and c is 0 or 1 and Y is OH or NH₂ and a+b+c is 3), OH, O(C1-C6 alkyl), F, Cl, Br and I.

The fourth aspect also provides for a C1 to Cn alkyl may to be chosen to be from a C2 to Cn alkyl. A C1 alkyl may also be chosen to be CN, CHO, CO₂H, CONH₂ or CHₐHal_{b}Y_{c} (wherein a is 0, 1, 2 or 3 and b is 3, 2, 1 or 0, each Hal is an independently selected halogen and c is 0 or 1 and Y is OH or NH₂ and a+b+c is 3). An example of a C1 alkyl is methyl.

For example, each R¹ may be independently selected from aryl, C2-C10 alkyl and CHₐHal_{b} (wherein a is 0, 1, 2 or 3 and b is 3, 2, 1 or 0, each Hal is an independently selected halogen and a+b is 3) and each R² is independently selected from aryl, C₂-C₁₀ alkyl, CN, CHO, CO₂H, CONH₂, CHₐHal_{b}Y_{c} (wherein a is 0, 1, 2 or 3 and b is 3, 2, 1 or 0, each Hal is an independently selected halogen and c is 0 or 1 and Y is OH or NH₂ and a+b+c is 3), NH₂, NHR³, NR^{3a}R^{3b}, oxo, OH, OR³, SH, SR³, SOR³, SO₂R³, SO₂NHR³, SO₂NR^{3a}R^{3b}, F, Cl, Br and I, wherein each R³, R^{3a} and R^{3b} is independently selected from C1-C10 alkyl, including wherein R^{3a} and R^{3b} are joined to one another to form a heterocycle that includes the nitrogen to which they are attached, and each R⁴ may be independently selected from aryl, C2-C10 alkyl and CHₐHal_{b} (wherein a is 0, 1, 2 or 3 and b is 3, 2, 1 or 0, each Hal is an independently selected halogen and a+b is 3) and each R⁵ is independently selected from aryl, C2-C10 alkyl, CN, CHO, CO₂H, CONH₂, oxo, CHₐHal_{b}Y_{c} (wherein a is 0, 1, 2 or 3 and b is 3, 2, 1 or 0, each Hal is an independently selected halogen and c is 0 or 1 and Y is OH or NH₂ and a+b+c is 3), NH₂, NHR⁶, NR^{6a}R^{6b}, OH, OR⁶, SH, SR⁶, SOR⁶, SO₂R⁶, SO₂NHR⁶, SO₂NR^{6a}R^{6b}, F, Cl, Br and I, wherein each R⁶, R^{6a} and R^{6b} is independently selected from C1-C10 alkyl, including wherein R^{6a} and R^{6b} are joined to one another to form a heterocycle that includes the nitrogen to which they are attached.

For example, an alkyl group may be substituted or unsubstituted. For example, the alkyl group may be methyl. The alkyl group may be substituted with one or more halogen atoms such as F, such as completely substituted with halogen atoms such as F (as in CF₃).

For example, the aryl group may contain a five- or six-membered heterocycle. For example, the aryl group may be a five-membered heterocycle containing 1 or 2 carbon atoms. Examples of suitable aryl groups include:

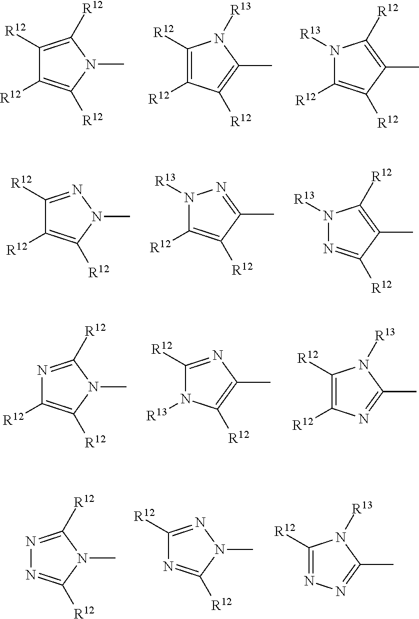

wherein each R¹² is independently selected from H or R² as defined herein and R¹³ is independently selected from H or R¹ as defined herein. For example, any or all of R¹² and R¹³ may be H. Other examples of suitable aryl groups include the oxygen and/or sulfur containing analogues of the pyrazoles, imidazoles and triazoles shown above, that is oxazoles, isoxazoles, thiazoles and isothiazoles and derivatives.

In whatever aspect, the compounds of the present invention may possess some aspect of stereochemistry. For example, the compounds may possess chiral centres and/or planes and/or axes. As such, the compounds may be provided as single stereoisomers, single diastereomers, mixtures of stereoisomers or as racemic mixtures. Stereoisomers are known in the art to be molecules that have the same molecular formula and sequence of bonded atoms, but which differ in their spatial orientations of their atoms and/or groups.

In addition, the compounds of the present invention may possess tautomerism. Each tautomeric form is intended to fall within the scope of the invention.

In addition, the compounds of the present invention may be provided as a pro-drug. Pro-drugs are transformed, generally in vivo, from one form to the active forms of the drugs described herein. For example, a prodrug may be formed by protecting the amine appending the cyclobutane as a physiological hydrolyzable amide. Alternatively or additionally, if D, E, F and/or G is NH, one or more of these may be protected as a physiological hydrolyzable amide.

In addition, the compounds of the present invention may be provided in the form of their pharmaceutically acceptable salts or as co-crystals. For example, the compounds may be provided having protonated amine groups.

The term “pharmaceutically acceptable salt” refers to ionic compounds formed by the addition of an acid to a base. The term refers to such salts that are considered in the art as being suitable for use in contact with a patient, for example in vivo and pharmaceutically acceptable salts are generally chosen for their non-toxic, non-irritant characteristics.

The term “co-crystal” refers to a multi-component molecular crystal, which may comprise non-ionic interactions.

Pharmaceutically acceptable salts and co-crystals may be prepared by ion exchange chromatography or by reacting the free base or acidic form of a compound with stoichiometric amounts or with an excess of the desired salt-forming inorganic or organic acid or base in one or more suitable solvents.

Salts known in the art to be generally suitable for use in contact with a patient include salts derived from inorganic and/or organic acids, including the hydrobromide, hydrochloride, sulphate, bisulphate, nitrate, acetate, oxalate, oleate, palmitate, stearate, laurate, benzoate, lactate, phosphate, tosylate, citrate, maleate, fumarate, succinate and tartrate. These may include cations based on the alkali and alkaline earth metals, such as sodium, potassium, calcium and magnesium, as well as ammonium, tetramethylammonium, tetraethylammonium. Further reference is made to the number of literature sources that survey suitable pharmaceutically acceptable salts, for example the Handbook of pharmaceutical salts published by IUPAC.

In addition, the compounds of the present invention may sometimes exist as zwitterions, which are considered as part of the invention.

The compounds of the present invention are useful in the treatment of medical conditions associated with disordered cell growth, including, but not restricted to, cancer, in particular cancers associated with overactivity of AKT occurring either from a direct change within the kinase itself such as may occur following a mutation within any of its subunits or from increased upstream activity including but not restricted to increased PI3K or PDK activity. Increased PI3K activity may have occurred through loss of the tumor suppressor PTEN.

For example, cancers include cardiac cancers, lung cancers, gastrointestinal cancers, genitourinary tract cancers, liver cancers, bone cancers, nervous system cancers, gynecological cancers, hematologic cancers, skin cancers and adrenal gland cancers.

For example, cancers include adrenal tumors, bile duct, bladder, blood, bone and connective tissue, brain and central nervous system, breast, cervical, colon and rectal (colorectal), endometrial, esophageal, gallbladder, head and neck, Hodgkin's Lymphoma, hypopharangeal, kidney, laryngeal, leukemias, liver, lung, lymphoma, mediastinal tumors, melanoma (malignant melanoma), mesothelioma, multiple myeloma, nasal cavity, nasopharyngeal, neuroendocrine tumors, non-Hodgkin's lymphoma, oral, oesophagus, oropharyngeal, ovarian, pancreas, paranasal sinus, parathyroid, penis, pituitary tumors, prostate, salivary gland, sarcoma, skin, spine, stomach, testicular, thyroid, urethra, uterine, vaginal and vulvar.

The compounds of the present invention are also useful in preparing a medicament that is useful in treating the diseases described above, in particular cancer.

The compounds of the present invention may selectively inhibit one or two of the AKT protein family over the other AKT isoform(s). For example, the compounds may selectively inhibit one or two of AKT1, AKT2 or AKT3 over the other isoform(s) of AKT.

For example, the compounds of the present invention may inhibit at least AKT1 and/or AKT2. For example, the compounds may selectively inhibit AKT1 and/or AKT2 over AKT3.

The present invention is further directed to a method of inhibiting AKT activity which comprises administering to a mammal in need thereof a pharmaceutically effective amount of the compound of the present invention.

The compounds of this invention may be administered to mammals, including humans, either alone or, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The present invention also includes within its scope the use of the compounds of the present invention in combination with a second drug in the treatment of cancer. The second drug may be a drug that is already known in the art in the treatment of cancer. The present invention also includes the use of the compounds of the invention in a regime including the step of radiotherapy

In particular, cancers often become resistant to therapy. The development of resistance may be delayed or overcome by the administration of a combination of drugs that includes the compounds of the present invention.

For example, drugs that may be used in combination with the compounds of the present invention may target the same or a similar biological pathway to that targeted by the compounds of the present invention or may act on a different or unrelated pathway.

Depending on the disease to be treated, a variety of combination partners may be coadministered with the compounds of the present invention. The second active ingredient may include, but is not restricted to: alkylating agents, including cyclophosphamide, ifosfamide, thiotepa, melphalan, chloroethylnitrosourea and bendamustine; platinum derivatives, including cisplatin, oxaliplatin, carboplatin and satraplatin; antimitotic agents, including vinca alkaloids (vincristine, vinorelbine and vinblastine), taxanes (paclitaxel, docetaxel), epothilones and inhibitors of mitotic kinases including aurora and polo kinases; topoisomerase inhibitors, including anthracyclines, epipodophyllotoxins, camptothecin and analogues of camptothecin; antimetabolites, including 5-fluorouracil, capecitabine, cytarabine, gemcitabine, 6-mercaptopurine, 6-thioguanine, fludarabine, methotrexate and premetrexed; protein kinase inhibitors, including imatinib, gefitinib, sorafenib, sunitinib, erlotinib, dasatinib, and lapatinib; proteosome inhibitors, including bortezomib; histone deacetylase inhibitors, including valproate and SAHA; antiangiogenic drugs, including bevacizumab; monoclonal antibodies, including trastuzumab, rituximab, alemtuzumab, tositumomab, cetuximab, panitumumab; conjugates of myoclonal antibodies, including Gemtuzumab ozogamicin, Ibritumomab tiuxetan; hormonal therapies, including antiestrogens (tamoxifen, raloxifen, anastrazole, letrozole, examestane) antiandrogens (Flutamide, Biclutamide) and Luteinisng Hormone Analogues or antagonists.

With regard to combination therapy the compounds of the present invention may be administered separately, sequentially, simultaneously, concurrently or may be chronologically staggered with one or more standard therapeutics such as any of those mentioned above.

The present invention also provides a pharmaceutical composition suitable for clinical use.

In particular, a pharmaceutical composition may comprise a pharmaceutical carrier and, dispersed therein, a therapeutically effective amount of the compounds of the invention. The composition may be solid or liquid. The pharmaceutical carrier is generally chosen based on the type of administration being used and the pharmaceutical carrier may for example be solid or liquid. The compounds of the invention may be in the same phase or in a different phase than the pharmaceutical carrier.

Pharmaceutical compositions may be formulated according to their particular use and purpose by mixing, for example, excipient, binding agent, lubricant, disintegrating agent, coating material, emulsifier, suspending agent, solvent, stabilizer, absorption enhancer and/or ointment base. The composition may be suitable for oral, injectable, rectal or topical administration.

For example, the pharmaceutical composition may be administered orally, such as in the form of tablets, coated tablets, hard or soft gelatine capsules, solutions, emulsions, or suspensions. Administration can also be carried out rectally, for example using suppositories, locally or percutaneously, for example using ointments, creams, gels or solution, or paenterally, for example using injectable solutions.

For the preparation of tablets, coated tablets or hard gelatine capsules, the compounds of the present invention may be admixed with pharmaceutically inert, inorganic or organic excipients. Examples of suitable excipients include lactose, mize starch or derivatives thereof, talc or stearic acid or salts thereof. Suitable excipients for use with soft gelatine capsules include, for example, vegetable oils, waxes, fats and semi-solid or liquid polyols.

For the preparation of solutions and syrups, excipients include, for example, water, polyols, saccharose, invert sugar and glucose.

For injectable solutions, excipients include, for example, water, alcohols, polyols, glycerine and vegetable oil.

For suppositories and for local and percutaneous application, excipients include, for example, natural or hardened oils, waxes, fats and semi-solid or liquid polyols.

The pharmaceutical compositions may also contain preserving agents, solublizing agents, stabilizing agents, wetting agents, emulsifiers, sweeteners, colorants, odorants, buffers, coating agents and/or antioxidants.

For combination therapies, the second drug may be provided in pharmaceutical composition with the present invention or may be provided separately.

Thus, a pharmaceutical formulation for oral administration may, for example, be granule, tablet, sugar coated tablet, capsule, pill, suspension or emulsion. For parenteral injection for, for example, intravenous, intramuscular or subcutaneous use, a sterile aqueous solution may be provided that may contain other substances including, for example, salts and/or glucose to make to solution isotonic. The anti-cancer agent may also be administered in the form of a suppository or pessary, or may be applied topically in the form of a lotion, solution, cream, ointment or dusting powder.

The present invention also relates to methods of treating or preventing cancer comprising administering a compound according to the present invention as described herein to a subject in need thereof.

### EXAMPLES

The present invention will now be described in relation to several examples.

Examples 1 to 153 were synthesised according to the methods described subsequently. Their IC₅₀ values were then determined as described below and are represented in the following table, in which the compound numbers correspond to the numbers in the examples.

| Example | IC50 in IMAP | IC50 in IMAP | IC50 in IMAP |
|---|---|---|---|
| Number | assay vs AKT1 | assay vs AKT2 | assay vs AKT3 |
| 1 | ++ | ++ | ++ |
| 2 | + | ++ | + |
| 3 | ++ | ++ | ++ |
| 4 | +++ | ++ | + |
| 5 | ++ | ++ | + |
| 6 | ++ | ++ | + |
| 7 | +++ | +++ | + |
| 8 | ++ | ++ | + |
| 9 | ++ | ++ | + |
| 10 | ++ | ++ | + |
| 11 | + | + | + |
| 12 | ++ | ++ | ++ |
| 13 | ++ | +++ | ++ |
| 14 | +++ | +++ | ++ |
| 15 | +++ | +++ | ++ |
| 16 | ++ | +++ | ++ |
| 17 | ++ | ++ | + |
| 18 | +++ | ++ | + |
| 19 | + | + | + |
| 20 | ++ | ++ | + |
| 21 | ++ | ++ | + |
| 22 | ++ | ++ | + |
| 23 | + | + | + |
| 24 | ++ | +++ | + |
| 25 | ++ | ++ | + |
| 26 | +++ | ++ | + |
| 27 | +++ | +++ | ++ |
| 28 | + | ++ | + |
| 29 | ++ | ++ | + |
| 30 | +++ | + | + |
| 31 | ++++ | +++ | + |
| 32 | ++++ | +++ | + |
| 33 | +++ | +++ | ++ |
| 34 | ++++ | +++ | ++ |
| 35 | +++ | +++ | ++ |
| 36 | ++++ | +++ | + |
| 37 | +++ | +++ | ++ |
| 38 | ++++ | +++ | + |
| 39 | +++ | +++ | ++ |
| 40 | +++ | +++ | + |
| 41 | ++++ | +++ | + |
| 42 | +++ | +++ | + |
| 43 | +++ | +++ | ++ |
| 44 | ++++ | +++ | + |
| 45 | +++ | +++ | ++ |
| 46 | +++ | +++ | +++ |
| 47 | ++ | ++ | + |
| 48 | ++++ | +++ | +++ |
| 49 | ++++ | +++ | + |
| 50 | ++ | +++ | + |
| 51 | +++ | +++ | + |
| 52 | ++ | +++ | + |
| 53 | +++ | +++ | + |
| 54 | ++++ | +++ | + |
| 55 | ++++ | +++ | + |
| 56 | +++ | ++++ | ++ |
| 57 | +++ | ++++ | ++ |
| 58 | +++ | +++ | ++ |
| 59 | +++ | ++++ | ++ |
| 60 | ++ | +++ | + |
| 61 | +++ | +++ | ++ |
| 62 | +++ | +++ | ++ |
| 63 | +++ | +++ | + |
| 64 | ++++ | +++ | ++ |
| 65 | +++ | +++ | ++ |
| 66 | +++ | +++ | + |
| 67 | +++ | ++++ | +++ |
| 68 | +++ | +++ | +++ |
| 69 | ++ | +++ | + |
| 70 | ++ | ++ | + |
| 71 | ++ | ++ | + |
| 72 | +++ | +++ | ++ |
| 73 | +++ | +++ | + |
| 74 | ++ | ++ | + |
| 75 | ++ | ++ | + |
| 76 | ++++ | ++++ | ++ |
| 77 | +++ | +++ | + |
| 78 | ++++ | +++ | + |
| 79 | +++ | ++ | + |
| 80 | +++ | ++ | + |
| 81 | +++ | ++ | + |
| 82 | +++ | ++ | ++ |
| 83 | +++ | ++ | ++ |
| 84 | ++ | +++ | + |
| 85 | +++ | ++++ | ++ |
| 86 | +++ | +++ | ++ |
| 87 | +++ | +++ | ++ |
| 88 | +++ | ++++ | ++ |
| 89 | +++ | +++ | ++ |
| 90 | +++ | +++ | + |
| 91 | ++ | ++ | + |
| 92 | +++ | ++++ | ++ |
| 93 | ++ | ++++ | ++ |
| 94 | ++ | ++++ | ++ |
| 95 | ++ | ++++ | ++ |
| 96 | ++++ | ++++ | ++ |
| 97 | +++ | +++ | + |
| 98 | +++ | +++ | + |
| 99 | ++ | ++++ | ++ |
| 100 | ++ | ++++ | ++ |
| 101 | +++ | +++ | ++ |
| 102 | ++ | +++ | ++ |
| 103 | +++ | ++++ | ++ |
| 104 | ++ | ++++ | ++ |
| 105 | ++ | +++ | ++ |
| 106 | ++ | ++++ | ++ |
| 107 | ++ | +++ | ++ |
| 108 | ++ | ++++ | ++ |
| 109 | ++ | ++++ | + |
| 110 | ++ | ++++ | ++ |
| 111 | ++++ | +++ | ++ |
| 112 | +++ | ++++ | + |
| 113 | +++ | +++ | ++ |
| 114 | +++ | +++ | ++ |
| 115 | +++ | ++ | + |
| 116 | ++++ | +++ | + |
| 117 | +++ | +++ | ++ |
| 118 | ++++ | +++ | ++ |
| 119 | +++ | +++ | + |
| 120 | +++ | +++ | + |
| 121 | +++ | ++++ | ++ |
| 122 | +++ | ++++ | ++ |
| 123 | +++ | ++++ | ++ |
| 124 | +++ | +++ | + |
| 125 | +++ | ++ | + |
| 126 | +++ | +++ | + |
| 127 | + | ++ | + |
| 128 | +++ | ++ | ++ |
| 129 | +++ | +++ | ++ |
| 130 | ++ | +++ | + |
| 131 | ++ | +++ | ++ |
| 132 | +++ | +++ | + |
| 133 | ++ | ++ | + |
| 134 | +++ | ++ | + |
| 135 | +++ | +++ | +++ |
| 136 | ++ | ++ | + |
| 137 | +++ | ++ | + |
| 138 | +++ | +++ | ++ |
| 139 | ++ | ++ | ++ |
| 140 | ++ | +++ | + |
| 141 | ++ | ++ | + |
| 142 | +++ | +++ | ++ |
| 143 | ++ | ++ | + |
| 144 | ++ | ++ | + |
| 145 | ++ | ++ | + |
| 146 | ++ | ++ | + |
| 147 | ++ | ++ | + |
| 148 | +++ | ++ | + |
| 149 | +++ | ++ | + |
| 150 | ++ | +++ | + |
| 151 | ++ | ++ | + |
| 152 | +++ | ++ | + |
| 153 | +++ | +++ | + |

Key
+ IC50 > 10 μM
++ 1 μM < IC50 ≦ 10 μM
+++ 0.1 μM < IC50 ≦ 1 μM
++++ IC50 ≦ 0.1 μM

In addition, the phosphorylation status of various members of the AKT/PI3K pathway was investigated via western blotting. For example, 15 shows inhibition of AKT phosphorylation for 48 h at 10 μM.

It can be seen from the Table that several Examples exhibit selectivity for one or more AKT isoforms over the other isoform(s). For example, a greater activity for AKT1 and/or AKT2 is observed compared to AKT3.

In addition, the activity of compounds in in vitro cell proliferation assays was investigated. Representative examples (eg. 1, 10, 12, 24, 32, 36 and 37) show inhibition of proliferation of PC3 and/or LnCaP cell lines with an IC50<10 μM.

Abbreviations

AcOH: Acetic acid; nBuLi: n-Butyllithium; BINAP: 2,2′-bis(diphenylphosphino)-1,1′-binaphthyl; DBU: 1,8-Diazabicyclo[5.4.0]undec-7-ene; DCM: Dichloromethane; DIPEA: Diisopropylethylamine; DMA: N,N-Dimethyl acetamide; DMAP: 4-Dimethylaminopyridine; DME: Dimethoxy ethane; DMF: N,N-Dimethylformamide; DMSO: Dimethylsulfoxide; EDCl: 1-Ethyl-3-(3′-dimethylaminopropyl)carbodiimide; EtOAc: Ethyl acetate; h: Hour: HATU: O-(7-Azabenzotriazol-1-yl)-N,N,N′,N′-tetramethyluronium hexafluorophosphate; HCl: Hydrochloric acid; HOBt: 1-Hydroxybenzotriazole; HPLC: High Pressure Liquid Chromatography; IMS: Industrial methylated spirits: M: Molar; MeOH: Methanol; NMP: N-Methyl-2-pyrrolidone; NMR: Nuclear Magnetic Resonance; Min: Minutes; RT: Room temperature; SCX: SCX-strong cation exchange; TBAF: Tetra-n-butylammonium fluoride; TEA: Triethylamine; TFA: Trifluoroacetic acid; THF: Tetrahydrofuran; TMSCl: Trimethylsilyl chloride.

General Experimental Conditions

¹H NMR spectra were recorded at ambient temperature using a Varian Unity Inova (400 MHz) spectrometer with a triple resonance 5 mm probe, a Bruker Avance DRX (400 MHz) with a 5 mm inverse detection triple resonance TXI probe, a Bruker Avance (500 MHz) spectrometer with a 5 mm QNP probe or a Bruker Avance DPX (300 MHz) spectrometer with a standard 5 mm dual frequency probe. Chemical shifts are expressed in ppm relative to tetramethylsilane.

High Pressure Liquid Chromatography—Mass Spectrometry (LCMS) experiments to determine retention times (R_{T}) and associated mass ions were performed using one of the following methods.

Method A: The system consists of ThermoFinnigan LCQ Advantage Mass Spectrometer with the Surveyor LC system and 200 position autosampler. The spectrometer has an electrospray source operating in positive and negative ion mode. An LCMS experiment is performed on each sample submitted using the following conditions: LC Column—Luna 3 micron C₁₈ 50×2 mm; Mobile phase—A) Water 0.1% Formic Acid B) Acetonitrile 0.1% Formic Acid

| Gradient - Time | flow | % A | % B |
|---|---|---|---|
| 0.00 | 0.6 | 95 | 5 |
| 7.00 | 0.6 | 5 | 95 |
| 8.00 | 0.6 | 5 | 95 |
| 8.20 | 0.6 | 95 | 5 |
| 11.00 | 0.6 | 95 | 5 |

Split—100 μl/min split to the ESI source with inline Surveyor DAD detection.

### Detection—MS, UV

MS ionisation method—Electrospray (positive and negative ion)
Total experiment time—11 mins (approx)

Method B: The system consists of a Waters ZMD quadrupole mass spectrometer linked to a Waters 1525 LC system with Waters 996 diode array detector. Sample injection is done by a Waters 2700 autosampler. The spectrometer has an electrospray source operating in positive and negative ion mode. Additional detection is achieved using a Sedex 85 evaporative light scattering detector.

An LCMS experiment is performed on each sample submitted using the following conditions: LC Column—Luna 3 micron C18(2) 30×4.6 mm or equivalent. Mobile phase—A) Water 0.1% Formic Acid B) Methanol 0.1% Formic Acid

| Gradient - Time | flow | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Split—200 μl/min split to the ESI source with inline Waters 996 DAD detection.

### Detection—MS, ELS, UV

MS ionisation method—Electrospray (positive and negative ion)
Total experiment time—6 mins (approx)

Method C: The system consists of a Waters Micromass ZQ2000 quadrupole mass spectrometer linked to a Waters Acquity UPLC system with a PDA UV detector. The spectrometer has an electrospray source operating in positive and negative ion mode. An LCMS experiment is performed on each sample submitted using the following conditions: LC Column—Acquity BEH C18 1.7 um 100×2.1 mm, maintained at 40° C. or Acquity BEH Shield RP18 1.7 um 100×2.1 mm, maintained at 40° C. Mobile phase—A) Water 0.1° A) Formic Acid B) Acetonitrile 0.1% Formic Acid

| Gradient - Time | flow ml/min | % A | % B |
|---|---|---|---|
| 0.00 | 0.4 | 95 | 5 |
| 0.40 | 0.4 | 95 | 5 |
| 6.00 | 0.4 | 5 | 95 |
| 6.80 | 0.4 | 5 | 95 |
| 7.00 | 0.4 | 95 | 5 |
| 8.00 | 0.4 | 95 | 5 |

### Detection—MS, UV PDA

MS ionisation method—Electrospray (positive/negative ion)

Method D: The system consists of an Agilent Technologies 6140 single quadrupole mass spectrometer linked to an Agilent Technologies 1290 Infinity LC system with UV diode array detector and autosampler. The spectrometer has a multimode ionization source (electrospray and atmospheric pressure chemical ionizations) operating in positive and negative ion mode. An LCMS experiment is performed on each sample submitted using the following conditions: LC Column—Zorbax Eclipse Plus C18 RRHD 1.8 micron 50×2.1 mm maintained at 40° C. Mobile phase—A) Water 0.1% Formic Acid B) Acetonitrile 0.1% Formic Acid.

| Gradient - Time | Flow mL/min | % A | % B |
|---|---|---|---|
| 0.00 | 0.6 | 95 | 5 |
| 7.00 | 0.6 | 0 | 95 |
| 8.00 | 0.6 | 0 | 95 |
| 8.10 | 0.6 | 95 | 5 |
| 10.0 | 0.6 | 95 | 5 |

### Detection—MS, UV

MS ionisation method—Multimode (positive and negative ion)
Total experiment time—10 mins (approx)

Method E: The system consists of an Agilent Technologies 6140 single quadrupole mass spectrometer linked to an Agilent Technologies 1290 Infinity LC system with UV diode array detector and autosampler. The spectrometer has a multimode ionization source (electrospray and atmospheric pressure chemical ionizations) operating in positive and negative ion mode. An LCMS experiment is performed on each sample submitted using the following conditions: LC Column—Zorbax Eclipse Plus C18 RRHD 1.8 micron 50×2.1 mm maintained at 40° C. Mobile phase—A) Water 0.1% Formic Acid B) Acetonitrile 0.1% Formic Acid.

| Gradient - Time | Flow mL/min | % A | % B |
|---|---|---|---|
| 0.00 | 1.0 | 95 | 5 |
| 1.80 | 1.0 | 0 | 100 |
| 2.20 | 1.0 | 0 | 100 |
| 2.21 | 1.0 | 95 | 5 |
| 2.50 | 1.0 | 95 | 5 |

### Detection—MS, UV

MS ionisation method—Multimode (positive and negative ion)
Total experiment time—2.50 mins (approx)

Method F: The system consists of a Agilent Technologies 6120 single quadrupole mass spectrometer linked to a Agilent Technologies 1200 Preparative LC system with Multiple Wavelength detector and autosampler. The spectrometer has a multimode ionization source (electrospray and atmospheric pressure chemical ionizations) operating in positive and negative ion mode. Fraction collection is mass-triggered. A purification experiment is performed on each sample submitted using the following conditions: LC Column—Waters XBridge™ Prep C18 5 μm OBD™ 19×50 mm column at room temperature. Mobile phase—A) Water 0.1% ammonium hydroxide B) Acetonitrile/Water 95: 5 with 0.1% ammonium hydroxide.

| Gradient - Time | Flow mL/min | % A | % B |
|---|---|---|---|
| 0.00 | 20.0 | 95 | 5 |
| 7.00 | 20.0 | 0 | 100 |
| 8.00 | 20.0 | 0 | 100 |
| 8.20 | 20.0 | 95 | 5 |

### Detection—MS, UV

MS ionisation method—Multimode (positive and negative ion)
Total experiment time—10 mins (approx)

Method G: The system consists of a Agilent Technologies 6120 single quadrupole mass spectrometer linked to a Agilent Technologies 1200 Preparative LC system with Multiple Wavelength detector and autosampler. The spectrometer has a multimode ionization source (electrospray and atmospheric pressure chemical ionizations) operating in positive and negative ion mode. Fraction collection is mass-triggered. A purification experiment is performed on each sample submitted using the following conditions: LC Column—Waters XBridge™ Prep C18 5 μm OBD™ 19×50 mm column at room temperature. Mobile phase—A) Water 0.1% ammonium hydroxide B) Acetonitrile/Water 95: 5 with 0.1% ammonium hydroxide.

| Gradient - Time | Flow mL/min | % A | % B |
|---|---|---|---|
| 0.00 | 20.0 | 88 | 12 |
| 3.00 | 20.0 | 57 | 43 |
| 5.00 | 20.0 | 57 | 43 |
| 7.00 | 20.0 | 0 | 100 |
| 8.0 | 20.0 | 0 | 100 |
| 8.20 | 20.0 | 88 | 12 |

### Detection—MS, UV

MS ionisation method—Multimode (positive and negative ion)
Total experiment time—10 mins (approx)

Method H: The system consists of a Agilent Technologies 6120 single quadrupole mass spectrometer linked to a Agilent Technologies 1200 Preparative LC system with Multiple Wavelength detector and autosampler. The spectrometer has a multimode ionization source (electrospray and atmospheric pressure chemical ionizations) operating in positive and negative ion mode. Fraction collection is mass-triggered. A purification experiment is performed on each sample submitted using the following conditions: LC Column—Waters XBridge™ Prep C18 5 μm OBD™ 19×50 mm column at room temperature. Mobile phase—A) Water 0.1% ammonium hydroxide B) Acetonitrile/Water 95: 5 with 0.1% ammonium hydroxide.

| Gradient - Time | Flow mL/min | % A | % B |
|---|---|---|---|
| 0.00 | 20.0 | 82 | 18 |
| 3.00 | 20.0 | 44 | 56 |
| 5.00 | 20.0 | 44 | 56 |
| 7.00 | 20.0 | 0 | 100 |
| 8.0 | 20.0 | 0 | 100 |
| 8.20 | 20.0 | 82 | 18 |

### Detection—MS, UV

MS ionisation method—Multimode (positive and negative ion)
Total experiment time—10 mins (approx)

Method I: The system consists of a Agilent Technologies 6120 single quadrupole mass spectrometer linked to a Agilent Technologies 1200 Preparative LC system with Multiple Wavelength detector and autosampler. The spectrometer has a multimode ionization source (electrospray and atmospheric pressure chemical ionizations) operating in positive and negative ion mode. Fraction collection is mass-triggered. A purification experiment is performed on each sample submitted using the following conditions: LC Column—Waters XBridge™ Prep C18 5 μm OBD™ 19×50 mm column at room temperature. Mobile phase—A) Water 0.1% ammonium hydroxide B) Acetonitrile/Water 95: 5 with 0.1% ammonium hydroxide.

| Gradient - Time | Flow mL/min | % A | % B |
|---|---|---|---|
| 0.00 | 20.0 | 68 | 32 |
| 3.00 | 20.0 | 31 | 69 |
| 5.00 | 20.0 | 31 | 69 |
| 7.00 | 20.0 | 0 | 100 |
| 8.0 | 20.0 | 0 | 100 |
| 8.20 | 20.0 | 68 | 32 |

### Detection—MS, UV

MS ionisation method—Multimode (positive and negative ion)
Total experiment time—10 mins (approx)

Method J: The system consists of a Agilent Technologies 6120 single quadrupole mass spectrometer linked to a Agilent Technologies 1200 Preparative LC system with Multiple Wavelength detector and autosampler. The spectrometer has a multimode ionization source (electrospray and atmospheric pressure chemical ionizations) operating in positive and negative ion mode. Fraction collection is mass-triggered. A purification experiment is performed on each sample submitted using the following conditions: LC Column—Waters XBridge™ Prep C18 5 μm OBD™ 30×100 mm column at room temperature. Mobile phase—A) Water 0.1% ammonium hydroxide B) Acetonitrile/Water 95: 5 with 0.1% ammonium hydroxide.

| Gradient - Time | Flow mL/min | % A | % B |
|---|---|---|---|
| 0.00 | 60.0 | 95 | 5 |
| 14.00 | 60.0 | 0 | 100 |
| 16.00 | 60.0 | 0 | 100 |
| 16.20 | 60.0 | 95 | 5 |

### Detection—MS, UV

MS ionisation method—Multimode (positive and negative ion)
Total experiment time—18 mins (approx)

Microwave experiments were carried out using CEM Discover™/Explorer24™ or Biotage Initator™ instruments. Temperatures from 60-300° C. can be achieved, and pressures of up to 20 bar can be reached.

Unless otherwise indicated, the nomenclature of structures was using “structure=name” from ChemBioDraw 11 (CambridgeSoft). Compound names marked with a superscript asterisk were named using AutoNom 2000 from MDL.

### Example 1

### 1-(4-(3-phenylfuro[3,2-c]pyridin-2-yl)phenyl)cyclobutanamine

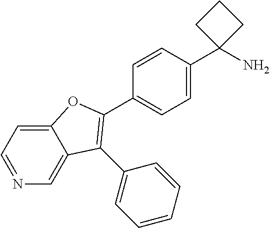

### Step 1: tert-Butyl 1-(4-ethynylphenyl)cyclobutylcarbamate

TMS-acetylene (36.2 ml, 254 mmol) was added to a pre-degassed (bubbling nitrogen) solution of tert-butyl 1-(4-bromophenyl)cyclobutylcarbamate (16.6 g, 50.9 mmol), bis(tri-tert-butylphosphine)palladium(0) (0.780 g, 1.53 mmol) and copper(I) iodide (0.194 g, 1.02 mmol) in 1,4-dioxane (42 ml)/diisopropylamine (42 ml, 295 mmol) at RT under an atmosphere of nitrogen. The temperature was increased to 80° C. After 20 hours, the reaction mixture was filtered through Celite®, washing with EtOAc (×3). The solvents were removed in vacuo to give the crude material of tert-butyl 1-(4-((trimethylsilyl)ethynyl)phenyl)cyclobutylcarbamatethe (ca. 17.5 g) that was dissolved in MeOH (85 ml) and potassium carbonate (8.44 g, 61.1 mmol) added. After 1 hour, analysis by LCMS showed complete conversion. The reaction mixture was filtered through Celite®, washing with EtOAc (×3) and the solvents were removed in vacuo. The remaining residue was purified by flash chromatography (SiO₂, 0-10%, EtOAc in cyclohexane) to give the title compound (13.5 g, 98%) as an orange oil that turned into a yellow solid on standing. ¹H NMR (500 MHz, CDCl₃): δ 7.49-7.44 (m, 2H), 7.38 (d, 2H), 5.08 (br s, 1H), 3.05 (s, 1H), 2.60-2.30 (m, 4H), 2.15-2.05 (m, 1H), 1.91-1.81 (m, 1H), 1.36 (br s, 9H).

### Step 2: tert-Butyl (1-(4-((4-methoxypyridin-3-yl)ethynyl)phenyl)cyclobutyl)carbamate

To a solution of 3-iodo-4-methoxypyridine (500 mg, 2.127 mmol) in anhydrous triethylamine (9 ml) was added bis(tert-butylphosphine)palladium(0) (65 mg, 0.128 mmol) and copper(I) iodide (12.2 mg, 0.064 mmol). The reaction mixture was degassed with N₂ for 10 min before being cooled to 0° C. A degassed solution of tert-butyl 1-(4-ethynylphenyl)cyclobutylcarbamate (635 mg, 2.34 mmol) in TEA (2 ml) was added dropwise to the cooled reaction mixture. The reaction was allowed to warm to RT and stirred for 18 h. The reaction mixture was concentrated in vacuo and the residue diluted with DCM (20 ml) and water (10 ml). The phases were separated and the organic layer dried (Na₂SO₄), filtered and concentrated affording a brown oil that was purified by silica gel chromatography (gradient 0 to 50% ethyl acetate in hexanes) yielding the title compound as a brown gum (140 mg, 17%). ¹H NMR (500 MHz, CDCl₃): δ 8.54 (br s, 1H), 8.38 (br s, 1H), 7.46 (d, 2H), 7.34 (d, 2H), 6.77 (d, 1H), 5.10 (s, 1H), 3.90 (s, 3H), 2.4-2.5 (m, 4H), 2.0-2.1 (m, 1H), 1.75-1.86 (m, 1H), 1.1-1.4 (br s, 9H).

Step 3: tert-Butyl (1-(4-(3-iodofuro[3,2-c]pyridin-2-yl)phenyl)cyclobutyl)carbamate: To a solution of tert-butyl 1-(4-((4-methoxypyridin-3-yl)ethynyl)phenyl)cyclobutylcarbamate (140 mg, 0.37 mmol) in dichloromethane (3 ml) was added iodine monochloride 1.0 M in DCM (0.740 ml, 0.740 mmol) dropwise at 0° C. under N₂. The reaction was allowed to warm to room temperature and stirred for 2 h before analysis by LCMS. The reaction was deemed not complete by LCMS, therefore a further 1 eq. ICI was added and stirring continued at RT for 2 h. On completion, the reaction was diluted with DCM (10 ml) and sat. Na₂S₂O₆ (5 ml) and extracted. The organic layer was concentrated in vacuo affording brown oil that was purified by silica gel chromatography (0 to 50% ethyl acetate in hexanes) yielding the title compound as a beige solid (95 mg, 52%). ¹H NMR (500 MHz, CDCl₃) δ 8.85 (br s, 1H), 8.65 (br s, 1H), 8.18 (d, 2H), 7.60 (d, 2H), 7.45 (s, 1H), 5.24 (s, 1H), 2.55-2.65 (m, 4H), 2.10-2.21 (m, 1H), 1.88-2.0 (m, 1H), 1.3-1.5 (br s, 9H).

Step 4: tert-Butyl (1-(4-(3-phenylfuro[3,2-c]pyridin-2-yl)phenyl)cyclobutyl)carbamate: To a solution of tert-butyl 1-(4-(3-iodofuro[3,2-c]pyridin-2-yl)phenyl)cyclobutylcarbamate (70 mg, 0.143 mmol) in dry 1,2-dimethoxyethane (3 ml) in a microwave vial was added phenylboronic acid (26 mg, 0.214 mmol), cesium fluoride (102 mg, 0.671 mmol) and triphenylphosphine (5.6 mg, 0.021 mmol). The mixture was degassed with N₂ for 10 min before the addition of palladium(II) acetete (1.6 mg, 7.14 μmol). The reaction was heated to 90° C. under microwave conditions for 30 mins. The reaction mixture was purified directly without workup by silica gel chromatography (gradient elution 0 to 50% ethyl acetate in hexanes) affording the title compound as a brown gum (40 mg, 64%). ¹H NMR (500 MHz, CDCl₃) 8.85 (br s, 1H), 8.54 (br s, 1H), 7.6 (d, 2H), 7.4 (d, 2H), 7.3 (s, 1H), 5.02 (s, 1H), 2.4-2.5 (m, 4H), 2.0-2.09 (m, 1H), 1.75-1.85 (m, 1H), 1.3-1.5 (br s, 9H).

Step 5: 1-(4-(3-Phenylfuro[3,2-c]pyridin-2-yl)phenyl)cyclobutanamine: To a solution of tert-butyl (1-(4-(3-phenylfuro[3,2-c]pyridin-2-yl)phenyl)cyclobutyl)carbamate (40 mg, 0.091 mmol) in DCM (3 ml) was added TFA (1 ml). The reaction was allowed to stir at RT for 30 min before concentration in vacuo. The residue was dissolved in DCM (10 ml) and sat. NaHCO₃ and the organic layer separated before purification by silica gel chromatography (gradient elution 0 to 10% MeOH in DCM). This yielded the title compound as a clear gum after drying by high vacuum (14 mg, 45%). LCMS (Method A): R_{T}=2.87 min, M+H⁺=341. ¹H NMR (500 MHz, CDCl₃) 8.77 (br s, 1H), 8.48 (br s, 1H), 7.6 (d, 2H), 7.35-7.49 (m, 6H), 7.3 (d, 2H), 2.4-2.5 (m, 2H), 2.05-2.13 (m, 2H), 1.95-2.04 (m, 1H), 1.7-2.0 (br s, 2H), 1.61-1.72 (m, 1H).

### Example 2

### 1-(4-(3-Phenylfuro[2,3-c]pyridin-2-yl)phenyl)cyclobutanamine

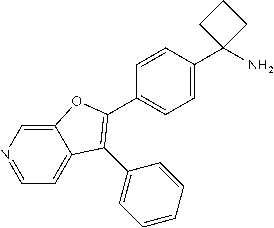

### Step 1: tert-Butyl (1-(4-((3-methoxypyridin-4-yl)ethynyl)phenyl)cyclobutyl)carbamate

Following the procedure of tert-butyl (1-(4-((4-methoxypyridin-3-yl)ethynyl)phenyl)cyclobutyl)carbamate, 4-bromo-3-methoxypyridine (0.2 g, 0.89 mmol) was reacted to afford the title compound (0.12 g, 36%). ¹H NMR (500 MHz, CDCl₃) 8.33 (s, 1H), 8.24 (d, 1H), 7.54 (d, 2H), 7.42 (d, 2H), 7.34 (d, 1H), 5.14 (s, 1H), 4.02 (s, 3H), 2.54-2.50 (m, 4H), 2.19-2.09 (m, 2H), 1.92-1.80 (m, 2H), 1.45-1.35 (br s, 9H).

### Step 2: tert-Butyl (1-(4-(3-iodofuro[2,3-c]pyridin-2-yl)phenyl)cyclobutyl)carbamate

Following the procedure of tert-butyl (1-(4-(3-iodofuro[3,2-c]pyridin-2-yl)phenyl)cyclobutyl)carbamate, tert-butyl (1-(4-((3-methoxypyridin-4-yl)ethynyl)phenyl)cyclobutyl)carbamate (0.12 g, 0.32 mmol) was reacted to afford the title compound (0.14 g, 90%). ¹H NMR (500 MHz, CDCl₃) 8.78 (s, 1H), 8.44 (d, 1H), 8.13 (dd, 2H), 7.52 (dd, 2H), 7.32 (d, 1H), 5.14 (s, 1H), 2.49-2.61 (m, 4H), 2.01-2.15 (m, 2H), 1.8-1.92 (m, 2H), 1.2-1.4 (br s, 9H).

### Step 3: tert-Butyl (1-(4-(3-phenylfuro[2,3-a]pyridin-2-yl)phenyl)cyclobutyl)carbamate

Following the procedure of tert-butyl (1-(4-(3-phenylfuro[3,2-c]pyridin-2-yl)phenyl)cyclobutyl)carbamate, tert-butyl (1-(4-(3-iodofuro[2,3-c]pyridin-2-yl)phenyl)cyclobutyl)carbamate (0.13 g, 0.27 mmol) was reacted to afford the title compound (91 mg, 78%). LCMS (Method A): R_{T}=5.69 min, M+H⁺=441.

### Step 4: 1-(4-(3-Phenylfuro[2,3-c]pyridin-2-yl)phenyl)cyclobutanamine

Following the procedure of 1-(4-(3-phenylfuro[3,2-c]pyridin-2-yl)phenyl)cyclobutanamine, tert-butyl (1-(4-(3-phenylfuro[2,3-c]pyridin-2-yl)phenyl)cyclobutyl)carbamate (25 mg, 0.06 mmol)) was reacted to afford the title compound (11 mg, 58%). LCMS (Method A): R_{T}=3.09 min, M+H⁺=341. ¹H NMR (500 MHz, CDCl₃) 8.66 (s, 1H), 8.35 (d, 1H), 7.61 (d, 2H), 7.3-7.45 (m, 8H), 2.45-2.52 (m, 2H), 2.07-2.15 (m, 2H), 1.88-2.05 (m, 3H), 1.65-1.75 (m, 1H).

### Example 3

### 1-(4-(3-Iodofuro[2,3-c]pyridin-2-yl)phenyl)cyclobutanamine

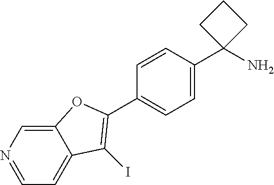

### Step 1: 1-(4-(3-Iodofuro[2,3-c]pyridin-2-yl)phenylcyclobutanamine

Following the procedure for 1-(4-(3-phenylfuro[3,2-c]pyridin-2-yl)phenyl)cyclobutanamine, tert-butyl (1-(4-(3-iodofuro[2,3-c]pyridin-2-yl)phenyl)cyclobutyl)carbamate (30 mg, 0.06 mmol) was reacted to afford the title compound (11.3 mg, 47%). LCMS (Method A): R_{T}=2.77 min, M+H⁺=390.9. ¹H NMR (500 MHz, CDCl₃) 8.79 (br s, 1H), 8.45 (br, s, 1H), 8.13 (d, 2H), 7.51 (d, 2H), 7.31 (d, 1H), 2.5-2.59 (m, 2H), 2.12-2.2 (m, 2H), 2.0-2.1 (m, 1H), 1.85-2.05 (br s, 2H), 1.7-1.8 (m, 1H).

### Example 4

### 2-(4-(1-Aminocyclobutyl)phenyl)-5-methyl-3-phenylfuro[3,2-c]pyridin-4(5H)-one

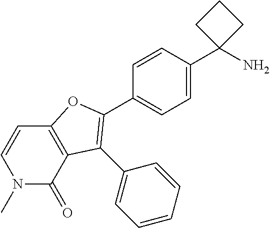

### Step 1: 2-(4-(1-Aminocyclobutyl)phenyl)-3-phenylfuro[3,2-q]pyridin-4(5H)-one: tert-Butyl

(1-(4-(4-methoxy-3-phenylfuro[3,2-c]pyridin-2-yl)phenyl)cyclobutyl)carbamate (500 mg, 1.06 mmol) was dissolved in 1,4-dioxane (15 ml) and 2N HCl (8 ml). The resultant solution was heated to 80° C. for 5 h before analysis by LCMS and TLC. The reaction was cooled to RT, neutralised with sat. Na₂CO₃ and extracted with DCM. The combined organic layers were then concentrated in vacuo affording a beige solid (250 mg, 66%). LCMS (Method A): R_{T}=3.46 min, M+H⁺=358.

### Step 2: tert-Butyl (1-(4-(4-oxo-3-phenyl-4,5-dihydrofuro[3,2-c]pyridin-2-yl)phenyl)cyclobutyl)carbamate

To a solution of 2-(4-(1-aminocyclobutyl)phenyl)-3-phenylfuro[3,2-c]pyridin-4(5H)-one (250 mg, 0.701 mmol) in dry THF (15 ml) was added di-tert-butyl dicarbonate (168 mg, 0.772 mmol) and DMAP (4 mg, 0.035 mmol). The reaction was the allowed to stir at RT under N₂ for 72 h. After this time, the reaction was concentrated, diluted with DCM (15 ml) and water (10 ml) and extracted. The organic extracts were concentrated and the residue triturated with Et₂O, filtered and dried affording tert-butyl 1-(4-(4-oxo-3-phenyl-4,5-dihydrofuro[3,2-c]pyridin-2-yl)phenyl)cyclobutylcarbamate (160 mg, 50%) as a beige solid. LCMS (Method A): R_{T}=6.6 min, M+H⁺=457.

### Step 3: tert-Butyl (1-(4-(5-methyl-4-oxo-3-phenyl-4,5-dihydrofuro[3,2-c]pyridin-2-yl)phenyl)cyclobutyl)carbamate

To a suspension of tert-butyl (1-(4-(4-oxo-3-phenyl-4,5-dihydrofuro[3,2-c]pyridin-2-yl)phenyl)cyclobutyl)carbamate (65 mg, 0.142 mmol) in DMF (2 ml) was added methyl iodide (10 μl, 0.16 mmol) and potassium carbonate (43 mg, 0.31 mmol). The reaction mixture was allowed to stir overnight at RT before analysis by TLC and LCMS. Reaction mixture was diluted with DCM (15 ml) and water (10 ml) and extracted. The DCM extracts were washed with brine (3×10 ml) and concentrated in vacuo followed by purification of the residue by silica gel chromatography (gradient 0 to 5% MeOH in DCM) affording the title compound as an off-white solid (45 mg, 67%). ¹H NMR (500 MHz, CDCl₃) 7.25-7.45 (m, 8H), 7.16 (d, 1H), 6.48 (d, 1H), 5.07 (s, 1H), 2.38-2.46 (m, 4H), 1.95-2.04 (m, 2H), 1.7-1.8 (m, 2H), 1.2-1.35 (m, 9H).

### Step 4: 2-(4-(1-Aminocyclobutyl)phenyl)-5-methyl-3-phenylfuro[3,2-c]pyridin-4(5H)-one

To a solution of tert-butyl (1-(4-(5-methyl-4-oxo-3-phenyl-4,5-dihydrofuro[3,2-c]pyridin-2-yl)phenyl)cyclobutyl)carbamate (45 mg, 0.096 mmol) in DCM (2 ml) was added TFA (1 ml). The reaction was allowed to stir at RT until deemed complete by TLC. The reaction was concentrated, diluted with DCM (15 ml) and sat. NaHCO₃ (10 ml) and the DCM extracts concentrated before purification by silica gel chromatography (0 to 6% MeOH in DCM). This afforded the title compound as an off-white solid (10 mg, 28%), LCMS (Method A): R_{T}=3.71 min, M+H⁺=372. ¹H NMR (500 MHz, CDCl₃) 7.4-7.46 (m, 4H), 7.28-7.38 (m, 3H), 7.22 (d, 2H), 7.18 (d, 1H), 6.49 (d, 1H), 3.48 (s, 3H), 2.4-2.49 (m, 2H), 2.04-2.12 (m, 2H), 1.95-2.03 (m, 1H), 1.8-1.95 (br s, 2H), 1.61-1.72 (m, 1H).

### Example 5

### 2-(4-(1-Aminocyclobutyl)phenyl)-3-phenylbenzofuran-6-carboxamide

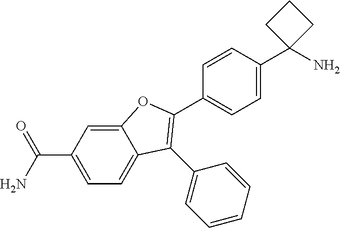

### Step 1: tert-Butyl (1-(4-(6-carbamoyl-3-phenylbenzofuran-2-yl)phenyl)cyclobutyl)carbamate

To a solution of tert-butyl (1-(4-(6-cyano-3-phenylbenzofuran-2-yl)phenyl)cyclobutyl)carbamate (50 mg, 0.107 mmol) in DMSO (0.125 mL) was added potassium carbonate (3 mg, 0.021 mmol). The reaction was heated at 40° C. and a 30% aqueous solution hydrogen peroxide (0.022 mL, 0.215 mmol) was added dropwise. The reaction mixture was heated at 70° C. for 2 hours. Upon total conversion the reaction mixture was cooled to 30° C., then water was slowly added (0.5 mL) and the reaction mixture was left to stir for 30 minutes. The residue was diluted with water (2 mL) and extracted with ethyl acetate (3×5 mL), concentrated in vacuo to yield a white solid (51.9 mg, 99%). ¹H NMR (500 MHz, CDCl₃) 7.98 (s, 1H), 7.62 (d, 1H), 7.57 (d, 2H), 7.45-7.31 (m, 8H), 5.1 (br s, 1H), 2.40-2.50 (m, 4H), 1.99-1.95 (m, 1H), 1.85-1.75 (m, 1H), 1.35-1.20 (m, 9H).

### Step 2: 2-(4-(1-Aminocyclobutyl)phenyl)-3-phenylbenzofuran-6-carboxamide

To a solution of tert-butyl(1-(4-(6-carbamoyl-3-phenylbenzofuran-2-yl)phenyl)cyclobutyl)carbamate carbamate (51.9 mg, 0.107 mmol) in DCM (3 mL) was added TFA (1 mL). The reaction mixture was stirred at room temperature for 1 hour then concentrated in vacuo and diluted in DCM (5 mL) and a saturated solution of NaHCO₃. A white precipitate formed that was filtered and dried in vacuo to afford the title compound (47 mg, 99%). LCMS (Method A): R_{T}=4.14 min, M+2H⁺=384. ¹H NMR (500 MHz, methanol-d₄) 8.15 (s, 1H), 7.83 (d, 1H), 7.78-7.72 (m, 2H), 7.55-7.41 (m, 8H), 2.82-2.76 (m, 2H), 2.68-2.59 (m, 2H), 2.30-2.21 (m, 1H), 2.04-1.96 (m, 1H).

### Example 6

### 2-(4-(1-Aminocyclobutyl)phenyl)-3-phenylbenzofuran-5-carbonitrile

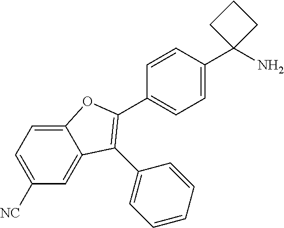

### Step 1: tert-Butyl (1-(4-((5-cyano-2-methoxyphenyl)ethynyl)phenyl)cyclobutyl)carbamate

Following the procedure for tert-butyl (1-(4-((4-cyano-2-methoxyphenyl)ethynyl)phenyl)cyclobutyl)carbamate, 3-bromo-4-methoxybenzonitrile (1.17 g, 5.52 mmol) was reacted to afford the title compound (1.14 g, 77%). ¹H NMR (500 MHz, CDCl₃) 7.80 (d, 1H), 7.62 (dd, 1H), 7.56 (dd, 2H), 7.44 (d, 2H), 6.98 (d, 1H), 5.10 (s, 1H), 4.00 (s, 3H), 2.57-2.45 (m, 4H), 2.20-2.10 (m, 1H), 1.95-1.85 (m, 1H), 1.45-1.35 (br s, 9H).

### Step 2: tert-Butyl (1-(4-(5-cyano-3-iodobenzofuran-2-yl)phenyl)cyclobutyl)carbamate

Following the procedure for tert-butyl (1-(4-(6-cyano-3-iodobenzofuran-2-yl)phenyl)cyclobutyl)carbamate, tert-butyl (1-(4-((5-cyano-2-methoxyphenyl)ethynyl)phenyl)cyclobutyl)carbamate (1.1 g, 2.73 mmol) was reacted to afford the title compound (0.91 g, 65%). ¹H NMR (500 MHz, CDCl₃) 8.19 (d, 2H), 7.82 (d, 1H), 7.66-7.59 (m, 4H), 5.19 (s, 1H), 2.65-2.55 (m, 4H), 2.21-2.16 (m, 1H), 1.98-1.92 (m, 1H), 1.50-1.30 (br s, 9H).

### Step 3: tert-Butyl (1-(4-(5-cyano-3-phenylbenzofuran-2-yl)phenyl)cyclobutyl)carbamate

A solution of tert-butyl (1-(4-(5-cyano-3-iodobenzofuran-2-yl)phenyl)cyclobutyl) carbamate (400 mg, 0.78 mmol) in anhydrous DME (10 ml) was degassed with N₂ for 10 min. Palladium(II)acetate (26 mg, 0.039 mmol), triphenylphosphine (31 mg, 0.117 mmol), phenyl boronic acid (142 mg, 1.17 mmol) and cesium fluoride (555 mg, 3.65 mmol) were added sequentially, and the reaction mixture was heated to 75° C. for 18 h. The reaction mixture was then concentrated in vacuo and the residue diluted with DCM (20 ml) and brine (10 ml). The phases were separated and the organic layer dried (MgSO₄), filtered and concentrated affording a brown oil that was purified by silica gel chromatography (gradient 0 to 20% ethyl acetate in hexanes) yielding the title compound as a pale orange solid (201 mg, 55%). ¹H NMR (500 MHz, CDCl₃) 7.83 (s, 1H), 7.67-7.61 (m, 4H), 7.54-7.47 (m, 5H), 7.42 (d, 2H), 5.09 (s, 1H), 2.56-2.45 (m, 4H), 2.2-2.1 (m, 1H), 1.85-1.95 (m, 1H), 1.5-1.3 (br s, 9H).

### Step 4: 2-(4-(1-Aminocyclobutyl)phenyl)-3-phenylbenzofuran-5-carbonitrile

Following the procedure for 2-(4-(1-aminocyclobutyl)phenyl)-5-methyl-3-phenylfuro[3,2-c]pyridin-4(5H)-one, tert-butyl (1-(4-(5-cyano-3-phenylbenzofuran-2-yl)phenyl)cyclobutyl)carbamate (50 mg, 0.107 mmol)) was reacted to afford the title compound (9 mg, 23%). LCMS (Method A): R_{T}=4.67 min, M+H⁺=366. ¹H NMR (500 MHz, CDCl₃) 7.73 (s, 1H), 7.62-7.51 (m, 4H), 7.50-7.36 (m, 5H), 7.36-7.32 (m, 2H), 2.50-2.45 (m, 2H), 2.20-2.15 (m, 2H), 2.10-2.03 (m, 1H), 1.76-1.66 (m, 1H).

### Example 7

### 2-(4-(1-Aminocyclobutyl)phenyl)-3-phenylbenzofuran-5-carboxamide

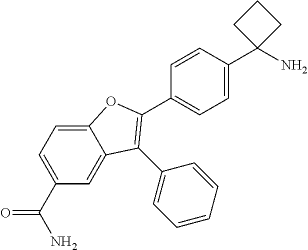

### Step 1: tert-Butyl (1-(4-(5-carbamoyl-3-phenylbenzofuran-2-yl)phenyl)cyclobutyl)carbamate

To a solution of tert-butyl (1-(4-(5-cyano-3-phenylbenzofuran-2-yl)phenyl)cyclobutyl)carbamate (150 mg, 0.32 mmol) in anhydrous DMSO (1 ml), was added potassium carbonate (9 mg, 0.064 mmol). The suspension was heated to 40° C., and hydrogen peroxide (30%, 66 μl, 0.64 mmol) was added slowly. The reaction temperature was increased to 70° C. for 1.5 h. After cooling to 30° C., water (3 ml) was added slowly, followed by DCM (5 ml). The layers were separated, and the aqueous layer extracted with DCM (5 ml). The combined organics were washed with dilute brine (5 ml), and concentrated in vacuo, before purification by silica gel chromatography (gradient elution 0 to 100% ethyl acetate in hexanes). This yielded the title compound as an off-white foam (68 mg, 43%). ¹H NMR (500 MHz, CDCl₃) 7.96 (s, 1H), 7.84 (dd, 1H), 7.67-7.61 (m, 3H), 7.53-7.46 (m, 5H), 7.41 (d, 2H), 6.1 (br s, 1H), 5.56 (br s, 1H), 5.08 (s, 1H), 2.6-2.52 (m, 4H), 2.2-2.1 (m, 1H), 1.95-1.85 (m, 1H), 1.5-1.3 (br s, 9H).

### Step 2: 2-(4-(1-Aminocyclobutyl)phenyl)-3-phenylbenzofuran-5-carboxamide

To a solution of tert-butyl (1-(4-(5-carbamoyl-3-phenylbenzofuran-2-yl)phenyl)cyclobutyl)carbamate (68 mg, 0.14 mmol) in DCM (1.5 ml) was added TFA (0.5 ml). The reaction was allowed to stir at RT for 1 h before concentration in vacuo. The residue was dissolved in DCM (2.5 ml), and sat. NaHCO₃ (2.5 ml) was added. The precipitate was filtered, and washed with DCM and hexanes. This yielded the title compound as a white solid after drying by high vacuum (28 mg, 52%). LCMS (Method A): R_{T}=3.84 min, M+H⁺=384. ¹H NMR (500 MHz, d₆-DMSO) 8.1 (br s, 1H), 8.01-7.96 (m, 4H), 7.76 (d, 1H), 7.67 (d, 2H), 7.6-7.5 (m, 8H), 7.35 (br s, 1H), 2.65-2.55 (m, 2H), 2.45-2.5 (m, 2H), 2.2-2.1 (m, 1H), 1.85-1.75 (m, 1H).

### Example 8

### 2-(4-(1-Aminocyclobutyl)phenyl)-3-phenylbenzofuran-7-carbonitrile

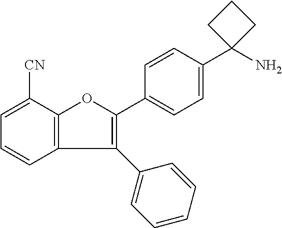

### Step 1: tert-Butyl (1-(4-((3-cyano-2-methoxyphenyl)ethynyl)phenyl)cyclobutyl)carbamate

To a degassed solution of 3-bromo-2-methoxybenzonitrile (146 mg, 0.69 mmol) in triethylamine (1.5 mL) at 0° C. was added Pd(P^{t}Bu₃)₂ (14.1 mg, 6 mol %), CuI (1.8 mg, 2 mol %) and tert-butyl (1-(4-ethynylphenyl)cyclobutyl)carbamate (125 mg, 0.46 mmol). The brown suspension was stirred for 16 hours at room temperature before the reaction mixture was suspended in dichloromethane and washed with brine, dried over magnesium sulphate, filtered and concentrated in vacuo. The resulting residue was purified by silica gel chromatography (gradient 10 to 25% EtOAc in hexane) to give the title compound as a white solid (167 mg, 60%). ¹H-NMR (500 MHz, CDCl₃) δ 7.69 (d, 1H), 7.50-7.56 (m, 3H), 7.42 (d, 2H), 7.11 (dd, 1H), 5.25 (bs, 1H), 4.23 (s, 3H), 2.51-2.56 (m, 4H), 2.09-2.15 (m, 1H), 1.86-1.90 (m, 1H), 1.37 (bs, 9H).

### Step 2: tert-Butyl (1-(4-(7-cyano-3-iodobenzofuran-2-yl)phenyl)cyclobutyl)carbamate

A solution of tert-butyl (1-(4-((3-cyano-2-methoxyphenyl)ethynyl)phenyl)cyclobutyl)carbamate (167 mg, 0.42 mmol) in DCM (6 mL) was cooled to 0° C. A solution of iodine monochloride (1M in DCM, 622 μL, 0.62 mmol) was added dropwise over 5 min. The mixture was then stirred whilst being allowed to reach RT. After 3 h, the mixture was diluted using DCM washed with 10% sodium thiosulphate solution, dried (Na₂SO₄), filtered and concentrated in vacuo. The resulting residue was purified by silica gel chromatography (gradient 10 to 25% EtOAc in hexane) to afford the title compound (152 mg, 70%). ¹H-NMR (500 MHz, CDCl₃) δ 8.21(d, 2H), 7.67 (m, 2H), 7.59 (d, 2H), 7.38 (dd, 1H), 5.21 (bs, 1H), 2.57-2.62 (m, 4H), 2.13-2.18 (m, 1H), 1.90-1.97 (m, 1H), 1.32 (bs, 9H).

### Step 3: tert-Butyl (1-(4-(7-cyano-3-phenylbenzofuran-2-yl)phenyl)cyclobutyl)carbamate

To a degassed solution of tert-butyl (1-(4-(7-cyano-3-iodobenzofuran-2-yl)phenyl)cyclobutyl)carbamate (150 mg, 0.29 mmol) in dimethoxyethane (6 mL) was added Pd(OAc)₂ (9.8 mg, 5 mol %), triphenylphosphine (11.4 mg, 15 ml %), cesium fluoride (207 mg, 1.36 mmol) and phenyl boronic acid (53 mg, 0.48 mmol). The brown suspension was stirred for 16 hours at 75° C. before the reaction mixture was suspended in dichloromethane and washed with brine, dried over magnesium sulphate, filtered and concentrated in vacuo. The resulting residue was purified by silica gel chromatography (gradient 10 to 25% EtOAc in hexane) to give the title compound as a white solid (94.3 mg, 70%). ¹H-NMR (500 MHz, CDCl₃) δ 7.59-7.69 (m, 3H), 7.50 (d, 1H), 7.26-7.45 (m, 5H), 7.42 (d, 2H), 7.29 (dd, 1H), 5.17 (bs, 1H), 2.50-2.55 (m, 4H), 2.08-2.15 (m, 1H), 1.82-1.89 (m, 1H), 1.31 (bs, 9H).

### Step 4: 2-(4-(1-Aminocyclobutyl)phenyl)-3-phenylbenzofuran-7-carbonitrile

To a solution of tert-butyl (1-(4-(7-cyano-3-phenylbenzofuran-2-yl)phenyl)cyclobutyl)carbamate (86 mg, 0.185 mmol) in tetrahydrofuran (0.5 mL) was added 4M HCl in 1,4-dioxane (1 mL). The reaction mixture was stirred for 16 hours at room temperature before being suspended in ethyl acetate and washed with a saturated solution of sodium hydrogen carbonate, dried over magnesium sulphate, filtered and concentrated in vacuo. The resulting residue was purified by silica gel chromatography (EtOAc with 1% triethylamine) to give a pale yellow solid (30 mg, 45%). ¹H-NMR (500 MHz, methanol-d₄) δ 7.57 (dd, 2H), 7.50 (d, 2H), 7.33-7.41 (m, 3H), 7.33-7.37 (m, 4H), 7.27 (dd, 1H), 2.40-2.46 (m, 2H), 2.11-2.17 (m, 2H), 1.91-2.02 (m, 1H), 1.60-1.69 (m, 1H). LCMS (Method A) R_{T}=4.99 min, M−NH₃=348.

### Example 9

### 2-(4-(1-Aminocyclobutyl)phenyl)-3-phenylbenzofuran-7-carboxamide.2HCl

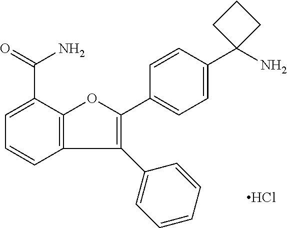

### Step 1: tert-Butyl (1-(4-(7-carbamoyl-3-phenylbenzofuran-2-yl)phenyl)cyclobutyl)carbamate

To a solution of tert-butyl (1-(4-(7-cyano-3-iodobenzofuran-2-yl)phenyl)cyclobutyl)carbamate (53 mg, 0.114 mmol) in dimethylsulfoxide (1 mL) was added potassium carbonate (3.2 mg, 0.023 mmol). The resulting solution was heated to 40° C. and an aqueous solution of hydrogen peroxide (30%), (23.3 μA, 0.228 mmol) was added. The resulting solution was heated at 70° C. for 3.5 h and then cooled to room temperature before being suspended in ethyl acetate and washed with a saturated solution of sodium hydrogen carbonate, dried over magnesium sulphate, filtered and concentrated in vacuo. The resulting residue was purified by silica gel chromatography (EtOAc) to give a white solid (42 mg, 76%). ¹H-NMR (500 MHz, CDCl₃) δ 8.15 (d, 1H), 7.65 (d, 1H), 7.60 (d, 2H), 7.40-7.50 (m, 7H), 7.37 (dd, 1H), 6.30 (bs, 1H), 5.17 (bs, 1H), 2.51-2.55 (m, 4H), 2.09-2.15 (m, 1H), 1.84-1.89 (m, 1H), 1.37 (bs, 9H).

### Step 2: 2-(4-(1-Aminocyclobutyl)phenyl)-3-phenylbenzofuran-7-carboxamide.2HCl

To a solution of tert-butyl (1-(4-(7-carbamoyl-3-phenylbenzofuran-2-yl)phenyl)cyclobutyl)carbamate (42 mg, 0.087 mmol) in tetrahydrofuran (1 mL) was added 4M HCl in 1,4-dioxane (1 mL). The reaction mixture was stirred for 16 hours at room temperature before being concentrated in vacuo to give a pale yellow solid (16 mg, 48%). ¹H-NMR (500 MHz, methanol-d₄) δ 7.82 (d, 1H), 7.71 (d, 2H), 7.52 (d, 1H), 7.36-7.45 (m, 7H), 7.29 (dd, 1H), 2.64-2.70 (m, 2H), 2.47-2.53 (m, 2H), 2.09-2.18 (m, 1H), 1.85-1.91 (m, 1H). LCMS (Method A) R_{T}=4.17 min, M+2H⁺=384.

### Example 10

### 1-(4-(7-Phenylfuro[2,3-b]pyrazin-6-yl)phenyl)cyclobutanamine.HCl

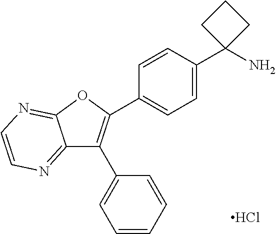

### Step 1: tert-Butyl (1-(4-((3-methoxypyrazin-2-yl)ethynyl)phenyl)cyclobutyl)carbamate

To a degassed solution of 2-iodo-3-methoxypyrazine (163 mg, 0.69 mmol) in triethylamine (1.5 mL) at 0° C. was added Pd(P^{t}Bu₃)₂ (14.1 mg, 6 mol %), CuI (1.8 mg, 2 mol %) and tert-butyl (1-(4-ethynylphenyl)cyclobutyl)carbamate (125 mg, 0.46 mmol). The brown suspension was stirred for 16 hours at room temperature before the reaction mixture was suspended in dichloromethane and washed with brine, dried over magnesium sulphate, filtered and concentrated in vacuo. The resulting residue was purified by silica gel chromatography (gradient 10 to 25% EtOAc in hexane) to give the title compound as a white solid (124 mg, 47%). ¹H-NMR (500 MHz, CDCl₃) δ 8.15 (s, 1H), 8.04 (s, 1H), 7.60 (d, 2H), 7.44 (d, 1H), 5.30 (bs, 1H), 4.11 (s, 3H), 2.51-2.56 (m, 4H), 2.09-2.14 (m, 1H), 1.84-1.91 (m, 1H), 1.37 (bs, 9H).

### Step 2: tert-Butyl (1-(4-(7-iodofuro[2,3-b]pyrazin-6-yl)phenyl)cyclobutyl)carbamate

A solution of tert-butyl (1-(4-((3-methoxypyrazin-2-yl)ethynyl)phenyl)cyclobutyl)carbamate (124 mg, 0.29 mmol) in DCM (6 mL) was cooled to 0° C. A solution of iodine monochloride (1M in DCM, 440 μL, 0.44 mmol) was added dropwise over 5 min. The mixture was then stirred whilst being allowed to reach RT. After 4 h the mixture was diluted using DCM, washed with 10% sodium thiosulphate solution, water, dried (Na₂SO₄), filtered and concentrated in vacuo. The resulting residue was purified by silica gel chromatography (gradient 10 to 25% EtOAc in hexane) to afford the title compound (106 mg, 74%). ¹H-NMR (500 MHz, CDCl₃) δ 8.59 (s, 1H), 8.30 (d, 2H), 8.30 (d, 1H), 8.27 (d, 1H), 5.39 (bs, 1H), 2.56-2.61 (m, 4H), 2.14-2.19 (m, 1H), 1.91-1.96 (m, 1H), 1.32 (bs, 9H).

### Step 3: tert-Butyl (1-(4-(7-phenylfuro[2,3-b]pyrazin-6-yl)phenyl)cyclobutyl)carbamate

To a degassed solution of tert-butyl (1-(4-(7-iodofuro[2,3-b]pyrazin-6-yl)phenyl)cyclobutyl)carbamate (106 mg, 0.216 mmol) in dimethoxyethane (5 mL) was added Pd(OAc)₂ (7.3 mg, 5 mol %), triphenylphosphine (8.5 mg, 15 ml %), cesium fluoride (154 mg, 1.01 mmol) and phenyl boronic acid (39.5 mg, 0.32 mmol). The brown suspension was stirred for 16 hours at 75° C. before the reaction mixture was suspended in dichloromethane and washed with brine, dried over magnesium sulphate, filtered and concentrated in vacuo. The resulting residue was purified by silica gel chromatography (gradient 10 to 25% EtOAc in hexane) to give the title compound as a white solid (52 mg, 55%). ¹H-NMR (500 MHz, CDCl₃) δ 8.55 (d, 1H), 8.27 (d, 1H), 7.77 (d, 2H), 7.63 (d, 2H), 7.50 (dd, 2H), 7.42-7.50 (m, 3H), 5.13 (bs, 1H), 2.49-2.57 (m, 4H), 2.10-2.15 (m, 1H), 1.86-1.92 (m, 1H), 1.37 (bs, 9H).

### Step 4: 1-(4-(7-Phenylfuro[2,3-b]pyrazin-6-yl)phenyl)cyclobutanamine.HCl

To a solution of tert-butyl (1-(4-(7-phenylfuro[2,3-b]pyrazin-6-yl)phenyl)cyclobutyl)carbamate (50 mg, 0.113 mmol) in tetrahydrofuran (0.5 mL) was added 4M HCl in 1,4-dioxane (1 mL). The reaction mixture was stirred for 16 hours at room temperature before being suspended in diethyl ether and filtered. The resulting solid was washed with diethyl ether to give a white solid (6 mg, 16%). ¹H-NMR (500 MHz, methanol-d₄) δ 8.60 (d, 1H), 8.40 (d, 1H), 7.89 (d, 2H), 7.59-7.63 (m, 4H), 7.50-7.56 (m, 3H), 2.80-2.86 (m, 2H), 2.61-2.67 (m, 2H), 2.25-2.32 (m, 1H), 1.98-2.05 (m, 1H). LCMS (Method A) R_{T}=8.01 min, M+H⁺=442.

### Example 11

### 1-(4-(5-Bromo-3-phenylfuro[2,3-b]pyridin-2-yl)phenyl)cyclobutanamine.HCl

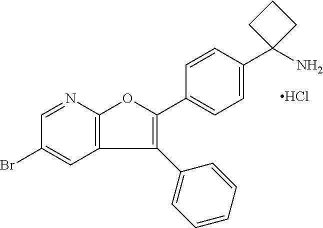

### Step 1: tert-Butyl (1-(4-((5-bromo-2-methoxypyridin-3-yl)ethynyl)phenyl)cyclobutyl)carbamate

To a degassed solution of 5-bromo-3-iodo-2-methoxypyridine (823 mg, 2.62 mmol) in triethylamine (8.0 mL) at 0° C. was added Pd(P^{t}Bu₃)₂ (110.4 mg, 6 mol %), CuI (5.0 mg, 1 mol %) and tert-butyl (1-(4-ethynylphenyl)cyclobutyl)carbamate (711 mg, 2.62 mmol). The brown suspension was stirred for 56 hours at room temperature before the reaction mixture was suspended in dichloromethane and washed with brine, dried over magnesium sulphate, filtered and concentrated in vacuo. The resulting residue was purified by silica gel chromatography (gradient 10 to 25% EtOAc in hexane) to give the title compound as a white solid (1.04 g, 87%). ¹H-NMR (500 MHz, CDCl₃) δ 8.14 (d, 1H), 7.84 (d, 1H), 7.52 (dd, 2H), 7.41 (d, 2H), 5.13 (bs, 1H), 4.01 (s, 3H), 2.50-2.56 (m, 4H), 2.09-2.14 (m, 1H), 1.84-1.90 (m, 1H), 1.36 (bs, 9H).

### Step 2: tert-Butyl (1-(4-(5-bromo-3-iodofuro[2,3-b]pyridin-2-yl)phenyl)cyclobutyl)carbamate

A solution of tert-butyl (1-(4-((5-bromo-2-methoxypyridin-3-yl)ethynyl)phenyl)cyclobutyl)carbamate (1.04 g, 2.27 mmol) in DCM (40 mL) was cooled to 0° C. A solution of iodine monochloride (1M in DCM, 3.4 mL, 3.41 mmol) was added dropwise over 5 min. The mixture was then stirred whilst being allowed to reach RT. After 6 h the mixture was diluted using DCM, washed with 10% sodium thiosulphate solution, dried (Na₂SO₄), filtered and concentrated in vacuo. The resulting residue was purified by silica gel chromatography (gradient 10 to 25% EtOAc in hexane) to afford the title compound (770 mg, 60%). ¹H-NMR (500 MHz, CDCl₃) δ 8.36 (d, 1H), 8.21 (d, 2H), 7.88 (d, 1H), 7.58 (d, 2H), 5.16 (bs, 1H), 2.52-2.59 (m, 4H), 2.13-2.18 (m, 1H), 1.90-1.95 (m, 1H), 1.31 (bs, 9H).

### Step 3: 1-(4-(5-Bromo-3-phenylfuro[2,3-b]pyridin-2-yl)phenyl)cyclobutanamine

To a degassed solution tert-butyl (1-(4-(5-bromo-3-iodofuro[2,3-b]pyridin-2-yl)phenyl)cyclobutyl)carbamate (770 mg, 1.35 mmol) in dimethoxyethane (30 mL) was added Pd(OAc)₂ (45.5 mg, 5 mol %), triphenylphosphine (53.1 mg, 15 mol %), cesium fluoride (964 mg, 6.35 mmol) and phenyl boronic acid (157 mg, 1.29 mmol). The brown suspension was stirred for 16 hours at 75° C. before the reaction mixture was suspended in dichloromethane and washed with brine, dried over magnesium sulphate, filtered and concentrated in vacuo. The resulting residue was purified by silica gel chromatography (gradient 10 to 25% EtOAc in hexane) to give the title compound as a white solid (356 mg, 51%). ¹H-NMR (500 MHz, CDCl₃) δ 8.36 (d, 1H), 7.88 (d, 1H), 7.58 (d, 2H), 7.43-7.49 (m, 5H), 7.38 (d, 2H), 5.10 (bs, 1H), 2.50-2.59 (m, 4H), 2.04-2.15 (m, 1H), 1.82-1.89 (m, 1H), 1.32 (bs, 9H).

### Step 4: 1-(4-(5-Bromo-3-phenylfuro[2,3-b]pyridin-2-yl)phenyl)cyclobutanamine.HCl

To a solution of tert-butyl (1-(4-(5-bromo-3-iodofuro[2,3-b]pyridin-2-yl)phenyl)cyclobutyl)carbamate (20 mg, 0.038 mmol) in tetrahydrofuran (0.5 mL) was added 4M HCl in 1,4-dioxane (1 mL). The reaction mixture was stirred for 16 hours at room temperature before being suspended in diethyl ether and filtered. The resulting solid was washed with diethyl ether to give a white solid (10 mg, 62%). ¹H-NMR (500 MHz, methanol-d₄) a 8.45 (d, 1H), 8.36 (d, 2H), 7.80 (d, 1H), 7.77 (dd, 2H), 7.73 (dd, 2H), 7.54-7.59 (m, 3H), 2.81-2.88 (m, 2H), 2.65-2.70 (m, 2H), 2.26-2.32 (m, 1H), 2.01-2.06 (m, 1H). LCMS (Method A) R_{T}=5.47 min, M+H(⁷⁹Br)⁺observed=420.1, calculated=520.4, M+H(⁸¹Br)⁺observed=422.0, calculated=422.4.

### Example 12

### 6-(4-(1-Aminocyclobutyl)phenyl)-5-phenylfuro[2,3-d]pyrimidin-4(3H)-one

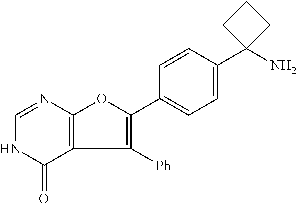

### Step 1: tert-Butyl (1-(4-(4-oxo-5-phenyl-3,4-dihydrofuro[2,3-d]pyrimidin-6-yl)phenyl)cyclobutyl-carbamate

To a solution of 6-iodo-5-phenylfuro[2,3-d]pyrimidin-4(3H)-one (65 mg, 0.19 mmol, prepared as described in WO2006/004658) in DMF (3 mL) was added tert-butyl (1-(4-(4,4,5,5-tetramethyl-1,3,2-dioxaborolan-2-yl)phenyl)cyclobutyl)carbamate (72 mg, 0.19 mmol, prepared as described in WO2008/070016), Pd(dppf)Cl₂ (16 mg, 0.02 mmol), K₂CO₃ (66 mg, 0.48 mmol) and water (1 mL). The reaction was then heated at 80° C. overnight under a nitrogen atmosphere. After cooling to RT, ethyl acetate and water was added, the layers were separated, and the aqueous extracted once more with ethyl acetate. The combined organic layers were then washed with water/brine 1:1, dried (Na₂SO₄), filtered, and concentrated in vacuo. The residue was triturated with DCM to provide the title compound as a white solid.

LRMS (ESI) M+Na⁺=480.

### Step 2: 6-(4-(1-Aminocyclobutyl)phenyl)-5-phenylfuro[2,3-d]pyrimidin-4(3H)-one

tert-butyl (1-(4-(4-oxo-5-phenyl-3,4-dihydrofuro[2,3-d]pyrimidin-6-yl)phenyl)cyclobutyl)carbamate (16.6 mg, 0.04 mmol) was stirred at RT in a 1:1 mixture of TFA/DCM (2 mL). After 20 min the solvent was removed in vacuo the residue taken up in a saturated solution of NaHCO₃, and extracted twice with ethyl acetate. The organic extracts were combined, dried (MgSO₄), filtered, and concentrated almost to dryness. The crude material was purified by flash chromatography (SiO₂, 0-15% methanol in dichloromethane), to give the title compound as a white solid (3 mg, 23%). ¹H NMR (400 MHz, DMSO) δ 8.18 (1H, s) 7.47-7.39 (10H, m), 2.51-2.34 (2H, m), 2.14-2.08 (2H, m), 2.00-1.97 (1H, m), 1.66-1.64 (1H, m).

### Example 13

### 1-(4-(4-Methoxy-2-morpholino-5-phenylfuro[2,3-d]pyrimidin-6-yl)phenyl)cyclobutanamine

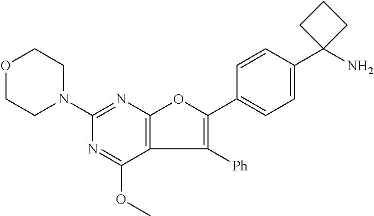

### Step 1: 2-(Methylthio)-5-phenylfuro[2,3-d]pyrimidin-4(3H)-one

To a solution of (2-chloro-2-nitrovinyl)benzene (3.7 g, 20 mmol, prepared as described in WO2006/004658) in ethanol (100 mL) was charged 6-hydroxy-2-(methylthio)pyrimidin-4(3H)-one (3.5 g, 22 mmol) and DBU (6 mL, 40 mmol). The reaction was heated to reflux under a nitrogen atmosphere until complete by LCMS (4 days). The reaction mixture was concentrated in vacuo and the brown solid taken up in ethyl acetate (250 mL) and washed with water (2×150 mL). The organic layer was then dried (MgSO₄), filtered and concentrated in vacuo to give the title compound as a brown solid. Material taken crude (˜70% pure) into the following step. LCMS R_{T}=5.38 min, M+H⁺=259.

### Step 2: 4-Chloro-2-(methylthio)-5-phenylfuro[2,3-d]pyrimidine

2-(Methylthio)-5-phenylfuro[2,3-d]pyrimidin-4(3H)-one (7.0 g, 27 mmol) in POCl₃ (85 mL) was heated at reflux for 90 min. After cooling to RT the POCl₃ was removed in vacuo. The residue was diluted with ice cold water and extracted three times with DCM. The organic layers were combined, dried (MgSO₄), filtered and concentrated in vacuo to give the title compound as a brown solid (6.1 g, 83%). LC-MS R_{T}=7.23 min, M+H⁺=277/279.

### Step 3: 4-Methoxy-2-(methylthio)-5-phenylfuro[2,3-d]pyrimidine

To a solution of 4-chloro-2-(methylthio)-5-phenylfuro[2,3-d]pyrimidine (4.6 g, 17 mmol) in DMF (60 mL) under a nitrogen atmosphere was charged a 25% solution of NaOMe in MeOH (3.6 mL, 17 mmol). The reaction mixture was heated at 80° C. for 1.5 h. An aliquot was worked for ¹H NMR to confirm reaction complete. After cooling to RT the reaction mixture was diluted with water (250 ml) and extracted twice with ethyl acetate (150 mL). The combined organic extracts were washed with brine:water 1:1 (5×150 mL), dried (MgSO₄), filtered and concentrated in vacuo. The crude material was subjected to flash chromatography (SiO₂, gradient 3-20% ethyl acetate in hexane) to give the title compound as a yellow solid (2.6 g, 58%). LCMS R_{T}=7.22 min, M+H⁺=273.

### Step 4: 6-Bromo-4-methoxy-2-(methylthio)-5-phenylfuro[2,3-d]pyrimidine

To a solution of 4-methoxy-2-(methylthio)-5-phenylfuro[2,3-d]pyrimidine (57 mg, 0.21 mmol), in dry THF (1 ml), cooled to −78° C. under a nitrogen atmosphere, was added 1.6 M nBuLi in hexanes (0.2 mL 0.31 mmol). The mixture was stirred for 1 h at −78° C., bromine (1.6 μL, 0.31 mmol) was added and the reaction stirred for 1 h at −78° C. and then allowed to warm to RT. The reaction was quenched by the addition of aq. sodium thiosulphate (0.05M, 5 mL) and was extracted with ethyl acetate (2×10 mL). The combined organic extracts were washed with H₂O:brine 1:1 (10 mL), dried (MgSO₄), filtered and concentrated in vacuo to afford an off white solid (70 mg, 94%). LC-MS R_{T}=7.84 min, M+H⁺=351/353.

### Step 5: tert-Butyl (1-(4-(4-methoxy-2-(methylthio)-5-phenylfuro[2,3-d]pyrimidin-6-yl)phenyl)cyclobutyl)carbamate

A solution of 6-bromo-4-methoxy-2-(methylthio)-5-phenylfuro[2,3-d]pyrimidine (0.8 g, 2.3 mmol), tert-butyl (1-(4-(4,4,5,5-tetramethyl-1,3,2-dioxaborolan-2-yl)phenyl)cyclobutyl)carbamate (1.27 g, 3.4 mmol, prepared as described in WO2008/070016), and potassium phosphate tribasic (1.45 g, 6.8 mmol) in DMF:H₂O 3:1 (20 mL) was degassed by bubbling N₂ through the reaction mixture for 10 min. Tetrakis(triphenylphosphine)palladium(0) (131 mg, 0.1 mmol) was then charged to the reaction and heated at 90° C. under a nitrogen atmosphere until complete by LCMS (1.5 h). The mixture was partitioned between water (40 mL) and EtOAc (30 mL). The aqueous phase was extracted once more with ethyl acetate (30 mL). The combined organic phases were washed with water:brine 1:1 (5×20 mL), dried (MgSO₄), filtered and concentrated in vacuo. The resultant residue was subjected to flash chromatography (SiO₂, gradient 0 to 20% ethyl acetate in hexane) to afford the title compound as a white solid (710 mg, 60%). LCMS R_{T}=8.88 min, M+H⁺=518.

### Step 6: tert-Butyl (1-(4-(4-methoxy-2-(methylsulfonyl)-5-phenylfuro[2,3-d]pyrimidin-6-yl)phenyl)cyclobutyl)carbamate

To a solution of tert-butyl (1-(4-(4-methoxy-2-(methylthio)-5-phenylfuro[2,3-d]pyrimidin-6-yl)phenyl)cyclobutyl)carbamate (250 mg, 0.5 mmol) in THF: MeOH 1:1 (10 mL) was added dropwise a solution of Oxone® (1.2 g, 1.9 mmol) in H₂O (10 mL). The reaction was then stirred at RT overnight and at 50° C. for 2 h. To the reaction mixture was added aq sodium hydrogen carbonate. The aqueous layer was extracted three times with ethyl acetate. The organic layers were combined, dried (MgSO₄), filtered and concentrated in vacuo to afford the title compound as a white solid (210 mg, 79%). LCMS (Method A) R_{T}=7.45 min, M+H⁺=550.

### Step 7: tert-Butyl (1-(4-(4-methoxy-2-morpholino-5-phenylfuro[2,3-d]pyrimidin-6-yl)phenyl)cyclobutyl)carbamate

A solution of tert-butyl (1-(4-(4-methoxy-2-(methylsulfonyl)-5-phenylfuro[2,3-d]pyrimidin-6-yl)phenyl)cyclobutyl)carbamate (105 mg, 0.2 mmol) and morpholine (0.5 mL, 5.7 mmol) in toluene (1 mL) was heated at 100° C. for 2 h in a sealed tube. The reaction mixture was concentrated in vacuo and purified by flash chromatography (SiO₂, gradient 10 to 50% ethyl acetate in hexane) to afford the title compound as a white gum (102 mg, 96%). LCMS (Method A) R_{T}=8.65 min, M+H⁺=557.

### Step 8: 1-(4-(4-Methoxy-2-morpholino-5-phenylfuro[2,3-d]pyrimidin-6-yl)phenyl)cyclobutanamine

To a solution of tert-butyl (1-(4-(4-methoxy-2-morpholino-5-phenylfuro[2,3-d]pyrimidin-6-yl)phenyl)cyclobutyl)carbamate (67 mg, 0.12 mmol) in DCM (2 mL) was charged TFA (0.2 mL). The reaction was stirred at RT for 1 h. The solvents were removed in vacuo, the residue was neutralised using satd. aq. NaHCO₃, DCM was added, and the resulting biphasic solution was separated using a phase separator (Isolute® SPE). The organic layer was concentrated in vacuo and the crude material purified by flash chromatography (SiO₂, gradient 2 to 10% methanol in dichloromethane) to afford the desired product as an off-white solid (36 mg, 65%). LCMS (Method A) R_{T}=4.75 min, M+H⁺=440/458. ¹H NMR (500 MHz, CDCl₃): δ 7.44-7.39 (4H, m), 7.35-7.29 (3H, m), 7.22-7.19 (2H, m), 3.81 (3H, s), 3.80-3.71 (4H, m), 2.47-2.42 (4H, m), 2.11-2.05 (2H, m). 2.03-1.95 (1H, m), 1.71-1.64 (1H, m).

### Example 14

### N¹-(6-(4-(1-Aminocyclobutyl)phenyl)-4-methoxy-5-phenylfuro[2,3-d]pyrimidin-2-yl)-N²,N²-dimethylethane-1,2-diamine

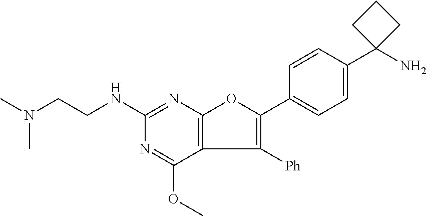

### Step 1: tert-Butyl (1-(4-(2-((2-(dimethylamino)ethyl)amino)-4-methoxy-5-phenylfuro[2,3-d]pyrimidin-6-yl)phenyl)cyclobutyl)carbamate

Following the procedure for tert-butyl (1-(4-(4-methoxy-2-morpholino-5-phenylfuro[2,3-d]pyrimidin-6-yl)phenyl)cyclobutyl)carbamate, tert-butyl (1-(4-(4-methoxy-2-(methylsulfonyl)-5-phenylfuro[2,3-d]pyrimidin-6-yl)phenyl)cyclobutyl)carbamate (105 mg, 0.2 mmol) and N,N-dimethylethylene diamine (0.5 mL, 4.6 mmol) was reacted to give the title compound (49 mg, 46%). LCMS (Method A) R_{T}-=5.13 min, M+H⁺=558.

### Step 2: N¹-(6-(4-(1-Aminocyclobutyl)phenyl)-4-methoxy-5-phenylfuro[2,3-d]pyrimidin-2-yl)-N²,N²-dimethylethane-1,2-diamine

Following the procedure for 1-(4-(4-methoxy-2-morpholino-5-phenylfuro[2,3-d]pyrimidin-6-yl)phenyl)cyclobutanamine, tert-butyl (1-(4-(2-((2-(dimethylamino)ethyl)amino)-4-methoxy-5-phenylfuro[2,3-d]pyrimidin-6-yl)phenyl)cyclobutyl)carbamate (42 mg, 0.07 mmol) was reacted to give the title compound (10 mg, 29%). LCMS (Method A) R_{T}=3.44 min, M+H⁺=459. ¹H NMR (500 MHz, CDCl₃): δ 7.43-7.38 (4H, m), 7.34-7.28 (3H, m), 7.23-7.19 (2H, m), 5.72 (1H, br. s), 3.79 (3H, s), 3.58-3.55 (2H, m), 2.66 (2H, m), 2.51-2.43 (2H, m). 2.35 (6H, s), 2.10 (2H, m), 2.05-2.00 (1H, m), 1.72-1.64 (1H, m).

### Example 15

### 6-(4-(1-Aminocyclobutyl)phenyl)-2-(methylthio)-5-phenylfuro[2,3-d]pyrimidin-4(3H)-one

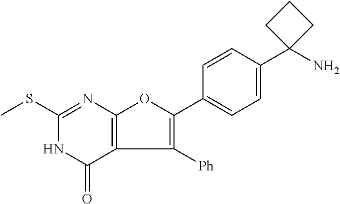

### Step 1: 6-Bromo-2-(methylthio)-5-phenylfuro[2,3-d]pyrimidin-4(3H)-one

To a sealed tube was charged 2-(methylthio)-5-phenylfuro[2,3-d]pyrimidin-4(3H)-one (500 mg, 1.9 mmol), NBS (379 mg, 2.1 mmol) and potassium acetate (570 mg, 5.8 mmol) in DMF (5 mL). The vial was purged with nitrogen sealed and heated at 80° C. overnight. The reaction was incomplete by LCMS (starting material R_{T}=5.37 min, M+H⁺=259 and product R_{T}=6.06 min, M+H⁺=337/339). The mixture was partitioned between water (20 mL) and ethyl acetate (20 mL). The layers separated and the aqueous phase was extracted further with ethyl acetate (20 mL). The combined organic phases were dried (MgSO₄), filtered and concentrated in vacuo. The material was used crude in the next step.

### Step 2: tert-Butyl (1-(4-(4-oxo-5-phenyl-4,4-a-dihydrofuro[2,3-d]pyrimidin-6-yl)phenyl)cyclobutyl)carbamate

To a microwave vial were charged 6-bromo-2-(methylthio)-5-phenylfuro[2,3-d]pyrimidin-4(3H)-one (30 mg, 0.09 mmol), tert-butyl (1-(4-(4,4,5,5-tetramethyl-1,3,2-dioxaborolan-2-yl)phenyl)cyclobutyl)carbamate (50 mg, 0.13 mmol, prepared as described in WO2008/070016), and potassium phosphate tribasic (57 mg, 0.27 mmol) in DMF:H₂O 5:1 (2.5 mL). The reaction mixture was degassed by bubbling N₂ through the reaction mixture for 10 min.

Tetrakis(triphenylphosphine)palladium(0) (5 mg, 4 μmol) was charged and the vial was heated in a microwave reactor at 80° C. for 20 min. The mixture was partitioned between water (2 mL) and ethyl acetate (3 mL). The layers separated and the aqueous phase was extracted once more with ethyl acetate (3 mL). Combined organic phases were washed with H₂O:brine 1:1 (4×3 mL), dried (MgSO₄), filtered and concentrated in vacuo. The resultant residue was subjected to flash chromatography (SiO₂, gradient 0 to 40% ethyl acetate in hexane) to afford the title compound (8 mg, 18%). LCMS R_{T}=7.26 min, M+H⁺=504.

### Step 3: 6-(4-(1-Aminocyclobutyl)phenyl)-5-phenylfuro[2,3-d]pyrimidin-4(4aH)-one hydrochloride

To a solution of tert-butyl (1-(4-(4-oxo-5-phenyl-4,4-a-dihydrofuro[2,3-d]pyrimidin-6-yl)phenyl)cyclobutyl)carbamate (8 mg, 0.02 μmol) in THF (2 mL) was charged 4M HCl in dioxane (2 mL). The reaction was stirred at RT for 3 hr and at 50° C. for 1 h. The reaction volume was reduced by half and diethyl ether added until a white precipitate formed. The solvent was decanted and the remaining solid washed again with diethyl ether and decanted. The solid was then dried under high vacuum overnight. This afforded the title compound as a white solid (3.7 mg, 49%). LCMS R_{T}=3.91 min, M−H⁺=402. ¹H NMR (500 MHz, methanol-d₄): δ 7.61-7.60 (2H, m), 7.50-7.42 (7H, m), 2.78-2.74 (2H, m), 2.69 (3H, s), 2.59-2.55 (2H, m), 2.24-2.16 (1H, m), 1.97-1.89 (1H, m).

### Example 16

### 6-(4-(1-Aminocyclobutyl)phenyl)-5-phenylthieno[2,3-d]pyrimidin-4(3H)-one

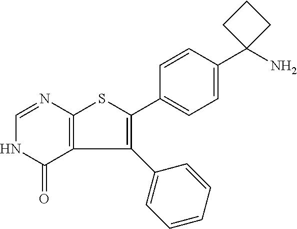

### Step 1: 6-Iodo-5-phenylthieno[2,3-d]pyrimidin-4(4aH)-one

5-phenylthieno[2,3-d]pyrimidin-4(4aH)-one (200 mg, 0.9 mmol), in CCl₄:CH₃CN 1:1 (20 mL) was charged with NIS (296 mg, 1.3 mmol). The reaction was heated at reflux overnight under a nitrogen atmosphere, a further 1 eq of NIS was charged to the reaction and this refluxed for a further 6 h. After allowing the reaction mixture to cool to RT, the reaction mixture was concentrated in vacuo. The residue taken up in ethyl acetate and 10% K₂CO₃ the layers were separated and the aqueous extracted twice more with ethyl acetate. The organic layers were combined, dried (Na₂SO₄) and concentrated in vacuo. The crude material was purified by flash chromatography (SiO₂, gradient 0 to 70% ethyl acetate in hexane) to afford the title compound (175 mg, 56%). LCMS R_{T}=5.26 min, M+H⁺=355.

### Step 2: tert-Butyl (1-(4-(4-oxo-5-phenyl-4,4-a-dihydrothieno[2,3-d]pyrimidin-6-yl)phenyl)cyclobutyl)carbamate

Following the procedure for tert-butyl (1-(4-(4-oxo-5-phenyl-4,4-a-dihydrofuro[2,3-d]pyrimidin-6-yl)phenyl)cyclobutyl)carbamate, 6-iodo-5-phenylthieno[2,3-d]pyrimidin-4(4aH)-one (50 mg, 0.14 mmol) and tert-butyl (1-(4-(4,4,5,5-tetramethyl-1,3,2-dioxaborolan-2-yl)phenyl)cyclobutyl)carbamate (79 mg, 0.21 mmol, prepared as described in WO2008/070016) was reacted to give the title compound (34 mg, 25%). LCMS R_{T}=6.55 min, M+H⁺=474.

### Step 3: 6-(4-(1-Aminocyclobutyl)phenyl)-5-phenylthieno[2,3-d]pyrimidin-4(3H)-one

Following the procedure for 6-(4-(1-aminocyclobutyl)phenyl)-5-phenylfuro[2,3-d]pyrimidin-4(4aH)-one hydrochloride, tert-butyl (1-(4-(4-oxo-5-phenyl-4,4-a-dihydrothieno[2,3-d]pyrimidin-6-yl)phenyl)cyclobutyl)carbamate (30 mg, 0.06 mmol) was reacted to afford the title compound. Except this material required further purification it was therefore taken up in 2M aq. NaHCO₃ and ethyl acetate. The layers were separated and the aqueous extracted twice more with ethyl acetate. The organic layers were combined, dried (MgSO₄) and concentrated in vacuo. The crude material was purified by flash chromatography (SiO₂, gradient 0 to 25% methanol in dichoromethane) to afford the title compound (5 mg, 21%). LCMS R_{T}=3.45 min, M−H⁺=372. ¹H NMR (500 MHz, DMSO): δ 8.14 (1H, s), 7.36-7.31 (5H, m), 7.26-7.26 (2H, m), 7.15 (2H, d), 2.38-2.33 (2H, m), 2.14-2.08 (2H, m), 2.00-1.97 (1H, m), 1.68-1.60 (1H, m).

### Example 17

### 1-(4-(4-Methoxy-3-phenylfuro[3,2-c]pyridin-2-yl)phenyl)cyclobutanamine

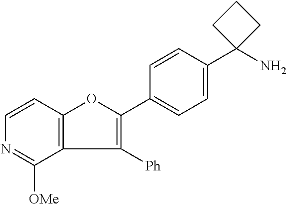

### Step 1: 4-Methoxyfuro[3,2-c]pyridine

To a solution of 4-chlorofuro[3,2-c]pyridine (3 g, 19.6 mmol) in DMF (30 mL) was charged NaOMe (25% in MeOH, 4.2 mL, 19.6 mmol). The reaction was then heated at 85° C. for 1 h. After cooling to RT the reaction mixture was diluted with water and extracted twice with ethyl acetate. The combined organic extracts were then washed five times with brine:water 1:1, dried (MgSO₄), filtered and concentrated in vacuo to afford the title compound as a yellow solid (2.7 g, 93%). ¹H NMR (400 MHz, CDCl₃): δ 7.94 (1H, d), 7.49 (1H, d), 7.03 (1H, dd), 6.76 (1H, dd), 4.01 (3H, s).

### Step 2: 2,3-Dibromo-4-methoxy-2,3-dihydrofuro[3,2-c]pyridine

To a solution of 4-methoxyfuro[3,2-c]pyridine (2.7 g, 18 mmol) in CCl₄ (30 mL) at RT was charged a solution of Br₂ (0.93 mL, 18 mmol) in CCl₄ (30 mL) slowly dropwise. The mixture was then stirred at RT for 1.5 h. The reaction was washed with aq. sodium thiosulphate and water. The organic layer was dried (MgSO₄), filtered and concentrated in vacuo to afford an off-white solid (5.6 g, 99%). LCMS R_{T}=6.02 min, M+H⁺=308/310/312.

### Step 3: 3-Bromo-4-methoxyfuro[3,2-c]pyridine

To a solution of 2,3-dibromo-4-methoxy-2,3-dihydrofuro[3,2-c]pyridine (5.6 g, 18 mmol) in THF (40 mL) was charged DBU (5.4 mL, 36 mmol) at RT. A white precipitate formed. After stirring at RT for 1 h, the reaction was diluted with water and extracted with DCM (3×). The organic extracts were combined and washed with brine, dried (MgSO₄), filtered and concentrated in vacuo to afford an off-white solid (3.2 g, 78%). LCMS R_{T}=5.61 min, M+H⁺=228/230.

### Step 4: 4-Methoxy-3-phenylfuro[3,2-c]pyridine

A solution of 3-bromo-4-methoxyfuro[3,2-d]pyridine (100 mg, 0.44 mmol), phenyl boronic acid (80 mg, 0.66 mmol) and sodium carbonate solution (2M in water, 0.7 uL, 1.3 mmol), in DME (2 mL) was degassed by bubbling N₂ through the reaction mixture for 10 min.

Tetrakis(triphenylphosphine)palladium(0) (25 mg, 0.02 mmol) was then charged to the reaction and this heated at 80° C. under a nitrogen atmosphere overnight. After allowing to cool to RT, the reaction mixture was concentrated in vacuo and the residue partitioned between water and ethyl acetate. The aqueous phase was extracted once more with ethyl acetate. The combined organic extracts were dried (MgSO₄), filtered and concentrated in vacuo. The resultant residue was subjected to flash chromatography (SiO₂, gradient 0 to 20% ethyl acetate in hexane) to afford the title compound as a white solid (60 mg, 61%). LCMS R_{T}=6.46 min, M+H⁺=226.

### Step 5: 2-Bromo-4-methoxy-3-phenylfuro[3,2-c]pyridine

Following the procedure for 6-bromo-4-methoxy-2-(methylthio)-5-phenylfuro[2,3-d]pyrimidine, 4-methoxy-3-phenylfuro[3,2-c]pyridine (80 mg, 0.35 mmol) was reacted to give the title compound as an off-white solid (93 mg, 86%). ¹H NMR (400 MHz, CDCl₃): δ 8.03 (1H, d), 7.59-7.57 (2H, m), 7.49-7.39 (3H, m), 7.10 (1H, d), 3.94 (3H, s).

### Step 6: tert-Butyl (1-(4-(4-methoxy-3-phenylfuro[3,2-c]pyridin-2-yl)phenyl)cyclobutyl)carbamate

Following the procedure for tert-butyl (1-(4-(4-oxo-5-phenyl-4,4-a-dihydrofuro[2,3-d]pyrimidin-6-yl)phenyl)cyclobutyl)carbamate, 2-bromo-4-methoxy-3-phenylfuro[3,2-c]pyridine (93 mg, 0.30 mmol) and tert-butyl (1-(4-(4,4,5,5-tetramethyl-1,3,2-dioxaborolan-2-yl)phenyl)cyclobutyl)carbamate (171 mg, 0.46 mmol, prepared as described in WO2008/070016) was reacted to give the title compound (85 mg, 59%). LRMS (ESI) M+Na⁺=493.

### Step 7: 1-(4-(4-Methoxy-3-phenylfuro[3,2-c]pyridin-2-yl)phenylcyclobutanamine

To a solution of tert-butyl (1-(4-(4-methoxy-3-phenylfuro[3,2-c]pyridin-2-yl)phenyl)cyclobutyl)carbamate (35 mg, 0.07 mmol) in DCM (2 mL) was charged TFA (0.2 mL). The reaction was then stirred at RT for 1.5 h. The solvent was removed in vacuo and the residue neutralised using aq. NaHCO₃. This was then extracted twice with ethyl acetate. The organic layers were combined, dried (MgSO₄), filtered and concentrated in vacuo. The product was then freeze dried (CH₃CN:H₂O) to afford the desired product as a white solid (21 mg, 76%). LCMS R_{T}=4.54 min, M−NH₃⁺=354. ¹H NMR (500 MHz, methanol-d₄): δ 7.93 (1H, d), 7.52-7.50 (2H, m), 7.35-7.32 (7H, m), 7.17 (1H, d), 3.75 (3H, s), 2.66-2.60 (2H, m), 2.46-2.40 (2H, m), 2.13-2.07 (1H, m), 1.86-1.80 (1H, m).

### Example 18

### 2-(4-(1-Aminocyclobutyl)phenyl)-3-phenylfuro[3,2-c]pyridin-4(5H)-one

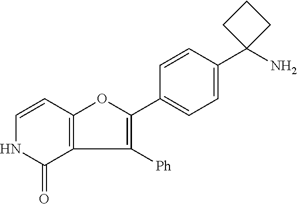

### Step 1: 2-(4-(1-Aminocyclobutyl)phenyl)-3-phenylfuro[3,2-c]pyridin-4(5H)-one

To a solution of tert-butyl (1-(4-(4-methoxy-3-phenylfuro[3,2-c]pyridin-2-yl)phenyl)cyclobutyl)carbamate (40 mg, 0.08 mmol) in 1,4 dioxane (2 mL) was charged 1.5 M aq.HCl (1 mL). The reaction was stirred at 80° C. for 6 h and allowed to cool to RT.

The reaction was neutralised using aq.NaHCO₃ and extracted three times with ethyl acetate. The organic layers were combined, dried (MgSO₄), filtered and concentrated in vacuo. The resultant residue was subjected to flash chromatography (SiO₂, gradient 0 to 10% methanol in dichloromethane) to afford the title compound as a white solid (5 mg, 17%). LRMS (ESI) M+H⁺=357. ¹H NMR (400 MHz, methanol-d₄): δ 7.53-7.38 (10H, m), 6.83 (1H, d), 2.60-2.53 (2H, m), 2.31-2.27 (2H, m), 2.18-2.04 (1H, m), 1.82-1.72 (1H, m).

### Example 19

### 1-(4-(3-Phenylfuro[2,3-b]pyridin-2-yl)phenyl)cyclobutanamine

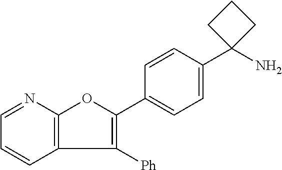

### Step 1: tert-Butyl (1-(44(2-methoxypyridin-3-yl)ethynyl)phenyl)cyclobutyl)carbamate

To a solution of tert-butyl (1-(4-ethynylphenyl)cyclobutyl)carbamate (93 mg, 0.34 mmol), and 3-Iodo-2-methoxypyridine (77 mg, 0.33 mmol) in Et₃N at 0° C. under a nitrogen atmosphere was charged PdCl₂(PPh₃)₂ (7 mg, 0.01 mmol) and copper (I) iodide (2 mg, 0.01 mmol). The reaction was stirred at 0° C. for 1 h and at RT for 2 h. The reaction mixture was diluted water and extracted with ethyl acetate twice. The organic layers were combined washed with water and brine, dried (MgSO₄) filtered and concentrated in vacuo. The crude material was purified by flash chromatography (SiO₂, gradient 0 to 20% ethyl acetate in hexane) to afford the title compound as a yellow solid (97 mg, 78%).

LCMS (Method A) R_{T}=7.51 min, M+H⁺=379.

### Step 2: tert-Butyl (1-(4-(3-iodofuro[2,3-b]pyridin-2-yl)phenyl)cyclobutyl)carbamate

A 1 M solution of ICI in DCM (0.38 mL, 0.38 mmol) was added dropwise to a stirred solution of 3-methoxy-2-(phenylethynyl)pyridine (97 mg, 0.26 mmol) in DCM (3 mL) at 0° C. under a nitrogen atmosphere. After 4 hours, the reaction mixture was quenched using satd. aq. sodium thiosulphate solution (3.0 mL). The resulting biphasic solution was separated using a phase separator (Isolute® SPE), washing using DCM (2×3.0 mL), and the solvents were removed in vacuo to give the title compound (92 mg, 73%) as an off-white solid. LCMS: R_{T}=7.82 min, M+H⁺=490.9.

### Step 3: tert-Butyl (1-(4-(3-phenylfuro[2,3-b]pyridin-2-yl)phenyl)cyclobutyl)carbamate

A solution of tert-butyl (1-(4-(3-iodofuro[2,3-b]pyridin-2-yl)phenyl)cyclobutyl)carbamate (92 mg, 0.19 mmol), phenyl boronic acid (34 mg, 0.28 mmol), and sodium carbonate (60 mg, 0.56 mmol) in toluene:EtOH:H₂O 20:5:1 (2 mL) was degassed by bubbling N₂ through the reaction mixture for 10 min. Tetrakis(triphenylphosphine)palladium(0) (11 mg, 0.009 mmol) was then charged to the reaction and was heated at 100° C. under N₂ overnight. The mixture was partitioned between water and ethyl acetate. The aqueous phase was extracted once more with ethyl acetate. The combined organic phases were washed with brine dried (MgSO₄), filtered and concentrated in vacuo. The resultant residue was subjected to flash chromatography (SiO₂, gradient 0 to 20% ethyl acetate in hexane) to afford the title compound as a yellow gum (74 mg, 90%). LCMS (Method A) R_{T}=8.03 min, M+H⁺=441.

### Step 4: 1-(4-(3-Phenylfuro[2,3-b]pyridin-2-yl)phenyl)cyclobutanamine

tert-butyl (1-(4-(3-Phenylfuro[2,3-b]pyridin-2-yl)phenyl)cyclobutyl)carbamate (36 mg, 0.08 mmol) in DCM (2 mL) was charged TFA (0.2 mL). The reaction was then stirred at RT for 1.5 h. The solvent was removed in vacuo and the residue neutralised using aq.NaHCO₃. This was then extracted twice with ethyl acetate. The organic layers were combined, dried (MgSO₄), filtered and concentrated in vacuo. The crude material was purified by flash chromatography (SiO₂, gradient 0 to 10% methanol in dichoromethane) to afford the title compound (5 mg, 18%). LCMS (Method A) R_{T}=4.25 min, M+2H⁺=342. ¹H NMR (500 MHz, CDCl₃): δ 8.34 (1H, dd), 7.82 (1H, dd), 7.71-7.69 (2H, m), 7.51-7.49 (4H, m), 7.45-7.43 (1H, m), 7.39-7.37 (2H, m), 7.24-7.22 (1H, m), 2.59-2.25 (2H, m), 2.19-2.14 (2H, m), 2.12-2.05 (1H, m), 1.81-1.72 (1H, m).

### Example 20

### 1-(4-(5-Phenylfuro[2,3-d]pyrimidin-6-yl)phenyl)cyclobutanamine

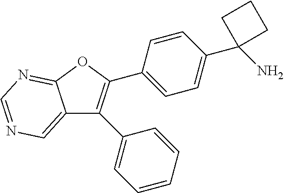

Step 5-Phenylfuro[2,3-d]pyrimidine: 4-Chloro-5-phenylfuro[2,3-d]pyrimidine was prepared according to a literature procedure [WO 2006/004658: compound 18]. 4-Chloro-5-phenylfuro[2,3-d]pyrimidine (240 mg, 1.04 mmol) was dissolved in anhydrous MeOH (5.0 mL) and 10% Pd—C (7.2 mg, 3% by wt.) was added. The mixture was degassed 3 times, flushing with H₂. Et₃N (174 μL, 1.25 mmol) was added and the temperature increased to 40° C. After 16 hours, the reaction mixture was filtered through Celite®, washing with MeOH and the solvents were removed in vacuo to give the title compound (ca. 200 mg) as a yellow solid that was carried through to the next step without further purification. ¹H NMR (400 MHz, CDCl₃): δ 9.30 (s, 1H), 9.06 (s, 1H), 7.93 (s, 1H), 7.68-7.63 (m, 2H), 7.56-7.50 (m, 2H), 7.48-7.42 (m, 1H).

### Step 2: 6-Iodo-5-phenylfuro[2,3-d]pyrimidine

5-Phenylfuro[2,3-d]pyrimidine (156 mg, 0.793 mmol) and N-iodosuccinimide (268 mg, 1.19 mmol) were stirred in anhydrous MeCN (3.0 mL) at reflux under an atmosphere of N₂. After 16 hours, a further equivalent of N-iodosuccinimide (176 mg, 0.793 mmol) was added due to incomplete reaction. After a further 6 hours, the reaction mixture was partitioned between EtOAc (5.0 mL) and 10% Na₂S₂O₅ (aq) solution (5.0 mL). The resulting biphasic mixture was separated, the aqueous layer extracted using EtOAc (3×5.0 mL), the combined organic phase dried (Na₂SO₄) and the solvents were removed in vacuo. The remaining residue was purified by flash chromatography (SiO₂, 0→20%, EtOAc in n-hexane) to give the title compound (47.5 mg, 19%) as a white solid and recovered starting material, 5-phenylfuro[2,3-d]pyrimidine (75.4 mg, 48%). ¹H NMR (400 MHz, CDCl₃): δ 9.09-8.94 (m, 2H), 7.70-7.61 (m, 2H), 7.76-7.46 (m, 3H).

### Step 3: tert-Butyl (1-(4-(5-phenylfuro[2,3-d]pyrimidin-6-yl)phenyl)cyclobutyl)carbamate

Pd(PPh₃)₄ (8.5 mg, 7.37 μmol) was added to a pre-degassed stirred solution of 6-iodo-5-phenylfuro[2,3-d]pyrimidine (47.5 mg, 0.148 mmol), K₃PO₄ (93.9 mg, 0.443 mmol) and tert-butyl(1-(4-(4,4,5,5-tetramethyl-1,3,2-dioxaborolan-2-yl)phenyl)cyclobutyl)carbamate (82.6 mg, 0.221 mmol) in 4:1, DMF/H₂O (2.5 mL). The temperature was increased to 80° C. After 3 hours, the reaction mixture was partitioned between EtOAc (5.0 mL) and H₂O (5.0 mL). The resulting biphasic mixture was separated, the aqueous layer extracted using EtOAc (2×5.0 mL), the combined organic phase was washed using brine (3×5.0 mL), dried (Na₂SO₄) and the solvents were removed in vacuo. The remaining residue was purified by flash chromatography (SiO₂, 0→20%, EtOAc in n-hexane) to give the title compound (61.0 mg, 94%) as a colourless oil. ¹H NMR (400 MHz, CDCl₃): δ 9.01 (s, 1H), 8.91 (s, 1H), 7.70 (d, 2H), 7.55-7.38 (m, 7H), 5.19 (s, 1H), 2.60-2.40 (m, 4H), 2.19-2.06 (m, 1H), 1.95-1.82 (m, 1H), 1.49-1.13 (m, 9H).

### Step 4: 1-(4-(5-Phenylfuro[2,3-d]pyrimidin-6-yl)phenyl)cyclobutanamine

TFA (0.5 mL) was added to stirred solution of tert-butyl (1-(4-(5-phenylfuro[2,3-d]pyrimidin-6-yl)phenyl)cyclobutyl)carbamate (30.0 mg, 0.0680 mmol) in DCM (0.5 mL) at RT under an atmosphere of N₂. After 1 hour, analysis by TLC indicated complete reaction. The solvents were removed in vacuo and the remaining reside was partitioned between EtOAc (5.0 mL) and satd. NaHCO₃ (aq) solution (5.0 mL). The resulting biphasic mixture was separated, the aqueous layer extracted using EtOAc (3×3.0 mL), the combined organic phase was dried (Na₂SO₄) and the solvents were removed in vacuo. The remaining residue was purified by flash chromatography (SiO₂, 0→10%, MeOH in EtOAc) to give the title compound (20.0 mg, 86%) as a colourless oil that was freeze-dried to give a white fluffy solid. LCMS (Method A): R_{T}=3.79 min, M+2⁺=343.10. ¹H NMR (400 MHz, methanol-d₄): δ 8.95 (s, 1H), 8.92 (s, 1H), 7.68 (d, 2H), 7.58-7.46 (m, 7H), 2.62-2.49 (m, 2H), 2.32-2.20 (m, 2H), 2.16-2.03 (m, 1H), 1.83-1.70 (m, 1H).

### Example 21

### 1-(4-(5-Nitro-3-phenylfuro[2,3-b]pyridin-2-yl)phenyl)cyclobutanamine

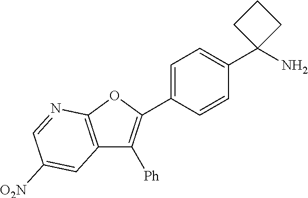

### Step 1: tert-Butyl (1-(4-((2-methoxy-5-nitropyridin-3-yl)ethynyl)phenyl)cyclobutyl) carbamate

3-Bromo-2-methoxy-5-nitropyridine (161 mg, 0.691 mmol) was stirred in 1:1, Et₃N/1,4-dioxane (1.5 mL) at RT and the reaction mixture degassed under an atmosphere of N₂. The temperature was reduced to 0° C. and Pd(^{t}Bu₃P)₂ (14.1 mg, 0.0276 mmol) was added. After 10 minutes, Cu(I)I (1.3 mg, 0.0092 mmol), followed by dropwise addition of tert-butyl (1-(4-ethynylphenyl)cyclobutyl)carbamate (125 mg, 0.461 mmol) in Et₃N (0.75 mL). After 4 hours, the solvents were removed in vacuo, coevaporating to remove residual Et₃N. The residue was dissolved in DCM, filtered through Celite®, washing with DCM. The solvents were removed in vacuo and the remaining residue was purified by flash chromatography (SiO₂, 0→25%, EtOAc in n-hexane) to give the title compound (127 mg, 65%) as a pale yellow solid. ¹H NMR (500 MHz, CDCl₃): δ 9.00 (d, 1H), 8.50 (d, 1H), 7.55 (d, 2H), 7.44 (d, 2H), 5.13 (s, 1H), 4.15 (s, 3H), 2.62-2.43 (m, 4H), 2.18-2.08 (m, 1H), 1.94-1.83 (m, 1H). 1.49-1.05 (m, 9H).

### Step 2: tert-Butyl (1-(4-(3-iodo-5-nitrofuro[2,3-b]pyridin-2-yl)phenyl)cyclobutyl) carbamate

A 1M solution of ICI in DCM (0.404 mL, 0.404 mmol) was added dropwise to a stirred solution of 1-(4-(5-nitro-3-phenylfuro[2,3-b]pyridin-2-yl)phenyl)cyclobutanamine (114 mg, 0.269 mmol) in DCM (2.5 mL) at 0° C. After 1 hour, the reaction mixture was quenched using satd. Na₂S₂O₃ (aq) solution. The resulting biphasic solution was separated, the aqueous layer was extracted using DCM (3×3.0 mL), the combined organic phase was dried (Na₂SO₄), and the solvents were removed in vacuo. The remaining residue was purified by flash chromatography (SiO₂, 0→20%, EtOAc in n-hexane) to give the title compound (130 mg, 80%) as a bright yellow solid. ¹H NMR (500 MHz, CDCl₃): δ 9.23 (d, 1H), 8.59 (d, 1H), 8.24 (d, 2H), 7.62 (d, 2H), 5.19 (s, 1H), 2.65-2.29 (m, 4H), 2.22-2.12 (m, 1H), 1.99-1.89 (m, 1H), 1.51-1.11 (m, 9H).

### Step 3: tert-Butyl (1-(4-(5-nitro-3-phenylfuro[2,3-b]pyridin-2-yl)phenyl)cyclobutyl) carbamate

tert-Butyl (1-(4-(3-iodo-5-nitrofuro[2,3-b]pyridin-2-yl)phenyl)cyclobutyl) carbamate (130 mg, 0.242 mmol) was dissolved in anhydrous DME (4.0 mL) and the resulting solution degassed under an atmosphere of N₂. Pd(OAc)₂ (8.2 mg, 0.0121 mmol), PPh₃ (9.5 mg, 0.0363 mmol), PhB(OH)₂ (44.3 mg, 0.364 mmol) and CsF (173 mg, 1.14 mmol) were added and the resulting mixture was heated to 75° C. After 16 hours, analysis by TLC indicated complete reaction. The solvents were removed in vacuo and the resulting residue was partitioned between DCM (10 mL) and brine (10 mL). The resulting biphasic mixture was separated, the aqueous layer extracted using DCM (2×5.0 mL), the combined organic phase was dried (Na₂SO₄), and the solvents were removed in vacuo. The remaining residue was purified by flash chromatography (SiO₂, 0→20%, EtOAc in n-hexane) to give the title compound (114 mg, 97%) as a yellow solid.

LCMS (Method A): R_{T}=8.30 min, M+H⁺=486.1.

### Step 4: 1-(4-(5-Nitro-3-phenylfuro[2,3-b]pyridin-2-yl)phenyl)cyclobutanamine bis HCl salt

tert-Butyl (1-(4-(5-nitro-3-phenylfuro[2,3-b]pyridin-2-yl)phenyl)cyclobutyl)carbamate (10.0 mg, 0.0206 mmol) was dissolved in diethyl ether (0.5 mL) and a 4 M solution of HCl in 1,4-dioxane (0.5 mL) added. After 2 hours, the precipitate was filtered off to give the title compound (3.0 mg, 32%) as a yellow solid. LCMS (Method A): R_{T}=8.31 min, M−NH₃+H⁺=369.0. ¹H NMR (500 MHz, methanol-d₄): δ 9.26 (d, 1H), 8.64 (d, 1H), 7.85-7.81 (m, 2H), 7.61-7.52 (m, 7H), 2.83-2.75 (m, 2H), 2.66-2.58 (m, 2H), 2.30-2.20 (m, 1H), 2.04-1.94 (m, 1H).

### Example 22

### 2-(4-(1-Aminocyclobutyl)phenyl)-3-phenylfuro[2,3-b]pyridin-5-amine

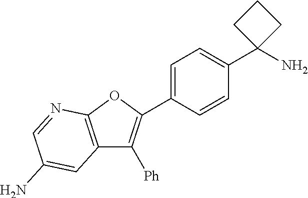

### Step 1: tert-Butyl (1-(4-(5-amino-3-phenylfuro[2,3-b]pyridin-2-yl)phenyl)cyclobutyl) carbamate

SnCl₂.2H₂O (92.9, 0.412 mmol) was added to a stirred solution of tert-butyl (1-(4-(5-nitro-3-phenylfuro[2,3-b]pyridin-2-yl)phenyl)cyclobutyl)carbamate (40.0 mg, 0.0824 mmol) in 4:1, DMF/AcOH (2.5 mL) at 80° C. After 30 minutes, a solution of NaOH (500 mg) in H₂O (5.0 mL) was added. After cooling to RT, DCM (5.0 mL) was added, the resulting biphasic mixture separated, the aqueous layer extracted using DCM (2×5.0 mL), the combined organic phase was dried (Na₂SO₄), and the solvents were removed in vacuo. The remaining residue was purified by flash chromatography (SiO₂, 0->50%, EtOAc in n-hexane) to give the title compound (25.9 mg, 69%). LCMS (Method A): R_{T}=6.85 min, M+H⁺=456.1.

### Step 2: 2-(4-(1-Aminocyclobutyl)phenyl)-3-phenylfuro[2,3-b]pyridin-5-amine tris HCl salt

TFA (0.5 mL) was added to a stirred solution of tert-butyl (1-(4-(5-amino-3-phenylfuro[2,3-b]pyridin-2-yl)phenyl)cyclobutyl)carbamate (1.5 mg, 0.0033 mmol) in DCM (0.5 mL) at RT under an atmosphere of N₂. After 30 minutes, analysis by LCMS showed complete conversion. The solvents were removed in vacuo and the remaining residue was partitioned between EtOAc (2.0 mL) and satd. NaHCO₃ (aq) solution (2.0 mL). The resulting biphasic mixture was separated, the aqueous layer extracted using EtOAc (2×2.0 mL), the combined organic phase was dried (Na₂SO₄) and the solvents were removed in vacuo. The remaining residue was dissolved in MeOH (0.25 mL) and a 2 M solution of HCl in diethyl ether added. After 30 minutes, the solvents were removed in vacuo to give the title compound (1.0 mg, 65%) as a pale yellow solid. LCMS (Method A): R_{T}=3.69 min, M+2⁺=357.14. ¹H NMR (500 MHz, methanol-d₄): δ 8.40 (d, 1H), 7.93 (d, 1H), 7.84-7.80 (m, 2H), 7.60-7.50 (m, 7H), 2.83-2.74 (m, 2H), 2.66-2.57 (m, 2H), 2.29-2.20 (m, 1H), 2.01-1.92 (m, 1H).

### Example 23

### 1-(4-(2-Phenylfuro[3,2-b]pyridin-3-yl)phenyl)cyclobutanamine

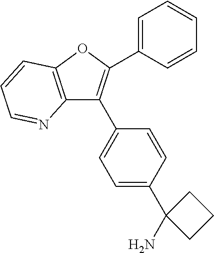

### Step 1: 3-Methoxy-2-(phenylethynyl)pyridine

2-Iodo-3-methoxypyridine (192 mg, 0.816 mmol) was stirred in 2:1, DMF/diisopropylamine (4.5 mL) and the resulting solution degassed under an atmosphere of N₂. PdCl₂(PPh₃)₂ (28.6 mg, 0.0408 mmol) and Cu(I)I (3.9 mg, 0.0204 mmol) were added, followed by phenylacetylene (108 μL, 0.979 mmol). The reaction vessel was sealed and heated to 120° C. for 10 minutes using microwave conditions. The reaction mixture was partitioned between EtOAc (5.0 mL) and brine (5.0 mL), the resulting biphasic mixture separated, the organic phase washed using further brine (2×5.0 mL) to remove residual DMF, filtered/dried using a phase separator (Isolute® SPE), and the solvents removed in vacuo. The remaining residue was purified by flash chromatography (SiO₂, 0→20%, EtOAc in n-hexane) to give the title compound (150 mg, 88%) as a yellow oil. ¹H NMR (500 MHz, CDCl₃): δ 8.24-8.22 (m, 1H), 7.66-7.61 (m, 2H), 7.38-7.33 (m, 3H), 7.25-7.20 (m, 2H), 3.94 (s, 3H).

### Step 2: 3-Iodo-2-phenylfuro[3,2-b]pyridine

A 1M solution of ICI in DCM (1.03 mL, 1.03 mmol) was added dropwise to a stirred solution of 3-methoxy-2-(phenylethynyl)pyridine (143 mg, 0.685 mmol) in DCM (3.0 mL) at 0° C. After 4 hours, the reaction mixture was quenched using satd. Na₂S₂O₃ (aq) solution (3.0 mL). The resulting biphasic solution was separated using a phase separator (Isolute® SPE), washing using DCM (2×3.0 mL), and the solvents were removed in vacuo. The remaining residue was purified by flash chromatography (SiO₂, 0→15%, EtOAc in n-hexane) to give the title compound (189 mg, 86%) as an off-white solid. LCMS (Method A): R_{T}=6.36 min, M+H⁺=322.1.

### Step 3: tert-Butyl (1-(4-(2-phenylfuro[3,2-b]pyridin-3-yl)phenyl)cyclobutyl)carbamate

Pd(PPh₃)₄ (18.0 mg, 0.0156 mmol) was added to a stirred solution of 3-iodo-2-phenylfuro[3,2-b]pyridine (100 mg, 0.311 mmol), K₃PO₄ (198 mg, 0.934 mmol) and tert-butyl (1-(4-(4,4,5,5-tetramethyl-1,3,2-dioxaborolan-2-yl)phenyl)cyclobutyl)carbamate (174 mg, 0.467 mmol, prepared as described in WO2008/070016) in 4:1, DMF/H₂O (5.0 mL).

The reaction vessel was sealed and heated to 120° C. for 20 minutes using microwave conditions. The reaction mixture was partitioned between EtOAc (5.0 mL) and brine (5.0 mL), the resulting biphasic mixture separated, the organic phase washed using further brine (2×5.0 mL) to remove residual DMF, filtered/dried using a phase separator (Isolute® SPE), and the solvents removed in vacuo. The remaining residue was purified by flash chromatography (SiO₂, 0→20%, EtOAc in n-hexane) to give the title compound (88.5 mg, 65%) as a white solid. LCMS (Method A): R_{T}=7.87 min, M+H⁺=441.0.

### Step 4: 1-(4-(2-Phenylfuro[3,2-b]pyridin-3-yl)phenyl)cyclobutanamine bis HCl salt

A 4 M solution of HCl in 1,4-dioxane (1.0 mL) was added to a stirred solution of tert-butyl (1-(4-(2-phenylfuro[3,2-b]pyridin-3-yl)phenyl)cyclobutyl)carbamate (40.0 mg, 0.0908 mmol) in THF (0.5 mL) at RT. After 1 hour, a white precipitate had formed and Et₂O (1.0 mL) was added to encourage further precipitation. The solvents were decanted from the precipitate, which was washed several times using Et₂O (4×2.0 mL), decanting each time. The resulting residue was dried in vacuo to give the title compound (33.4 mg, 89%) as an off-white solid. LCMS (Method A): R_{T}=4.25 min, M+2⁺=342.2. ¹H NMR (500 MHz, methanol-d₄): δ 8.77 (d, 1H), 8.65 (dd, 1H), 7.92 (dd, 1H), 7.81-7.72 (m, 6H), 7.56-7.51 (m, 1H), 7.47-7.42 (m, 2H), 2.91-2.84 (m, 2H), 2.75-2.68 (m, 2H), 2.37-2.27 (m, 1H), 2.09-1.99 (m, 1H).

### Example 24

### 1-(4-(2-(Methylthio)-5-phenylfuro[2,3-d]pyrimidin-6-yl)phenyl)cyclobutanamine

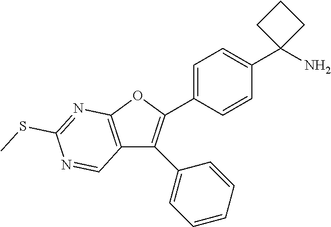

### Step 1: tert-Butyl (1-(4-(2-(methylthio)furo[2,3-d]pyrimidin-6-yl)phenyl)cyclobutyl) carbamate

5-Iodo-2-(methylthio)pyrimidin-4(1H)-one was prepared according to a literature procedure [Synthesis, 2003(7), p1039-1042]. 5-Iodo-2-(methylthio)pyrimidin-4(1H)-one (148 mg, 0.553 mmol) was stirred in 2:1, DMF/diisopropylamine (3.0 mL) and the resulting solution degassed under an atmosphere of N₂. Pd(^{t}Bu₃P)₂ (11.8 mg, 0.0230 mmol) and Cu(I)I (1.6 mg, 0.0115 mmol) were added, followed by tert-butyl (1-(4-ethynylphenyl)cyclobutyl)carbamate (125 mg, 0.461 mmol). The reaction vessel was sealed and heated to 120° C. for 10 minutes using microwave conditions. The reaction mixture was partitioned between EtOAc (5.0 mL) and brine (5.0 mL), the resulting biphasic mixture separated, the organic phase washed using further brine (2×5.0 mL) to remove residual DMF, filtered/dried using a phase separator (Isolute® SPE), and the solvents removed in vacuo. The remaining residue was purified by silica gel chromatography (0→20%, EtOAc in n-hexane) to give the title compound (68.4 mg, 36%) as an orange oil. LCMS (Method A): R_{T}=7.45 min, M+H⁺=412.1.

### Step 2: tert-Butyl (1-(4-(5-bromo-2-(methylthio)furo[2,3-d]pyrimidin-6-yl)phenyl)cyclobutyl)carbamate

N-Bromosuccinimide (35.5 mg, 0.119 mmol) was added to a stirred solution of tert-butyl (1-(4-(2-(methylthio)furo[2,3-d]pyrimidin-6-yl)phenyl)cyclobutyl)carbamate (68.4 mg, 0.166 mmol) in MeCN (1.0 mL) at 60° C. under an atmosphere of N₂. After 16 hours, the solvents were removed in vacuo and the remaining residue was purified by flash chromatography (SiO₂, 0→15%, EtOAc in n-hexane) to yield the title compound (13.0 mg, 17%) as a yellow solid. LCMS (Method A): R_{T}=8.21 min, M+H⁺=489.9, 491.9.

### Step 3: tert-Butyl (1-(4-(2-(methylthio)-5-phenylfuro[2,3-d]pyrimidin-6-yl)phenyl)cyclobutyl)carbamate

Pd(PPh₃)₄ (1.6 mg, 1.42 μmol) was added to a stirred solution of tert-butyl (1-(4-(5-bromo-2-(methylthio)furo[2,3-d]pyrimidin-6-yl)phenyl)cyclobutyl)carbamate (13.9 mg, 0.0283 mmol), K₃PO₄ (18.0 mg, 0.0850 mmol) and phenylboronic acid (5.2 mg, 0.0425 mmol) in 4:1, DMF/H₂O (1.25 mL). The reaction vessel was sealed and heated to 120° C. for 20 minutes using microwave conditions. The reaction mixture was partitioned between EtOAc (5.0 mL) and brine (5.0 mL), the resulting biphasic mixture separated, the organic phase washed using further brine (2×5.0 mL) to remove residual DMF, filtered/dried using a phase separator (Isolute® SPE), and the solvents removed in vacuo. The remaining residue was purified by flash chromatography (SiO₂, 0→20%, EtOAc in n-hexane) to give the title compound (6.4 mg, 46%) as a white colourless gum. ¹H NMR (500 MHz, CDCl₃): δ 8.67 (s, 1H), 7.65 (d, 2H), 7.51-7.43 (m, 5H), 7.39 (d, 2H), 5.07 (s, 1H), 2.68 (s, 3H), 2.57-2.38 (m, 4H), 2.16-2.06 (m, 1H), 1.92-1.82 (m, 1H), 1.48-1.12 (m, 9H).

### Step 4: 1-(4-(2-(Methylthio)-5-phenylfuro[2,3-d]pyrimidin-6-yl)phenyl)cyclobutanamine bis HCl salt

A 4 M solution of HCl in 1,4-dioxane (0.50 mL) was added to a stirred solution of tert-butyl (1-(4-(2-(methylthio)-5-phenylfuro[2,3-d]pyrimidin-6-yl)phenyl)cyclobutyl)carbamate (6.4 mg, 0.0131 mmol) in THF (0.25 mL) at RT. After 1 hour, a white precipitate had formed and Et₂O (0.50 mL) was added to encourage further precipitation. The solvents were decanted from the precipitate, which was washed several times using Et₂O (4×1.0 mL), decanting each time. The resulting residue was dried in vacuo to give the title compound (1.2 mg, 20%) as an off-white solid. LCMS (Method A): R_{T}=4.58 min, M+H⁺=389.2. ¹H NMR (500 MHz, methanol-d₄): δ 8.76 (s, 1H), 7.79 (d, 2H), 7.56-7.49 (m, 7H), 2.83-2.75 (m, 2H), 2.68 (s, 3H), 2.65-2.57 (m, 2H), 2.30-2.20 (m, 1H), 2.03-1.93 (m, 1H).

### Example 25

### 2-(4-(1-Aminocyclobutyl)phenyl)-3-phenylbenzofuran-6-carbonitrile

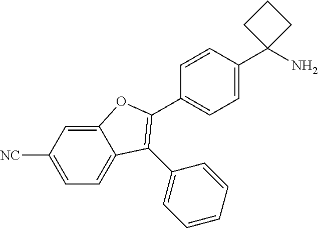

### Step 1: tert-Butyl (1-(4-((4-cyano-2-methoxyphenyl)ethynyl)phenyl)cyclobutyl)carbamate

To a solution of 4-bromo-3-methoxybenzonitrile (1.92 g, 7.075 mmol) in anhydrous triethylamine (8 mL) and 1,4-dioxane (8 mL) was added bis(tert-butylphosphine)palladium(0) (0.144 g, 0.283 mmol) and copper(I) iodide (13 mg, 0.094 mmol). The reaction mixture was then degassed with N₂ for 10 minutes before being cooled to 0° C. A solution of tert-butyl 1-(4-ethynylphenyl)cyclobutylcarbamate (1.00 g, 4.72 mmol) in TEA (8 mL) was added dropwise to the cooled reaction mixture. The reaction was allowed to warm to RT and stirred for 18 hours. The reaction mixture was concentrated in vacuo and the residue diluted with DCM (20 mL) and filtered through Celite. The resulting filtrate was concentrated in vacuo to afford a brown oil that was purified by silica gel chromatography (ethyl acetate/hexane gradient, 0-<50%) yielding the title compound as an off-white solid (890 mg, 47%). ¹H NMR (500 MHz, CDCl₃): δ 7.55-7.53 (m, 3H), 7.43 (d, 2H), 7.23 (d, 1H), 7.12 (s, 1H), 5.25 (s, 1H), 3.94 (s, 3H), 2.54-2.51 (m, 4H), 2.11-2.13 (m, 1H), 1.90-1.86 (m, 1H), 1.34-1.25 (br s, 9H).

### Step 2: tert-Butyl (1-(4-(6-cyano-3-iodobenzofuran-2-yl)phenyl)cyclobutyl)carbamate

To a solution of tert-butyl (1-(4-((4-cyano-2-ethoxyphenyl)ethynyl)phenyl)cyclobutyl)carbamate (890 mg, 2.21 mmol) in dichloromethane (20 mL) in a 50 mL round-bottomed flask was added iodine monochloride 1.0M in DCM (3.32 mL, 3.32 mmol) dropwise at 0° C. under N₂. The reaction was allowed to warm to room temperature and stirred for 30 minutes. The reaction was partitioned between DCM (5 mL) and a saturated solution of Na₂S₂O₆ (10 mL) and extracted. The organic layer was concentrated in vacuo affording an oil that was purified by silica gel chromatography (ethyl acetate/hexane gradient, 0/100→20/80) yielding the title compound as a beige solid (840 mg, 74%). ¹H NMR (500 MHz, CDCl₃) 8.09 (d, 2H), 7.69 (s, 1H), 7.52-7.48 (m, 3H), 7.43 (d, 1H), 5.19 (br s, 1H), 2.54-2.49 (br m, 4H), 2.10-2.07 (m, 1H), 1.86-1.84 (m, 1H), 1.21-1.17 (br s, 9H).

### Step 3: tert-Butyl (1-(4-(6-cyano-3-phenylbenzofuran-2-yl)phenyl)cyclobutyl)carbamate

To a solution of tert-butyl (1-(4-(6-cyano-3-iodobenzofuran-2-yl)phenyl)cyclobutyl)carbamate (300 mg, 0.583 mmol) in dry and degassed 1,2-dimethoxyethane (9.7 ml) was added phenylboronic acid (107 mg, 0.875 mmol), cesium fluoride (416 mg, 2.74 mmol), triphenylphosphine (23 mg, 0.087 mmol) and palladium(II) acetate (19 mg, 0.029 mmol). The reaction was heated to 75° C. for 5 hours. The reaction mixture was concentrated in vacuo and the residues were partitioned between dichloromethane and brine, extracted, dried over Na₂SO₄, filtered and concentrated in vacuo. The reaction mixture was purified by silica gel chromatography (ethyl acetate/hexane gradient, 5/95→30/70) affording the title compound as a pale yellow solid (247 mg, 91%). ¹H NMR (500 MHz, CDCl₃) 7.83 (s, 1H), 7.64 (d, 2H), 7.56-7.40 (m, 9H), 5.25 (br s, 1H), 2.53-2.50 (m, 4H), 2.15-2.05 (m, 1H), 1.95-1.85 (m, 1H), 1.45-1.30 (br s, 9H).

### Step 4: 2-(4-(1-Aminocyclobutyl)phenyl)-3-phenylbenzofuran-6-carbonitrile

To a solution of tert-butyl (1-(4-(6-cyano-3-phenylbenzofuran-2-yl)phenyl)cyclobutyl)carbamate (70 mg, 0.150 mmol) in DCM (3 mL) was added TFA (1 ml). The reaction was allowed to stir at RT for 2 hours. The reaction mixture was monitored and a large amount of starting material was still present. TFA (1 mL) was added and the reaction was stirred for 2 hours. The reaction mixture was then concentrated in vacuo. The residue was partitioned between DCM (10 mL) and a saturated solution of NaHCO₃ (10 mL). The organic layer was separated before purification by silica gel chromatography (gradient elution 5% MeOH in DCM) to yield the title compound as a white solid after drying by high vacuum (15 mg, 27%). ¹H NMR (500 MHz, CDCl₃) 7.77 (s, 1H), 7.56 (d, 2H), 7.47-7-19 (m, 9H), 2.50-2.44 (m, 2H), 2.13-2.10 (m, 2H), 2.09-1.93 (m, 1H), 1.93-1.73 (br s, 2H), 1.73-1.65 (m, 1H).

### Example 26

### 6-(4-(1-Aminocyclobutyl)phenyl)-4-methoxy-N,N-dimethyl-5-phenylfuro[2,3-d]pyrimidin-2-amine

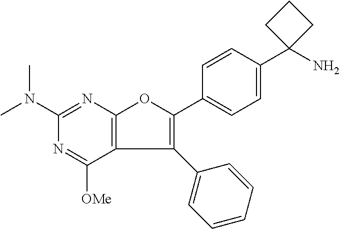

### Step 1: tert-Butyl (1-(4-(2-(dimethylamino)-4-methoxy-5-phenylfuro[2,3-d]pyrimidin-6-yl)phenyl)cyclobutyl)carbamate

A solution of tert-butyl (1-(4-(4-methoxy-2-(methylsulfonyl)-5-phenylfuro[2,3-d]pyrimidin-6-yl)phenyl)cyclobutyl)carbamate (100 mg, 0.182 mmol) and dimethylamine (2 M in tetrahydrofuran, 1.8 mL, 3.64 mmol) was heated at 80° C. for 48 h in a sealed tube. The reaction mixture was concentrated in vacuo and purified by flash chromatography (SiO₂, gradient 10 to 50% ethyl acetate in hexane) to afford the title compound as a white solid (82 mg, 88%). LCMS (Method A) R_{T}=9.10 min, M+H⁺=515.

### Step 2: 6-(4-(1-Aminocyclobutyl)phenyl)-4-methoxy-N,N-dimethyl-5-phenylfuro[2,3-d]pyrimidin-2-amine

To a solution of tert-butyl (1-(4-(4-methoxy-2-morpholino-5-phenylfuro[2,3-d]pyrimidin-6-yl)phenyl)cyclobutyl)carbamate (82 mg, 0.16 mmol) in DCM (2 mL) was charged TFA (0.2 mL). The reaction was then stirred at RT for 1 h. The solvent was removed in vacuo and the residue suspended in ethyl acetate and washed with a saturated solution of sodium hydrogen carbonate, dried over magnesium sulphate, filtered and concentrated in vacuo. The crude mixture was purified by flash chromatography (SiO₂, gradient 2 to 10% methanol and 1% triethylamine in dichloromethane) to afford the desired product as an off-white solid (41 mg, 62%). ¹H NMR (500 MHz, methanol-d₄): δ 7.30 (d, 2H), 7.25 (m, 5H), 7.18 (d, 2H), 3.72 (s, 3H), 3.09 (s, 6H), 2.37-2.34 (m, 2H), 2.09-2.14 (m, 2H), 1.91-1.98 (m, 1H), 1.59-1.66 (m, 1H).

LCMS (Method A) R_{T}=5.15 min, M+2H⁺=416.

### Example 27

### 1-(4-(2-(3-(Aminomethyl)azetidin-1-yl)-4-methoxy-5-phenylfuro[2,3-d]pyrimidin-6-yl)phenyl)cyclobutanamine

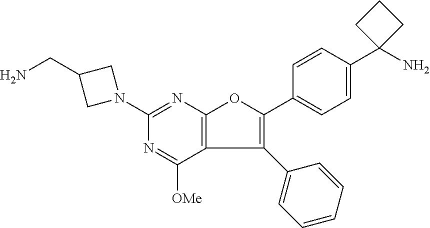

### Step 1: [1-(4-(2-[3-(tert-Butoxycarbonylamino-methyl)-azetidin-1-yl]-4-methoxy-5-phenyl-furo[2,3-d]pyrimidin-6-yl)-phenyl)-cyclobutyl]-carbamic acid tert-butyl ester

A solution of tert-butyl (1-(4-(4-methoxy-2-(methylsulfonyl)-5-phenylfuro[2,3-d]pyrimidin-6-yl)phenyl)cyclobutyl)carbamate (100 mg, 0.182 mmol) and tert-butyl (azetidin-3-ylmethyl)carbamate (339 mg, 1.82 mmol) in toluene (2 mL) was heated at 100° C. for 2 h in a sealed tube. The reaction mixture was concentrated in vacuo and purified by flash chromatography (SiO₂, gradient 10 to 50% ethyl acetate in hexane) to afford the title compound as a white gum (96 mg, 80%). LCMS (Method A) R_{T}=8.84 min, M+H⁺=656.

### Step 2: 1-(4-(2-(3-(Aminomethyl)azetidin-1-yl)-4-methoxy-5-phenylfuro[2,3-d]pyrimidin-6-yl)phenyl)cyclobutanamine

To a solution of tert-butyl (1-(4-(4-methoxy-2-morpholino-5-phenylfuro[2,3-d]pyrimidin-6-yl)phenyl)cyclobutyl)carbamate (96 mg, 0.17 mmol) in DCM (2 mL) was charged TFA (0.2 mL). The reaction was stirred at RT for 1 h. The solvent was removed in vacuo and the residue suspended in ethyl acetate and washed with a saturated solution of sodium hydrogen carbonate, dried over magnesium sulphate, filtered and concentrated in vacuo. The crude mixture was purified by flash chromatography (SiO₂, gradient 2 to 20% methanol and 1% triethylamine in dichloromethane) to afford the desired product as an off-white solid (40 mg, 56%). ¹H NMR (500 MHz, methanol-d₄): δ 7.50 (d, 2H), 7.39-7.41 (m, 7H), 4.33 (dd, 2H), 3.94 (dd, 2H), 3.89 (s, 3H), 3.33 (d, 2H), 3.01-3.05 (m, 1H), 2.62-2.68 (m, 2H), 2.45-2.50 (m, 2H), 2.16-2.22 (m, 1H), 1.81-1.86 (m, 1H). LCMS (Method A) R_{T}=4.02 min, M+H⁺=456.

### Example 28

### 1-(4-(5-Morpholino-3-phenylfuro[2,3-b]pyridin-2-yl)phenyl)cyclobutanamine

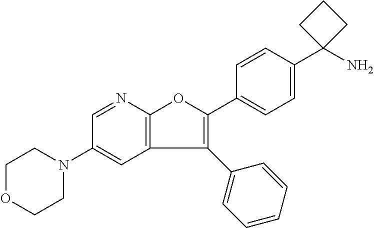

### Step 1: tert-Butyl (1-(4-(5-morpholino-3-phenylfuro[2,3-b]pyridin-2-yl)phenyl)cyclobutyl)carbamate

To a solution of tert-butyl (1-(4-(5-bromo-3-phenylfuro[2,3-b]pyridin-2-yl)phenyl)cyclobutyl)carbamate (106 mg, 0.204 mmol) in toluene (2 mL) in a Schlenk, was added palladium(II)acetate (0.5 mg, 0.002 mmol), 2,8,9-triisopropyl-2,5,8,9-tetraaza-1-phosphabicyclo[3.3.3]undecane (1.23 mg, 4.08 μmol) and morpholine (0.021 ml, 0.241 mmol). The solution was degassed for 10 minutes and stirred for 16 hours at 100° C. before the reaction mixture was suspended in dichloromethane and washed with brine, dried over magnesium sulphate, filtered and concentrated in vacuo. The resulting residue was purified by silica gel chromatography (gradient 10 to 25% EtOAc in hexane) to give the title compound as a white solid (62 mg, 58%). LCMS (Method A) R_{T}=8.11 min, M+H⁺=526.

### Step 2: 1-(4-(5-Morpholino-3-phenylfuro[2,3-b]pyridin-2-yl)phenyl)cyclobutanamine

To a solution of tert-butyl (1-(4-(5-morpholino-3-phenylfuro[2,3-b]pyridin-2-yl)phenyl)cyclobutyl)carbamate (40 mg, 0.076 mmol) in tetrahydrofuran (0.5 mL) was added 4M HCl in 1,4-dioxane (1 mL). The reaction mixture was stirred for 16 hours at room temperature before being suspended in ethyl acetate and washed with a saturated solution of sodium hydrogen carbonate, dried over magnesium sulphate, filtered and concentrated in vacuo. The resulting residue was purified by silica gel chromatography (dichloromethane with 5% MeOH and 1% triethylamine) to give the product as an off-white solid (21 mg, 65%). ¹H-NMR (500 MHz, CDCl₃) δ 7.99 (d, 1H), 7.58 (d, 2H), 7.35-7.44 (m, 4H), 7.22 (d, 1H), 7.19 (d, 2H), 3.78-3.82 (m, 2H), 3.08-3.12 (m, 2H), 2.42-2.49 (m, 2H), 2.15-2.20 (m, 2H), 2.06-2.12 (m, 1H), 1.61-1.66 (m, 1H). LCMS (Method A) R_{T}=4.22 min, M+2H⁺=427.

### Example 29

### 2-[4-(1-Amino-cyclobutyl)-phenyl]-3-phenyl-chromen-4-one hydrogen chloride*

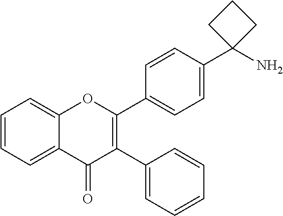

### Step 1: (1-{-4-[3-Hydroxy-3-(2-methoxy-phenyl)-prop-1-ynyl]-phenyl}-cyclobutyl)-carbamic acid tert-butyl ester*

To a solution of [1-(4-ethynyl-phenyl)-cyclobutyl]-carbamic acid tert-butyl ester (91 mg, 0.34 mmol) in anhydrous THF (1.5 ml) at −78° C. was added n-butyl lithium solution (2.5 M in hexane, 0.30 ml, 0.75 mmol) and the reaction mixture was stirred at −78° C. After 30 minutes, a solution of 2-methoxy-benzaldehyde (46 mg, 0.34 mmol) in anhydrous THF (1.5 ml), was added dropwise. The reaction mixture was stirred at −78° C. for 45 minutes and was then allowed to reach RT. After 45 minutes, the reaction mixture was quenched with saturated ammonium chloride solution (3 mL) and was then extracted with EtOAc (20 mL). The organic extract was dried (Na₂SO₄), filtered and concentrated in vacuo. The resultant residue was subjected to flash chromatography (SiO₂, gradient 0 to 40% ethyl acetate in cyclohexane) to afford the title compound as a yellow solid (81 mg, 59%). ¹H NMR (400 MHz, CDCl₃): δ 7.65 (1H, dd, J=7.5 and 1.7 Hz), 7.52-7.42 (2H, m), 7.40-7.30 (3H, m), 7.01 (1H, dt, J=7.6 and 1.1 Hz), 6.94 (1H, dd, J=8.2 and 0.8 Hz), 5.93 (1H, br s), 5.05 (1H, s), 3.93 (3H, s), 3.03 (1H, br s), 2.60-2.36 (4H, m), 2.16-2.03 (1H, m), 1.91-1.78 (1H, m), 1.35 (9H, br s).

### Step 2: (1-{4-[3-(2-Methoxy-phenyl)-3-oxo-prop-1-ynyl]-phenyl}-cyclobutyl)-carbamic acid tert-butyl ester*

To a solution of (1-{-4-3-hydroxy-3-(2-methoxy-phenyl)-prop-1-ynyl]-phenyl}-cyclobutyl)-carbamic acid tert-butyl ester (110 mg, 0.27 mmol) in DCM (2 ml) was added manganese dioxide (235 mg, 2.70 mmol) and the reaction mixture was stirred at RT. After 18 h, the reaction mixture was filtered through Celite and the eluent was concentrated in vacuo to afford the title compound as a yellow solid (109 mg, 100%). ¹H NMR (400 MHz, CDCl₃): δ 8.01 (1H, dd, J=7.8 and 1.8 Hz), 7.56-7.51 (2H, m), 7.50-7.44 (1H, m), 7.42-7.35 (2H, m), 7.02-6.93 (2H, m), 5.06 (1H, s), 3.90 (3H, s), 2.53-2.24 (4H, m), 2.12-1.99 (1H, m), 1.88-1.74 (1H, m), 1.28 (9H, br s).

### Step 3: (1-[4-(3-Iodo-4-oxo-4H-chromen-2-yl)-phenyl]-cyclobutyl)-carbamic acid tert-butyl ester

A solution of (1-{4-[3-(2-methoxy-phenyl)-3-oxo-prop-1-ynyl]-phenyl}-cyclobutyl)-carbamic acid tert-butyl ester (109 mg, 0.27 mmol) in DCM (2 mL) was cooled to −78° C. A solution of iodine monochloride (1M in DCM, 0.41 mL, 0.41 mmol) was added dropwise over 5 minutes. The reaction mixture was allowed to reach RT and was stirred for 3 h. After this time, the reaction mixture was diluted using DCM (5 mL), washed with 10% sodium thiosulphate solution (5 mL), water (5 mL), brine (5 mL), dried (Na₂SO₄), filtered and concentrated in vacuo. The resultant residue was subjected to flash chromatography (SiO₂, gradient 0 to 10% methanol in DCM) to afford the title compound as a white solid (10 mg, 7%). LCMS (Method A): R_{T}=4.19 min, M+H⁺=518.

### Step 4: {1-[4-(4-Oxo-3-phenyl-4H-chromen-2-yl)-phenyl]-cyclobutyl}-carbamic acid tert-butyl ester*

To a solution of {1-[4-(3-iodo-4-oxo-4H-chromen-2-yl)-phenyl]-cyclobutyl}-carbamic acid tert-butyl ester (10 mg, 19 μmol), phenylboronic acid (5.0 mg, 39 μmol) and sodium carbonate (7.0 mg, 67 μmol) in DME (0.40 mL) and water (0.10 ml) contained in a microwave vial, was added tetrakis(triphenylphosphine)palladium(0) (3.0 mg, 2 μmol). The reaction mixture was purged using argon and was then heated in a microwave reactor at 130° C. for 30 min. The reaction mixture was partitioned between water (0.5 mL) and EtOAc (3 mL) and the aqueous phase was further extracted with ethyl acetate (3 mL). The combined organic phases were washed with brine (3 mL), dried (Na₂SO₄), filtered and concentrated in vacuo to afford the title compound as a brown solid (9 mg, 100%). LCMS (Method A): R_{T}=4.80 min, M+H⁺=468.

### Step 5: 2-[4-(1-Amino-cyclobutyl)-phenyl]-3-phenyl-chromen-4-one hydrogen chloride*

To a solution of {1-[4-(4-oxo-3-phenyl-4H-chromen-2-yl)-phenyl]-cyclobutyl}-carbamic acid tert-butyl ester (9.0 mg, 19 μmol) in DCM (1.0 mL) was added TFA (0.25 mL). The reaction mixture was stirred at RT for 1 h and then loaded directly onto an SCX cartridge (2 g, pre-equilibrated with DCM). The cartridge was washed with MeOH:DCM (1:1) and then the product was eluted using NH₃ solution (2M in MeOH). The appropriate fractions were concentrated in vacuo. The resultant residue was chromatographed on a 5 g C18 cartridge [gradient 0 to 100% MeOH in water+1M HCl (0.06 ml in each 10 ml of eluent)] to afford the title compound as a white solid (2.6 mg, 37%). LCMS (Method B): R_{T}=3.30 min, M+H⁺=368. ¹H NMR (400 MHz, DMSO-d₆): δ 8.63 (3H, s), 8.08 (1H, dd, J=7.9 and 1.6 Hz), 7.86-7.80 (1H, m), 7.70 (1H, d, J=8.6 Hz), 7.54-7.43 (5H, m), 7.32-7.23 (3H, m), 7.20-7.16 (2H, m), 2.52-2.40 (4H, m), 2.17-2.05 (1H, m), 1.80-1.67 (1H, m).

### Example 30

### 2-(4-(3-Aminoazetidin-3-yl)phenyl)-3-phenylbenzofuran-7-carbonitrile

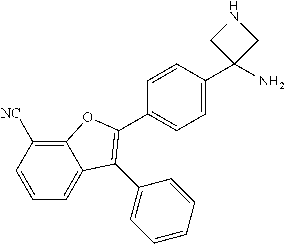

### Step 1: tert-Butyl 3-((tert-butylsulfinyl)imino)azetidine-1-carboxylate

To a solution of 2-methylpropane-2-sulfinamide (1.94 g, 16.0 mmol) in DCE (50 ml) was added tert-butyl 3-oxoazetidine-1-carboxylate (2.5 g, 14.6 mmol) and Ti(OEt)₄ (3.4 ml, 16.0 mmol). The reaction mixture was heated to 80° C. for 20 h. After cooling to room temperature, the reaction mixture was diluted with DCM (150 ml), and poured into a stirring solution of sat. aq. NaHCO₃ (200 ml). After stirring for 5 min, the layers were separated, and the aqueous layer extracted with DCM (3×50 ml). The combined organics were washed (aq. NaHCO₃), dried over magnesium sulphate, filtered and concentrated in vacuo to give the title compound as a yellow solid (4.11 g, >100%). ¹H NMR (400 MHz, CDCl₃): δ 5.13 (br d, 1H), 5.01 (br d, 1H), 4.78 (br s, 2H), 1.48 (s, 9H), 1.28 (s, 9H).

### Step 2: tert-Butyl 3-(1,1-dimethylethylsulfinamido)-3-(4-((trimethylsilyl)ethynyl)phenyl)azetidine-1-carboxylate

A solution of ((4-bromophenyl)ethynyl)trimethylsilane (210 mg, 0.83 mmol) in THF (2 ml) was cooled to −78° C., and n-BuLi (0.7 ml, 1.6M in hex, 1.08 mmol) was added slowly. The reaction mixture was stirred at −78° C. for 20 min. Meanwhile, a solution of tert-butyl 3-((tert-butylsulfinyl)imino)azetidine-1-carboxylate (250 mg, 0.91 mmol) in THF (2 ml) was cooled to −78° C., and trimethylaluminium (0.5 ml, 2M in toluene, 1.00 mmol) was added. This solution was then added slowly to the first solution. After stirring at −78° C. for 1 h, the reaction mixture was slowly warmed to 0° C. over 2 h. The reaction was quenched with aq. NH₄Cl (5 ml), extracted with DCM (2×10 ml), and concentrated in vacuo. The resultant residue was subjected to flash chromatography (SiO₂, gradient 0 to 100% EtOAc in hexanes) to afford the title compound as a yellow foam (121 mg, 33%).

¹H NMR (500 MHz, CDCl₃): δ 7.36 (d, 2H), 7.17 (d, 2H), 4.34 (d, 1H), 4.25 (d, 1H), 4.18 (m, 2H), 3.70 (br s, 1H), 1.30 (s, 9H), 1.04 (s, 9H), 0.12 (s, 9H).

### Step 3: tert-Butyl 3-(1,1-dimethylethylsulfinamido)-3-(4-ethynylphenyl)azetidine-1-carboxylate

To a solution of tert-butyl 3-(1,1-dimethylethylsulfinamido)-3-(4-((trimethylsilyl)ethynyl)phenyl)azetidine-1-carboxylate (270 mg, 0.60 mmol) in methanol (5 ml) was added potassium carbonate (124 mg, 0.90 mmol). The suspension was stirred at room temperature for 4 h. Water (5 ml) was added, and the mixture was extracted with DCM (3×10 ml). The combined organics were concentrated in vacuo to give the title compound as a pale yellow oil (250 mg, >100%). ¹H NMR (500 MHz, CDCl₃): δ 7.37 (d, 2H), 7.19 (d, 2H), 4.35 (d, 1H), 4.22 (m, 3H), 3.70 (br s, 1H), 1.30 (s, 9H), 1.08 (s, 9H).

### Step 4: tert-Butyl 3-(4-((3-cyano-2-methoxyphenyl)ethynyl)phenyl)-3-(1,1-dimethylethylsulfinamido)azetidine-1-carboxylate

Following the procedure of tert-butyl (1-(4-((4-methoxypyridin-3-yl)ethynyl)phenyl)cyclobutyl)carbamate, 3-bromo-2-methoxybenzonitrile (190 mg, 0.90 mmol) was reacted to afford the title compound (190 mg, 62%). ¹H NMR (500 MHz, CDCl₃): δ 7.72 (dd, 1H), 7.59 (m, 3H), 7.42 (m, 2H), 7.17 (t, 1H), 4.52 (d, 1H), 4.39 (m, 3H), 4.26 (s, 3H), 3.90 (br s, 1H), 1.48 (s, 9H), 1.23 (s, 9H).

### Step 5: tert-Butyl 3-((tert-butoxycarbonyl)amino)-3-(4-(7-cyano-3-phenylbenzofuran-2-yl)phenyl)azetidine-1-carboxylate

Following the procedure of tert-butyl (1-(4-(3-iodofuro[3,2-c]pyridin-2-yl)phenyl)cyclobutyl)carbamate, tert-butyl 3-(4-((3-cyano-2-methoxyphenyl)ethynyl)phenyl)-3-(1,1-dimethylethylsulfinamido)azetidine-1-carboxylate (0.19 g, 0.37 mmol) was reacted to afford an intermediate tert-butyl 3-amino-3-(4-(7-cyano-3-iodobenzofuran-2-yl)phenyl)azetidine-1-carboxylate (89 mg). This was dissolved in anhydrous DME (2.5 ml), and degassed with N₂ for 10 min. Palladium(II)acetate (6 mg, 0.0086 mmol), triphenylphosphine (7 mg, 0.0258 mmol), phenyl boronic acid (32 mg, 0.259 mmol) and cesium fluoride (122 mg, 0.808 mmol) were added sequentially, and the reaction mixture was heated to 75° C. for 18 h. The reaction mixture was then concentrated in vacuo. The residue was dissolved in 1,4-dioxane (2 ml), and triethylamine (48 μl, 0.344 mmol) and di-tert-butyl dicarbonate (57 mg, 0.259 mmol) were added. The reaction mixture was stirred at room temperature for 4 h, then concentrated in vacuo. The resultant residue was subjected to flash chromatography (SiO₂, gradient 0 to 50% EtOAc in hexanes) to afford the title compound (17 mg, 17%). ¹H NMR (500 MHz, CDCl₃): δ 7.63 (m, 3H), 7.55 (dd, 1H), 7.41 (m, 5H), 7.34 (d, 2H), 7.24 (t, 1H), 5.15 (br s, 1H), 4.15 (m, 4H), 1.41 (s, 9H), 1.37 (br s, 9H).

### Step 6: 2-(4-(3-Aminoazetidin-3-yl)phenyl)-3-phenylbenzofuran-7-carbonitrile

Following the procedure of 2-(4-(1-aminocyclobutyl)phenyl)-3-phenylbenzofuran-5-carboxamide, tert-butyl 3-((tert-butoxycarbonyl)amino)-3-(4-(7-cyano-3-phenylbenzofuran-2-yl)phenyl)azetidine-1-carboxylate (17 mg, 0.03 mmol) was reacted to afford the title compound (7.4 mg, 67%). LCMS (Method A): R_{T}=3.74 min, M+H⁺=349. ¹H NMR (500 MHz, DMSO-d₆): δ 7.91 (d, 1H), 7.81 (d, 1H), 7.67 (br s, 1H), 7.51 (m, 9H), 3.80 (br s, 1H), 3.70 (br s, 1H), 3.61 (br s, 2H).

### Example 31

### 6-(4-(1-Aminocyclobutyl)phenyl)-4-methoxy-5-phenylfuro[2,3-d]pyrimidin-2-amine

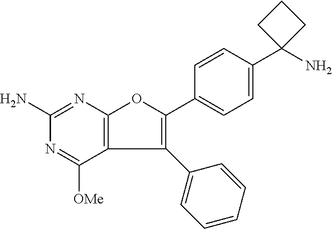

### Step 1: tert-Butyl (1-(4-(2-amino-4-methoxy-5-phenylfuro[2,3-d]pyrimidin-6-yl)phenyl)cyclobutyl)carbamate

A solution of tert-butyl (1-(4-(4-methoxy-2-(methylsulfonyl)-5-phenylfuro[2,3-d]pyrimidin-6-yl)phenyl)cyclobutyl)carbamate (100 mg, 0.182 mmol) and ammonium hydroxide (4 mL) was heated at 80° C. for 48 h in a sealed tube before being suspended in ethyl acetate and washed with a saturated solution of brine, dried over magnesium sulphate, filtered and concentrated in vacuo. The reaction mixture was purified by flash chromatography (SiO₂, gradient 10 to 50% ethyl acetate in hexane) to afford the title compound as a white solid (56 mg, 63%). LCMS (Method A) R_{T}=7.96 min, M+H⁺=487.

### Step 2: 6-(4-(1-Aminocyclobutyl)phenyl)-4-methoxy-5-phenylfuro[2,3-d]pyrimidin-2-amine

To a solution of tert-butyl (1-(4-(2-amino-4-methoxy-5-phenylfuro[2,3-d]pyrimidin-6-yl)phenyl)cyclobutyl)carbamate (56 mg, 0.12 mmol) in DCM (2 mL) was charged TFA (0.2 mL). The reaction was stirred at RT for 7 h. The mixture was neutralised with solid sodium hydrogen carbonate and concentrated in vacuo. The crude mixture was purified by flash chromatography (SiO₂, gradient 2 to 10% methanol and 1% triethylamine in dichloromethane) to afford the desired product as a white solid (18 mg, 40%). ¹H NMR (500 MHz, methanol-d₄): δ 7.42 (dd, 2H), 7.28-7.31 (m, 7H), 3.77 (s, 3H), 2.58-2.64 (m, 2H), 2.39-2.45 (m, 2H), 2.07-2.11 (m, 1H), 1.80-1.84 (m, 1H). LCMS (Method A) R_{T}=4.10 min, M+2H⁺=388.

### Example 32

### 6-(4-(1-Aminocyclobutyl)phenyl)-4-methoxy-N-methyl-5-phenylfuro[2,3-d]pyrimidin-2-amine

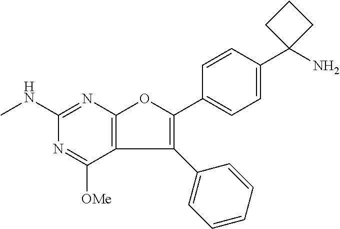

### Step 1: tert-Butyl (1-(4-(4-methoxy-2-(methylamino)-5-phenylfuro[2,3-d]pyrimidin-6-yl)phenyl)cyclobutyl)carbamate

A solution of tert-butyl (1-(4-(4-methoxy-2-(methylsulfonyl)-5-phenylfuro[2,3-d]pyrimidin-6-yl)phenyl)cyclobutyl)carbamate (50 mg, 0.09 mmol) and methylamine 2M in THF (1 mL, 2 mmol), was heated at 80° C. for 2 h in a sealed tube. The reaction mixture was concentrated in vacuo and purified by flash chromatography (SiO₂, gradient 0 to 20% 2M ammonia in methanol in dichloromethane) to afford the title compound as a colourless gum (27 mg, 60%). LCMS (Method A) R_{T}=8.11 min, M+H⁺=501.

### Step 2: 6-(4-(1-Aminocyclobutyl)phenyl)-4-methoxy-N-methyl-5-phenylfuro[2,3-d]pyrimidin-2-amine

To a solution of tert-butyl (1-(4-(4-methoxy-2-(methylamino)-5-phenylfuro[2,3-d]pyrimidin-6-yl)phenyl)cyclobutyl)carbamate (27 mg, 0.05 mmol) in DCM (2 mL) was charged TFA (0.2 mL). The reaction was stirred at RT for 1 h. The solvent was removed in vacuo and the residue neutralised using saturated aq. NaHCO₃. DCM was added and the resulting biphasic solution was separated using a phase separator (Isolute® SPE). The organic layer was concentrated in vacuo and the crude material purified by recrystallisation from methanol to afford the desired product as a pale yellow solid (4 mg, 19%). LCMS (Method A) R_{T}=4.49 min, MS=100% M−NH₃⁺=384, 50% M+2H⁺=402. ¹H NMR (500 MHz, methanol-d₄): δ 7.49-7.36 (9H, m), 3.90 (3H, s), 3.00 (3H, s), 2.58-2.53 (2H, m), 2.29-2.23 (2H, m), 2.12-2.06 (1H, m), 1.81-1.72 (1H, m).

### Example 33

### 1-(4-(2-(4-(2-(Dimethylamino)ethyl)piperazin-1-yl)-4-methoxy-5-phenylfuro[2,3-d]pyrimidin-6-yl)phenyl)cyclobutanamine

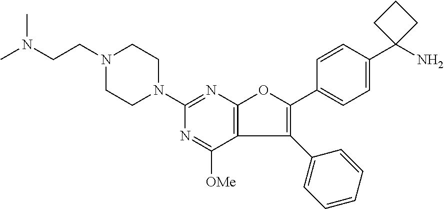

### Step 1: tert-Butyl (1-(4-(2-(4-(2-(dimethylamino)ethyl)piperazin-1-yl)-4-methoxy-5-phenylfuro[2,3-d]pyrimidin-6-yl)phenyl)cyclobutyl)carbamate

Following the procedure for tert-butyl (1-(4-(4-methoxy-2-morpholino-5-phenylfuro[2,3-d]pyrimidin-6-yl)phenyl)cyclobutyl)carbamate, tert-butyl (1-(4-(4-methoxy-2-(methylsulfonyl)-5-phenylfuro[2,3-d]pyrimidin-6-yl)phenyl)cyclobutyl)carbamate (50 mg, 0.091 mmol) was reacted with N,N-dimethyl-2-(piperazin-1-yl)ethanamine to provide the title compound as an orange solid (34 mg, 60%). LCMS (Method A) R_{T}=5.11, M+H⁺=627.

### Step 2: 1-(4-(2-(4-(2-(Dimethylamino)ethyl)piperazin-1-yl)-4-methoxy-5-phenylfuro[2,3-d]pyrimidin-6-yl)phenyl)cyclobutanamine

Following the procedure for 1-(4-(4-methoxy-2-morpholino-5-phenylfuro[2,3-d]pyrimidin-6-yl)phenyl)cyclobutanamine, tert-butyl (1-(4-(2-(4-(2-(dimethylamino)ethyl)piperazin-1-yl)-4-methoxy-5-phenylfuro[2,3-d]pyrimidin-6-yl)phenyl)cyclobutyl)carbamate (34 mg, 0.034 mmol) was reacted to give the title compound (8 mg, 28%). LCMS (Method A) R_{T}=3.51, M+H⁺=527. ¹H NMR (500 MHz, CDCl₃): δ 7.39-7.48 (m, 4H), 7.26-7.35 (m, 3H), 7.19 (m, 2H).

### Example 34

### 6-(4-(1-Aminocyclobutyl)phenyl)-4-methoxy-N-(2-(4-methylpiperazin-1-yl)ethyl)-5-phenylfuro[2,3-d]pyrimidin-2-amine

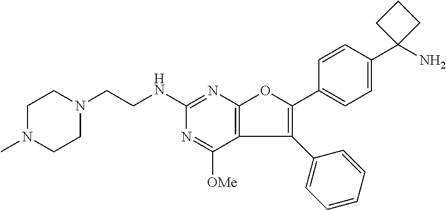

### Step 1: tert-Butyl (1-(4-(4-methoxy-2-((2-(4-methylpiperazin-1-yl)ethyl)amino)-5-phenylfuro[2,3-d]pyrimidin-6-yl)phenyl)cyclobutyl)carbamate

Following the procedure for tert-butyl (1-(4-(4-methoxy-2-morpholino-5-phenylfuro[2,3-d]pyrimidin-6-yl)phenyl)cyclobutyl)carbamate, tert-butyl (1-(4-(4-methoxy-2-(methylsulfonyl)-5-phenylfuro[2,3-d]pyrimidin-6-yl)phenyl)cyclobutyl)carbamate (50 mg, 0.091 mmol) was reacted with 2-(4-methylpiperazin-1-yl)ethanamine to provide the title compound as an orange solid (41 mg, 74%). LCMS (Method A) R_{T}=4.99, M+H⁺=613.

### Step 2: 6-(4-(1-Aminocyclobutyl)phenyl)-4-methoxy-N-(2-(4-methylpiperazin-1-yl)ethyl)-5-phenylfuro[2,3-d]pyrimidin-2-amine

Following the procedure for 1-(4-(4-methoxy-2-morpholino-5-phenylfuro[2,3-d]pyrimidin-6-yl)phenyl)cyclobutanamine, tert-butyl (1-(4-(4-methoxy-2-((2-(4-methylpiperazin-1-yl)ethyl)amino)-5-phenylfuro[2,3-d]pyrimidin-6-yl)phenyl)cyclobutyl)carbamate (41 mg, 0.067 mmol) was reacted to give the title compound (10.2 mg, 30%). LCMS (Method A) R_{T}=3.39, M+H⁺=513. ¹H NMR (500 MHz, CDCl₃): δ 7.45-7.52 (m, 4H), 7.35-7.45 (m, 3H), 7.3 (m, 2H), 5.6 (m, 1H), 3.9 (s, 3H), 3.6 (m, 2H), 2.5-2.7 (m, 7H), 2.37 (s, 3H), 1.95-2.2 (m, 6H), 1.7-1.8 (m, 1H).

### Example 35

### (S)-1-(6-(4-(1-Aminocyclobutyl)phenyl)-4-methoxy-5-phenylfuro[2,3-d]pyrimidin-2-yl)pyrrolidin-3-ol

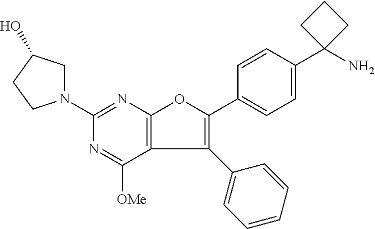

### Step 1: (S)-tert-Butyl (1-(4-(2-(3-hydroxypyrrolidin-1-yl)-4-methoxy-5-phenylfuro[2,3-d]pyrimidin-6-yl)phenyl)cyclobutyl)carbamate

Following the procedure for tert-butyl (1-(4-(4-methoxy-2-morpholino-5-phenylfuro[2,3-d]pyrimidin-6-yl)phenyl)cyclobutyl)carbamate, tert-butyl (1-(4-(4-methoxy-2-(methylsulfonyl)-5-phenylfuro[2,3-d]pyrimidin-6-yl)phenyl)cyclobutyl)carbamate (75 mg, 0.136 mmol) was reacted with (S)-pyrrolidin-3-ol to provide the title compound as an off-solid (41 mg, 54%). LCMS (Method A) R_{T}=7.81, M+H⁺=557.

### Step 2: (S)-1-(6-(4-(1-Aminocyclobutyl)phenyl)-4-methoxy-5-phenylfuro[2,3-d]pyrimidin-2-yl)pyrrolidin-3-ol

Following the procedure for 1-(4-(4-methoxy-2-morpholino-5-phenylfuro[2,3-d]pyrimidin-6-yl)phenyl)cyclobutanamine, (S)-tert-butyl (1-(4-(2-(3-hydroxypyrrolidin-1-yl)-4-methoxy-5-phenylfuro[2,3-d]pyrimidin-6-yl)phenyl)cyclobutyl)carbamate (41 mg, 0.074 mmol) was reacted to give the title compound (9.5 mg, 28%). LCMS (Method A) R_{T}=4.44, M−H₂O=440. ¹H NMR (500 MHz, DMSO-d₆): O 7.4-7.49 (m, 5H), 7.3-7.4 (m, 4H), 4.4 (br s, 1H), 3.85 (s, 3H), 3.5-3.72 (m, 3H), 2.3-2.39 (m, 2H), 1.85-2.1 (m, 6H), 1.58-1.69 (m, 1H).

### Example 36

### 2-((6-(4-(1-Aminocyclobutyl)phenyl)-4-methoxy-5-phenylfuro[2,3-d]pyrimidin-2-yl)oxy)ethanol

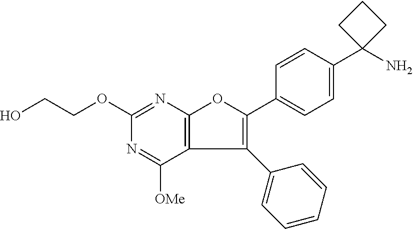

### Step 1: tert-Butyl (1-(4-(2-(2-hydroxyethoxy)-4-methoxy-5-phenylfuro[2,3-d]pyrimidin-6-yl)phenyl)cyclobutyl)carbamate

tert-Butyl (1-(4-(4-methoxy-2-(methylsulfonyl)-5-phenylfuro[2,3-d]pyrimidin-6-yl)phenyl)cyclobutyl)carbamate (50 mg, 0.091 mmol) was dissolved in a mixture of toluene (1.0 ml) and DMF (0.5 ml) before addition of ethylene glycol (0.1 ml, 1.82 mmol) and Hunig's base (0.048 ml, 0.27 mmol). The reaction mixture was then heated at 100° C. for 24 h before cooling to RT and diluting with DCM (10 ml) and brine (5 ml). The organic extracts were washed further with brine (2×5 ml) before concentration in vacuo affording an oil that was purified by silica gel chromatography (gradient elution 0 to 5% MeOH in DCM). This yielded the title compound as a yellow oil (22 mg, 46%). LCMS (Method A) R_{T}=7.27, M+H⁺=532.

### Step 2: 2-((6-(4-(1-Aminocyclobutyl)phenyl)-4-methoxy-5-phenylfuro[2,3-d]pyrimidin-2-yl)oxy)ethanol

Following the procedure for 1-(4-(4-methoxy-2-morpholino-5-phenylfuro[2,3-d]pyrimidin-6-yl)phenyl)cyclobutanamine, tert-butyl (1-(4-(2-(2-hydroxyethoxy)-4-methoxy-5-phenylfuro[2,3-d]pyrimidin-6-yl)phenyl)cyclobutyl)carbamate (22 mg, 0.041 mmol) was reacted to give the title compound (6 mg, 34%). LCMS (Method A) R_{T}=4.09, M+H⁺=433. ¹H NMR (500 MHz, CDCl₃): δ 7.3-7.5 (m, 7H), 7.26 (d, 2H), 4.5 (m, 2H), 3.97 (m, 2H), 3.9 (s, 3H), 2.4-2.5 (m, 2H), 2.05-2.12 (m, 2H), 1.95-2.04 (m, 1H), 1.55-1.7 (m, 4H).

### Example 37

### 1-(4-(4-Methoxy-5-phenyl-2-(piperazin-1-yl)furo[2,3-d]pyrimidin-6-yl)phenyl)cyclobutanamine

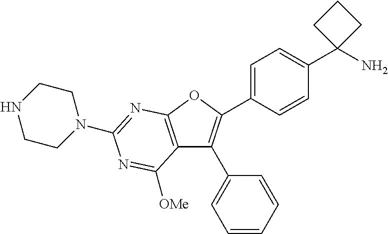

### Step 1: tert-Butyl (1-(4-(4-methoxy-5-phenyl-2-(piperazin-1-yl)furo[2,3-d]pyrimidin-6-yl)phenyl)cyclobutyl)carbamate

tert-Butyl (1-(4-(4-methoxy-2-(methylsulfonyl)-5-phenylfuro[2,3-d]pyrimidin-6-yl)phenyl)cyclobutyl)carbamate (100 mg, 0.181 mmol), and piperazine (312 mg, 3.62 mmol) were stirred in toluene (2.0 mL) at 100° C. in a sealed tube. After 16 hours, analysis by TLC indicated complete reaction. The reaction mixture was loaded directly onto a silica column and purified by flash chromatography (SiO₂, 0→20→40% EtOAc in n-hexane) to give the title compound (87.4 mg, 87%) as a pale yellow oil. LCMS (Method A): R_{T}=5.27 min, M+H⁺=556.20.

### Step 2: 1-(4-(4-Methoxy-5-phenyl-2-(piperazin-1-yl)furo[2,3-d]pyrimidin-6-yl)phenyl)cyclobutanamine bis HCl salt

A 4 M solution of HCl in 1,4-dioxane (1.00 mL) was added to a stirred solution of tert-butyl (1-(4-(4-methoxy-5-phenyl-2-(piperazin-1-yl)furo[2,3-d]pyrimidin-6-yl)phenyl)cyclobutyl)carbamate (49.1 mg, 0.0884 mmol) in THF (0.50 mL) at RT. After 1 hour, a white precipitate had formed and Et₂O (0.50 mL) was added to encourage further precipitation. The resulting suspension was triturated several times using Et₂O (4×1.0 mL). The resulting residue was dried in vacuo to give the title compound (38.1 mg, 82%) as a pale yellow solid. LCMS (Method X): R_{T}=3.65 min, M+2⁺=457.14. ¹H NMR (500 MHz, methanol-d₄): δ 7.59-7.36 (m, 9H), 4.19-4.10 (m, 4H), 3.92 (s, 3H), 3.36-3.27 (m, 4H), 2.79-2.71 (m, 2H), 2.62-2.53 (m, 2H), 2.27-2.17 (m, 1H), 2.00-1.90 (m, 1H).

### Example 38

### 2-((6-(4-(1-Aminocyclobutyl)phenyl)-4-methoxy-5-phenylfuro[2,3-d]pyrimidin-2-yl)amino)ethanol

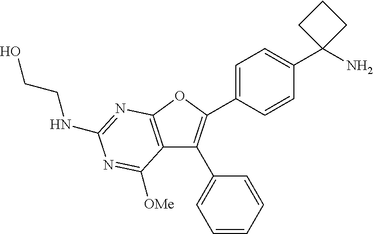

### Step 1: tert-Butyl (1-(4-(2-((2-hydroxyethyl)amino)-4-methoxy-5-phenylfuro[2,3-d]pyrimidin-6-yl)phenyl)cyclobutyl)carbamate

tert-butyl (1-(4-(4-methoxy-2-(methylsulfonyl)-5-phenylfuro[2,3-d]pyrimidin-6-yl)phenyl)cyclobutyl)carbamate (100 mg, 0.181 mmol), and 2-aminoethanol (218 μL, 3.62 mmol) were stirred in toluene (2.0 mL) at 100° C. in a sealed tube. After 16 hours, analysis by TLC indicated complete reaction. The reaction mixture was loaded directly onto a silica column and purified by flash chromatography (SiO₂, 0 to 60% EtOAc in n-hexane) to give the title compound (87.4 mg, 91%) as a pale yellow oil. LCMS (Method A): R_{T}=7.23 min, M+H⁺=531.14.

### Step 2: 2-((6-(4-(1-Aminocyclobutyl)phenyl)-4-methoxy-5-phenylfuro[2,3-d]pyrimidin-2-yl)amino)ethanol

A 4 M solution of HCl in 1,4-dioxane (1.00 mL) was added to a stirred solution of tert-butyl (1-(4-(2-((2-hydroxyethyl)amino)-4-methoxy-5-phenylfuro[2,3-d]pyrimidin-6-yl)phenyl)cyclobutyl)carbamate (35.7 mg, 0.0673 mmol) in THF (0.50 mL) at RT. After 1 hour, the solvents were removed in vacuo and the remaining residue partitioned between DCM (5.0 mL) and satd. NaHCO₃ (aq) solution (5.0 mL). The layers were separated using a phase separator (Isolute® SPE), washed using DCM (3×1.0 mL) and the solvents removed in vacuo. The remaining residue was purified by flash chromatography (SiO₂, 0 to 20% MeOH+1% 7N NH₃/MeOH in EtOAc) to give the title compound (12.5 mg, 43%) as a pale yellow solid. LCMS (Method A): R_{T}=4.14 min, M−NH₃+H⁺=414.16. ¹H NMR (500 MHz, methanol-d₄): δ 7.48-7.31 (m, 9H), 3.87 (s, 3H), 3.76 (t, 2H), 3.57 (t, 2H), 2.58-2.49 (m, 2H), 2.29-2.20 (m, 2H), 2.11-2.01 (m, 1H), 1.79-1.69 (m, 1H).

### Example 39

### 1-(6-(4-(1-Aminocyclobutyl)phenyl)-4-methoxy-5-phenylfuro[2,3-d]pyrimidin-2-yl)piperidin-4-amine

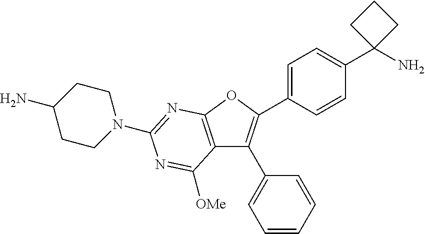

### Step 1: tert-Butyl (1-(4-(2-(4-N-Boc-aminopiperidin-1-yl)-4-methoxy-5-phenylfuro[2,3-d]pyrimidin-6-yl)phenyl)cyclobutyl)carbamate

tert-Butyl (1-(4-(4-methoxy-2-(methylsulfonyl)-5-phenylfuro[2,3-d]pyrimidin-6-yl)phenyl)cyclobutyl)carbamate (75.0 mg, 0.136 mmol), and 4-N-Boc-aminopiperidine (546 mg, 2.73 mmol) were stirred in toluene (2.0 mL) at 100° C. in a sealed tube. After 16 hours, analysis by TLC indicated complete reaction. The reaction mixture was loaded directly onto a silica column and purified by flash chromatography (SiO₂, 0 to 20% EtOAc in n-hexane) to give the title compound (79.7 mg, 87%) as a pale yellow solid, LCMS (Method A): R_{T}=9.11 min, M+H⁺=670.11.

### Step 2: 1-(6-(4-(1-Aminocyclobutyl)phenyl)-4-methoxy-5-phenylfuro[2,3-d]pyrimidin-2-yl)piperidin-4-amine

TFA (0.20 mL) was added to a stirred solution of tert-butyl (1-(4-(2-(4-N-Boc-aminopiperidin-1-yl)-4-methoxy-5-phenylfuro[2,3-d]pyrimidin-6-yl)phenyl)cyclobutyl)carbamate (79.7 mg, 0.170 mmol) in DCM (2.0 mL) at RT under an atmosphere of N₂. After 1 hour, analysis by LCMS showed complete conversion. The solvents were removed in vacuo and the remaining residue was partitioned between DCM (15 mL) and satd. NaHCO₃ (aq) solution (15 mL). [Note: insoluble material, therefore required sonication to dissolve material]. The resulting biphasic mixture was separated using a phase separator (Isolute® SPE), washed using DCM (3×1.0 mL) and the solvents removed in vacuo. The remaining residue was purified by flash chromatography (SiO₂, 0 to 20% MeOH+1% 7N NH₃/MeOH in EtOAc) to give the title compound (33.4 mg, 42%) as a pale yellow solid. LCMS (Method A): R_{T}=3.73 min, M+2⁺=471.16. ¹H NMR (500 MHz, methanol-d₄): δ 7.46-7.30 (m, 9H), 4.79-4.70 (m, 2H), 3.86 (s, 3H), 3.06-2.90 (m, 3H), 2.55-2.46 (m, 2H), 2.25-2.16 (m, 2H), 2.09-1.99 (m, 1H), 1.98-1.87 (m, 2H), 1.77-1.67 (m, 1H), 1.42-1.30 (m, 2H).

### Example 40

### 1-(6-(4-(1-Aminocyclobutyl)phenyl)-4-methoxy-5-phenylfuro[2,3-d]pyrimidin-2-yl)piperidin-4-ol

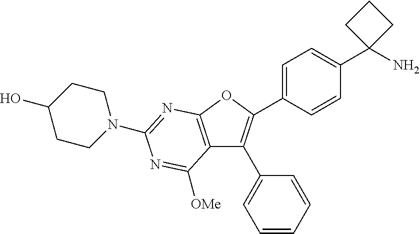

### Step 1: tert-Butyl (1-(4-(2-(4-hydroxypiperidin-1-A-4-methoxy-5-phenylfuro[2,3-d]pyrimidin-6-yl)phenyl)cyclobutyl)carbamate

tert-Butyl (1-(4-(4-methoxy-2-(methylsulfonyl)-5-phenylfuro[2,3-d]pyrimidin-6-yl)phenyl)cyclobutyl)carbamate (75.0 mg, 0.136 mmol), and 4-hydroxypiperidine (276 mg, 2.73 mmol) were stirred in toluene (2.0 mL) at 100° C. in a sealed tube. After 16 hours, analysis by TLC indicated complete reaction. The reaction mixture was loaded directly onto a silica column and purified by flash chromatography (SiO₂, 0 to 20 to 40% EtOAc in n-hexane) to give the title compound (58.8 mg, 76%) as a pale yellow oil. LCMS (Method A): R_{T}=7.96 min, M+H⁺=571.28.

### Step 2: 1-(6-(4-(1-Aminocyclobutyl)phenyl)-4-methoxy-5-phenylfuro[2,3-d]pyrimidin-2-yl)piperidin-4-ol

TFA (0.20 mL) was added to a stirred solution of tert-butyl (1-(4-(2-(4-hydroxypiperidin-1-yl)-4-methoxy-5-phenylfuro[2,3-d]pyrimidin-6-yl)phenyl)cyclobutyl)carbamate (58.8 mg, 0.103 mmol) in DCM (2.0 mL) at RT under an atmosphere of N₂. After 1 hour, analysis by LCMS showed complete conversion. The solvents were removed in vacuo and the remaining residue was partitioned between DCM (10 mL) and satd. NaHCO₃ (aq) solution (10 mL). The resulting biphasic mixture was separated using a phase separator (Isolute® SPE), washed using DCM (3×1.0 mL) and the solvents removed in vacuo. The remaining residue was purified by flash chromatography (SiO₂, 0 to 10% MeOH+1% 7N NH₃/MeOH in EtOAc) to give the title compound (19.3 mg, 40%) as a pale yellow solid. LCMS (Method A): R_{T}=4.55 min, M-NH₃+H⁺=454.22. ¹H NMR (500 MHz, methanol-d₄): δ 7.47-7.31 (m, 9H), 4.50-4.42 (m, 2H), 3.92-3.84 (m, 4H), 3.38-3.30 (m, 2H), 2.56-2.48 (m, 2H), 2.26-2.18 (m, 2H), 2.10-2.00 (m, 1H), 1.98-1.90 (m, 2H), 1.78-1.68 (m, 1H), 1.56-1.46 (m, 2H).

### Example 41

### 3-((6-(4-(1-Aminocyclobutyl)phenyl)-4-methoxy-5-phenylfuro[2,3-d]pyrimidin-2-yl)amino)propan-1-ol

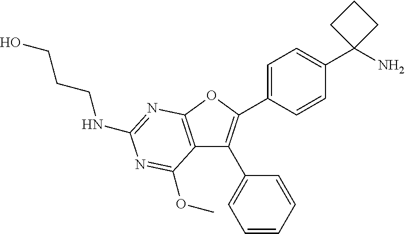

### Step 1: tert-Butyl (1-(4-(2-((3-hydroxypropyl)amino)-4-methoxy-5-phenylfuro[2,3-d]pyrimidin-6-yl)phenyl)cyclobutyl)carbamate

tert-Butyl (1-(4-(4-methoxy-2-(methylsulfonyl)-5-phenylfuro[2,3-d]pyrimidin-6-yl)phenyl)cyclobutyl)carbamate (75.0 mg, 0.136 mmol) and 3-aminopropanol (104 μL, 1.36 mmol) were stirred in DMF (2.0 mL) at 100° C. in a sealed tube under microwave conditions for 20 minutes (CEM Explorer/Discover). Analysis by TLC indicated complete reaction. The reaction mixture was loaded directly onto a silica column and purified by flash chromatography (SiO₂, 0 to 40% EtOAc in n-hexane) to give the title compound (58.2 mg, 79%) as a pale yellow oil. LCMS (Method A): R_{T}=7.38 min, M+H⁺=545.26.

### Step 2: 3-((6-(4-(1-Aminocyclobutyl)phenyl)-4-methoxy-5-phenylfuro[2,3-d]pyrimidin-2-yl)amino)propan-1-ol

TFA (0.20 mL) was added to a stirred solution of tert-butyl (1-(4-(2-((3-hydroxypropyl)amino)-4-methoxy-5-phenylfuro[2,3-d]pyrimidin-6-yl)phenyl)cyclobutyl)carbamate (58.2 mg, 0.107 mmol) in DCM (2.0 mL) at RT under an atmosphere of N₂. After 1 hour, analysis by LCMS showed complete conversion. The solvents were removed in vacuo and the remaining residue was partitioned between DCM (10 mL) and satd. NaHCO₃ (aq) solution (10 mL). The resulting biphasic mixture was separated using a phase separator (Isolute® SPE), washed using DCM (3×1.0 mL) and the solvents removed in vacuo. The remaining residue was purified by flash chromatography (SiO₂, 0 to 10% MeOH+1% 7N NH₃/MeOH in EtOAc) to give the title compound (40.2 mg, 85%) as a pale yellow solid. LCMS (Method A): R_{T}=4.26 min, M−NH₃+H⁺=428.19. ¹H NMR (500 MHz, methanol-d₄): δ 7.47-7.31 (m, 9H), 3.87 (s, 3H), 3.69 (t, 2H), 3.52 (t, 2H), 2.56-2.48 (m, 2H), 2.27-2.19 (m, 2H), 2.11-2.01 (m, 1H), 1.87 (dt, 2H), 1.78-1.68 (m, 1H).

### Example 42

### 6-(4-(1-Aminocyclobutyl)phenyl)-4-methoxy-N-(2-methoxyethyl)-5-phenylfuro[2,3-d]pyrimidin-2-amine

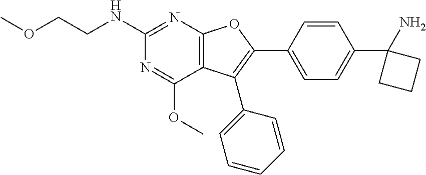

### Step 1: tert-Butyl 1-(4-(4-methoxy-2-(2-methoxyethylamino)-5-phenylfuro[2,3-d]pyrimidin-6-yl)phenyl)cyclobutylcarbamate

In a 10 ml microwave vial was added tert-butyl 1-(4-(4-methoxy-2-(methylsulfonyl)-5-phenylfuro[2,3-d]pyrimidin-6-yl)phenyl)cyclobutylcarbamate (75 mg, 0.136 mmol) and 2-methoxyethanamine (0.237 ml, 2.73 mmol) in dry DMF (1 ml). Reaction mixture was heated to 100° C. for 20 min under microwave heating. TLC analysis indicated reaction was complete. The reaction mixture was then dilted with DCM (15 ml) and water (10 ml). The organic extracts were then concentrated in vacuo affording an oil that was purified by silica gel chromatography (gradient elution 0 to 5% MeOH in DCM). This yielded the title compound as an orange/brown residue (56 mg, 75%). LCMS (Method A): R_{T}=8.18 min, M+H⁺=545.2.

### Step 2: 6-(4-(1-Aminocyclobutyl)phenyl)-4-methoxy-N-(2-methoxyethyl)-5-phenylfuro[2,3-d]pyrimidin-2-amine

To a solution of tert-butyl 1-(4-(4-methoxy-2-(2-methoxyethylamino)-5-phenylfuro[2,3-d]pyrimidin-6-yl)phenyl)cyclobutylcarbamate (56 mg, 0.103 mmol) in DCM (2 ml) in a 5 mL round-bottomed flask was added TFA (1 ml). The reaction was allowed to stir at RT for 2 h before analysis by TLC. The reaction was then concentrated in vacuo, diluted with DCM (10 ml) and sat. NaHCO₃ (5 ml) and extracted. DCM extracts were concentrated affording a residue that was purified by silica gel chromatography (gradient elution 0 to 5% MeOH in DCM). Purest fractions were combined and concentrated affording the title compound (21 mg, 46%) as an off white solid. LCMS (Method A): R_{T}=4.54 min, M−NH₃=428.

### Example 43

### N¹-(6-(4-(1-Aminocyclobutyl)phenyl)-4-methoxy-5-phenylfuro[2,3-d]pyrimidin-2-yl)ethane-1,2-diamine

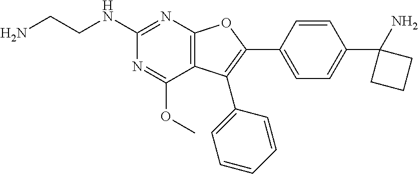

### Step 1: tert-Butyl 1-(4-(2-(2-aminoethylamino)-4-methoxy-5-phenylfuro[2,3-d]pyrimidin-6-yl)phenyl)cyclobutylcarbamate

In a 10 ml microwave vial was added tert-butyl 1-(4-(4-methoxy-2-(methylsulfonyl)-5-phenylfuro[2,3-d]pyrimidin-6-yl)phenyl)cyclobutylcarbamate (75 mg, 0.136 mmol) and ethane-1,2-diamine (0.456 ml, 6.82 mmol) in dry DMF (1 ml). The reaction mixture was then heated to 100° C. for 20 min in the microwave. TLC analysis indicated reaction was complete. Reaction mixture diluted with DCM (15 ml) and water (10 ml). Organic extracts were concentrated in vacuo affording an oil that was purified by silica gel chromatography (gradient elution 0 to 10% MeOH in DCM). This yielded the title compound as an orange/brown residue (63 mg, 87%). LCMS (Method A): R_{T}=5.12 min, M+H⁺=530.2.

### Step 2: N¹-(6-(4-(1-Aminocyclobutyl)phenyl)-4-methoxy-5-phenylfuro[2,3-d]pyrimidin-2-yl)ethane-1,2-diamine

To a solution of tert-butyl 1-(4-(2-(2-aminoethylamino)-4-methoxy-5-phenylfuro[2,3-d]pyrimidin-6-yl)phenyl)cyclobutylcarbamate (63 mg, 0.119 mmol) in DCM (2 ml) in a 5 mL round-bottomed flask was added TFA (1 ml, 12.98 mmol). The reaction was allowed to stir at RT for 2 h before analysis by TLC. The reaction mixture was concentrated in vacuo, diluted with DCM (10 ml) and sat. NaHCO₃ (5 ml) and extracted. DCM extracts were concentrated affording a residue that was purified by silica gel chromatography (gradient elution 0 to 15% 2M NH₃/MeOH in DCM). Purest fractions were combined and concentrated affording the title compound as an orange solid (19 mg, 37%). LCMS (Method A): R_{T}=3.41 min, M+H⁺=430.14.

### Example 44

### 1-(4-(2-(1H-Imidazol-1-yl)-4-methoxy-5-phenylfuro[2,3-d]pyrimidin-6-yl)phenyl)cyclobutanamine

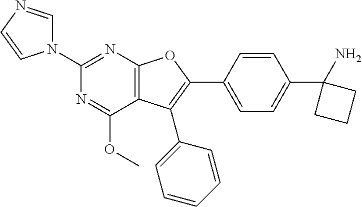

### Step 1: tert-Butyl 1-(4-(2-(1H-imidazol-1-yl)-4-methoxy-5-phenylfuro[2,3-d]pyrimidin-6-yl)phenyl)cyclobutylcarbamate

To a suspension of tert-butyl 1-(4-(4-methoxy-2-(methylsulfonyl)-5-phenylfuro[2,3-d]pyrimidin-6-yl)phenyl)cyclobutylcarbamate (0.1 g, 0.182 mmol) in trifluorotoluene (3 ml) in a 10 ml microwave vial was added imidazole (0.124 g, 1.82 mmol). The suspension was heated for 2 h at 95° C. The reaction mixture was then diluted with DCM, washed with water, separated and concentrated in vacuo affording the title compound as a brown oil (0.074 g, 76%). LCMS (Method A) R_{T}=6.77 min, M+H⁺=538.

### Step 2: 1-(4-(2-(1H-Imidazol-1-yl)-4-methoxy-5-phenylfuro[2,3-d]pyrimidin-6-yl)phenyl)cyclobutanamine

To a solution of tert-butyl 1-(4-(2-(1H-imidazol-1-yl)-4-methoxy-5-phenylfuro[2,3-d]pyrimidin-6-yl)phenyl)cyclobutylcarbamate (0.074 g, 0.138 mmol) in DCM (Volume: 3 ml) was added TFA (1 ml, 12.98 mmol). The solution was stirred at RT for 1 h. TLC analysis indicated that the reaction was complete. The reaction mixture was then concentrated in vacuo, diluted with DCM, washed with sat. NaHCO₃, separated and concentrated in vacuo affording a brown oil that was purified by silica gel chromatography (gradient 0 to 10% 1M NH₃/MeOH in DCM) affording 1-(4-(2-(1H-imidazol-1-yl)-4-methoxy-5-phenylfuro[2,3-d]pyrimidin-6-yl)phenyl)cyclobutanamine (0.021 g, 35%) as an off-white solid. LCMS (Method A) R_{T}=3.77 min, M+H⁺=438. ¹H NMR (500 MHz, CDCl₃): δ 8.65 (s, 1H), 7.94 (s, 1H), 7.55 (d, 2H), 7.4-7.5 (m, 5H), 7.35 (d, 2H), 7.18 (s, 1H), 4.04 (s, 3H), 2.48-2.58 (m, 2H), 2.05-2.2 (m, 3H), 1.8-1.95 (br s, 2H), 1.7-1.8 (m, 1H).

### Example 45

### 1-(4-(6-(4-(1-Aminocyclobutyl)phenyl)-4-methoxy-5-phenylfuro[2,3-d]pyrimidin-2-yl)piperazin-1-yl)ethanone

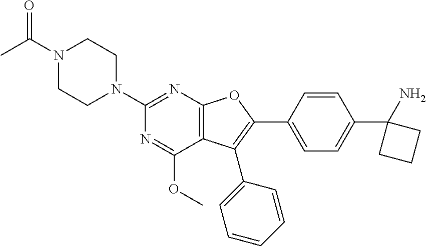

### Step 1: tert-Butyl 1-(4-(2-(4-acetylpiperazin-1-yl)-4-methoxy-5-phenylfuro[2,3-d]pyrimidin-6-3/1)phenyl)cyclobutylcarbamate

To a suspension of tert-butyl 1-(4-(4-methoxy-2-(methylsulfonyl)-5-phenylfuro[2,3-d]pyrimidin-6-yl)phenyl)cyclobutylcarbamate (0.1 g, 0.182 mmol) in trifluorotoluene (3 ml) in a 10 ml microwave vial was added 1-(piperazin-1-yl)ethanone (0.233 g, 1.819 mmol). The suspension was then heated in the microwave for 20 min at 120° C. TLC analysis indicated that the reaction was complete. The reaction mixture was then loaded directly onto a silica cartridge and purified (gradient elution 0 to 10% 1M NH₃/MeOH/DCM) affording the title compound as a beige solid (0.085 g, 78%).

LCMS (Method A) R_{T}=7.86 min, M+H⁺=598.2.

### Step 2: 1-(4-(6-(4-(1-Aminocyclobutyl)phenyl)-4-methoxy-5-phenylfuro[2,3-d]pyrimidin-2-yl)piperazin-1-yl)ethanone

To a solution of tert-butyl 1-(4-(2-(4-acetylpiperazin-1-yl)-4-methoxy-5-phenylfuro[2,3-d]pyrimidin-6-yl)phenyl)cyclobutylcarbamate (0.085 g, 0.142 mmol) in dichloromethane (3 ml) was added TFA (1 mL, 12.98 mmol). The reaction mixture was then stirred at RT for 1 h. The reaction mixture concentrated in vacuo, diluted with DCM/sat. NaHCO₃, extracted and concentrated. The residue was then purified by silica gel chromatography (gradient 0 to 5% 1M NH₃/MeOH in DCM) affording 1-(4-(6-(4-(1-aminocyclobutyl)phenyl)-4-methoxy-5-phenylfuro[2,3-d]pyrimidin-2-yl)piperazin-1-yl)ethanone (20 mg, 28%) as an off-white solid after drying under high vacuum. LCMS (Method A) R₁=4.47 min, M−NH₃+H⁺=481.2. ¹H NMR (500 MHz, CDCl₃): δ 7.45-7.55 (m, 4H), 7.33-7.44 (m, 3H), 7.26-7.32 (m, 2H), 3.85-3.95 (m, 8H), 3.71 (m, 2H), 3.55 (m, 2H), 2.5 (m, 2H). 2.19 (s, 3H), 2.0-2.18 (m, 3H), 1.7-1.8 (m, 1H).

### Example 46

### 1-(4-(4-Ethoxy-5-phenyl-2-(piperazin-14)furo[2,3-d]pyrimidin-6-yl)phenyl)cyclobutanamine, HCl

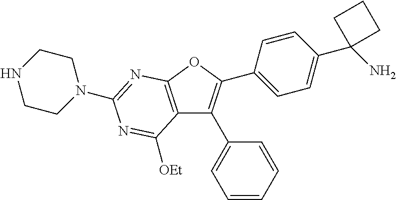

The title compound was isolated during the purification of 1-(4-(4-methoxy-5-phenyl-2-(piperazin-1-yl)furo[2,3-d]pyrimidin-6-yl)phenyl)cyclobutanamine [Example 37] by preparative HPLC (Method F). This by-product was determined to exist by the use of sodium methoxide contaminated with sodium ethoxide in an earlier step of the synthesis. The solvents were removed in vacuo and the remaining residue was treated with 2M HCl in diethyl ether. The solvents were removed in vacuo to give the title compound as a pale yellow solid. ¹H NMR (500 MHz, methanol-d₄): δ 7.59 (d, 2H), 7.48-7.39 (m, 7H), 4.41 (q, 2H), 4.14 (br s, 4H), 3.33 (br s, 4H), 2.79-2.71 (m, 2H), 2.63-2.55 (m, 2H), 2.28-2.18 (m, 1H), 2.00-1.90 (m, 1H), 1.23 (t, 3H). LCMS (Method A): R_{T}=3.76 min, M+2⁺=471.2.

### Example 47

### 1-(4-(4-Methoxy-5-phenyl-2-(piperidin-1-yl)furo[2,3-d]pyrimidin-6-yl)phenyl)cyclobutanamine

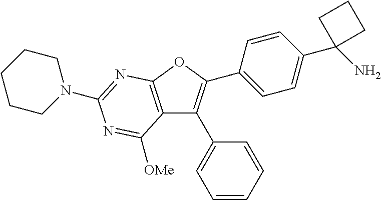

### Step 1: tert-Butyl (1-(4-(4-methoxy-5-phenyl-2-(piperidin-1-yl)furo[2,3-d]pyrimidin-6-yl)phenyl)cyclobutyl)carbamate

A solution of piperidine (135 μl, 1.37 mmol) and tert-butyl (1-(4-(4-methoxy-2-(methylsulfonyl)-5-phenylfuro[2,3-d]pyrimidin-6-yl)phenyl)cyclobutyl)carbamate (75.0 mg, 0.136 mmol) in DMF (1.0 ml) was heated in a sealed tube at 100° C. under microwave conditions for 20 minutes. Analysis by TLC showed complete consumption of starting material. The reaction was partitioned between EtOAc and brine, separated, dried (Phase Separator), and the solvents were removed in vacuo. The remaining residue was purified by flash chromatography (SiO₂, O to 20%, EtOAc in n-hexane) to give the title compound (53.8 mg, 71%) as a pale yellow oil. LCMS (Method A): R_{T}=9.63 min, M+H⁺=555.2.

### Step 2: 1-(4-(4-Methoxy-5-phenyl-2-(piperidin-1-yl)furo[2,3-d]pyrimidin-6-yl)phenyl)cyclobutanamine

Following the procedure used to prepare 1-(6-(4-(1-aminocyclobutyl)phenyl)-4-methoxy-5-phenylfuro[2,3-d]pyrimidin-2-yl)piperidin-4-amine, tert-butyl (1-(4-(4-methoxy-5-phenyl-2-(piperidin-1-yl)furo[2,3-d]pyrimidin-6-yl)phenyl)cyclobutyl)carbamate (53.8 mg, 0.097 mmol) was reacted and purified by flash chromatography (SiO₂, 0%, then 5% MeOH in EtOAc containing 1% 7N NH₃ in MeOH) to give the title compound (36.9 mg, 84%) as a pale yellow solid (free base). LCMS (Method A): R_{T}=5.53 min, M−NH₃+H⁺=438.2. ¹H NMR (500 MHz, CDCl₃): δ 7.54-7.46 (m, 4H), 7.42-7.35 (m, 3H), 7.29-7.24 (m, 2H), 3.87 (s, 3H), 3.87-3.81 (m, 4H), 2.55-2.47 (m, 2H), 2.16-2.00 (m, 3H), 1.79-1.52 (m, 7H).

### Example 48

### N-(6-(4-(1-Aminocyclobutyl)phenyl)-4-methoxy-5-phenylfuro[2,3-d]pyrimidin-2-yl)acetamide

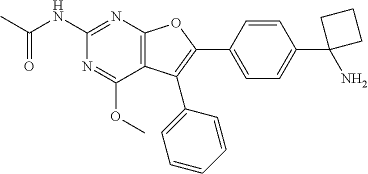

### Step 1: 6-(4-(1-Aminocyclobutyl)phenyl)-4-methoxy-5-phenylfuro[2,3-d]pyrimidin-2-amine

To a sealed tube was charged tert-butyl 1-(4-(4-methoxy-2-(methylsulfonyl)-5-phenylfuro[2,3-d]pyrimidin-6-yl)phenyl)cyclobutylcarbamate (200 mg, 0.364 mmol) 1,4-Dioxane (3 ml) and ammonium hydroxide (10 ml, 257 mmol). The reaction was then heated at 60° C. for 3 h. The reaction mixture was concentrated in vacuo and the residue was partitioned between water and ethyl acetate. The layers were separated and the organic layer dried (MgSO₄), filtered and concentrated in vacuo. The resultant residue was subjected to flash chromatography (SiO₂, gradient 0 to 100% ethyl acetate in hexane) to afford the title compound as a brown solid (74 mg, 42%). LCMS (Method A) R_{T}=7.46 min, M+H⁺=487.

### Step 2: tert-Butyl 1-(4-(2-acetamido-4-methoxy-5-phenylfuro[2,3-d]pyrimidin-6-yl)phenyl)cyclobutylcarbamate

To a solution of tert-butyl 1-(4-(2-amino-4-methoxy-5-phenylfuro[2,3-d]pyrimidin-6-yl)phenyl)cyclobutylcarbamate (58 mg, 0.119 mmol) in anhydrous pyridine (1 ml) at 0° C. was charged acetyl chloride (0.042 ml, 0.596 mmol) dropwise. On addition a white precipitate formed, the reaction was stirred for 1 h at 0° C. and allowed to warm to RT. The reaction was quenched with ice and the aqueous extracted with ethyl acetate. The combined organic extracts were dried (MgSO₄), filtered and concentrated in vacuo. The crude residue was purified by flash chromatography (SiO₂, gradient 0 to 40% ethyl acetate in hexane) to afford the title compound (33 mg, 52%) as a white solid. LCMS (Method A) R_{T}=7.44 min, M+H⁺=529.

### Step 3: N-(6-(4-(1-Aminocyclobutyl)phenyl)-4-methoxy-5-phenylfuro[2,3-d]pyrimidin-2-yl)acetamide

To a solution of tert-butyl 1-(4-(2-acetamido-4-methoxy-5-phenylfuro[2,3-d]pyrimidin-6-yl)phenyl)cyclobutylcarbamate (47 mg, 0.089 mmol) in DCM (2 ml) was charged TFA (2 mL, 26.0 mmol). The reaction was stirred at RT for 15 min. The reaction was concentrated in vacuo and the residue neutralised using aq NaHCO₃. To this was added DCM and the biphasic mixture separated through a phase separator. The solvent was removed in vacuo and the resulting white solid slurried in diethyl ether and dried. This yielded the title compound (22 mg, 58%) as a white solid. LCMS (Method A) R_{T}=4.15 min, M−NH₃+H⁺=412, M+2H⁺=430. ¹H NMR (500 MHz, CDCl₃): δ 7.94 (s, 1H), 7.53-7.55 (m, 2H), 7.43-7.50 (m, 5H), 7.34-7.35 (m, 2H), 3.97 (s, 3H), 2.66 (s, 3H), 2.52-2.57 (m, 2H), 2.05-2.21 (m, 3H), 1.71-1.82 (m, 1H).

### Example 49

### 1-(4-(2,4-Dimethoxy-5-phenylfuro[2,3-d]pyrimidin-6-yl)phenyl)cyclobutanamine

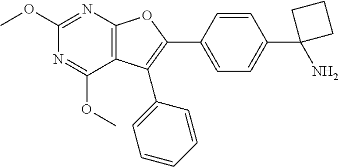

### Step 1: tert-Butyl (1-(4-(2,4-dimethoxy-5-phenylfuro[2,3-d]pyrimidin-6-yl)phenyl)cyclobutyl)carbamate

To a solution of tert-butyl (1-(4-(4-methoxy-2-(methylsulfonyl)-5-phenylfuro[2,3-d]pyrimidin-6-yl)phenyl)cyclobutyl)carbamate (75 mg, 0.14 mmol) in methanol (2 ml) was charged a solution of NaOMe in methanol (25%, 118 μl, 0.55 mmol). The reaction was heated at 70° C. until complete by LCMS. The reaction was concentrated in vacuo and the residue diluted with ethyl acetate and water. The layers were separated and the organic layer washed with brine, dried (MgSO₄), filtered and concentrated in vacuo. The crude material was purified by flash column chromatography (SiO₂, 0 to 50% ethyl acetate in hexane) to afford the title compound as an off white solid (36 mg, 53%). LCMS (Method A) R_{T}=8.24, M+H⁺=502.

### Step 2: 1-(4-(2,4-Dimethoxy-5-phenylfuro[2,3-d]pyrimidin-6-yl)phenyl)cyclobutanamine

To a solution of tert-butyl (1-(4-(2,4-dimethoxy-5-phenylfuro[2,3-d]pyrimidin-6-yl)phenyl)cyclobutyl)carbamate in DCM (2 ml) was charged TFA (0.25 ml). The reaction was then stirred at RT under a nitrogen atmosphere for 30 min. The solvent was removed in vacuo and the residue neutralised using aq. NaHCO₃. This mixture was extracted twice with ethyl acetate. The organic layers were combined, dried (MgSO₄), filtered and concentrated in vacuo. The crude material was purified by preparative HPLC (Method F) to afford the title compound (7 mg, 24%) as a white solid. LCMS (Method A) R_{T}=4.53 min, M−NH₃+H⁺=385, M+2H⁺=403. ¹H NMR (500 MHz, methanol-d₄): δ 7.61 (d, 2H), 7.45-7.46 (m, 7H), 4.09 (s, 3H), 3.99 (s, 3H), 2.72-2.78 (m, 2H), 2.52-2.58 (m, 2H), 2.18-2.26 (m, 1H), 1.91-1.99 (m, 1H).

### Example 50

### 1-(6-(4-(1-Aminocyclobutyl)phenyl)-5-phenylfuro[2,3-d]pyrimidin-2-yl)piperidin-4-amine

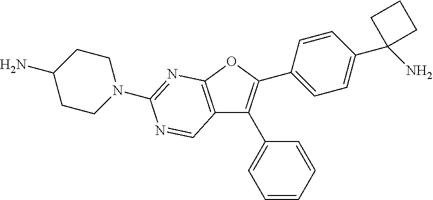

### Step 1: tert-Butyl N-[1-[4-[2-[4-(tert-butoxycarbonylamino)-1-piperidyl]-5-phenyl-furo[2,3-d]pyrimidin-6-yl]phenyl]cyclobutyl]carbamate*

A solution of 4-(N-Boc-amino)piperidine (215 mg, 1.07 mmol) and tert-butyl (1-(4-(2-(methylsulfonyl)-5-phenylfuro[2,3-d]pyrimidin-6-yl)phenyl)cyclobutyl)carbamate (55.7 mg, 0.107 mmol) in α,α,α-trifluorotoluene (2.0 ml) was heated in a sealed tube at 100° C. under microwave conditions for 20 minutes. Due to incomplete reaction (analysed by LCMS), the reaction was rerun twice at 120° C. under microwave conditions for 20 minutes, until almost no starting material was observed. The reaction mixture was loaded directly onto a column and purified by flash chromatography (SiO₂, 0-50%, EtOAc in n-hexane) to give the title compound (55.9 mg, 82%) as a white solid. LCMS (Method A): R_{T}=8.84 min, M+H⁺=640.1.

### Step 2. 1-(6-(4-(1-Aminocyclobutyl)phenyl)-5-phenylfuro[2,3-d]pyrimidin-2-yl)piperidin-4-amine bis HCl salt

4M HCl in 1,4-dioxane (1.5 ml) was added to a stirred solution of tert-butyl N-[1-[4-[2-[4-(tert-butoxycarbonylamino)-1-piperidyl]-5-phenyl-furo[2,3-d]pyrimidin-6-yl]phenyl]cyclobutyl]carbamate* (55.9 mg, 0.0874 mmol) in THF (0.5 ml) at RT under an atmosphere of nitrogen. After 1 hour, analysis by TLC showed no residual starting material. Diethyl ether (1.0 ml) was added and the precipitate that formed was triturated using further diethyl ether (5×2.0 ml) to remove impurities. The residual solvents were removed in vacuo to give the title compound (38.8 mg, 87%) as a pale yellow solid (bis HCl salt). ¹H NMR (500 MHz, methanol-d₄): δ 8.52 (s, 1H), 7.75-7.70 (m, 2H), 7.56-7.47 (m, 7H), 4.87 (br d, 2H) o/l with NMR solvent peak, 3.55-3.46 (m, 1H), 3.22 (t, 2H), 2.82-2.72 (m, 2H), 2.66-2.56 (m, 2H), 2.29-2.12 (m, 3H), 2.02-1.92 (m, 1H), 1.69 (ddt, 2H).

LCMS (Method A): R_{T}=3.48 min, M+2⁺=441.1

### Example 51

### 2-((6-(4-(1-Aminocyclobutyl)phenyl)-5-phenylfuro[2,3-d]pyrimidin-2-yl)amino)ethanol

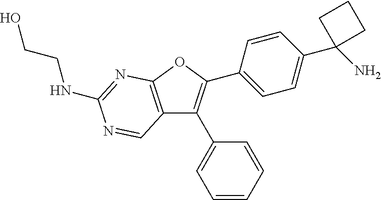

### Step 1: tert-Butyl (1-(4-(2-((2-hydroxyethyl)amino)-5-phenylfuro[2,3-d]pyrimidin-6-yl)phenyl)cyclobutyl)carbamate

2-Aminoethanol (0.058 ml, 0.962 mmol) was added to a solution of tert-butyl 1-(4-(2-(methylsulfonyl)-5-phenylfuro[2,3-d]pyrimidin-6-yl)phenyl)cyclobutylcarbamate (50.0 mg, 0.096 mmol) in a 10 ml microwave vial. The reaction mixture was heated under microwave conditions (CEM Explorer 24/Discover) at 150° C. for 20 minutes. Analysis by LCMS showed complete reaction. The solvents were removed in vacuo and the remaining residue was purified by flash chromatography (SiO₂, 0-75%, EtOAc in n-hexane) to give the title compound (41.8 mg, 87%) as a white solid.

LCMS (Method A): R_{T}=6.59 min, M+H⁺=501.2.

### Step 2: 2-((6-(4-(1-Aminocyclobutyl)phenyl)-5-phenylfuro[2,3-d]pyrimidin-2-yl)amino)ethanol, 2HCl

4M HCl in 1,4-dioxane (1.5 ml, 6.00 mmol) was added to a stirred solution of tert-butyl (1-(4-(2-((2-hydroxyethyl)amino)-5-phenylfuro[2,3-d]pyrimidin-6-yl)phenyl)cyclobutyl)carbamate (41.8 mg, 0.084 mmol) in THF (0.5 ml) at RT under an atmosphere of nitrogen. After 1 hour, diethyl ether (1.0 ml) was added and the precipitate that formed was triturated using further diethyl ether (5×3 ml) to remove impurities and the residual solvents were removed in vacuo to give the title compound (17.8 mg, 45%) as a pale yellow solid (bis HCl salt). ¹H NMR (500 MHz, methanol-d₄): δ 8.52 (s, 1H), 7.77-7.73 (m, 2H), 7.58-7.50 (m, 7H), 3.82 (t, 2H), 3.74-3.66 (m, 2H), 2.82-2.74 (m, 2H), 2.65-2.57 (m, 2H), 2.30-2.20 (m, 1H), 2.03-1.93 (m, 1H). LCMS (Method A): R_{T}=3.76 min, M+H⁺=401.2.

### Example 52

### 1-(4-(2-Morpholino-5-phenylfuro[2,3-d]pyrimidin-6-yl)phenyl)cyclobutanamine

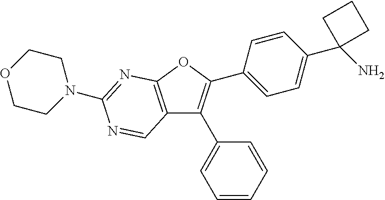

### Step 1: tert-Butyl (1-(4-(2-morpholino-5-phenylfuro[2,3-d]pyrimidin-6-yl)phenyl)cyclobutyl)carbamate

Morpholine (0.084 ml, 0.962 mmol) was added to a solution of tert-butyl 1-(4-(2-(methylsulfonyl)-5-phenylfuro[2,3-d]pyrimidin-6-yl)phenyl)cyclobutylcarbamate (50 mg, 0.096 mmol) in α,α,α-trifluorotoluene (1.0 ml) in a 10 mL microwave vial. The reaction mixture was heated under microwave conditions (CEM Explorer 24/Discover) at 150° C. for 20 min. Analysis by LCMS showed complete reaction. The reaction mixture was eluted directly onto a column, washing with some DCM and purified by flash chromatography (SiO₂, 0→20%, EtOAc in n-hexane) to give the title compound (49.1 mg, 97%) as a pale yellow oil. LCMS (Method A): R_{T}=8.33 min, M+H⁺=527.2.

### Step 2: 1-(4-(2-Morpholino-5-phenylfuro[2,3-d]pyrimidin-6-yl)phenyl)cyclobutanamine, 2HCl

4M HCl in 1,4-dioxane (1.5 ml, 6.00 mmol) was added dropwise to a stirred solution of tert-butyl (1-(4-(2-morpholino-5-phenylfuro[2,3-d]pyrimidin-6-yl)phenyl)cyclobutyl)carbamate (49.1 mg, 0.093 mmol) in tetrahydrofuran (0.5 ml) at RT under an atmosphere of nitrogen. After 1 hour, diethyl ether (1.0 ml) was added and the precipitate that formed was triturated using further diethyl ether (5×3 ml) to remove impurities and the residual solvents were removed in vacuo to give the title compound (44.7 mg, 96%) as a pale yellow solid (bis HCl salt). ¹H NMR (500 MHz, methanol-d₄): δ 8.51 (s, 1H), 7.73 (d, 2H), 7.57-7.47 (m, 7H), 3.90 (t, 4H), 3.83 (t, 4H), 2.83-2.73 (m, 2H), 2.66-2.56 (m, 2H), 2.30-2.20 (m, 1H), 2.02-1.92 (m, 1H). LCMS (Method A): R_{T}=4.56 min, M+H⁺=427.3.

### Example 53

### 1-(4-(5-Phenyl-2-(piperazin-1-yl)furo[2,3-d]pyrimidin-6-yl)phenyl)cyclobutanamine

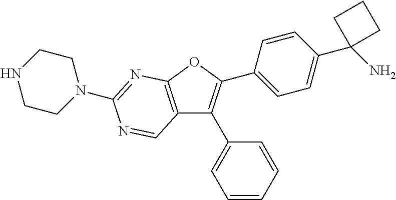

### Step 1: tert-Butyl (1-(4-(5-phenyl-2-(piperazin-1-yl)furo[2,3-d]pyrimidin-6-yl)phenyl)cyclobutyl)carbamate

Piperazine (83 mg, 0.962 mmol) was added to a solution of tert-butyl 1-(4-(2-(methylsulfonyl)-5-phenylfuro[2,3-d]pyrimidin-6-yl)phenyl)cyclobutylcarbamate (50.0 mg, 0.096 mmol) in α,α,α-trifluorotoluene (1.0 ml) in a 10 ml microwave vial. The reaction mixture was heated under microwave conditions (CEM Explorer 24/Discover) at 150° C. for 20 minutes. Analysis by LCMS showed no residual starting material. The reaction mixture was eluted directly onto a column, washing with some DCM and purified by flash chromatography (SiO₂, 0-6%, methanol in DCM) to give the title compound (46.2 mg, 91%) as a pale yellow oil. LCMS (Method A): R_{T}=5.14 min, M+H⁺=526.22.

### Step 2: 1-(4-(5-Phenyl-2-(piperazin-1-yl)furo[2,3-d]pyrimidin-6-yl)phenyl)cyclobutanamine, 2HCl

4 M HCl in 1,4-dioxane (1.5 ml, 6.00 mmol) was added to a stirred solution of tert-butyl 1-(4-(5-phenyl-2-(piperazin-1-yl)furo[2,3-d]pyrimidin-6-yl)phenyl)cyclobutylcarbamate (46.2 mg, 0.088 mmol) in tetrahydrofuran (0.5 ml) at RT under an atmosphere of nitrogen. After 1 hour, diethyl ether (1.0 ml) was added and the precipitate that formed was triturated using further diethyl ether (5×3 mL) to remove impurities. The solvents were removed in vacuo to give the title compound (34.1 mg, 78%) as a pale yellow solid (bis HCl salt). ¹H NMR (500 MHz, methanol-d₄): δ 8.59 (s, 1H), 7.74-7.70 (m, 2H), 7.56-7.46 (m, 7H), 4.19 (t, 4H), 3.36 (t, 4H), 2.82-2.74 (m, 2H), 2.65-2.57 (m, 2H), 2.29-2.19 (m, 1H), 2.02-1.92 (m, 1H). LCMS (Method A): R_{T}=3.40 min, M+2⁺=427.10.

### Example 54

### 2-((6-(4-(1-Aminocyclobutyl)phenyl)-5-phenylfuro[2,3-d]pyrimidin-2-yl)oxy)ethanol

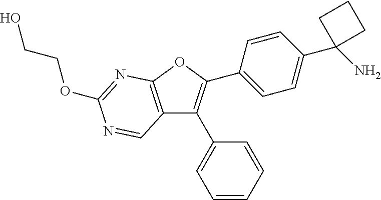

### Step 1: tert-Butyl (1-(4-(2-(2-hydroxyethoxy)-5-phenylfuro[2,3-d]pyrimidin-6-yl)phenyl)cyclobutyl)carbamate

Ethane-1,2-diol (0.054 ml, 0.962 mmol) and N,N-diisopropylethylamine (0.168 ml, 0.962 mmol) were added to a stirred solution of tert-butyl 1-(4-(2-(methylsulfonyl)-5-phenylfuro[2,3-d]pyrimidin-6-yl)phenyl)cyclobutylcarbamate (50 mg, 0.096 mmol) in α,α,α-trifluorotoluene (1.0 ml) in a 10 ml microwave vial. The reaction mixture was heated under microwave conditions (CEM Explorer 24/Discover) at 150° C. for 20 minutes. Analysis by LCMS showed incomplete reaction and therefore was further heated under microwave conditions at 180° C. for 20 minutes. Analysis by LCMS showed ca. 40% conversion to product. The reaction mixture was further heated under microwave conditions at 180° C. for 60 minutes. Analysis by LCMS showed almost no starting material, but some decomposition to compound with Boc group removed. The reaction mixture was eluted directly onto a column, washing with some DCM and purified by flash chromatography (SiO₂, EtOAc) to give the title compound (12.4 mg, 26%) as a pale yellow oil. LCMS (Method A): R_{T}=6.84 min, M+H⁺=502.15.

### Step 2: 2-((6-(4-(1-Aminocyclobutyl)phenyl)-5-phenylfuro[2,3-d]pyrimidin-2-yl)oxy)ethanol, 2HCl

4 M HCl in 1,4-dioxane (1.5 ml, 6.00 mmol) was added to a stirred solution of tert-butyl 1-(4-(2-(2-hydroxyethoxy)-5-phenylfuro[2,3-d]pyrimidin-6-yl)phenyl)cyclobutylcarbamate (12.4 mg, 0.025 mmol) in tetrahydrofuran (0.5 ml) at RT under an atmosphere of nitrogen. After 2 hours, precipitate had formed and the suspension was triturated using diethyl ether (5×3 ml) to remove impurities. The residual solvents were removed in vacuo to give the title compound (7.6 mg, 65%) as an off-white solid (bis HCl salt). LCMS (Method A): R_{T}=3.86 min, M+2⁺=403.2. ¹H NMR (500 MHz, methanol-d₄): δ 8.75 (s, 1H), 7.77 (d, 2H), 7.58-7.48 (m, 7H), 4.59 (t, 2H), 3.96 (t, 2H), 2.83-2.75 (m, 2H), 2.64-2.56 (m, 2H), 2.29-2.19 (m, 1H), 2.03-1.93 (m, 1H).

### Example 55

### 6-(4-(1-Aminocyclobutyl)phenyl)-N-methyl-5-phenylfuro[2,3-d]pyrimidin-2-amine

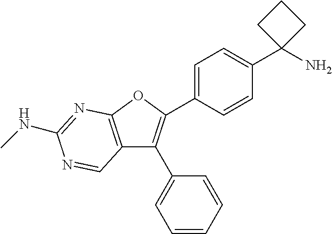

### Step 1: tert-Butyl (1-(4-(2-(methylamino)-5-phenylfuro[2,3-d]pyrimidin-6-yl)phenyl)cyclobutyl)carbamate

A solution of tert-butyl 1-(4-(2-(methylsulfonyl)-5-phenylfuro[2,3-d]pyrimidin-6-yl)phenyl)cyclobutylcarbamate (50 mg, 0.096 mmol) in 2M methylamine in THF (1.0 ml, 2.00 mmol) in a 10 ml microwave tube was heated with stirring under microwave conditions (CEM Explorer 24/Discover) at 80° C. for 10 min. Analysis by LCMS showed complete conversion to product. The solvents were removed in vacuo and the remaining residue was purified by flash chromatography (SiO₂, 0-50%, EtOAc in n-hexane) to give the title compound (43.6 mg, 96%) as a yellow solid. LCMS (Method A): R_{T}=7.45 min, M+H⁺=471.23.

### Step 2: 6-(4-(1-Aminocyclobutyl)phenyl)-N-methyl-5-phenylfuro[2,3-d]pyrimidin-2-amine, 2HCl

4 M HCl in 1,4-dioxane (1.5 ml, 6.00 mmol) was added to a stirred solution of tert-butyl (1-(4-(2-(methylamino)-5-phenylfuro[2,3-d]pyrimidin-6-yl)phenyl)cyclobutyl)carbamate (43.6 mg, 0.093 mmol) in tetrahydrofuran (0.5 ml) at RT under an atmosphere of nitrogen. After 2 hours, precipitate had formed and the suspension was triturated using diethyl ether (5×3 mL) to remove impurities. The residual solvents were removed in vacuo and resulting reside was freeze-dried to give the title compound (33.2 mg, 81%) as a fluffly pale yellow solid (bis HCl salt). ¹H NMR (500 MHz, methanol-d₄): δ 8.47 (s, 1H), 7.76-7.72 (m, 2H), 7.56-7.48 (m, 7H), 3.09 (s, 3H), 2.82-2.74 (m, 2H), 2.65-2.57 (m, 2H), 2.29-2.19 (m, 1H), 2.03-1.93 (m, 1H). LCMS (Method A): R_{T}=3.97 min, M+H⁺=370.1.

### Example 56

### (S)-6-(4-(1-aminocyclobutyl)phenyl)-2-(3-hydroxypyrrolidin-1-yl)-3-methyl-5-phenylfuro[2,3-d]pyrimidin-4(3H)-one

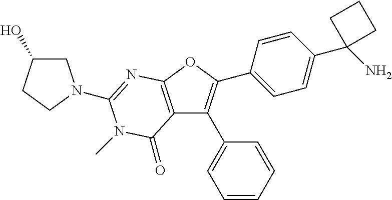

### Step 1: 3-Methyl-2-(methylthio)-5-phenylfuro[2,3-d]pyrimidin-4(3H)-one

(N.B. Starting material contains 48% w/w DBU type impurity). To a solution of 2-(methylthio)-5-phenylfuro[2,3-d]pyrimidin-4(3H)-one (10 g, 38.7 mmol) (prepared as described for Example 13) in dry DMF (40 ml) was charged potassium carbonate (16.1 g, 116 mmol) followed by iodomethane (2.66 ml, 42.6 mmol). The reaction was stirred at RT for 2 h, diluted with water and extracted twice with ethyl acetate. The ethyl acetate layers were combined washed with water/brine 1:1 four times, dried (MgSO₄), filtered and concentrated in vacuo to afford the title compound as a brown solid 5.2 g. LCMS (Method E) R_{T}=1.416 min, M+H⁺=273.

### Step 2: 6-Bromo-3-methyl-2-(methylthio)-5-phenylfuro[2,3-d]pyrimidin-4(3H)-one

To a solution of 3-methyl-2-(methylthio)-5-phenylfuro[2,3-d]pyrimidin-4(31)-one (6.4 g, 23.50 mmol) in DMF (150 ml) at 0° C. was charged bromine (1.33 ml, 25.9 mmol). The reaction was then stirred for 1 h at 0° C. To the reaction mixture at 0° C. was charged a 1:1 mixture of sat. aq,NaHCO₃ and 5% aq.Na₂S₂O₃. The reaction mixture was extracted with EtOAc/THF 1:1 twice. The organic layers were combined and washed with 1:1 water:brine (×4). The organic layer was then dried (MgSO₄) filtered and concentrated in vacuo to afford the desired product as a brown solid (6.9 g, 84%). LCMS (Method E) R_{T}=1.56 min, M+H⁺=351/353.

### Step 3: tert-Butyl (1-(4-(3-methyl-2-(methylthio)-4-oxo-5-phenyl-3,4-dihydrofuro[2,3-d]pyrimidin-6-yl)phenyl)cyclobutyl)carbamate

To 6-bromo-3-methyl-2-(methylthio)-5-phenylfuro[2,3-d]pyrimidin-4(3H)-one (4.2 g, 11.96 mmol) and tert-butyl 1-(4-(4,4,5,5-tetramethyl-1,3,2-dioxaborolan-2-yl)phenyl)cyclobutylcarbamate (5.36 g, 14.35 mmol, prepared as described in WO2008/070016) in DME (100 ml) was charged a solution of potassium carbonate (16.53 g, 120 mmol) in water (25.00 ml). The reaction was degassed with N₂ for 5 min and Pd(PPh₃)₄ (1.38 g, 1.2 mmol) added. The reaction was then heated at reflux for 48 h. After cooling to RT the reaction mixture was diluted with water and ethyl acetate. An insoluble solid precipitate formed that was collected by filtration through a sintered funnel, washed with ethyl acetate and dried to afford the title compound (6 g, 97%) as a yellow solid. LCMS (Method E) R_{T}=1.80 min, M+H⁺=518.

### Step 4: tert-Butyl (1-(4-(3-methyl-2-(methylsulfonyl)-4-oxo-5-phenyl-3,4-dihydrofuro[2,3-d]pyrimidin-6-yl)phenyl)cyclobutyl)carbamate and tert-butyl (1-(4-(3-methyl-2-(methylsulfinyl)-4-oxo-5-phenyl-3,4-dihydrofuro[2,3-d]pyrimidin-6-yl)phenyl)cyclobutyl)carbamate

Following the procedure for tert-butyl 1-(4-(2-(methylsulfonyl)-4-morpholino-5-phenylfuro[2,3-d]pyrimidin-6-yl)phenyl)cyclobutylcarbamate, tert-butyl 1-(4-(3-methyl-2-(methylthio)-4-oxo-5-phenyl-3,4-dihydrofuro[2,3-d]pyrimidin-6-yl)phenyl)cyclobutylcarbamate (1 g, 1.93 mmol) was reacted to afford the title compounds as an orange foamy solid. LCMS (Method E) sulfone R_{T}=1.66 min, M+H⁺=550, sulfoxide R_{T}=1.44, M+H⁺=534.

### Step 5: (S)-tert-Butyl (1-(4-(2-(3-hydroxypyrrolidin-1-yl)-3-methyl-4-oxo-5-phenyl-3,4-dihydrofuro[2,3-d]pyrimidin-6-yl)phenyl)cyclobutyl)carbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (200 mg, 0.364 mmol) in DMF (2 ml) was charged (S)-pyrrolidin-3-ol (159 mg, 1.82 mmol). The reaction was heated at 90° C. for 1 h in a sealed tube. The crude material was diluted with water and extracted with ethyl acetate, the organic layer was dried (MgSO₄) filtered and concentrated in vacuo. The crude material was purified by flash chromatography (SiO₂, gradient 0-5% methanol in DCM) to afford the title compound (67 mg, 33%) as a colorless gum. LCMS (Method E) R_{T}=1.49 min, M+H⁺=557.

### Step 6: (S)-6-(4-(1-Aminocyclobutyl)phenyl)-2-(3-hydroxypyrrolidin-1-yl)-3-methyl-5-phenylfuro[2,3-d]pyrimidin-4(3H)-one

To a solution of (S)-tert-butyl 1-(4-(2-(3-hydroxypyrrolidin-1-yl)-3-methyl-4-oxo-5-phenyl-3,4-dihydrofuro[2,3-d]pyrimidin-6-yl)phenyl)cyclobutylcarbamate (65 mg, 0.117 mmol) in DCM (3 ml) was charged TFA (1 ml). The reaction was stirred for 5 min at RT under N₂ concentrated in vacuo and neutralised using aq, sat. NaHCO₃. This was then extracted with ethyl acetate, the layers separated the organic dried (MgSO₄), filtered and concentrated in vacuo. The residue was loaded onto a Biotage SCX-2 (1 g) column, the column was washed with MeOH the product eluted with 2M NH₃ in MeOH. The eluent was concentrated in vacuo and the material freeze-dried (MeOH/THF/H₂O) to afford the title compound (16 mg, 30%) as a white solid. LCMS (Method E) R_{T}=0.79 min, M+H⁺=457. ¹H NMR (500 MHz, DMSO-d₆): δ 7.24-7.36 (m, 9H), 4.98 (d, 1H), 4.29 (m, 1H), 3.68-3.76 (m, 2H), 3.42-3.47 (m, 1H), 3.30 (s, 3H), 2.23-2.30 (m, 2H), 1.85-1.99 (m, 4H), 1.78-1.81 (m, 1H), 1.50-1.58 (m, 1H).

### Example 57

### (R)-6-(4-(1-Aminocyclobutyl)phenyl)-2-(3-hydroxypyrrolidin-1-yl)-3-methyl-5-phenylfuro[2,3-d]pyrimidin-4(3H)-one

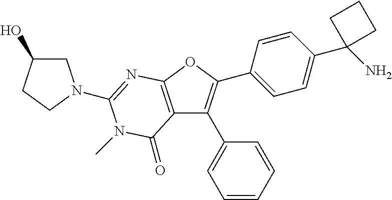

### Step 1: (R)-tert-Butyl (1-(4-(2-(3-hydroxypyrrolidin-1-yl)-3-methyl-4-oxo-5-phenyl-3,4-dihydrofuro[2,3-d]pyrimidin-6-yl)phenyl)cyclobutyl)carbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (200 mg, 0.364 mmol) in DMF (2 ml) was charged (R)-pyrrolidin-3-ol (159 mg, 1.819 mmol). The reaction was heated at 90° C. for 1 h in a sealed tube. The crude material was concentrated in vacuo and purified by flash chromatography (SiO₂, gradient 0-5% methanol in DCM) to afford the title compound (130 mg, 64%) as a brown gum. LCMS (Method E) R_{T}=1.49 min, M+H⁺=557.

### Step 2: (R)-6-(4-(1-Aminocyclobutyl)phenyl)-2-(3-hydroxypyrrolidin-1-yl)-3-methyl-5-phenylfuro[2,3-d]pyrimidin-4(3H)-one hydrochloride

To a solution of (R)-tert-butyl 1-(4-(2-(3-hydroxypyrrolidin-1-yl)-3-methyl-4-oxo-5-phenyl-3,4-dihydrofuro[2,3-d]pyrimidin-6-yl)phenyl)cyclobutylcarbamate (130 mg, 0.234 mmol) in DCM (3 ml) was charged trifluoroacetic acid (1 ml). The reaction was stirred for 5 min at RT under N₂. The reaction was concentrated in vacuo and neutralised using aq. sat. NaHCO₃. This was then extracted with ethyl acetate, the layers separated the organic dried (MgSO₄), filtered and concentrated in vacuo. The crude material was then purified by flash chromatography (SiO₂, gradient 0-10% NH₃/methanol (1M solution of NH₃ in MeOH) in DCM), to afford the title compound (35 mg, 30%) as a white solid. LCMS (Method E) R_{T}=0.77 min, M+H⁺=457. ¹H NMR (500 MHz, DMSO-d₆): δ 7.32-7.43 (m, 9H), 5.05 (d, 1H), 4.37 (m, 1H), 3.75-3.84 (m, 2H), 3.50-3.54 (m, 1H), 3.38 (s, 3H), 2.31-2.37 (m, 2H), 2.03-2.08 (m, 2H), 1.92-2.00 (m, 2H), 1.83-1.90 (m, 1H), 1.58-1.66 (m, 1H).

### Example 58

### 6-(4-(1-Aminocyclobutyl)phenyl)-3-methyl-5-phenyl-2-(pyrrolidin-1-yl)furo[2,3-d]pyrimidin-4(3H)-one hydrochloride

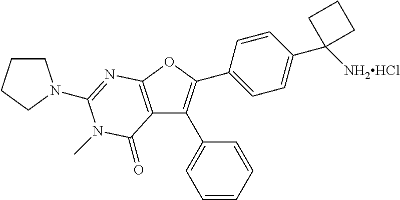

### Step 1: tert-Butyl (1-(4-(3-methyl-4-oxo-5-phenyl-2-(pyrrolidin-1-yl)-3,4-dihydrofuro[2,3-d]pyrimidin-6-yl)phenyl)cyclobutyl)carbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (150 mg, 0.273 mmol) in THF (1 ml) was charged pyrrolidine (194 mg, 2.73 mmol) and the reaction was stirred at RT for 2 h, concentrated in vacuo and purified by flash chromatography (SiO₂, gradient 0-5% MeOH in DCM). This afforded the title compound (90 mg, 61%) as a white solid HPLC-MS (Method E) R_{T}=1.76 min, M+H⁺=541.

### Step 2: 6-(4-(1-Aminocyclobutyl)phenyl)-3-methyl-5-phenyl-2-(pyrrolidin-1-yl)furo[2,3-d]pyrimidin-4(3H)-one hydrochloride

A solution of tert-butyl 1-(4-(3-methyl-4-oxo-5-phenyl-2-(pyrrolidin-1-yl)-3,4-dihydrofuro[2,3-d]pyrimidin-6-yl)phenyl)cyclobutylcarbamate (90 mg, 0.16 mmol) in DCM (2 ml) and TFA (1 ml) was stirred at RT for 5 min the reaction was concentrated in vacuo and the residue neutralised using aq. NaHCO₃. The aqueous layer was extracted twice with ethyl acetate, the organic extracts combined, dried (MgSO₄), filtered and concentrated in vacuo. The HCl salt of the deprotected material was prepared by dissolving the material in 1,4-dioxane and adding to this a freshly prepared solution of HCl in MeOH. (2 eq prepared from MeOH/acetyl chloride). The desired product precipitated from solution and was collected by filtration through a sintered funnel and washed with diethyl ether. The material was dried under high vacuum to afford the title compound (21 mg, 27%) as a white solid. LCMS, (Method E) R_{T}=0.93 min, M+H⁺=441. ¹H NMR (500 MHz, DMSO-d₆): δ 8.60 (br s, 2H), 7.40-7.47 (m, 9H), 3.54-3.57 (m, 4H), 3.38 (s, 3H), 2.49-2.58 (m, 4H), 2.10-2.19 (m, 1H), 1.88-1.91 (m, 4H), 1.72-1.81 (m, 1H).

### Example 59

### 6-(4-(1-Aminocyclobutyl)phenyl)-2-(4-hydroxypiperidin-1-yl)-3-methyl-5-phenylfuro[2,3-d]pyrimidin-4(3H)-one hydrochloride

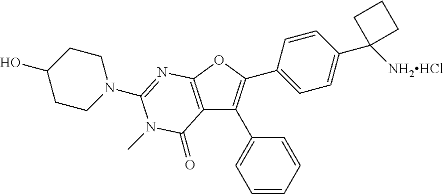

### Step 1: tert-Butyl (1-(4-(2-(4-hydroxypiperidin-1-yl)-3-methyl-4-oxo-5-phenyl-3,4-dihydrofuro[2,3-d]pyrimidin-6-yl)phenyl)cyclobutyl)carbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (200 mg, 0.364 mmol) in THF (2 ml) was charged piperidin-4-ol (184 mg, 1.819 mmol). The reaction was stirred at RT for 2 h. The reaction was concentrated in vacuo and the residue purified by flash chromatography (SiO₂-gradient 0-10% methanol in DCM) to afford the title compound (115 mg, 55%) as an off white solid. LCMS (Method E) R_{T}=1.52, M+H⁺=571.

### Step 2: 6-(4-(1-aminocyclobutyl)phenyl)-2-(4-hydroxypiperidin-1-O-3-methyl-5-phenylfuro[2,3-d]pyrimidin-4(3H)-one hydrochloride

Following the procedure for 6-(4-(1-aminocyclobutyl)phenyl)-3-methyl-5-phenyl-2-(pyrrolidin-1-yl)furo[2,3-d]pyrimidin-4(3H)-one hydrochloride, tert-butyl 1-(4-(2-(4-hydroxypiperidin-1-yl)-3-methyl-4-oxo-5-phenyl-3,4-dihydrofuro[2,3-d]pyrimidin-6-yl)phenyl)cyclobutylcarbamate (105 mg, 0.184 mmol) was reacted to afford the title compound (25 mg, 27%) as a white solid LCMS (Method E) R_{T}=0.81 min, M+H⁺=471. ¹H NMR (500 MHz, DMSO): δ 7.34-7.43 (m, 9H), 3.65-3.69 (m, 1H), 3.39-3.42 (m, 2H), 3.32 (s, 3H), 2.93-2.97 (m, 2H), 2.44-2.49 (m, 4H), 2.05-2.13 (m, 1H), 1.80-1.83 (m, 2H), 1.66-1.75 (m, 1H), 1.47-1.53 (m, 2H).

### Example 60

### 2-((6-(4-(1-Aminocyclobutyl)phenyl)-3-methyl-4-oxo-5-phenyl-3,4-dihydrofuro[2,3-d]pyrimidin-2-yl)amino)-N-methylacetamide hydrochloride

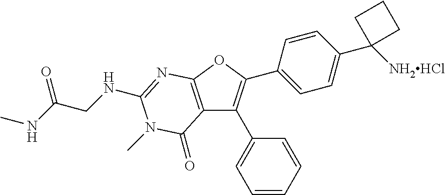

### Step 1: tert-Butyl (1-(4-(3-methyl-2-((2-(methylamino)-2-oxoethyl)amino)-4-oxo-5-phenyl-3,4-dihydrofuro[2,3-d]pyrimidin-6-yl)phenyl)cyclobutyl)carbamate

A solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (300 mg, 0.546 mmol) in DMF (2 ml) was added to a suspension of 2-amino-N-methylacetamide hydrochloride (245 mg, 1.96 mmol) and triethylamine (0.27 ml, 1.97 mmol) in DMF (2 ml). Reaction was complete after 10 min at RT. The reaction was diluted with ethyl acetate and water, the layers were separated and the aqueous extracted once more with ethyl acetate. The combined organic extracts were washed with water, dried (MgSO₄), filtered and concentrated in vacuo. The solid residue was purified by slurrying in ethyl acetate to afford the title compound (100 mg, 33%) as a white solid. LCMS (Method E) R_{T}=1.38 min, M+H^{#}=558.

### Step 2: 2-((6-(4-(1-Aminocyclobutyl)phenyl)-3-methyl-4-oxo-5-phenyl-3,4-dihydrofuro[2,3-d]pyrimidin-2-yl)amino)-N-methylacetamide hydrochloride

A solution of tert-butyl 1-(4-(3-methyl-2-(2-(methylamino)-2-oxoethylamino)-4-oxo-5-phenyl-3,4-dihydrofuro[2,3-d]pyrimidin-6-yl)phenyl)cyclobutylcarbamate (100 mg, 0.179 mmol) in DCM (2 ml) and TFA (1 ml) was stirred at RT for 5 min the reaction was concentrated in vacuo and the residue neutralised using aq.NaHCO₃. The aqueous layer was extracted twice with ethyl acetate, the organic extracts combined, dried (MgSO₄), filtered and concentrated in vacuo. The residue was purified by flash chromatography (SiO₂, gradient 0-15% 2M NH₃/MeOH in DCM). The HCl salt of this purified material was prepared by dissolving the material in 1,4-dioxane and adding 2 eq of HCl in methanol (freshly prepared from acetyl chloride/MeOH). A white solid precipitated this was collected by filtration washed with diethyl ether and dried to afford the title compound (15 mg, 17%). LCMS (Method E) R_{T}=0.73 min, M+H⁺=458 ¹H NMR (500 MHz, DMSO-d₆) 8.54 (br s, 2H), 7.88-7.91 (m, 1H), 7.80-7.82 (m, 1H), 7.42 (m, 9H), 3.95 (d, 2H), 3.36 (s, 3H), 2.47-2.59 (m, 4H), 2.63 (d, 3H), 2.10-2.18 (m, 1H), 1.74-1.82 (m, 1H).

### Example 61

### 6-(4-(1-Aminocyclobutyl)phenyl)-2-(3-hydroxyazetidin-1-yl)-3-methyl-5-phenylfuro[2,3-d]pyrimidin-4(3H)-one

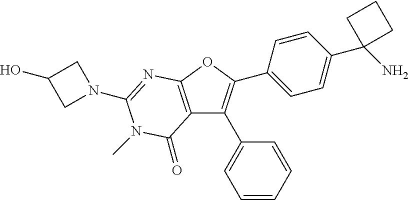

### Step 1: tert-Butyl (1-(4-(2-(3-hydroxyazetidin-1-yl)-3-methyl-4-oxo-5-phenyl-3,4-dihydrofuro[2,3-d]pyrimidin-6-yl)phenyl)cyclobutyl)carbamate

To a suspension of azetidin-3-ol, HCl (159 mg, 1.456 mmol) and DIPEA (0.254 ml, 1.456 mmol) in THF (2 ml) was charged a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (200 mg, 0.364 mmol) in THF (1 ml). The reaction was stirred at RT for 2 h. The THF was removed in vacuo and the residue taken up in dichloromethane and water the resulting biphasic solution was separated using a phase separator (Isolute® SPE). The organic layer was concentrated in vacuo and the crude material purified by flash chromatography (SiO₂, gradient 40-80% ethyl acetate in cyclohexane) to afford the title compound (110 mg, 56%) as a white solid. LCMS (Method E) R_{T}=1.47 min, M+H⁺=543.

### Step 2: 6-(4-(1-Aminocyclobutyl)phenyl)-2-(3-hydroxyazetidin-1-yl)-3-methyl-5-phenylfuro[2,3-d]pyrimidin-4(3H)-one

To a suspension of tert-butyl 1-(4-(2-(3-hydroxyazetidin-1-yl)-3-methyl-4-oxo-5-phenyl-3,4-dihydrofuro[2,3-d]pyrimidin-6-yl)phenyl)cyclobutylcarbamate (100 mg, 0.184 mmol) in DCM (3 ml) was charged TFA (1 ml). The resultant solution was then stirred at RT for 5 min. The reaction mixture was concentrated in vacuo and the residue neutralised with aq. NaHCO₃ and extracted into DCM. The biphasic solution was separated using a phase separator (Isolute® SPE). The organic layer was concentrated in vacuo and the crude material purified by preparative. HPLC (Method G) to afford the title compound as a white solid (8 mg, 10%). HPLC-MS (Method E) R_{T}=0.77 min, M+H⁺=443. ¹H NMR (500 MHz, methanol-d₄): δ 7.32-7.47 (m, 9H), 4.65-4.68 (m, 1H), 4.51-4.54 (m, 2H), 4.12-4.14 (m, 2H), 3.42 (s, 3H), 2.56-2.61 (m, 2H), 2.26-2.31 (m, 2H) 2.03-2.13 (m, 1H), 1.75-1.83 (m, 1H).

### Example 62

### (R)-6-(4-(1-Aminocyclobutyl)phenyl)-2-((2-hydroxypropyl)amino)-3-methyl-5-phenylfuro[2,3-d]pyrimidin-4(3H)-one

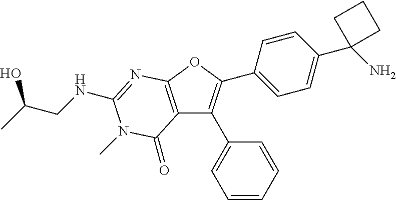

### Step 1: (R)-tert-Butyl (1-(4-(24(2-hydroxypropyl)amino)-3-methyl-4-oxo-5-phenyl-3,4-dihydrofuro[2,3-d]pyrimidin-6-yl)phenyl)cyclobutyl)carbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (200 mg, 0.36 mmol) in THF (2 ml) was charged (R)-1-aminopropan-2-ol (134 mg, 1.82 mmol) and the reaction was stirred at RT for 2 h. The reaction mixture was concentrated in vacuo and the residue purified by flash chromatography (SiO₂, gradient 60-100% ethyl acetate in cyclohexane) to afford the title compound (91 mg, 46%) as a white solid. HPLC-MS (Method E) R_{T}=1.49 min, M+H⁺=545.

### Step 2: (R)-6-(4-(1-Aminocyclobutyl)phenyl)-2-(2-hydroxypropyl)amino)-3-methyl-5-phenylfuro[2,3-d]pyrimidin-4(3H)-one

Following the procedure for 2-((6-(4-(1-aminocyclobutyl)phenyl)-3-methyl-4-oxo-5-phenyl-3,4-dihydrofuro[2,3-d]pyrimidin-2-yl)amino)-N-methylacetamide hydrochloride, (R)-tert-butyl 1-(4-(2-(2-hydroxypropylamino)-3-methyl-4-oxo-5-phenyl-3,4-dihydrofuro[2,3-d]pyrimidin-6-yl)phenyl)cyclobutylcarbamate was reacted to afford the title compound (8 mg, 12%) as a white solid. UPLC-MS (Method E) R_{T}=0.78 min, M+H⁺=445. ¹H NMR (500 MHz, methanol-d₄) 7.29-7.44 (m, 9H), 3.99-4.05 (m, 1H), 3.31-3.50 (m, 5H), 2.62-2.68 (m, 2H), 2.43-2.49 (m, 2H), 2.07-2.16 (m, 1H) 1.81-1.90 (m, 1H), 1.17 (d, 3H).

### Example 63

### 2-((6-(4-(1-Aminocyclobutyl)phenyl)-3-methyl-4-oxo-5-phenyl-3,4-dihydrofuro[2,3-d]pyrimidin-2-yl)amino)acetamide

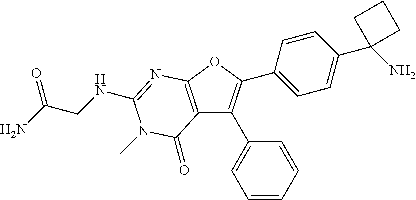

Step 1 tert-Butyl (1-(4-(2-((2-amino-2-oxoethyl)amino)-3-methyl-4-oxo-5-phenyl-3,4-dihydrofuro[2,3-d]pyrimidin-6-yl)phenyl)cyclobutyl)carbamate

A solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (200 mg, 0.364 mmol) in DMF (2 ml) was added to a suspension of 2-aminoacetamide hydrochloride (145 mg, 1.31 mmol) and triethylamine (183 μl, 1.31 mmol) in DMF (2 ml). After stirring for 10 min at RT, the reaction mixture was diluted with ethyl acetate and water. The layers were separated and the aqueous extracted once more with ethyl acetate. The combined organic extracts were washed four times with water/brine 1:1, dried (MgSO₄), filtered and concentrated in vacuo. In dissolving the material again in ethyl acetate for purification a white precipitate formed, this was collected by filtration and dried to afford the title compound (45 mg, 23%) as a white solid. UPLC-MS (Method E) R_{T}=1.33 min, M+H⁺=544.

### Step 2: 2-((6-(4-(1-Aminocyclobutyl)phenyl)-3-methyl-4-oxo-5-phenyl-3,4-dihydrofuro[2,3-d]pyrimidin-2-yl)amino)acetamide

To a suspension of tert-butyl (1-(4-(2-((2-amino-2-oxoethyl)amino)-3-methyl-4-oxo-5-phenyl-3,4-dihydrofuro[2,3-d]pyrimidin-6-yl)phenyl)cyclobutyl)carbamate (45 mg, 0.08 mmol) in DCM (3 ml) was charged TFA (1 ml). The resultant solution was stirred at RT for 5 min. The reaction mixture was concentrated in vacuo and the residue neutralised with aqNaHCO₃ and extracted into DCM. The biphasic solution was separated using a phase separator (Isolute® SPE). The organic layer was concentrated in vacuo and the crude material purified by flash chromatography (SiO₂, gradient 5-15% 2MNH₃/methanol in DCM) to afford the title compound as a light brown solid (10 mg, 30%). UPLC-MS (Method E) R_{T}=0.74 min, M+H⁺=444. ¹H NMR (500 MHz, methanol-d₄): δ 7.32-7.47 (m, 9H), 4.15 (s, 2H), 3.47 (s, 3H), 2.59-2.64 (m, 2H), 2.31-2.37 (m, 2H) 2.07-2.16 (m, 1H), 1.78-1.87 (m, 1H).

### Example 64

### 6-(4-(1-Aminocyclobutyl)phenyl)-2-(2-hydroxyethylamino)-3-methyl-5-phenylfuro[2,3-d]pyrimidin-4(3H)-one hydrochloride

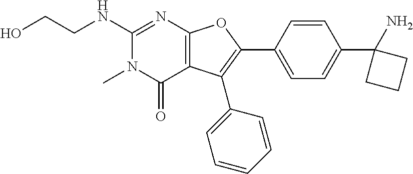

### Step 1: 6-Bromo-3-methyl-2-(methylthio)-5-phenylfuro[2,3-d]pyrimidin-4(3H)-one

To a solution of 6-bromo-2-(methylthio)-5-phenylfuro[2,3-d]pyrimidin-4(3H)-one (1 g, 2.97 mmol) in DMF (2 ml) was added potassium carbonate (0.902 g, 6.52 mmol) followed by MeI (0.204 ml, 3.26 mmol). The reaction mixture was then heated at 60° C. and maintained at this temperature for 2 h. The reaction mixture was then diluted with EtOAc (25 ml) and brine (25 ml) and extracted. The organic extracts were washed with brine (3×15 ml), dried (Na₂SO₄) and concentrated in vacuo affording the crude product. The residue was slurried in Et₂O, filtered and dried affording the title compound (0.812 g, 2.31 mmol, 78%) as a beige solid. NMR (500 MHz, CDCl₃): δ 7.66 (dd, 2H), 7.45 (t, 2H), 7.4 (m, 1H), 3.57 (s, 3H), 2.66 (s, 3H).

### Step 2: tert-Butyl 1-(4-(3-methyl-2-(methylthio)-4-oxo-5-phenyl-3,4-dihydrofuro[2,3-d]pyrimidin-6-yl)phenyl)cyclobutylcarbamate

To a degassed solution of 6-bromo-3-methyl-2-(methylthio)-5-phenylfuro[2,3-d]pyrimidin-4(3H)-one (1.5 g, 4.27 mmol) and tert-butyl 1-(4-(4,4,5,5-tetramethyl-1,3,2-dioxaborolan-2-yl)phenyl)cyclobutylcarbamate (1.91 g, 5.13 mmol, prepared as described in WO2008/070016) in DME (35 ml) was added potassium carbonate (5.90 g, 42.7 mmol) in water (8.75 ml). After further degassing, Pd(PPh₃)₄ (0.494 g, 0.427 mmol) was added and then heated to reflux and maintained at this temperature overnight. The reaction mixture was cooled to room temperature, diluted with EtOAc and water and extracted. The organic extracts were washed with brine (3×15 ml), dried and concentrated in vacuo. The resultant residue was slurried in Et₂O, filtered and dried affording the title compound (1.55 g, 2.99 mmol, 70%) as a beige solid. LCMS (Method A) R_{T}=8.16 min, M+H⁺=518.2.

### Step 3: tert-Butyl 1-(4-(3-methyl-2-(methylsulfonyl)-4-oxo-5-phenyl-3,4-dihydrofuro[2,3-d]pyrimidin-6-yl)phenyl)cyclobutylcarbamate

To a solution of tert-butyl 1-(4-(3-methyl-2-(methylthio)-4-oxo-5-phenyl-3,4-dihydrofuro[2,3-d]pyrimidin-6-yl)phenyl)cyclobutylcarbamate (0.18 g, 0.348 mmol) in THF (4 ml) and MeOH (2 ml) was added Oxone® (0.855 g, 1.39 mmol) in water (2 ml) dropwise over 10 min. The reaction was stirred at 50° C. for 2 h and then diluted with water and DCM, extracted and concentrated in vacuo. The resultant residue was purified by silica gel chromatography (0->100% EtOAc/cyclohexane) affording crude tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (0.06 g, 0.109 mmol, 31%) as a yellow gum. LCMS (Method A) R_{T}=7.58 min, M+H⁺=550.

### Step 4: tert-Butyl 1-(4-(2-(2-hydroxyethylamino)-3-methyl-4-oxo-5-phenyl-3,4-dihydrofuro[2,3-d]pyrimidin-6-yl)phenyl)cyclobutylcarbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (0.035 g, 0.064 mmol) in 1,1,1-trifluorotoluene (3 ml) and DMF (1.00 ml) was added ethanolamine (0.039 ml, 0.637 mmol). The reaction mixture was heated to 80° C. and maintained at this temperature for 2 h. The reaction mixture was then cooled to room temperature, diluted with EtOAc and water and extracted. The organic extracts were washed with brine (3×15 ml), dried and concentrated in vacuo affording tert-butyl 1-(4-(2-(2-hydroxyethylamino)-3-methyl-4-oxo-5-phenyl-3,4-dihydrofuro[2,3-d]pyrimidin-6-yl)phenyl)cyclobutylcarbamate (0.025 g, 0.047 mmol, 74%) as an oil. LCMS (Method A) R_{T}=6.5 min, M+H⁺=531.

### Step 5: 6-(4-(1-Aminocyclobutyl)phenyl)-2-(2-hydroxyethylamino)-3-methyl-5-phenylfuro[2,3-d]pyrimidin-4(3H)-one HCl

To a solution of tert-butyl 1-(4-(2-(2-hydroxyethylamino)-3-methyl-4-oxo-5-phenyl-3,4-dihydrofuro[2,3-b]pyrimidin-6-yl)phenyl)cyclobutylcarbamate (0.025 g, 0.047 mmol) in tetrahydrofuran (3 ml) was added HCl (4M in dioxane) (2 ml) dropwise. The reaction mixture was stirred at room temperature for 6 h before being concentrated to ca. half of the volume in vacuo. The reaction mixture was then diluted with Et₂O before collection of the precipitated solid by filtration. This afforded 6-(4-(1-aminocyclobutyl)phenyl)-2-(2-hydroxyethylamino)-3-methyl-5-phenylfuro[2,3-d]pyrimidin-4(3H)-one, HCl (15 mg, 0.032 mmol, 87%) as an off-white solid. LCMS (Method A) R_{T}=3.69 min, M−NH₃+H⁺=414. NMR (500 MHz, methanol-d₄): δ 7.45 (m, 2H), 7.25-35 (m, 7H), 3.7 (m, 2H), 3.58 (m, 2H), 3.3 (s, 3H), 2.63 (m, 2H), 2.45 (m, 2H), 2.1 (m, 1H), 1.8 (m, 1H).

### Example 65

### 6-(4-(1-Aminocyclobutyl)phenyl)-3-methyl-2-(methylamino)-5-phenylfuro[2,3-d]pyrimidin-4(3H)-one hydrochloride

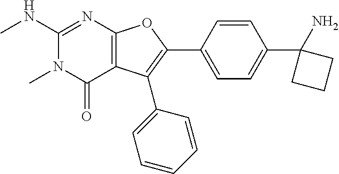

### Step 1: tert-Butyl 1-(4-(3-methyl-2-(methylamino)-4-oxo-5-phenyl-3,4-dihydrofuro[2,3-d]pyrimidin-6-yl)phenyl)cyclobutylcarbamate

tert-Butyl 1-(4-(3-methyl-2-(methylsulfonyl)-4-oxo-5-phenyl-3,4-dihydrofuro[2,3-d]pyrimidin-6-yl)phenyl)cyclobutylcarbamate (0.0350 g, 0.064 mmol) was dissolved in methylamine (2M in THF) (2 ml, 4.00 mmol) and stirred at RT for 1 h. The reaction mixture was concentrated in vacuo and purified by silica gel chromatography (gradient elution 0 to 10% MeOH in DCM) affording tert-butyl 1-(4-(3-methyl-2-(methylamino)-4-oxo-5-phenyl-3,4-dihydrofuro[2,3-d]pyrimidin-6-yl)phenyl)cyclobutylcarbamate (0.018 g, 0.036 mmol, 56%) as a white solid. LCMS (Method A) R_{T}=7.09 min, M+H⁺=501.2.

### Step 2: 6-(4-(1-Aminocyclobutyl)phenyl)-3-methyl-2-(methylamino)-5-phenylfuro[2,3-d]pyrimidin-4(3H)-one hydrochloride

To a solution of tert-butyl 1-(4-(3-methyl-2-(methylamino)-4-oxo-5-phenyl-3,4-dihydrofuro[2,3-d]pyrimidin-6-yl)phenyl)cyclobutylcarbamate (18 mg, 0.036 mmol) in tetrahydrofuran (2 ml) was added HCl (4M in dioxane) (2 ml, 65.8 mmol). The reaction mixture was stirred at RT for 4 h until deemed complete by TLC and LCMS. The reaction was concentrated in vacuo and triturated with Et₂O and the resultant solid collected by filtration. The solid was dried under high vacuum affording 6-(4-(1-aminocyclobutyl)phenyl)-3-methyl-2-(methylamino)-5-phenylfuro[2,3-d]pyrimidin-4(3H)-one, HCl (9 mg, 57%) as a yellow solid. LCMS (Method A) R_{T}=4.06 min, M−NH₃+H⁺=384. NMR (500 MHz, methanol-d₄): δ 7.42 (d, 2H), 7.25-7.35 (m, 7H), 3.3 (s, 3H), 2.92 (s, 3H), 2.65 (m, 2H), 2.5 (m, 2H), 2.12 (m, 1H), 1.85 (m, 1H).

### Example 66

### 6-(4-(1-Aminocyclobutyl)phenyl)-3-methyl-5-phenylfuro[2,3-d]pyrimidin-4(3H)-one

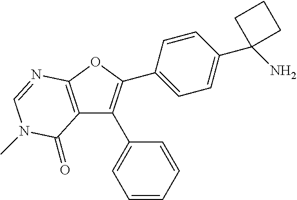

### Step 1: tert-Butyl 1-(4-(4-oxo-5-phenyl-3,4-dihydrofuro[2,3-d]pyrimidin-6-yl)phenyl)cyclobutylcarbamate

To a solution of tert-butyl 1-(4-(4-methoxy-5-phenylfuro[2,3-d]pyrimidin-6-yl)phenyl)cyclobutylcarbamate (0.175 g, 0.371 mmol) in dioxane (2 ml) was added 2M NaOH (aq) (2 ml). The reaction mixture was heated to reflux and maintained at this temperature overnight. TLC and LCMS indicated that the reaction was partially complete. Heating was continued for a further 18 h before cooling. The reaction was diluted with EtOAc and brine and the organic extracts dried (Na₂SO₄) and concentrated in vacuo affording tert-butyl 1-(4-(4-oxo-5-phenyl-3,4-dihydrofuro[2,3-d]pyrimidin-6-yl)phenyl)cyclobutylcarbamate (80 mg, 0.175 mmol, 47%) as a white solid.

LCMS (Method E) R_{T}=1.405 min, M+H⁺=458.2.

### Step 2: tert-Butyl 1-(4-(3-methyl-4-oxo-5-phenyl-3,4-dihydrofuro[2,3-d]pyrimidin-6-yl)phenyl)cyclobutylcarbamate

To a solution of tert-butyl 1-(4-(4-oxo-5-phenyl-3,4-dihydrofuro[2,3-d]pyrimidin-6-yl)phenyl)cyclobutylcarbamate (80 mg, 0.175 mmol) in DMF (1 ml) was added K₂CO₃ (53.2 mg, 0.385 mmol) and MeI (0.012 ml, 0.192 mmol).

The reaction mixture was stirred at room temperature for 18 h before being diluted with EtOAc (15 ml) and brine (15 ml) and extracted. The organic extracts were then washed with brine (3×10 ml), dried (Na₂SO₄) and concentrated in vacuo affording crude product as a beige solid. The solid was slurried in Et₂O, filtered and dried yielding tert-butyl 1-(4-(3-methyl-4-oxo-5-phenyl-3,4-dihydrofuro[2,3-d]pyrimidin-6-yl)phenyl)cyclobutylcarbamate (66.0 mg, 0.140 mmol, 80%) as a beige solid. LCMS (Method D) R_{T}=4.93 min, M+H⁺=473.

### Step 3: 6-(4-(1-Aminocyclobutyl)phenyl)-3-methyl-5-phenylfuro[2,3-d]pyrimidin-4(3H)-10 one

To a solution of tert-butyl 1-(4-(3-methyl-4-oxo-5-phenyl-3,4-dihydrofuro[2,3-d]pyrimidin-6-yl)phenyl)cyclobutylcarbamate (0.050 g, 0.106 mmol) in THF (2 ml) was added HCl (4.0M in 1,4-dioxane) (2 ml). The reaction mixture was stirred at room temperature for 5 h. The resultant precipitate was collected by filtration, washed with Et₂O and dried. The residue was partitioned between DCM and sat. Na₂CO₃, extracted and the DCM portion concentrated. The residue was purified by silica gel chromatography (gradient elution 0 to 5% MeOH/DCM) affording 6-(4-(1-aminocyclobutyl)phenyl)-3-methyl-5-phenylfuro[2,3-d]pyrimidin-4(3H)-one (0.017 g, 43%) as a white solid. LCMS (Method E) R_{T}=0.639 min, M+H⁺=372.1. NMR (500 MHz, CDCl₃): δ 8.03 (s, 1H), 7.53 (m, 4H), 7.4 (m, 3H), 7.31 (d, 2H), 3.6 (s, 3H), 2.53 (m, 2H), 2.15 (m, 2H), 2.09 (m, 1H), 1.75 (m, 1H).

### Example 67

### 6-(4-(1-Aminocyclobutyl)phenyl)-3-(2-methoxyethyl)-2-(methylthio)-5-phenylfuro[2,3-d]pyrimidin-4(3H)-one, HCl

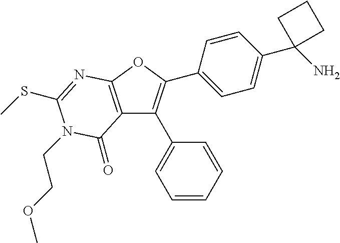

### Step 1: 6-Bromo-3-(2-methoxyethyl)-2-(methylthio)-5-phenylfuro[2,3-d]pyrimidin-4(3H)-one

To a solution of 6-bromo-2-(methylthio)-5-phenylfuro[2,3-d]pyrimidin-4(3H)-one (0.5 g, 1.483 mmol) in DMF (2 ml) was added 1-bromo-2-methoxyethane (0.153 ml, 1.631 mmol) and NaI (0.022 g, 0.148 mmol). The reaction mixture was stirred at 80° C. for 3 h before being cooled to room temperature, diluted with EtOAc and water and extracted. The organic extracts were washed with brine (3×15 ml), dried (Na₂SO₄) and concentrated in vacuo. The resultant residue was purified by silica gel chromatography (gradient elution 0 to 30% EtOAc/hexane) affording 6-bromo-3-(2-methoxyethyl)-2-(methylthio)-5-phenylfuro[2,3-d]pyrimidin-4(3H)-one (0.155 g, 0.392 mmol, 26.4%) as an oil. LCMS (Method A) R_{T}=7.32 min, M+H⁺=396.97.

### Step 2: tert-Butyl 1-(4-(3-(2-methoxyethyl)-2-(methylthio)-4-oxo-5-phenyl-3,4-dihydrofuro[2,3-d]pyrimidin-6-yl)phenyl)cyclobutylcarbamate

To a degassed solution of 6-bromo-3-(2-methoxyethyl)-2-(methylthio)-5-phenylfuro[2,3-d]pyrimidin-4(3H)-one (155 mg, 0.392 mmol) and tert-butyl 1-(4-(4,4,5,5-tetramethyl-1,3,2-dioxaborolan-2-yl)phenyl)cyclobutylcarbamate (176 mg, 0.471 mmol, prepared as described in WO2008/070016) in DME (3 ml) was added potassium carbonate (542 mg, 3.92 mmol) in water (0.75 ml). After further degassing, Pd(PPh₃)₄ (45 mg, 0.039 mmol) was added and the reaction mixture heated to reflux and maintained at this temperature overnight. The reaction mixture was cooled to room temperature, diluted with EtOAc and water and extracted. The organic extracts were washed with brine (3×15 ml), dried and concentrated in vacuo. The resultant residue was purified by silica gel chromatography (gradient elution 0 to 100% EtOAc/hexane) affording tert-butyl 1-(4-(3-(2-methoxyethyl)-2-(methylthio)-4-oxo-5-phenyl-3,4-dihydrofuro[2,3-d]pyrimidin-6-yl)phenyl)cyclobutylcarbamate (0.177 g, 0.315 mmol, 80%) as an off-white solid. LCMS (Method A) R_{T}=8.38 min, M+H⁺=562.2.

### Step 3: 6-(4-(1-Aminocyclobutyl)phenyl)-3-(2-methoxyethyl)-2-(methylthio)-5-phenylfuro[2,3-d]pyrimidin-4(3H)-one HCl

To a solution of tert-butyl 1-(4-(3-(2-methoxyethyl)-2-(methylthio)-4-oxo-5-phenyl-3,4-dihydrofuro[2,3-d]pyrimidin-6-yl)phenyl)cyclobutylcarbamate (0.05 g, 0.089 mmol) in THF (2 ml) was added HCl (4M in dioxane) (2 ml, 8.00 mmol). The reaction mixture was stirred at room temperature for 3 h and the resultant precipitate was collected by filtration, washed with Et₂O and dried under high vacuum affording 6-(4-(1-aminocyclobutyl)phenyl)-3-(2-methoxyethyl)-2-(methylthio)-5-phenylfuro[2,3-d]pyrimidin-4(3H)-one, HCl (0.03 g, 0.060 mmol, 68%) as a white solid. LCMS (Method A) R_{T}=4.61 min, M+H⁺⁼463.15. ¹H NMR (500 MHz, methanol-d₄): δ 7.6 (d, 2H), 7.4-7.5 (m, 7H), 4.35 (t, 2H), 3.68 (t, 3H), 3.4 (s, 3H), 2.7-2.85 (m, 5H), 2.6 (m, 2H), 2.25 (m, 1H), 2.0 (m, 1H).

### Example 68

### 6-(4-(1-Aminocyclobutyl)phenyl)-N-methyl-2-(methylthio)-5-phenylfuro[2,3-d]pyrimidin-4-amine, HCl

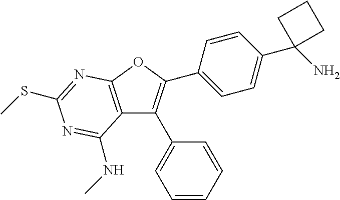

### Step 1: tert-Butyl 1-(4-(4-(methylamino)-2-(methylthio)-5-phenylfuro[2,3-d]pyrimidin-6-yl)phenyl)cyclobutylcarbamate

To a solution of 6-(4-(1-(tert-butoxycarbonylamino)cyclobutyl)phenyl)-2-(methylthio)-5-phenylfuro[2,3-d]pyrimidin-4-yl trifluoromethanesulfonate (0.09 g, 0.142 mmol) in THF (2 ml) was added methanamine (2.0M in THF) (3 ml, 6.00 mmol). The reaction mixture was stirred at room temperature for 2 h and concentrated in vacuo affording tert-butyl 1-(4-(4-(methylamino)-2-(methylthio)-5-phenylfuro[2,3-d]pyrimidin-6-yl)phenyl)cyclobutylcarbamate (0.07 g, 0.135 mmol, 95%) as an oil. LCMS (Method A) R_{T}=8.62 min, M+H⁺=517.16.

### Step 2: 6-(4-(1-Aminocyclobutyl)phenyl)-N-methyl-2-(methylthio)-5-Phenylfuro[2,3-c]pyrimidin-4-amine, HCl

To a solution of tert-butyl 1-(4-(4-(methylamino)-2-(methylthio)-5-phenylfuro[2,3-d]pyrimidin-6-yl)phenyl)cyclobutylcarbamate (0.043 g, 0.083 mmol) in THF (2 ml) was added HCl (4M in dioxane) (2 ml, 8.00 mmol). The reaction mixture was stirred at room temperature for 3 h and the resultant precipitate that had formed was collected by filtration, washed with Et₂O and dried under high vacuum affording 6-(4-(1-aminocyclobutyl)phenyl)-N-methyl-2-(methylthio)-5-phenylfuro[2,3-d]pyrimidin-4-amine, HCl (0.028 g, 0.062 mmol, 75%) as a white solid. LCMS (Method A) R_{T}=4.83 min, M-NH₃+H⁺=400.2. ¹H NMR (500 MHz, methanol-d₄): δ 7.63 (m, 3H), 7.51 (m, 4H), 7.48 (d, 2H), 3.05 (s, 3H), 2.7-2.8 (m, 5H), 2.6 (m, 2H), 2.25 (m, 1H), 1.95 (m, 1H).

### Example 69

### 6-(4-(1-Aminocyclobutyl)phenyl)-3-(2-methoxyethyl)-2-(methylamino)-5-phenylfuro[2,3-d]pyrimidin-4(3H)-one, HCl

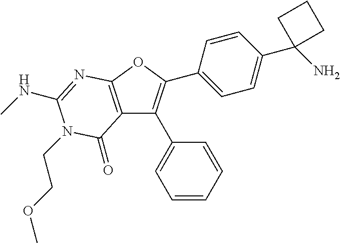

### Step 1: tert-Butyl 1-(4-(3-(2-methoxyethyl)-2-(methylsulfonyl)-4-oxo-5-phenyl-3,4-dihydrofuro[2,3-d]pyrimidin-6-yl)phenyl)cyclobutylcarbamate

To a solution of tert-butyl 1-(4-(3-(2-methoxyethyl)-2-(methylthio)-4-oxo-5-phenyl-3,4-dihydrofuro[2,3-d]pyrimidin-6-yl)phenyl)cyclobutylcarbamate (0.127 g, 0.226 mmol) in THF (3 ml) and MeOH (1.5 ml) was added Oxone® (0.556 g, 0.904 mmol) in water (1.5 ml). The reaction mixture was stirred at room temperature until complete by LCMS. On completion, the reaction mixture was then diluted with EtOAc (15 ml) and brine (15 ml) and extracted. The organic extracts were washed with brine (3×10 ml), dried (Na₂SO₄) and concentrated in vacuo affording a mixture of tert-butyl 1-(4-(3-(2-methoxyethyl)-2-(methylsulfonyl)-4-oxo-5-phenyl-3,4-dihydrofuro[2,3-d]pyrimidin-6-yl)phenyl)cyclobutylcarbamate (0.044 g, 0.074 mmol, 33%) and tert-butyl (1-(4-(3-(2-methoxyethyl)-2-(methylsulfinyl)-4-oxo-5-phenyl-3,4-dihydrofuro[2,3-d]pyrimidin-6-yl)phenyl)cyclobutyl)carbamate (0.025 g, 0.043 mmol, 19%) as an oil that was used without further purification. LCMS (Method A) R_{T}=6.88 min, M+H⁺=578.13 (sulfoxide) & R_{T}=7.72 min, M+H⁺=594.13 (sulfone).

### Step 2: tert-Butyl 1-(4-(3-(2-methoxyethyl)-2-(methylamino)-4-oxo-5-phenyl-3,4-dihydrofuro[2,3-d]pyrimidin-6-yl)phenyl)cyclobutylcarbamate

A solution of tert-butyl 1-(4-(3-(2-methoxyethyl)-2-(methylsulfonyl)-4-oxo-5-phenyl-3,4-dihydrofuro[2,3-d]pyrimidin-6-yl)phenyl)cyclobutylcarbamate (44 mg, 0.074 mmol) in methanamine (2.0M in THF) (3 ml, 6.00 mmol) was stirred at RT for 2 h before analysis by LCMS—reaction complete. The reaction mixture was concentrated in vacuo before purification by silica gel chromatography (0 to 5% MeOH/DCM) affording tert-butyl 1-(4-(3-(2-methoxyethyl)-2-(methylamino)-4-oxo-5-phenyl-3,4-dihydrofuro[2,3-d]pyrimidin-6-yl)phenyl)cyclobutylcarbamate (29.4 mg, 0.054 mmol, 90%) as an oil. LCMS (Method A) R_{T}=7.64 min, M+H⁺=545.22.

### Step 3: 6-(4-(1-Aminocyclobutyl)phenyl)-3-(2-methoxyethyl)-2-(methylamino)-5-phenylfuro[2,3-d]pyrimidin-4(3H)-one, HCl

To a solution of tert-butyl 1-(4-(3-(2-methoxyethyl)-2-(methylamino)-4-oxo-5-phenyl-3,4-dihydrofuro[2,3-d]pyrimidin-6-yl)phenyl)cyclobutylcarbamate (30 mg, 0.055 mmol) in THF (2 ml) was added HCl (4M in dioxane) (2 ml, 65.8 mmol). The reaction mixture was stirred at room temperature for 3 h. The solid that had formed was collected by filtration, washed with Et₂O before freeze-drying (from water). This afforded 6-(4-(1-aminocyclobutyl)phenyl)-3-(2-methoxyethyl)-2-(methylamino)-5-phenylfuro[2,3-d]pyrimidin-4(3H)-one, HCl (0.021 g, 0.044 mmol, 79%) as an off-white solid. LCMS (Method A) R_{T}=4.07 min, M−NH₃+H⁼=428.19. ¹H NMR (500 MHz, methanol-d₄): δ 7.57 (d, 2H), 7.45 (m, 7H), 4.23 (t, 2H), 3.66 (t, 2H), 3.4 (s, 3H), 3.03 (s, 3H), 2.75 (m, 2H), 2.6 (m, 2H), 2.25 (m, 1H), 1.96 (m, 1H).

### Example 70

### 2-(6-(4-(1-Aminocyclobutyl)phenyl)-4-(dimethylamino)-5-phenylfuro[2,3-d]pyrimidin-2-ylamino)ethanol, HCl

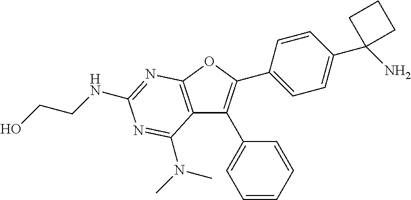

### Step 1: tert-Butyl 1-(4-(4-(dimethylamino)-2-(methylthio)-5-phenylfuro[2,3-d]pyrimidin-6-yl)phenyl)cyclobutylcarbamate

To a solution of 6-(4-(1-(tert-butoxycarbonylamino)cyclobutyl)phenyl)-2-(methylthio)-5-phenylfuro[2,3-d]pyrimidin-4-yl trifluoromethanesulfonate (50 mg, 0.079 mmol) in THF (1 ml) was added dimethylamine (2.0M in THF) (2 ml, 0.079 mmol). The reaction mixture was stirred at room temperature for 1 h. The reaction mixture was concentrated in vacuo and purified by silica gel chromatography (0 to 25% EtOAc/cyclohexane) affording tert-butyl 1-(4-(4-(dimethylamino)-2-(methylthio)-5-phenylfuro[2,3-d]pyrimidin-6-yl)phenyl)cyclobutylcarbamate (32 mg, 0.060 mmol, 77%) as an oil. LCMS (Method A) R_{T}=8.85 min, M+H⁺=531.19.

### Step 2: tert-Butyl 1-(4-(4-(dimethylamino)-2-(methylsulfonyl)-5-phenylfuro[2,3-d]pyrimidin-6-yl)phenyl)cyclobutylcarbamate

To a solution of tert-butyl 1-(4-(4-(dimethylamino)-2-(methylthio)-5-phenylfuro[2,3-d]pyrimidin-6-yl)phenyl)cyclobutylcarbamate (0.165 g, 0.311 mmol) in THF (4 ml) and methanol (2 ml) was added Oxone (0.765 g, 1.24 mmol) in water (2 ml). The reaction mixture was stirred at 50° C. for 1 h. The reaction was diluted with DCM and water and extracted. The DCM extracts were dried (Na₂SO₄) and concentrated yielding the title compound as an off-white solid. ¹H NMR (500 MHz, CDCl₃): δ 7.2-7.43 (m, 9H), 4.98 (s, 1H), 3.3 (s, 3H), 2.72 (s, 6H), 2.4 (m, 4H), 2.0 (m, 1H), 1.6 (m, 1H), 1.25 (br s, 9H).

### Step 3: tert-Butyl 1-(4-(4-(dimethylamino)-2-(2-hydroxyethylamino)-5-phenylfuro[2,3-d]pyrimidin-6-yl)phenyl)cyclobutylcarbamate

To a solution of tert-butyl 1-(4-(4-(dimethylamino)-2-(methylsulfonyl)-5-phenylfuro[2,3-d]pyrimidin-6-yl)phenyl)cyclobutylcarbamate (0.161 g, 0.286 mmol) in α,α,α-trifluorotoluene (2 ml) and DMF (0.5 ml) was added 2-aminoethanol (0.350 g, 5.72 mmol). The reaction mixture was stirred at 80° C. for 1 h, cooled to room temperature, diluted with EtOAc and water and extracted. The organic extracts were washed with brine (3×10 ml), dried (Na₂SO₄) and concentrated in vacuo. The resultant residue was purified by silica gel chromatography (0 to 50% EtOAc/hexane) affording tert-butyl 1-(4-(4-(dimethylamino)-2-(2-hydroxyethylamino)-5-phenylfuro[2,3-d]pyrimidin-6-yl)phenyl)cyclobutylcarbamate (0.085 g, 0.156 mmol, 55%) as an off-white solid. LCMS (Method A) R_{T}=7.30 min, M+H⁺=544.24.

### Step 4: 2-(6-(4-(1-Aminocyclobutyl)phenyl)-4-(dimethylamino)-5-phenylfuro[2,3-d]pyrimidin-2-ylamino)ethanol, HCl

To a solution of tert-butyl 1-(4-(4-(dimethylamino)-2-(2-hydroxyethylamino)-5-phenylfuro[2,3-d]pyrimidin-6-yl)phenyl)cyclobutylcarbamate (85 mg, 0.156 mmol) in THF (1 ml) was added HCl (4M in dioxane) (1 ml, 32.9 mmol). The reaction mixture was stirred at room temperature for 4 h and the resultant solid that formed was collected by filtration and washed with Et₂O before drying under high vacuum yielding 2-(6-(4-(1-aminocyclobutyl)phenyl)-4-(dimethylamino)-5-phenylfuro[2,3-a]pyrimidin-2-ylamino)ethanol, HCl (36 mg, 0.075 mmol, 48%) as an off-white solid.

LCMS (Method A) R_{T}=4.19 min, M−NH₃+H⁺=427.23. ¹H NMR (500 MHz, methanol-d₄): δ 7.44 (m, 3H), 7.3 (m, 6H), 3.7 (t, 2H), 3.55 (t, 2H), 2.76 (s, 6H), 2.64 (m, 2H), 2.45 (m, 2H), 2.1 (m, 1H), 1.8 (m, 1H).

### Example 71

### 2-((6-(4-(1-Aminocyclobutyl)phenyl)-4-morpholino-5-phenylfuro[2,3-d]pyrimidin-2-yl)amino)ethanol

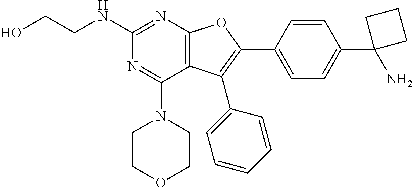

### Step 1: 6-(4-(1-(tert-Butoxycarbonylamino)cyclobutyl)phenyl)-2-(methylthio)-5-phenylfuro[2,3-d]pyrimidin-4-yl trifluoromethanesulfonate

Following the procedure for 2-(4-(1-((tert-butoxycarbonyl)amino)cyclobutyl)phenyl)-3-phenylfuro[3,2-c]pyridin-4-yl trifluoromethanesulfonate, tert-butyl 1-(4-(2-(methylthio)-4-oxo-5-phenyl-3,4-dihydrofuro[2,3-d]pyrimidin-6-yl)phenyl)cyclobutylcarbamate (1.5 g, 2.98 mmol was reacted, the crude product was purified by flash chromatography (SiO₂, gradient 0-55% ethyl acetate in hexane) to afford the title compound (0.9 g, 47%) as a white solid. ¹H NMR (500 MHz, CDCl₃): δ 7.59 (d, 2H), 7.49-7.51 (m, 3H), 7.44-7.47 (m, 2H), 7.39 (d, 2H), 5.09 (s, 1H), 2.67 (s, 3H), 2.50-2.56 (m, 4H), 2.09-2.17 (m, 1H), 1.84-1.93 (m, 1H), 1.38 (br s, 9H).

### Step 2: tert-Butyl 1-(4-(2-(methylthio)-4-morpholino-5-phenylfuro[2,3-d]pyrimidin-6-yl)phenyl)cyclobutylcarbamate

To a solution of 6-(4-(1-(tert-butoxycarbonylamino)cyclobutyl)phenyl)-2-(methylthio)-5-phenylfuro[2,3-d]pyrimidin-4-yl trifluoromethanesulfonate (200 mg, 0.315 mmol) in THF (1 ml) was charged morpholine (0.274 ml, 3.15 mmol). The reaction was then stirred at 50° C. for 5 h. The reaction was concentrated in vacuo to give a white solid, which was slurried in 1:1, DCM/Et₂O, washed with Et₂O and dried to afford the title compound (160 mg, 89%) as a white solid. LCMS (Method A) R_{T}=9.55 min, M+H⁺=573.

### Step 3: tert-Butyl 1-(4-(2-(methylsulfonyl)-4-morpholino-5-phenylfuro[2,3-d]pyrimidin-6-yl)phenyl)cyclobutylcarbamate

To a solution of tert-butyl 1-(4-(2-(methylthio)-4-morpholino-5-phenylfuro[2,3-d]pyrimidin-6-yl)phenyl)cyclobutylcarbamate (160 mg, 0.279 mmol) in THF: MeOH 1:1 (12 mL) was added dropwise a solution of Oxone® (1.4 g, 2.2 mmol) in H₂O (6 mL). The reaction was stirred at RT for 4 h. To the reaction mixture was added aq. sodium hydrogen carbonate. The aqueous layer was extracted three times with ethyl acetate. The organic layers were combined, dried (MgSO₄), filtered and concentrated in vacuo to afford the title compound (47 mg, 28%). LCMS (Method A) R_{T}=7.90 min, M+H⁺=605.

### Step 4: tert-Butyl 1-(4-(2-(2-hydroxyethylamino)-4-morpholino-5-phenylfuro[2,3-d]pyrimidin-6-yl)phenyl)cyclobutylcarbamate

A solution of tert-butyl 1-(4-(2-(methylsulfonyl)-4-morpholino-5-phenylfuro[2,3-d]pyrimidin-6-yl)phenyl)cyclobutylcarbamate (45 mg, 0.074 mmol) and ethanolamine (0.045 mL, 0.744 mmol) in toluene (2 mL) and DMF (1 mL) was heated to 100° C. The reaction was then stirred a further 2 h at 100° C. incomplete. A further 10 eq of ethanolamine was charged to the reaction and this heated at 100° C. overnight complete by LCMS. The reaction was concentrated in vacuo and the crude product was purified by flash chromatography (SiO₂, gradient 0-30% ethyl acetate in hexane) to afford the title compound as an off-white solid, LCMS (Method A) R_{T}=7.56 min, M+H⁺=586.

### Step 5: 2-(6-(4-(1-Aminocyclobutyl)phenyl)-4-morpholino-5-phenylfuro[2,3-d]pyrimidin-2-ylamino)ethanol hydrochloride

To a solution of tert-butyl 1-(4-(2-(2-hydroxyethylamino)-4-morpholino-5-phenylfuro[2,3-d]pyrimidin-6-yl)phenyl)cyclobutylcarbamate (35 mg, 0.060 mmol) in THF (1 ml) was charged 4M HCl in dioxane (2 ml, 4.00 mmol) at RT under N₂. The reaction was stirred at RT overnight during which time a white precipitate formed. To the reaction was charged diethyl ether (3 ml) and the solvent decanted from the solid. The white solid was then washed with diethyl ether and dried to afford the title compound as the hydrochloride salt (25 mg, 80%). LCMS (Method A) R_{T}=4.01 min, M-NH₃⁺=469, M+H⁺=486. ¹H NMR (500 MHz, methanol-d₄): a 7.39-7.61 (m, 9H), 3.76-3.81 (m, 2H), 3.56-3.64 (m, 2H), 3.30-3.40 (m, 4H), 3.22-3.28 (m, 4H), 2.73-2.79 (m, 2H), 2.55-2.61 (m, 2H), 2.20-2.27 (m, 1H), 1.93-1.99 (m, 1H).

### Example 72

### 2-((6-(4-(1-Aminocyclobutyl)phenyl)-4-methyl-5-phenylfuro[2,3-d]pyrimidin-2-yl)amino)ethanol

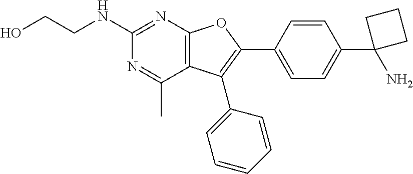

### Step 1: tert-Butyl 1-(4-(4-methyl-2-(methylthio)-5-phenylfuro[2,3-d]pyrimidin-6-yl)phenyl)cyclobutylcarbamate

To a solution of 6-(4-(1-(tert-butoxycarbonylamino)cyclobutyl)phenyl)-2-(methylthio)-5-phenylfuro[2,3-d]pyrimidin-4-yl trifluoromethanesulfonate (250 mg, 0.393 mmol) and iron(III) acetylacetonate (6.9 mg, 0.020 mmol) in THF (10 ml) and NMP (2 ml) at 0° C. under N₂ was charged methylmagnesium chloride 3.0M in THF (0.275 ml, 0.826 mmol). The reaction was stirred at RT for 15 min and diluted with water, the pH was adjusted to 3 using 1M HCl (aq). This was extracted twice with ethyl acetate. The organic layers were combined, dried (MgSO₄) filtered and concentrated in vacuo to afford the title compound as an orange oil. LCMS (Method A) R_{T}=9.50 min, M+H⁺=502.

### Step 2: tert-Butyl 1-(4-(4-methyl-2-(methylsulfonyl)-5-phenylfuro[2,3-d]pyrimidin-6-yl)phenyl)cyclobutylcarbamate

Following the procedure for tert-butyl 1-(4-(2-(methylsulfonyl)-4-morpholino-5-phenylfuro[2,3-d]pyrimidin-6-yl)phenyl)cyclobutylcarbamate, tert-butyl 1-(4-(4-methyl-2-(methylthio)-5-phenylfuro[2,3-d]pyrimidin-6-yl)phenyl)cyclobutylcarbamate (130 mg, 0.259 mmol) was reacted to afford the title compound as a white foamy solid. LCMS (Method A) R_{T}=7.96 min, M+H⁺=534.

### Step 3: tert-Butyl 1-(4-(2-(2-hydroxyethylamino)-4-methyl-5-phenylfuro[2,3-d]pyrimidin-6-yl)phenyl)cyclobutylcarbamate

A solution of tert-butyl 1-(4-(4-methyl-2-(methylsulfonyl)-5-phenylfuro[2,3-d]pyrimidin-6-yl)phenyl)cyclobutylcarbamate (115 mg, 0.216 mmol) and ethanolamine (0.261 ml, 4.31 mmol) in α,α,α-trifluorotoluene (2 ml) was heated at 100° C. for 30 min under microwave conditions. The reaction mixture was concentrated in vacuo and the residue purified by flash chromatography (SiO₂, gradient 20-80% ethyl acetate in hexane) to afford the title compound (60 mg, 54%) as a colorless gum. ¹H NMR (500 MHz, CDCl₃): δ 7.39-7.48 (m, 7H), 7.29 (d, 2H), 5.58 (br s, 1H), 5.05 (s, 1H), 3.87-3.89 (m, 2H), 3.65-3.68 (m, 2H), 2.45-2.48 (m, 4H), 2.09 (s, 3H), 2.04-2.08 (m, 1H), 1.79-1.84 (m, 1H), 1.35 (br s, 9H).

### Step 4: 2-(6-(4-(1-Aminocyclobutyl)phenyl)-4-methyl-5-phenylfuro[2,3-d]pyrimidin-2-ylamino)ethanol hydrochloride

To a solution of tert-butyl 1-(4-(2-(2-hydroxyethylamino)-4-methyl-5-phenylfuro[2,3-d]pyrimidin-6-yl)phenyl)cyclobutylcarbamate (60 mg, 0.117 mmol) in THF (1.5 ml) at RT under N₂ was charged 4M HCl in dioxane (1.5 mL, 6.00 mmol). The reaction was stirred at RT overnight. A white precipitate had formed during the reaction, the reaction was diluted with diethyl ether and the solvent decanted from the solid. The solid was washed with diethyl ether and dried to afford the title compound (38 mg, 72%) as an off-white solid. LCMS (Method A) R_{T}=3.94 min, M+H⁺=415. ¹H NMR (500 MHz, methanol-d₄): δ 7.59-7.65 (m, 5H), 7.49-7.54 (m, 4H), 3.73 (t, 2H), 3.84 (t, 2H), 2.74-2.79 (m, 2H), 2.57-2.63 (m, 2H), 2.29 (s, 3H), 2.21-2.29 (m, 1H), 1.92-2.01 (m, 1H).

### Example 73

### 2-((6-(4-(1-Aminocyclobutyl)phenyl)-5-phenyl-4-(trifluoromethyl)furo[2,3-d]pyrimidin-2-yl)amino)ethanol

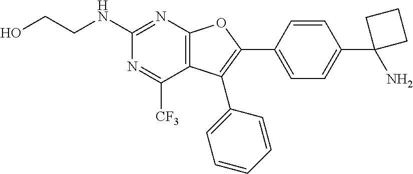

### Step 1: 4-Bromo-2-(methylthio)-5-phenylfuro[2,3-d]pyrimidine

Material contains ˜50% w/w of a DBU type impurity from the previous step. A mixture of 2-(methylthio)-5-phenylfuro[2,3-d]pyrimidin-4(3H)-one (5 g, 19.4 mmol), diisopropylethylamine (3.38 ml, 19.4 mmol) and phosphorus oxybromide (13.9 g, 48.4 mmol) in toluene (200 ml) was heated at reflux overnight under N₂. The reaction was allowed to cool to RT and neutralised using aq sodium hydrogencarbonate the undissolved solids were removed by filtration through Celite®. The biphasic mixture was diluted with ethyl acetate and the layers were separated, the organic layer was dried (MgSO₄), filtered, and concentrated in vacuo to afford the title compound as a brown gum (crude yield 7.07 g) [also contains di-brominated product]. LCMS (Method A) mono-brominated (4-bromo-2-(methylthio)-5-phenylfuro[2,3-d]pyrimidine) R_{T}=7.29 min, M+H⁺=321/323, di-brominated (4,6-dibromo-2-(methylthio)-5-phenylfuro[2,3-d]pyrimidine) R_{T}=7.86 min, M+H⁺=400/401/403.

### Step 2: 2-(Methylthio)-5-phenyl-4-(trifluoromethyl)furo[2,3-d]pyrimidine

A mixture of KF (72 mg, 1.25 mmol) and CuI (237 mg, 1.25 mmol) was heated with a heat gun under vacuum in a Schlenk tube until the solid turned green. The solid was allowed to cool to RT and the solid heated again with the heat gun under vacuum. After allowing the solid to cool to RT a solution of 4-bromo-2-(methylthio)-5-phenylfuro[2,3-d]pyrimidine (100 mg, 0.31 mmol) in NMP (1 ml) was charged to the solid followed by trimethyl(trifluoromethyl)silane (177 mg, 1.25 mmol). The reaction was heated at 50° C. overnight. The reaction mixture was added to a solution of aqueous ammonia and extracted twice with ethyl acetate, the combined organic extracts were dried (MgSO₄) filtered and concentrated in vacuo. The crude material was purified by flash chromatography (SiO₂, gradient 0-20% ethyl acetate in hexane) to afford the title compound (30 mg, 31%) as an orange gum. LCMS (Method A) R_{T}=7.44 min, M+H⁺=311.

### Step 3: 6-Bromo-2-(methylthio)-5-phenyl-4-(trifluoromethyl)furo[2,3-d]pyrimidine

To a solution of 2-(methylthio)-5-phenyl-4-(trifluoromethyl)furo[2,3-d]pyrimidine (700 mg, 2.26 mmol) in DMF (10 ml) at 0° C. was charged bromine (0.174 ml, 3.38 mmol). The reaction was stirred for 1 h at 0° C. To the reaction mixture at 0° C. was charged a 1:1 mixture of sat. aq. sodium hydrogencarbonate and 5% aq. sodium thiosulphate. The reaction was extracted twice with ethyl acetate. The organic phase was combined and washed with 1:1, water:brine (×4). The organic layer was dried (MgSO₄) filtered and concentrated in vacuo. The crude material was purified by flash chromatography (SiO₂, gradient 0-7% ethyl acetate in cyclohexane) to afford the title compound (814 mg, 93%) as an orange gum. LCMS (Method E) R_{T}=1.76 min, M+H⁺=389/391.

### Step 4: tert-Butyl 1-(4-(2-(methylthio)-5-phenyl-4-(trifluoromethyl)furo[2,3-d]pyrimidin-6-yl)phenyl)cyclobutylcarbamate

tert-Butyl 1-(4-(4,4,5,5-tetramethyl-1,3,2-dioxaborolan-2-yl)phenyl)cyclobutylcarbamate (1.1 g, 2.97 mmol, prepared as described in WO2008/070016), 6-bromo-2-(methylthio)-5-phenyl-4-(trifluoromethyl)furo[2,3-d]pyrimidine (770 mg, 1.98 mmol), and potassium phosphate tribasic (1.26 g, 5.94 mmol) in DMF (30 ml) Water (6 ml) was degassed with N₂ for 15 min. Pd(PPh₃)₄ (114 mg, 0.099 mmol) was added and the reaction mixture heated at 90° C. for 90 min. After cooling to RT the reaction mixture was diluted with water and extracted twice with ethyl acetate. The ethyl acetate layers were combined and washed with 1:1, water:brine (×4). The organic layer was dried (MgSO₄), filtered and concentrated in vacuo. The crude material was purified by flash chromatography (SiO₂, gradient 0-50% ethyl acetate in cyclohexane) to afford the desired product as a yellow foamy solid. LCMS (Method E) R_{T}=1.96 min, M+H⁺=556.

### Step 5: tert-Butyl (1-(4-(2-(methylsulfonyl)-5-phenyl-4-(trifluoromethyl)furo[2,3-d]pyrimidin-6-yl)phenyl)cyclobutyl)carbamate

Following the procedure for tert-butyl 1-(4-(2-(methylsulfonyl)-4-morpholino-5-phenylfuro[2,3-d]pyrimidin-6-yl)phenyl)cyclobutylcarbamate, tert-butyl 1-(4-(2-(methylthio)-5-phenyl-4-(trifluoromethyl)furo[2,3-d]pyrimidin-6-yl)phenyl)cyclobutylcarbamate was reacted to afford the title compound (220 mg, 0.374 mmol, 69%) as a white solid that was used in the next step without purification.

### Step 6: tert-Butyl (1-(4-(2-((2-hydroxyethyl)amino)-5-phenyl-4-(trifluoromethyl)furo[2,3-d]pyrimidin-6-yl)phenyl)cyclobutyl)carbamate

A solution of tert-butyl 1-(4-(2-(methylsulfonyl)-5-phenyl-4-(trifluoromethyl)furo[2,3-d]pyrimidin-6-yl)phenyl)cyclobutylcarbamate (150 mg, 0.255 mmol) and ethanolamine (0.5 mL, 8.27 mmol) in THF (3 mL) was stirred at RT over the weekend. The reaction mixture was concentrated in vacuo and purified by flash chromatography (SiO₂, gradient 25-60% ethyl acetate in cyclohexane) to afford the title compound (105 mg, 45%) as a white gum.

LCMS (Method E) R_{T}=1.77 min, M+H⁺=569.

### Step 7: 2-((6-(4-(1-Aminocyclobutyl)phenyl)-5-phenyl-4-(trifluoromethyl)furo[2,3-d]pyrimidin-2-yl)amino)ethanol hydrochloride

To a solution of tert-butyl 1-(4-(2-(2-hydroxyethylamino)-5-phenyl-4-(trifluoromethyl)furo[2,3-d]pyrimidin-6-yl)phenyl)cyclobutylcarbamate (100 mg, 0.176 mmol) in THF (2 ml) was charged 4M HCl in dioxane (2 ml, 8.00 mmol) at RT under N₂. The reaction was then stirred at RT overnight. To the reaction mixture was charged diethyl ether and the precipitate which formed was collected by filtration. The solid was then washed with diethyl ether and dried to afford the title compound as an off-white solid (58 mg, 65%). LCMS (Method E) R_{T}=0.95 min, M+H⁺=469. ¹H NMR (500 MHz, methanol-d₄): δ 7.38-7.55 (m, 9H), 3.79 (t, 2H), 3.64 (t, 2H), 2.71-2.77 (m, 2H), 2.53-2.59 (m, 2H), 2.18-2.26 (m, 1H), 1.90-2.00 (m, 1H).

### Example 74

### 1-(4-(2-(Methylthio)-5-phenyl-4-(trifluoromethyl)furo[2,3-d]pyrimidin-6-yl)phenyl)cyclobutanamine hydrochloride

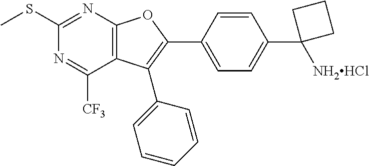

### Step 1: 1-(4-(2-(Methylthio)-5-phenyl-4-(trifluoromethyl)furo[2,3-d]pyrimidin-6-yl)phenyl)cyclobutanamine hydrochloride

To a solution of tert-butyl 1-(4-(2-(methylthio)-5-phenyl-4-(trifluoromethyl)furo[2,3-d]pyrimidin-6-yl)phenyl)cyclobutylcarbamate (50 mg, 0.090 mmol) prepared as described for 2-((6-(4-(1-aminocyclobutyl)phenyl)-4-methyl-5-phenylfuro[2,3-d]pyrimidin-2-yl)amino)ethanol in THF (1 ml) was charged 4M HCl in dioxane (1 ml, 4.00 mmol) at RT under N₂. The reaction was stirred at RT for 6 h, diluted with diethyl ether and the solid collected by filtration. The solid was washed with diethyl ether and freeze-dried (MeOH/H₂O) to afford the title compound as a white solid. LCMS (Method E) R_{T}=1.29 min, M+H⁺=456. ¹H NMR (500 MHz, methanol-d₄): δ 7.43-7.65 (m, 9H), 2.72-2.79 (m, 5H), 2.55-2.61 (m, 2H), 2.19-2.28 (m, 1H), 1.93-2.01 (m, 1H).

### Example 75

### 1-(4-(4-Iodo-2-(methylthio)-5-phenylfuro[2,3-d]pyrimidin-6

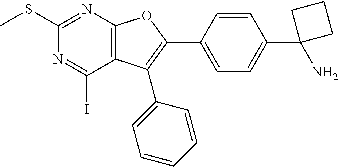

### Step 1: 1-(4-(4-Iodo-2-(methylthio)-5-phenylfuro[2,3-d]pyrimidin-6-yl)phenyl)cyclobutanamine

To a solution of 6-(4-(1-(tert-butoxycarbonylamino)cyclobutyl)phenyl)-2-(methylthio)-5-phenylfuro[2,3-d]pyrimidin-4-yl trifluoromethanesulfonate (265 mg, 0.417 mmol) in acetonitrile (2 ml) was charged sodium iodide (250 mg, 1.67 mmol) in one portion followed by the dropwise addition of a 4M solution of HCl in dioxane (0.104 ml, 0.417 mmol) over 10 min at 5° C. The reaction was stirred overnight at RT, quenched with aq. NaHCO₃, extracted with ethyl acetate, dried (MgSO₄) filtered and concentrated in vacuo. The residue was purified by flash chromatography (SiO₂, gradient 0 to 10% methanol in dichloromethane), to afford the title compound (8 mg, 4%) as a white solid. LCMS (Method E) R_{T}-=1.15 min, M+H⁺=514 ¹H NMR (500 MHz, DMSO-d₆): δ 7.40-7.60 (m, 9H), 2.60 (s, 3H), 2.30-2.38 (m, 2H), 2.02-2.07 (m, 2H), 1.92-2.00 (m, 1H), 1.58-1.66 (m, 1H).

### Example 76

### 6-(4-(1-Aminocyclobutyl)phenyl)-2-((2-hydroxyethyl)amino)-5-phenylfuro[2,3-d]pyrimidin-4(3H)-one

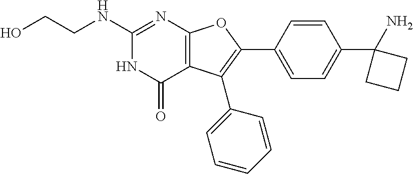

2-(6-(4-(1-Aminocyclobutyl)phenyl)-4-methoxy-5-phenylfuro[2,3-d]pyrimidin-2-ylamino)ethanol (40 mg) was dissolved in 1,4-dioxane (2 ml) followed by the addition of 4M HCl in 1,4-dioxane (3 eq) under N₂. The mixture was stirred for 10 min before the resultant precipitate was collected by filtration and dried under high vacuum affording an off-white solid (35 mg). The solid was partitioned between in sat. NaHCO₃ (aq)/DCM and extracted using further DCM. The combined organic phase was dried (Na₂SO₄), filtered and concentrated in vacuo affording a residue that was purified by prep TLC (10% 2N NH₃/MeOH in DCM eluent)—lower R_{f} band identified as pyrimidinone byproduct that was collected and triturated with EtOAc affording the title compound as an off-white solid (3 mg, 8%). LCMS (Method A) R_{T}=3.46 min, M−NH₃+H⁺=400.14.

### Example 77

### 2,2′-((6-(4-(1-Aminocyclobutyl)phenyl)-5-phenylfuro[2,3-d]pyrimidine-2,4-diyl)bis(azanediyl))diethanol

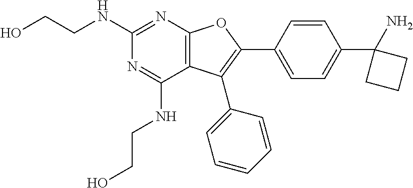

### Step 1: tert-Butyl (1-(4-(2,4-bis((2-hydroxyethyl)amino)-5-phenylfuro[2,3-d]pyrimidin-6-yl)phenyl)cyclobutyl)carbamate

Lower R_{f} by-product isolated from Example 38, Step 1. (35 mg, 9%). LCMS (Method A) R_{T}=5.86 min, M+H⁺=560.2.

### Step 2: 2,2′-((6-(4-(1-Aminocyclobutyl)phenyl)-5-phenylfuro[2,3-d]pyrimidine-2,4-diyl)bis(azanediyl))diethanol

Following a similar deprotection procedure as in Example 38 the title compound was isolated as an off-white solid. LCMS (Method E) R_{T}=0.70 min, M+H⁺=460.2 1H NMR (500 MHz, methanol-d₄): δ 7.32-7.59 (m, 9H), 3.74-3.76 (m, 2H), 3.49-3.62 (m, 6H), 2.50-2.56 (m, 2H), 2.22-2.26 (m, 2H), 2.05-2.07 (m, 1H), 1.73-1.75 (m, 1H).

### Example 78

### 1-(4-(4-Methoxy-5-phenylfuro[2,3-d]pyrimidin-6-yl)phenyl)cyclobutanamine

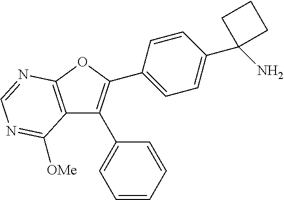

### Step 1: tert-Butyl 1-(4-(4-methoxy-5-phenylfuro[2,3-d]pyrimidin-6-yl)phenyl)cyclobutylcarbamate

To a solution of tert-butyl 1-(4-(4-methoxy-2-(methylsulfonyl)-5-phenylfuro[2,3-d]pyrimidin-6-yl)phenyl)cyclobutylcarbamate (150 mg, 0.273 mmol) in DCM (1 ml) and ethanol (1 ml) was added NaBH₄ (21 mg, 0.546 mmol). The reaction mixture was stirred at room temperature for 2 h followed by dilution with DCM and 1N HCl (aq). The combined DCM extracts were dried (Na₂SO₄) and concentrated in vacuo before purification by silica gel chromatography (0 to 50% EtOAc/cyclohexane) affording tert-butyl 1-(4-(4-methoxy-5-phenylfuro[2,3-d]pyrimidin-6-yl)phenyl)cyclobutylcarbamate (105 mg, 0.223 mmol, 82%) as a white solid. LCMS (Method A) R_{T}=8.68 min, M+H⁺=472.2.

### Step 2: 1-(4-(4-Methoxy-5-phenylfuro[2,3-d]pyrimidin-6-yl)phenyl)cyclobutanamine

To a solution of tert-butyl 1-(4-(4-methoxy-5-phenylfuro[2,3-d]pyrimidin-6-yl)phenyl)cyclobutylcarbamate (105 mg, 0.223 mmol) in DCM (2 ml) was added TFA (1 ml, 12.98 mmol). The reaction mixture was stirred at room temperature for 1 h. The solvents were removed in vacuo. The remaining residue was partitioned between DCM and sat. NaHCO₃ (aq). DCM extracts were dried (Na₂SO₄) and concentrated affording an off-white solid that was purified by silica gel chromatography (0 to 10% 1N NH₃/MeOH in DCM) affording the title compound (30 mg, 0.081 mmol, 36%) as a white solid after freeze-drying. LCMS (Method A) R_{T}=4.56 min, M+H⁺=373.15. ¹H NMR (500 MHz, methanol-d₄): δ 8.43 (s, 1H), 7.46 (d, 2H), 7.3-7.4 (m, 7H), 3.9 (s, 3H), 2.45 (m, 2H), 2.15 (m, 2H), 2.0 (m, 1H), 1.65 (m, 1H).

### Example 79

### 2-(6-(4-(1-Aminocyclobutyl)phenyl)-4-(methylamino)-5-phenylfuro[2,3-d]pyrimidin-2-ylamino)ethanol, HCl

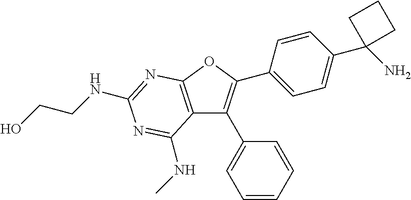

### Step 1: tert-Butyl 1-(4-(2-(2-hydroxyethylamino)-4-(methylamino)-5-phenylfuro[2,3-d]pyrimidin-6-yl)phenyl)cyclobutylcarbamate

To a solution of tert-butyl 1-(4-(4-(methylamino)-2-(methylsulfonyl)-5-phenylfuro[2,3-d]pyrimidin-6-yl)phenyl)cyclobutylcarbamate (0.094 g, 0.171 mmol) in THF (2 ml) was added 2-aminoethanol (0.105 g, 1.71 mmol). The reaction mixture was then stirred at 50° C. for 72 h before analysis by TLC indicated some residual starting material. α,α,α-Trifluorotoluene (2 ml) was added and the reaction heated to 90° C. in the microwave for 30 min. Analysis by TLC indicated reaction was complete. The reaction mixture was concentrated in vacuo and remaining residue was purified by silica gel chromatography (0 to 100% EtOAc/cyclohexane eluent) affording the title compound (0.07 g, 0.132 mmol, 77%) as a clear gum. LCMS (Method A) R_{T}=6.24 min, M+H⁺=530.23.

### Step 2: 2-(6-(4-(1-Aminocyclobutyl)phenyl)-4-(methylamino)-5-phenylfuro[2,3-d]pyrimidin-2-ylamino)ethanol, HCl

To a solution of tert-butyl 1-(4-(2-(2-hydroxyethylamino)-4-(methylamino)-5-phenylfuro[2,3-d]pyrimidin-6-yl)phenyl)cyclobutylcarbamate (0.06 g, 0.113 mmol) in THF (2 ml) was added HCl (4M in dioxane) (2 ml, 65.8 mmol). The reaction mixture was stirred at room temperature for 3 h. Et₂O (2 ml) was added and the mixture was stirred for 15 min before filtration. The collected solid was washed with Et₂O and dried under high vacuum affording the title compound (0.03 g, 57%) as a white solid.

LCMS (Method A) R_{T}=3.93 min, M−NH₃+H⁺=413.28. NMR (500 MHz, methanol-d₄): δ 7.62 (m, 3H), 7.42-7.52 (m, 6H), 3.8 (t, 2H), 3.65 (t, 2H), 3.02 (s, 3H), 2.75 (m, 2H), 2.6 (m, 2H), 2.24 (m, 1H), 1.95 (m, 1H).

### Example 80

### 1-(4-(7-Bromo-4-methoxy-3-phenylfuro[3,2-c]pyridin-2-yl)phenyl)cyclobutanamine

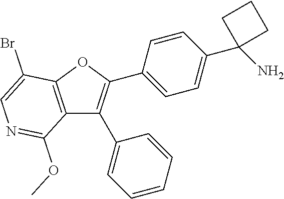

### Step 1: tert-Butyl 1-(4-(7-bromo-4-methoxy-3-phenylfuro[3,2-c]pyridin-2-yl)phenyl)cyclobutylcarbamate

To a solution of tert-butyl 1-(4-(4-methoxy-3-phenylfuro[3,2-c]pyridin-2-yl)phenyl)cyclobutylcarbamate (0.1 g, 0.213 mmol) in CCl₄ (1 ml) was added bromine (0.012 ml, 0.234 mmol). The reaction mixture was heated to 50° C. and maintained at this temperature for 72 h. LCMS analysis indicated residual starting material and therefore a further 1 eq Br₂ added and heating was continued overnight. The reaction mixture was concentrated in vacuo and purified by silica gel chromatography (gradient elution 0 to 30% EtOAc/cyclohexane) affording the title compound (0.040 g, 34%) as a gum. LCMS (Method C) R_{T}=2.02 min, M+H⁺=551. [N.B. tert-Butyl 1-(4-(7-bromo-4-oxo-3-phenyl-4,5-dihydrofuro[3,2-c]pyridin-2-yl)phenyl)cyclobutylcarbamate (25 mg, 22%) was also isolated as a lower R_{f} product.

LCMS (Method E) R_{T}=1.577 min, M+H⁺=537].

### Step 2: 1-(4-(7-Bromo-4-methoxy-3-phenylfuro[3,2-c]pyridin-2-yl)phenyl)cyclobutanamine

To a solution of tert-butyl 1-(4-(7-bromo-4-methoxy-3-phenylfuro[3,2-c]pyridin-2-yl)phenyl)cyclobutylcarbamate (30 mg, 0.055 mmol) in DCM (2 ml) was added TFA (1 ml). The reaction mixture was stirred at room temperature for 1 h. The reaction mixture was concentrated in vacuo, diluted with DCM and sat. Na₂CO₃ (aq) and separated. The combined DCM extracts were concentrated and the residue purified by silica gel chromatography (0 to 10% 1N NH₃/MeOH in DCM) yielding the title compound (0.017 g, 69%) as a white solid. LCMS (Method E) R_{T}=1.086 min, M−NH₃+H⁺=434. ¹H NMR (500 MHz, methanol-d₄): δ 8.11 (s, 1H), 7.63 (d, 2H), 7.45 (m, 7H), 3.83 (s, 3H), 2.74 (m, 2H), 2.58 (m, 2H), 2.23 (m, 1), 1.95)ₘ, 1H).

### Example 81

### 2-(6-(4-(1-Aminocyclobutyl)phenyl)-4-(ethylamino)-5-phenylfuro[2,3-d]pyrimidin-2-ylamino)ethanol, HCl

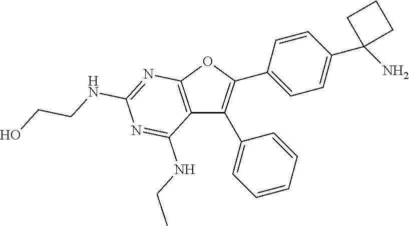

### Step 1: tert-Butyl 1-(4-(4-(ethylamino)-2-(methylthio)-5-phenylfuro[2,3-d]pyrimidin-6-yl)phenyl)cyclobutylcarbamate

To a solution of 6-(4-(1-(tert-butoxycarbonylamino)cyclobutyl)phenyl)-2-(methylthio)-5-phenylfuro[2,3-d]pyrimidin-4-yl trifluoromethanesulfonate (200 mg, 0.315 mmol) was added ethanamine (2M in MeOH) (2 ml, 0.315 mmol). The reaction mixture was then stirred at 50° C. for 2 h. The reaction mixture was concentrated in vacuo and purified by silica gel chromatography (0 to 25% EtOAc/cyclohexane) affording the title compound (100 mg, 60%) as a gum. LCMS (Method E) R_{T}=1.953 min, M+H⁺=531.2.

### Step 2: tert-Butyl 1-(4-(4-(ethylamino)-2-(methylsulfonyl)-5-phenylfuro[2,3-d]pyrimidin-6-yl)phenyl)cyclobutylcarbamate

To a solution of tert-butyl 1-(4-(4-(ethylamino)-2-(methylthio)-5-phenylfuro[2,3-d]pyrimidin-6-yl)phenyl)cyclobutylcarbamate (0.1 g, 0.188 mmol) in tetrahydrofuran (3 ml) and methanol (1.5 ml) was added Oxone® (0.463 g, 0.754 mmol) in water (1.5 ml). The reaction mixture was heated to 50° C. and maintained at this temperature for 2 h. Analysis by TLC (1:1, EtOAc/cyclohexane) shown that the reaction was complete. The reaction was cooled and diluted with EtOAc and water. EtOAc extracts were dried (Na₂SO₄) and concentrated in vacuo affording the title compound (0.077 g, 73%) as a brown gum. LCMS (Method E) R_{T}=1.656 min, M+H⁺=563.2.

### Step 3: tert-Butyl 1-(4-(4-(ethylamino)-2-(2-hydroxyethylamino)-5-phenylfuro[2,3-d]pyrimidin-6-yl)phenyl)cyclobutylcarbamate

To a solution of tert-butyl 1-(4-(4-(ethylamino)-2-(methylsulfonyl)-5-phenylfuro[2,3-d]pyrimidin-6-yl)phenyl)cyclobutylcarbamate (0.075 g, 0.133 mmol) in α,α,α-trifluorotoluene (3 ml) was added 2-aminoethanol (0.163 g, 2.67 mmol). The reaction mixture was heated at 105° C. under microwave conditions for 45 min. Reaction was deemed complete by TLC after this time. The reaction mixture was diluted with EtOAc and water and extracted. Organic extracts were washed with water (2×10 ml), dried (Na₂SO₄) and concentrated in vacuo affording the crude title compound as an oil (31 mg, 43%). LCMS (Method E) R_{T}=1.561 min, M+H⁺=544.2.

### Step 4: 2-(6-(4-(1-Aminocyclobutyl)phenyl)-4-(ethylamino)-5-phenylfuro[2,3-d]pyrimidin-2-ylamino)ethanol, HCl

To a solution of tert-butyl 1-(4-(4-(ethylamino)-2-(2-hydroxyethylamino)-5-phenylfuro[2,3-d]pyrimidin-6-yl)phenyl)cyclobutylcarbamate (0.031 g, 0.057 mmol) in DCM (2 ml) was added TFA (1 ml). The reaction mixture was stirred at room temperature for 1 h. The reaction mixture was concentrated in vacuo, diluted with DCM (15 ml) and sat. Na₂CO₃ (aq) (10 ml) and extracted. The DCM extracts were dried (Na₂SO₄), filtered and concentrated affording crude product as a gum. The residue was taken in 1,4-dioxane and 2 eq. HCl in MeOH added. The resultant precipitate was collected by filtration and dried affording the title compound (0.012 g, 44%) as a white solid. LCMS (Method E) R_{T}=0.871 min, M−NH₃+H⁺=444.2. NMR (500 MHz, methanol-d₄): δ 7.5 (m, 3H), 7.4 (m, 4H), 7.3 (d, 2H), 3.7 (t, 2H), 3.55 (t, 2H), 3.3 (q, 2H), 2.6 (m, 2H), 2.48 (m, 2H), 2.12 (m, 1H), 1.8 (m, 1H), 1.0 (t, 3H).

### Example 82

### 6-(4-(1-Aminocyclobutyl)phenyl)-3-methyl-2-morpholino-5-phenylfuro[2,3-d]pyrimidin-4(3H)-one

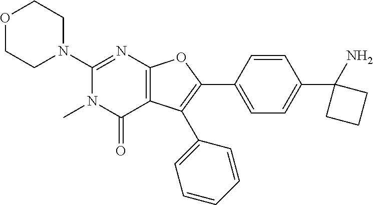

### Step 1: 6-(4-(1-Aminocyclobutyl)phenyl)-3-methyl-2-(methylsulfinyl)-5-phenylfuro[2,3-d]pyrimidin-4(3H)-one

To a solution of tert-butyl 1-(4-(3-methyl-2-(methylthio)-4-oxo-5-phenyl-3,4-dihydrofuro[2,3-d]pyrimidin-6-yl)phenyl)cyclobutylcarbamate (0.7 g, 1.35 mmol) in THF (14 ml) and MeOH (7.0 ml) was added Oxone (3.33 g, 5.41 mmol) in water (7.0 ml) dropwise over 10 min. The reaction was stirred at 50° C. and monitored by LCMS. After 2 h the reaction was diluted with water and DCM, extracted and concentrated in vacuo. Resultant solid was slurried in Et₂O and filtered yielding the title compound as a beige solid (75 mg, 12%). LCMS (Method E) R_{T}=0.895 min, M+H⁺=434.

### Step 2: 6-(4-(1-Aminocyclobutyl)phenyl)-3-methyl-2-morpholino-5-phenylfuro[2,3-d]pyrimidin-4(3H)-one, HCl

To a solution of 6-(4-(1-aminocyclobutyl)phenyl)-3-methyl-2-(methylsulfinyl)-5-phenylfuro[2,3-d]pyrimidin-4(3H)-one (0.075 g, 0.173 mmol) in DMF (1 ml) was added morpholine (0.151 ml, 1.73 mmol). The reaction mixture was stirred at 90° C. for 1 h. The reaction mixture was cooled, diluted with EtOAC and brine and extracted (×3). The organic extracts were dried (Na₂SO₄), filtered and concentrated in vacuo. The residue was purified by silica gel chromatography (0 to 10% 1N NH₃/MeOH in DCM) affording crude product as a cream solid. The solid was dissolved in THF (1 ml) and treated with 4N HCl in 1,4-dioxane and the resultant solid was collected by filtration and washed with Et₂O. The solid was freeze-dried affording the title compound (0.02 g, 0.041 mmol, 23%) as an off-white solid. LCMS (Method E) R_{T}=0.861 min, M−NH₃+H⁺=440.2. ¹H NMR (500 MHz, methanol-d₄): δ 7.5 (d, 2H), 7.3-7.4 (m, 7H), 3.75 (m, 4H), 3.48 (s, 3H), 3.25 (m, 4H), 2.65 (m, 2H), 2.5 (m, 2H), 2.15 (m, 1H), 1.85 (m, 1H).

### Example 83

### 6-(4-(1-Aminocyclobutyl)phenyl)-2-(2-methoxyethylamino)-3-methyl-5-phenylfuro[2,3-d]pyrimidin-4(3H)-one

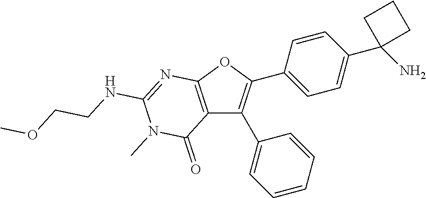

### Step 1: tert-Butyl 1-(4-(2-(2-methoxyethylamino)-3-methyl-4-oxo-5-phenyl-3,4-dihydrofuro[2,3-d]pyrimidin-6-yl)phenyl)cyclobutylcarbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (0.11 g, 0.20 mmol) in THF (3 ml) was added 2-methoxyethanamine (0.150 g, 2.00 mmol). The reaction mixture was stirred at room temperature for 15 min. The solvents were removed in vacuo and the residue was purified by silica gel chromatography (0 to 100% EtOAc/cyclohexane eluent) affording tert-butyl 1-(4-(2-(2-methoxyethylamino)-3-methyl-4-oxo-5-phenyl-3,4-dihydrofuro[2,3-d]pyrimidin-6-yl)phenyl)cyclobutylcarbamate (0.035 g, 0.064 mmol, 32%) as an off-white solid. LCMS (Method E) R_{T}=1.61 min, M+H⁺=545.2.

### Step 2: 6-(4-(1-Aminocyclobutyl)phenyl)-2-(2-methoxyethylamino)-3-methyl-5-phenylfuro[2,3-d]pyrimidin-4(3H)-one

To a solution of tert-butyl 1-(4-(2-(2-methoxyethylamino)-3-methyl-4-oxo-5-phenyl-3,4-dihydrofuro[2,3-d]pyrimidin-6-yl)phenyl)cyclobutylcarbamate (0.035 g, 0.064 mmol) in DCM (2 ml) was added TFA (1 ml). The reaction mixture was stirred at room temperature for 1 h. The reaction mixture was concentrated in vacuo, diluted with DCM and sat. Na₂CO₃ (aq) and extracted. The organic phase was concentrated and purified by silica gel chromatography (0 to 10% 1N NH₃/MeOH in DCM) affording the title compound (0.015 g, 53%) as an off-white solid after drying under high vacuum. LCMS (Method E) R_{T}=0.808 min, M−NH₃+H⁺=428.2.

¹H NMR (500 MHz, CDCl₃): δ 7.45 (m, 4H), 7.25-7.35 (m, 3H), 7.2 (m, 2H), 5.1 (t, 1H), 3.65 (m, 2H), 3.57 (m, 2H), 3.35 (s, 3H), 3.34 (s, 3H), 2.45 (m, 2H), 2.14 (m, 2H), 2.05 (m, 1H), 1.7 (m, 1H).

### Example 84

### 6-(4-(1-Aminocyclobutyl)phenyl)-2-((2-hydroxyethyl)(methyl)amino)-3-methyl-5-phenylfuro[2,3-d]pyrimidin-4(3H)-one

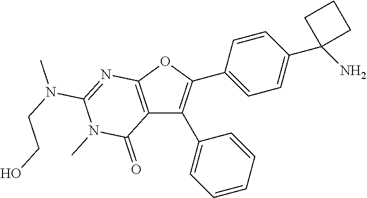

### Step 1: tert-Butyl 1-(4-(2-((2-hydroxyethyl)(methyl)amino)-3-methyl-4-oxo-5-phenyl-3,4-dihydrofuro[2,3-d]pyrimidin-6-yl)phenyl)cyclobutylcarbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (0.1 g, 0.182 mmol) in tetrahydrofuran (2 ml) was added 2-(methylamino)ethanol (0.137 g, 1.82 mmol). The reaction mixture was stirred at RT for 30 min. The reaction mixture was concentrated in vacuo, diluted with DCM and brine and extracted (×3). The combined organic extracts were dried (Na₂SO₄), filtered and concentrated in vacuo. The residue was purified by silica gel chromatography (0 to 100% EtOAc in cyclohexane) affording the title compound (0.035 g, 35%) as an off-white solid.

LCMS (Method E) R_{T}=1.5 min, M+H⁺=545.2.

### Step 2: 6-(4-(1-Aminocyclobutyl)phenyl)-2-((2-hydroxyethyl)(methyl)amino)-3-methyl-5-phenylfuro[2,3-d]pyrimidin-4(3H)-one

To a solution of tert-butyl 1-(4-(2-((2-hydroxyethyl)(methyl)amino)-3-methyl-4-oxo-5-phenyl-3,4-dihydrofuro[2,3-d]pyrimidin-6-yl)phenyl)cyclobutylcarbamate (0.035 g, 0.064 mmol) in DCM (2 ml) was added TFA (1 ml). The reaction mixture was stirred at room temperature for 1 h. The reaction mixture was concentrated in vacuo, diluted with DCM (15 ml) and sat. Na₂CO₃ (aq) (10 ml) and extracted. The DCM extracts were dried (Na₂SO₄), concentrated and the residue purified by silica gel chromatography (0 to 10% 2N NH₃/MeOH in DCM) affording the title compound (0.006 g, 21%) as a white solid after drying under high vacuum. LCMS (Method E) R_{T}=0.751 min, M−NH₃+H⁺=428.2. ¹H NMR (500 MHz, DMSO-d₆): δ 7.3-7.45 (m, 9H), 3.65 (m, 2H), 3.4 (s, 3H), 3.37 (m, 2H), 2.96 (s, 3H), 2.35 (m, 2H), 2.05 (m, 2H), 2.0 (m, 1H), 1.64 (m, 1H).

### Example 85

### 6-(4-(1-Aminocyclobutyl)phenyl)-2-(2-hydroxyethoxy)-3-methyl-5-phenylfuro[2,3-d]pyrimidin-4(3H)-one, HCl

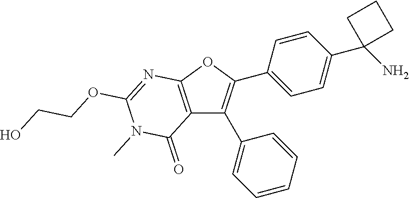

### Step 1: tert-Butyl 1-(4-(2-(2-hydroxyethoxy)-3-methyl-4-oxo-5-phenyl-3,4-dihydrofuro[2,3-d]pyrimidin-6-yl)phenyl)cyclobutylcarbamate

To a suspension of NaH (0.037 g, 1.55 mmol) in DMF (1 ml) at 0° C. was added ethylene glycol (0.043 ml, 0.773 mmol) dropwise. After 10 min, tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (0.085 g, 0.155 mmol) in DMF (0.5 ml) was added dropwise. The reaction was then stirred at 0° C. for 10 min. The reaction was quenched with water and extracted with EtOAc. The EtOAc extracts were washed with brine (×3), dried (Na₂SO₄) and concentrated in vacuo affording a clear gum. The residue was purified by silica gel chromatography (0 to 100% EtOAc/cyclohexane) affording the title compound (0.065 g, 79%) as a white solid. LCMS (Method A) R_{T}=6.79 min, M+H⁺=532.18.

### Step 2: 6-(4-(1-Aminocyclobutyl)phenyl)-2-(2-hydroxyethoxy)-3-methyl-5-phenylfuro[2,3-d]pyrimidin-4(3H)-one, HCl

To a solution of tert-butyl 1-(4-(2-(2-hydroxyethoxy)-3-methyl-4-oxo-5-phenyl-3,4-dihydrofuro[2,3-d]pyrimidin-6-yl)phenyl)cyclobutylcarbamate (0.065 g, 0.122 mmol) in THF (2 ml) was added HCl (4.0M in dioxane) (2 ml, 65.8 mmol). The reaction mixture was stirred at room temperature for 2 h. Analysis by LCMS indicated that the reaction was partially incomplete. Further 4M HCl in dioxane (1 ml) was added and the reaction was allowed to stir overnight. The resultant precipitate was collected by filtration, washed with Et₂O and dried under high vacuum the title compound (0.017 g, 30%) as a white solid. LCMS (Method E) R_{T}=0.763 min, M−NH₃+H⁺=428.2.

1H NMR (500 MHz, DMSO-d₆): 7.4-7.5 (m, 9H), 5.04 (t, 1H), 4.5 (m, 2H), 3.7 (m, 2H), 3.35 (s, 3H), 2.59 (m, 2H), 2.5 (m, 2H), 2.1 (m, 1H), 1.78 (m, 1H).

### Example 86

### (S)-6-(4-(1-Aminocyclobutyl)phenyl)-2-((2-hydroxypropyl)amino)-3-methyl-5-phenylfuro[2,3-d]pyrimidin-4(3H)-one

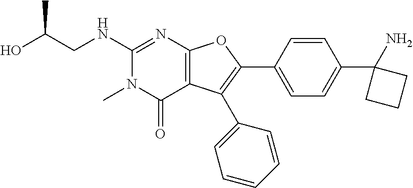

### Step 1: (S)-tert-Butyl 1-(4-(2-(2-hydroxypropylamino)-3-methyl-4-oxo-5-phenyl-3,4-dihydrofuro[2,3-d]pyrimidin-6-yl)phenyl)cyclobutylcarbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (0.15 g, 0.273 mmol) in THF (3 ml) was added (S)-1-aminopropan-2-ol (0.205 g, 2.73 mmol). The reaction mixture was stirred at room temperature for 15 min. The reaction mixture was concentrated in vacuo, diluted with DCM and purified by silica gel chromatography (0 to 5% DCM/MeOH) to afford (S)-tert-butyl 1-(4-(2-(2-hydroxypropylamino)-3-methyl-4-oxo-5-phenyl-3,4-dihydrofuro[2,3-d]pyrimidin-6-yl)phenyl)cyclobutylcarbamate (0.105 g, 71%) as a gum. LCMS (Method E) R_{T}=1.485 min, M+H⁺=545.2.

### Step 2: (S)-6-(4-(1-Aminocyclobutyl)phenyl)-2-((2-hydroxypropyl)amino)-3-methyl-5-phenylfuro[2,3-d]pyrimidin-4(3H)-one

To a solution of (S)-tert-butyl 1-(4-(2-(2-hydroxypropylamino)-3-methyl-4-oxo-5-phenyl-3,4-dihydrofuro[2,3-d]pyrimidin-6-yl)phenyl)cyclobutylcarbamate (0.105 g, 0.193 mmol) in DCM (3 ml) at 0° C. was added TFA (1 ml). The reaction mixture was stirred at room temperature for 5 min before concentration in vacuo followed by dilution with DCM (15 ml) and sat. Na₂CO₃ (aq) (10 ml). The DCM extracts were dried (Na₂SO₄), concentrated and the residue purified by silica gel chromatography (0 to 10% 1N NH₃/MeOH in DCM) affording (S)-6-(4-(1-aminocyclobutyl)phenyl)-2-(2-hydroxypropylamino)-3-methyl-5-phenylfuro[2,3-d]pyrimidin-4(3H)-one (0.055 g, 0.124 mmol, 64%) as a clear gum. The gum was dissolved in 1,4-dioxane (2 ml), cooled to 0° C. under N₂ and HCl in MeOH (2 eq) added (prepared from acetyl chloride in MeOH). The resultant precipitate was collected by filtration, washed with Et₂O and freeze-dried affording the title compound as a white solid (55 mg, 64%). LCMS (Method E) R_{T}=0.781 min, M−NH₃+H⁺=428.2. ¹H NMR (500 MHz, methanol-d₄): a 7.42 (m, 2H), 7.25-7.35 (m, 7H), 4.0 (m, 1H), 3.49 (dd, 1H), 3.3 (m, 4H), 2.65 (m, 2H), 2.47 (m, 2H), 2.1 (m, 1H), 1.83 (m, 1H), 1.15 (d, 3H).

### Example 87

### 6-(4-(1-Aminocyclobutyl)phenyl)-2,3-dimethyl-5-phenylfuro[2,3-d]pyrimidin-4(3H)-one

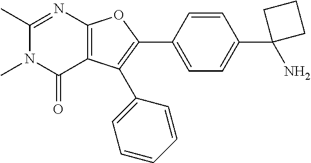

### Step 1: tert-Butyl 1-(4-(2,3-dimethyl-4-oxo-5-phenyl-3,4-dihydrofuro[2,3-d]pyrimidin-6-yl)phenyl)cyclobutylcarbamate

To a stirring solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (200 mg, 0.364 mmol) in anhydrous THF (8 ml) at −78° C. was added methylmagnesium chloride (3.0M in THF) (0.61 ml, 1.82 mmol). The reaction was warmed to room temperature and quenched by addition of ammonium chloride solution (10% w/w, 20 ml). The THF was removed in vacuo and the remaining aqueous extracted with DCM (4×12.5 ml). The combined organic phases were concentrated in vacuo and the residue purified by silica gel chromatography (0 to 5% methanol in DCM) to afford tert-butyl 1-(4-(2,3-dimethyl-4-oxo-5-phenyl-3,4-dihydrofuro[2,3-d]pyrimidin-6-yl)phenyl)cyclobutylcarbamate (7.9 mg). This compound was dissolved in DCM (0.5 ml) and placed on a Biotage® SCX-2 silica cartridge. After 30 minutes the cartridge was flushed with methanol (3 ml) then 2M ammonia in methanol (3 ml). The ammonia/methanol fraction was concentrated in vacuo. The residue was dissolved in methanol (2 ml) and treated with a solution of HCl in methanol (0.092 ml, 0.28M). After 1 minute the solvent was removed in vacuo. The residue was dissolved in water and freeze-dried affording the title compound (5.3 mg, 3%). LCMS (Method E) R_{T}=0.79 min, M+H⁺=386.2. ¹H NMR (500 MHz, DMSO) δ 8.64 (br s, 2H), 7.48-7.53 (m, 4H), 7.45 (m, 5H), 3.49 (s, 3H), 3.40 (s, 3H), 3.40 (m, 4H), 2.10-2.20 (m, 1H), 1.73-1.83 (m, 1H)

### Example 88

### 6-(4-(1-Aminocyclobutyl)phenyl)-2-(ethylamino)-3-methyl-5-phenylfuro[2,3-d]pyrimidin-4(3H)-one, HCl

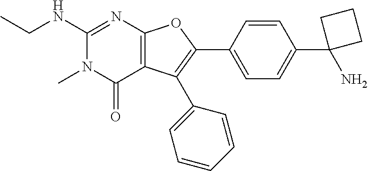

### Step 1: tert-Butyl 1-(4-(2-(ethylamino)-3-methyl-4-oxo-5-phenyl-3,4-dihydrofuro[2,3-d]pyrimidin-6-yl)phenyl)cyclobutylcarbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (223 mg, 0.406 mmol) in anhydrous THF (8 ml) was added 2-Methylamine in methanol (1.01 ml, 2.029 mmol). The reaction was stirred for 3 h after which the solvent was removed in vacuo. The residue was dissolved in 5% methanol in DCM (v/v, 5 ml) then concentrated in vacuo to 2.5 ml. Hexane was added (5 ml) precipitating a solid which was collected by filtration affording the title compound (99.7 mg, 48%). The filtrate was concentrated in vacuo and the residue purified by silica gel chromatography (gradient 0 to 50% EtOAc in cyclohexane) to afford a further portion of the title compound (33.9 mg, 16%). LCMS (of both portions) (Method E) R_{T}=1.85 min, M+H⁺=515.2.

### Step 2: 6-(4-(1-Aminocyclobutyl)phenyl)-2-(ethylamino)-3-methyl-5-phenylfuro[2,3-d]pyrimidin-4(3H)-one, HCl

4M HCl in 1,4-dioxane (3 ml, 12.0 mmol)) was added to a stirring suspension of tert-butyl 1-(4-(2-(ethylamino)-3-methyl-4-oxo-5-phenyl-3,4-dihydrofuro[2,3-d]pyrimidin-6-yl)phenyl)cyclobutylcarbamate (99.7 mg, 0.194 mmol) in anhydrous THF (1 ml). The mixture was stirred for 1.5 h. Diethyl ether (10 ml) was added to the suspension and the solid was collected by vacuum filtration after 10 min. The cake was washed with diethyl ether (10 ml). The solid was dissolved in hot water/ethanol (5 ml: 1.5 ml) and cooled with stirring forming a fine precipitate which was collected by vacuum filtration. The cake was washed with cold acetonitrile (3 ml) and diethyl ether (5 ml) and dried under vacuum to afford the title compound (44.9 mg, 38%). LCMS (Method E) R_{T}=0.84 min, M+H⁺=415.2. ¹H NMR (500 MHz, methanol-d₄) δ 7.55 (d, 2H), 7.46-7.48 (m, 2H), 7.41-7.42 (m, 5H), 3.58 (q, 2H), 3.40 (s, 3H), 2.74-2.80 (m, 2H), 2.58-2.62 (m, 2H), 2.20-2.26 (m, 1H), 1.94-1.99 (m, 1H), 1.33 (t, 3H).

### Example 89

### 6-(4-(1-Aminocyclobutyl)phenyl)-4-(2-fluoroethoxy)-N-methyl-5-phenylfuro[2,3-d]pyrimidin-2-amine, HCl

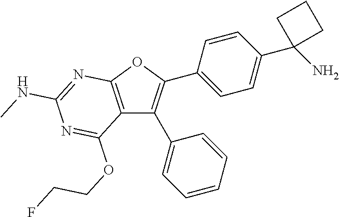

### Step 1: tert-Butyl 1-(4-(4-(2-fluoroethoxy)-2-(methylamino)-5-phenylfuro[2,3-d]pyrimidin-6-yl)phenyl)cyclobutylcarbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (150 mg, 0.258 mmol) in anhydrous THF (5 ml) was added 2M methylamine in methanol (0.65 ml, 1.289 mmol). The reaction was stirred for 21 h after which the solvent was removed in vacuo. The residue was dissolved in 5% methanol in DCM (v/v, 1 ml). Diethyl ether was added (5 ml) and the mixture stirred for 40 min precipitating a solid which was collected by filtration and dried under vacuum affording the title compound (81.7 mg, 60%). LCMS (Method E) R_{T}=1.75 min, M+H⁺=533.2.

### Step 2: 6-(4-(1-Aminocyclobutyl)phenyl)-4-(2-fluoroethoxy)-N-methyl-5-phenylfuro[2,3-d]pyrimidin-2-amine, HCl

4M HCl in 1,4-dioxane (2 ml, 8.0 mmol) was added to a stirring suspension of tert-butyl 1-(4-(4-(2-fluoroethoxy)-2-(methylamino)-5-phenylfuro[2,3-d]pyrimidin-6-yl)phenyl)cyclobutylcarbamate (81.7 mg, 0.153 mmol) in anhydrous THF (1 ml). The mixture was stirred for 1 h. Diethyl ether (5 ml) was added to the suspension and the solid was collected by vacuum filtration after 1 h. The cake was washed with diethyl ether (5 ml). The solid was suspended in hot water and filtered through a 0.2 μm filter. The filtrate was freeze-dried to afford the title compound (46.6 mg, 65%). LCMS (Method E) R_{T}=0.95 min, M+H⁺=433.2. ¹H NMR (500 MHz, methanol-d₄) δ 7.61 (d, 2H), 7.46-7.49 (m, 2H), 7.42-7.44 (m, 5H), 4.60-4.63 (m, 2H), 4.56-4.58 (m, 1H), 4.51-4.53 (m, 1H), 3.01 (s, 3H), 2.75-2.80 (m, 2H), 2.59-2.64 (m, 2H), 2.23-2.26 (m, 1H), 1.96-1.98 (m, 1H).

### Example 90

### 6-(4-(1-Aminocyclobutyl)phenyl)-2-cyclopropyl-3-methyl-5-phenylfuro[2,3-d]pyrimidin-4(3H)-one

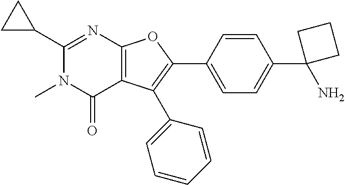

To a stirred solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (200 mg, 0.364 mmol) in anhydrous THF (8 ml) at −78° C. was added cyclopropylmagnesium chloride (5×0.73 ml, 5×0.364 mmol) at 5 minute intervals. The reaction was warmed to room temperature and quenched by addition of ammonium chloride solution (10% w/w, 20 ml). The THF was removed in vacuo and the remaining aqueous extracted with DCM (4×12.5 ml). The combined organic phases were concentrated in vacuo and the residue purified by silica gel chromatography (0 to 40% EtOAc in cyclohexane) to afford tert-butyl (1-(4-(2-cyclopropyl-3-methyl-4-oxo-5-phenyl-3,4-dihydrofuro[2,3-d]pyrimidin-6-yl)phenyl)cyclobutyl)carbamate (31.7 mg). This compound was dissolved in DCM (2 ml) and placed on a Biotage® SCX-2 silica cartridge. After 1 h, the cartridge was flushed with methanol (6 ml), then 2M ammonia in methanol (6 ml). The ammonia/methanol fraction was concentrated in vacuo. The residue was purified by preparative HPLC (Method H) and the solvents removed in vacuo to afford the title compound (13.9 mg, 9%). LCMS (Method E) R_{T}=0.91 min, M+H⁺=412.2. ¹H NMR (500 MHz, methanol-d₄) δ 7.46-7.48 (m, 4H), 7.38-7.43 (m, 5H), 3.77 (s, 3H), 2.52-2.57 (m, 2H), 2.31-2.34 (m, 1H), 2.22-2.27 (m, 2H), 2.07-2.09 (m, 1H), 1.74-1.76 (m, 1H), 1.27-1.30 (m, 2H), 1.18-1.26 (m, 2H).

### Example 91

### 2-(4-(1-Aminocyclobutyl)phenyl)-5-(2-fluoroethyl)-3-phenylfuro[3,2-c]pyridin-4(5H)-one

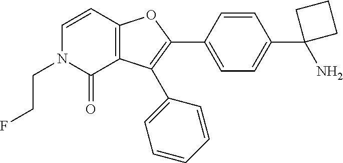

### Step 1: tert-Butyl 1-(4-(5-(2-fluoroethyl)-4-oxo-3-phenyl-4,5-dihydrofuro[3,2-c]pyridin-2-yl)phenyl)cyclobutylcarbamate

1-Fluoro-2-iodoethane (11.57 mg, 0.067 mmol) was added to a stirring suspension of tert-butyl 1-(4-(4-oxo-3-phenyl-4,5-dihydrofuro[3,2-c]pyridin-2-yl)phenyl)cyclobutylcarbamate (25.3 mg, 0.055 mmol) and potassium carbonate (18.4 mg, 0.133 mmol) in DMF (0.5 ml) at 60° C. under a nitrogen atmosphere. The mixture was stirred for 24 h then cooled to room temperature. Brine/water (1:1 v/v, 10 ml) was added and the aqueous extracted with ethyl acetate (4×8 ml). The combined organic fractions were washed with brine/water (1:1, 4×10 ml), dried (Na₂SO₄), filtered and concentrated in vacuo. The residue was purified by silica gel chromatography (gradient 0 to 50% EtOAc in cyclohexane) to afford crude title compound (33.5 mg) which was used without further purification. LCMS (Method E) R_{T}=1.64 min, M+H⁺=503.2.

### Step 2: 2-(4-(1-Aminocyclobutyl)phenyl)-5-(2-fluoroethyl)-3-phenylfuro[3,2-c]pyridin-4(5H)-one

A Biotage® SCX-2 silica cartridge was flushed with a solution of methanol in DCM (20% v/v, 20 ml). tert-Butyl 1-(4-(5-(2-fluoroethyl)-4-oxo-3-phenyl-4,5-dihydrofuro[3,2-c]pyridin-2-yl)phenyl)cyclobutylcarbamate (28.0 mg) was dissolved in a minimum of DCM and placed on the cartridge. After 16 h, the cartridge was flushed with methanol in DCM (20% v/v, 20 ml) then 7M ammonia in methanol in DCM (20% v/v, 20 ml). The ammonia/methanol/DCM phase was concentrated in vacuo. The residue was dissolved in acetonitrile (0.5 ml) and DMSO (1 ml) then diluted with water (5 ml) and freeze-dried to afford the title compound (12.0 mg, 54%). LCMS (Method E) R_{T}=0.82 min, M+H⁺=403.2. ¹H NMR (500 MHz, DMSO) δ 7.68 (d, 1H), 7.36-7.42 (m, 9H), 6.83 (d, 1H), 4.61 (t, 1H) 4.71 (t, 1H), 4.24 (t, 1H) 4.29 (t, 1H), 2.55 (s, 2H), 2.33-2.39 (m, 2H), 2.07-2.12 (m, 2H), 1.97-2.01 (m, 1H), 1.63-1.67 (m, 1H).

### Example 92

### 6-(4-(1-Aminocyclobutyl)phenyl)-3-ethyl-2-(methylamino)-5-phenylfuro[2,3-d]pyrimidin-4(3H)-one

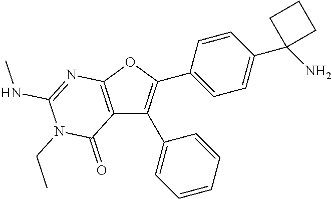

### Step 1: tert-Butyl (1-(4-(3-ethyl-2-(methylthio)-4-oxo-5-phenyl-3,4-dihydrofuro[2,3-d]pyrimidin-6-yl)phenyl)cyclobutyl)carbamate

To a solution of tert-butyl 1-(4-(2-(methylthio)-4-oxo-5-phenyl-3,4-dihydrofuro[2,3-d]pyrimidin-6-yl)phenyl)cyclobutylcarbamate (570 mg, 1.13 mmol) in DMF (10 ml) was charged potassium carbonate (469 mg, 3.40 mmol) and ethyl iodide (0.10 ml, 1.25 mmol). The reaction was complete after stirring at RT for 2 h. The reaction mixture was diluted with water and extracted twice with ethyl acetate. The organic layers were combined, washed with 1:1, water/brine (×4)four, dried (MgSO₄), filtered, and concentrated in vacuo. The residue was purified by flash chromatography (SiO₂ gradient 0-30% ethyl acetate in cyclohexane) to afford the title compound (270 mg, 45%) as a yellow solid. UPLC-MS (Method E) R_{T}=1.88 min, M+H⁺=532. (N.B. Also isolated was the O-alkylated product (202 mg, 34%).

### Step 2: tert-Butyl (1-(4-(3-ethyl-2-(methylsulfonyl)-4-oxo-5-phenyl-3,4-dihydrofuro[2,3-d]pyrimidin-6-yl)phenyl)cyclobutyl)carbamate and tert-butyl (1-(4-(3-ethyl-2-(methylsulfinyl)-4-oxo-5-phenyl-3,4-dihydrofuro[2,3-d]pyrimidin-6-yl)phenyl)cyclobutyl)carbamate

Following the procedure for tert-butyl 1-(4-(2-(methylsulfonyl)-4-morpholino-5-phenylfuro[2,3-d]pyrimidin-6-yl)phenyl)cyclobutylcarbamate, tert-butyl 1-(4-(3-ethyl-2-(methylthio)-4-oxo-5-phenyl-3,4-dihydrofuro[2,3-d]pyrimidin-6-yl)phenyl)cyclobutylcarbamate (270 mg, 0.508 mmol) was reacted to afford the title compounds (250 mg, 87%) as an orange foamy solid. LCMS (Method E) sulfone R_{T}=1.73, M+H⁺=564, sulfoxide R_{T}=1.52, M+H⁺=548.

### Step 3: tert-Butyl (1-(4-(3-ethyl-2-(methylamino)-4-oxo-5-phenyl-3,4-dihydrofuro[2,3-d]pyrimidin-6-yl)phenyl)cyclobutyl)carbamate

To a solution of tert-butyl 1-(4-(3-ethyl-2-(methylsulfonyl)-4-oxo-5-phenyl-3,4-dihydrofuro[2,3-d]pyrimidin-6-yl)phenyl)cyclobutylcarbamate (140 mg, 0.25 mmol) in THF (5 ml) was charged methylamine 2.0M solution in MeOH (0.62 ml, 1.24 mmol) the reaction was stirred at RT for 2 h. The reaction mixture was concentrated in vacuo and the resulting solid slurried in DCM. The solid was collected by filtration and dried to afford the title compound (55 mg, 43%) as a white solid. LCMS (Method E) R_{T}=1.63 min, M+H⁺=515.

### Step 4: 6-(4-(1-Aminocyclobutyl)phenyl)-3-ethyl-2-(methylamino)-5-phenylfuro[2,3-d]pyrimidin-4(3H)-one

Following the procedure for 2-(4-(1-aminocyclobutyl)phenyl)-6-methyl-3-phenylfuro[2,3-c]pyridin-7(6H)-one, tert-butyl 1-(4-(3-ethyl-2-(methylamino)-4-oxo-5-phenyl-3,4-dihydrofuro[2,3-d]pyrimidin-6-yl)phenyl)cyclobutylcarbamate (30 mg, 0.058 mmol) was reacted, the crude material was purified by preparative HPLC (Method H) to afford the title compound (10 mg, 41%) as a white solid. UPLC-MS (method E) R_{T}=0.85 min, M+H⁺=415. ¹H NMR (500 MHz, DMSO) δ 7.31-7.47 (m, 9H), 3.95 (q, 2H), 2.92 (d, 3H), 2.32-2.37 (m, 2H), 2.04-2.09 (m, 2H), 1.94-2.01 (m, 1H), 1.59-1.67 (m, 1H), 1.12 (t, 3H).

### Example 93

### 6-(4-(1-Aminocyclobutyl)phenyl)-3-ethyl-2-((2-hydroxyethyl)amino)-5-phenylfuro[2,3-d]pyrimidin-4(3H)-one

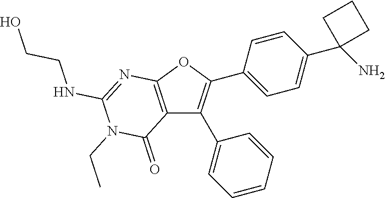

### Step 1: tert-Butyl (1-(4-(3-ethyl-2-((2-hydroxyethyl)amino)-4-oxo-5-phenyl-3,4-dihydrofuro[2,3-d]pyrimidin-6-yl)phenyl)cyclobutyl)carbamate

To a solution of tert-butyl 1-(4-(3-ethyl-2-(methylsulfonyl)-4-oxo-5-phenyl-3,4-dihydrofuro[2,3-d]pyrimidin-6-yl)phenyl)cyclobutylcarbamate (140 mg, 0.248 mmol) in THF (5 ml) was charged 2-aminoethanol (0.075 ml, 1.24 mmol) the reaction was stirred at RT for 2 h. The reaction was concentrated in vacuo and the residue purified by flash chromatography (SiO₂, gradient 60-100% ethyl acetate in cyclohexane) to afford the title compound (60 mg, 44%) as a white solid. LCMS (Method E) R_{T}=1.49 min, M+H⁺=545.

### Step 2: 6-(4-(1-Aminocyclobutyl)phenyl)-3-ethyl-2-((2-hydroxyethyl)amino)-5-phenylfuro[2,3-d]pyrimidin-4(3H)-one

Following the procedure for 2-(4-(1-aminocyclobutyl)phenyl)-6-methyl-3-phenylfuro[2,3-c]pyridin-7(6H)-one, tert-butyl 1-(4-(3-ethyl-2-(2-hydroxyethylamino)-4-oxo-5-phenyl-3,4-dihydrofuro[2,3-d]pyrimidin-6-yl)phenyl)cyclobutylcarbamate (60 mg, 0.110 mmol) was deprotected to afford the product which was purified by flash chromatography (SiO₂, gradient 0-5% 2M NH₃/MeOH in DCM). The product was freeze dried (CH₃CN:H₂O) to afford the desired product as a white solid (8 mg, 16%). LCMS (Method E) R_{T}=0.80 min, M+H⁺=445. ¹H NMR (500 MHz, DMSO-d₆) δ 7.30-7.46 (m, 9H), 4.79 (t, 1H), 3.95-4.00 (m, 2H), 3.59-3.63 (m, 2H), 3.46-3.49 (m, 2H) 2.31-2.37 (m, 2H), 2.03-2.09 (m, 2H), 1.92-2.00 (m, 1H), 1.58-1.66 (m, 1H) 1.12 (t, 3H).

### Example 94

### 6-(4-(1-Aminocyclobutyl)phenyl)-3-(cyclopropyl)methyl)-2-(methylamino)-5-phenylfuro[2,3-d]pyrimidin-4(3H)-one

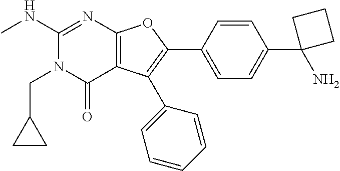

### Step 1: 6-Bromo-3-(cyclopropylmethyl)-2-(methylthio)-5-phenylfuro[2,3-d]pyrimidin-4(3H)-one and 6-bromo-4-(cyclopropylmethoxy)-2-(methylthio)-5-phenylfuro[2,3-d]pyrimidine

A solution of lithium hydroxide (0.426 g, 17.8 mmol) in water (13.5 ml) was added concurrently with a solution of (bromomethyl)cyclopropane (1.70 ml, 17.8 mmol) in 1,4-dioxane (10 ml) to a stirring suspension of 6-bromo-2-(methylthio)-5-phenylfuro[2,3-d]pyrimidin-4(3H)-one (3 g, 8.90 mmol) in 1,4-dioxane (50 ml) preheated to 70° C. After 8 h, a solution of lithium hydroxide (0.213 g, 8.90 mmol) in water (2 ml) was added followed by (bromomethyl)cyclopropane (0.83 ml, 8.90 mmol). After 16 h, a solution of lithium hydroxide (0.106 g, 4.5 mmol) in water (1 ml) was added followed by (bromomethyl)cyclopropane (0.43 ml, 4.5 mmol). After 6 h the organic solvent was removed in vacuo, the remaining aqueous was acidified to pH 1 using 2M hydrochloric acid and extracted with DCM (4×25 ml). The combined organic phases were dried (phase separator) and concentrated in vacuo. The residue was purified by silica gel chromatography (gradient 0 to 35% EtOAc in cyclohexane) to afford the title compounds as a mixture (626.5 mg, 18%). LCMS (Method E) R_{T}=1.78 min, M+H(⁷⁹Br)⁺=391.0, M+H(⁸¹Br)⁺=393.0, R_{T}=1.89 min, M+H(⁷⁹Br)⁺=391.0, M+H(⁸¹Br)⁺=393.0.

### Step 2: tert-Butyl 1-(4-(3-(cyclopropylmethyl)-2-(methylthio)-4-oxo-5-phenyl-3,4-dihydrofuro[2,3-d]pyrimidin-6-yl)phenyl)cyclobutylcarbamate

A stirred suspension of 6-bromo-3-(cyclopropylmethyl)-2-(methylthio)-5-phenylfuro[2,3-d]pyrimidin-4(3H)-one compound with 6-bromo-4-(cyclopropylmethoxy)-2-(methylthio)-5-phenylfuro[2,3-d]pyrimidine (1:1) (694 mg, 0.887 mmol), tert-butyl 1-(4-(4,4,5,5-tetramethyl-1,3,2-dioxaborolan-2-yl)phenyl)cyclobutylcarbamate (497 mg, 1.330 mmol, prepared as described in WO2008/070016) and potassium phosphate (565 mg, 2.660 mmol) in dimethylacetamide (13.6 ml) and water (4.1 ml) was degassed with N₂ for 30 min. Tetrakis(triphenylphosphine)palladium(0) (51.2 mg, 0.044 mmol) was added and the suspension degassed with N₂ for 5 min. The mixture was heated to 80° C. for 70 min. Tetrakis(triphenylphosphine)palladium(0) (51.2 mg, 0.044 mmol) was added and the reaction heated for 16 h. The mixture was cooled, quenched by addition of brine/water (1:1 v/v, 100 ml) and extracted with ethyl acetate (4×25 ml). The combined organic phases were washed with brine/water (1:1 v/v, 4×25 ml), dried (Na₂SO₄), filtered and concentrated in vacuo. The residue was purified by silica gel chromatography (gradient 0 to 10% EtOAc in cyclohexane) to afford the title compound (344 mg, 35%). LCMS (Method A) R_{T}=8.97 min, M+H⁺=558.2.

### Step 3: tert-Butyl 1-(4-(3-(cyclopropylmethyl)-2-(methylsulfonyl)-4-oxo-5-phenyl-3,4-dihydrofuro[2,3-d]pyrimidin-6-yl)phenyl)cyclobutylcarbamate

A solution of Oxone® (1.52 g, 2.469 mmol) in water (8.8 ml) was added to a stirring solution of tert-butyl 1-(4-(3-(cyclopropylmethyl)-2-(methylthio)-4-oxo-5-phenyl-3,4-dihydrofuro[2,3-d]pyrimidin-6-yl)phenyl)cyclobutylcarbamate (344 mg, 0.617 mmol) in THF (17.6 ml) and methanol (4.4 ml) at 0° C. The mixture was stirred for 16 h while warming to room temperature. Water (30 ml) was added and the mixture extracted with ethyl acetate (3×20 ml). The combined organic phases were dried (Na₂SO₄), filtered and concentrated in vacuo to give crude material of the title compound (ca. 335 mg) that was carried through to the next step without further purification.

### Step 4: tert-Butyl (1-(4-(3-(cyclopropylmethyl)-2-(methylamino)-4-oxo-5-phenyl-3,4-dihydrofuro[2,3-d]pyrimidin-6-yl)phenyl)cyclobutyl)carbamate

Methylamine (2M in THF) (1.55 ml, 3.09 mmol) was added to a stirred solution of tert-butyl (1-(4-(3-(cyclopropylmethyl)-2-(methylsulfonyl)-4-oxo-5-phenyl-3,4-dihydrofuro[2,3-d]pyrimidin-6-yl)phenyl)cyclobutyl)carbamate (182 mg, 0.309 mmol) in anhydrous THF (20 ml). After 45 minutes, the solvent was removed in vacuo and the residue purified by silica gel chromatography (gradient 0 to 35% EtOAc in cyclohexane) to afford the title compound (90.1 mg, 54%). LCMS (Method E) R_{T}=1.73 min, M+H⁺=541.2.

### Step 5: 6-(4-(1-Aminocyclobutyl)phenyl)-3-(cyclopropylmethyl)-2-(methylamino)-5-phenylfuro[2,3-d]pyrimidin-4(3H)-one

4.0M HCl in 1,4-dioxane (3 ml, 12.0 mmol) was added to a stirring suspension of tert-butyl 1-(4-(3-(cyclopropylmethyl)-2-(methylamino)-4-oxo-5-phenyl-3,4-dihydrofuro[2,3-d]pyrimidin-6-yl)phenyl)cyclobutylcarbamate (57 mg, 0.105 mmol) in anhydrous THF (1 ml). After 16 h, water (10 ml) was added and the pH adjusted to 8 using saturated sodium hydrogen carbonate solution. The aqueous phase was extracted with DCM (4×5 ml). The combined organic phases were dried (phase separator) and concentrated in vacuo. The residue was purified by silica gel chromatography (gradient 0 to 100% EtOAc in cyclohexane) to afford the title compound (27.9 mg, 60%). LCMS (Method E) R_{T}=0.92 min, M+H⁺=441.2. ¹H NMR (500 MHz, DMSO-d₆) δ 7.36-7.42 (m, 7H), 7.31 (d, 2H), 4.10 (br s, 1H), 3.87 (d, 2H), 3.18 (s, 2H), 2.92 (d, 3H), 2.31-2.36 (m, 2H), 2.01-2.08 (m, 3H), 1.91-1.99 (m, 1H), 1.58-1.66 (m, 1H), 0.41-0.45 (m, 2H), 0.33-0.37 (m, 2H).

### Example 95

### 6-(4-(1-Aminocyclobutyl)phenyl)-3-(cyclopropylmethyl)-2-((2-hydroxyethyl)amino)-5-phenylfuro[2,3-d]pyrimidin-4(3H)-one

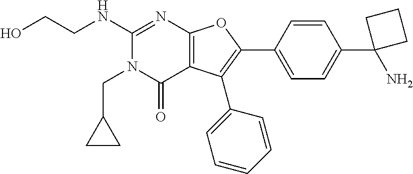

### Step 1: tert-Butyl 1-(4-(3-(cyclopropylmethyl)-2-(2-hydroxyethylamino)-4-oxo-5-phenyl-3,4-dihydrofuro[2,3-d]pyrimidin-6-yl)phenyl)cyclobutylcarbamate

To a stirred solution of tert-butyl 1-(4-(3-(cyclopropylmethyl)-2-(methylsulfonyl)-4-oxo-5-phenyl-3,4-dihydrofuro[2,3-d]pyrimidin-6-yl)phenyl)cyclobutylcarbamate (182 mg, 0.309 mmol) in anhydrous THF (20 ml) was added ethanolamine (0.19 ml, 3.09 mmol) and the solution stirred for 45 minutes. The solvent was removed in vacuo. The residue was taken up in DCM (20 ml) and washed with water (10 ml). The organic phase was separated and dried (phase separator) and concentrated in vacuo. The residue was purified by silica gel chromatography (gradient 0 to 50% EtOAc in cyclohexane) to afford the title compound (51.4 mg, 29%). LCMS (Method E) R_{T}=1.60 min, M+H⁺=571.2.

### Step 2: 6-(4-(1-Aminocyclobutyl)phenyl)-3-(cyclopropylmethyl)-2-((2-hydroxyethyl)amino)-5-phenylfuro[2,3-]pyrimidin-4(3H)-one

A Biotage® SCX-2 silica cartridge was flushed with a solution of methanol in DCM (10% v/v, 100 ml). tert-Butyl (1-(4-(3-(cyclopropylmethyl)-2-((2-hydroxyethyl)amino)-4-oxo-5-phenyl-3,4-dihydrofuro[2,3-d]pyrimidin-6-yl)phenyl)cyclobutyl)carbamate (51.4 mg, 0.090 mmol) was dissolved in a minimum of DCM/methanol (20% v/v) and placed on the cartridge. After 16 h, the cartridge was flushed with methanol in DCM (10% v/v, 50 ml) then 7M ammonia in methanol in DCM (15% v/v, 50 ml). The ammonia/methanol/DCM phase was concentrated in vacuo. The residue was purified by preparative HPLC (Method J) and the solvents removed in vacuo to afford the title compound (23.9 mg, 56%). LCMS (Method E) R_{T}=0.88 min, M+H⁺=471.2. ¹H NMR (500 MHz, methanol-d₄) δ 7.33-7.35 (m, 2H), 7.25-7.30 (m, 5H), 7.22-7.24 (m, 2H), 3.86 (d, 2H), 3.71 (t, 2H), 3.55 (t, 2H), 2.39-2.44 (m, 2H) 2.08-2.14 (m, 2H), 1.90-1.98 (m, 1H), 1.61-1.63 (m, 1H), 1.14-1.19 (m, 1H), 0.40-0.44 (m, 2H), 0.32-0.34 (m, 2H).

### Example 96

### 2-(6-(4-(1-Aminocyclobutyl)phenyl)-2-(methylthio)-4-oxo-5-phenylfuro[2,3-d]pyrimidin-3(4H)-yl)acetonitrile

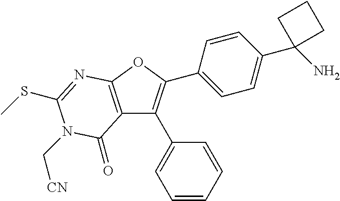

### Step 1: 2-(6-Bromo-2-(methylthio)-4-oxo-5-phenylfuro[2,3-d]pyrimidin-3(4H)-yl)acetonitrile

To a solution of 6-bromo-2-(methylthio)-5-phenylfuro[2,3-d]pyrimidin-4(3H)-one (0.12 g, 0.356 mmol) in DMF (1 ml) was added K₂CO₃ (0.108 g, 0.783 mmol) followed by 2-bromoacetonitrile (0.027 ml, 0.391 mmol). The reaction mixture was stirred at 50° C. for 1 h. Analysis by LCMS showed 2 new products, both with desired mass (mixture of O- and N-alkylated products). The reaction mixture was diluted with EtOAc and brine. The organic extracts were washed further with brine (×3) before drying and concentration in vacuo. The residue was purified by silica gel chromatography (0 to 50% EtOAc/cyclohexane) to afford the title compound (higher R_{f} product) (20 mg, 15%) as a gum. LCMS (Method E) R_{T}=1.576 min, M+H⁺=378.

### Step 2: tert-Butyl 1-(4-(3-(cyanomethyl)-2-(methylthio)-4-oxo-5-phenyl-3,4-dihydrofuro[2,3-d]pyrimidin-6-yl)phenyl)cyclobutylcarbamate

To a degassed solution of 2-(6-bromo-2-(methylthio)-4-oxo-5-phenylfuro[2,3-d]pyrimidin-3(4H)-yl)acetonitrile (0.045 g, 0.120 mmol) and tert-butyl 1-(4-(4,4,5,5-tetramethyl-1,3,2-dioxaborolan-2-yl)phenyl)cyclobutylcarbamate (0.067 g, 0.179 mmol, prepared as described in WO2008/070016) and in DME (3 ml) was added K₂CO₃ (0.165 g, 1.196 mmol) in water (0.75 ml) After further degassing, Pd(PPh₃)₄ (0.494 g, 0.427 mmol) was added and the reaction mixture was heated to reflux for 2 h, and then room temperature overnight. The reaction mixture was diluted with EtOAc and water and extracted. The organic extracts were washed with brine (3×15 ml), dried and concentrated in vacuo. The resultant residue was purified by silica gel chromatography (0 to 100% EtOAc/hexane) affording the title compound (0.045 g, 69%) as a gum. LCMS (Method E) R_{T}-=1.798 min, M+H⁺=543.2.

### Step 3: 2-(6-(4-(1-Aminocyclobutyl)phenyl)-2-(methylthio)-4-oxo-5-phenylfuro[2,3-d]pyrimidin-3(4H)-yl)acetonitrile

To a solution of tert-butyl 1-(4-(3-(cyanomethyl)-2-(methylthio)-4-oxo-5-phenyl-3,4-dihydrofuro[2,3-d]pyrimidin-6-yl)phenyl)cyclobutylcarbamate (0.045 g, 0.083 mmol) in DCM (3 ml) at 0° C. was added TFA (1 ml). The reaction mixture was stirred at 0° C. for 1 min and concentrated in vacuo. The resultant residue was pardoned between DCM and sat. Na₂CO₃ (aq), separated and the DCM extracts were concentrated. The residue was purified by silica gel chromatography (0 to 10% 1N NH₃/MeOH in DCM) affording 2-(6-(4-(1-aminocyclobutyl)phenyl)-2-(methylthio)-4-oxo-5-phenylfuro[2,3-d]pyrimidin-3(4H)-yl)acetonitrile (higher R_{f} product) (0.012 g, 33%) as an off-white solid after drying under high vacuum. LCMS (Method E) R_{T}=0.95 min, M−NH₃+H⁺=427. ¹H NMR (500 MHz, methanol-d₄): δ 7.3-7.48 (m, 9H), 5.1 (s, 2H), 2.6 (s, 3H), 2.45 (m, 2H), 2.14 (m, 2H), 1.98 (m, 1H), 1.63 (m, 1H).

### Example 97

### 2-(6-(4-(1-Aminocyclobutyl)phenyl)-2-(methylthio)-4-oxo-5-phenylfuro[2,3-d]pyrimidin-3(4H)-yl)acetamide

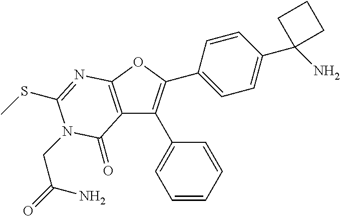

Isolated from Example 96, Step 3 as a minor by-product (lower R_{f} compound) (12 mg, 31%). LCMS (Method E) R_{T}⁻=0.872 min, M−NH₃+H⁺=461.2. NMR (500 MHz, methanol-d₄): δ 7.4-7.6 (m, 9H), 4.8 (s, 2H), 2.64 (s, 3H), 2.58 (m, 2H), 2.25 (m, 2H), 2.1 (m, 1H), 1.75 (m, 1H).

### Example 98

### (S)-6-(4-(1-Aminocyclobutyl)phenyl)-2-(1-hydroxypropan-2-ylamino)-3-methyl-5-phenylfuro[2,3-d]pyrimidin-4(3H)-one

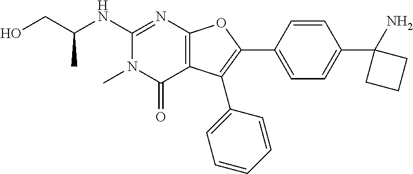

### Step 1: (S)-tert-Butyl 1-(4-(2-(1-hydroxypropan-2-ylamino)-3-methyl-4-oxo-5-phenyl-3,4-dihydrofuro[2,3-d]pyrimidin-6-yl)phenyl)cyclobutylcarbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (0.15 g, 0.273 mmol) in THF (3 ml) was added (S)-2-aminopropan-1-ol (0.205 g, 2.73 mmol). The reaction mixture was stirred at room temperature for 1 h. The reaction mixture was then concentrated in vacuo, diluted with DCM (15 ml) and water (10 ml) and extracted. The DCM extracts were dried (Na₂SO₄), concentrated and the residue purified by silica gel chromatography (0 to 5% DCM/MeOH) to afford the title compound (0.08 g, 54%) as a gum. LCMS (Method E) R_{T}=1.499 min, M+H⁺=545.2.

### Step 2: (S)-6-(4-(1-Aminocyclobutyl)phenyl)-2-(1-hydroxypropan-2-ylamino)-3-methyl-5-phenylfuro[2,3-d]pyrimidin-4(3H)-one

To a solution of (S)-tert-butyl 1-(4-(2-(1-hydroxypropan-2-ylamino)-3-methyl-4-oxo-5-phenyl-3,4-dihydrofuro[2,3-d]pyrimidin-6-yl)phenyl)cyclobutylcarbamate (0.08 g, 0.147 mmol) in DCM (3 ml) at 0° C. was added TFA (1 ml, 12.98 mmol). The reaction mixture was stirred at 0° C. for 1 min before concentration in vacuo. The residue was partitioned between DCM and sat. NaHCO₃ (aq) and the DCM extracts were dried, separated and concentrated. The residue was dissolved in 1,4-dioxane (2 ml), cooled to 0° C. and HCl (1.2 eq.) in MeOH (1.2 eq. acetyl chloride in 0.3 ml MeOH) added. The resultant solid that formed was collected by filtration, washed with dioxane and Et₂O and freeze-dried affording the title compound as a white solid (21 mg, 32%). LCMS (Method E) R_{T}=0.786 min, M−NH₃+H⁺=428.2. ¹H NMR (500 MHz, methanol-d₄): δ 7.45 (d, 2H), 7.25-7.4 (m, 7H), 4.25 (m, 1H), 3.6 (m, 2H), 3.35 (s, 3H), 2.65 (m, 2H), 2.48 (m, 2H), 2.1 (m, 1H), 1.8 (m, 1H), 1.23 (d, 3H).

### Example 99

### 6-(4-(1-Aminocyclobutyl)phenyl)-3-methyl-2-(3-oxopiperazin-1-yl)-5-phenylfuro[2,3-d]pyrimidin-4(3H)-one

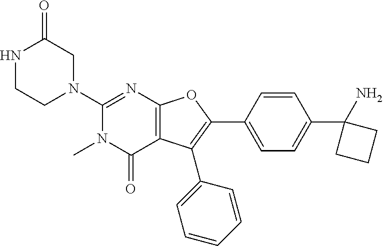

### Step 1: tert-Butyl 1-(4-(3-methyl-4-oxo-2-(3-oxopiperazin-1-yl)-5-phenyl-3,4-dihydrofuro[2,3-d]pyrimidin-6-yl)phenyl)cyclobutylcarbamate

To a solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (0.2 g, 0.364 mmol) in THF (3 ml) was added piperazin-2-one. TFA (0.390 g, 1.82 mmol) and Hunig's base (0.318 ml, 1.82 mmol). The reaction was stirred at 50° C. for 1 h, then at RT overnight. The reaction mixture was diluted with EtOAc and sat. NaHCO₃ (aq), separated, the organic extracts dried (Na₂SO₄), and concentrated. The residue was purified by silica gel chromatography (0 to 100% EtOAc/cyclohexane) to afford the title compound (0.028 g, 14%) as a gum. LCMS (Method E) R_{T}=1.391 min, M+H⁺=570.2.

### Step 2: 6-(4-(1-Aminocyclobutyl)phenyl)-3-methyl-2-(3-oxopiperazin-1-yl)-5-phenylfuro[2,3-d]pyrimidin-4(3H)-one

To a solution of tert-butyl 1-(4-(3-methyl-4-oxo-2-(3-oxopiperazin-1-yl)-5-phenyl-3,4-dihydrofuro[2,3-d]pyrimidin-6-yl)phenyl)cyclobutylcarbamate (0.028 g, 0.049 mmol) in DCM (2 ml) was added TFA (1 ml, 12.98 mmol). The reaction mixture was stirred at room temperature for 10 min. The reaction mixture was concentrated in vacuo followed by dilution with DCM (15 ml) and sat. Na₂CO₃ (aq) (10 ml). The DCM extracts were then dried (Na₂SO₄), concentrated and the residue purified by silica gel chromatography (0 to 10% 1N NH₃/MeOH in DCM) affording the title compound (0.003 g, 13%) as an off-white solid after drying. LCMS (Method E) R_{T}=0.747 min, M−NH₃+H⁺=453.2.

### Example 100

### 6-(4-(1-Aminocyclobutyl)phenyl)-3-(cyclopropyl)methyl)-5-phenylfuro[2,3-d]pyrimidin-4(3H)-one, HCl

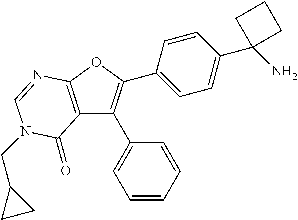

### Step 1: tert-Butyl 1-(4-(3-(cyclopropylmethyl)-4-oxo-5-phenyl-3,4-dihydrofuro[2,3-d]pyrimidin-6-yl)phenyl)cyclobutylcarbamate

To a solution of tert-butyl 1-(4-(4-oxo-5-phenyl-3,4-dihydrofuro[2,3-d]pyrimidin-6-yl)phenyl)cyclobutylcarbamate (0.1 g, 0.219 mmol) in DMF (1 ml) was added K₂CO₃ (0.066 g, 0.481 mmol) and NaI (3.3 mg, 0.022 mmol). The reaction mixture was cooled to 0° C. before the addition of (bromomethyl)cyclopropane (0.024 ml, 0.240 mmol). The reaction was stirred at room temperature for 1 h. The reaction mixture was diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford the title compound (0.09 g, 80%) as a gum. LCMS (Method E) R_{T}=1.703 min, M+H⁺=512.2.

### Step 2: 6-(4-(1-Aminocyclobutyl)phenyl)-3-(cyclopropylmethyl)-5-phenylfuro[2,3-d]pyrimidin-4(3H)-one, HCl

To a solution of tert-butyl 1-(4-(3-(cyclopropylmethyl)-4-oxo-5-phenyl-3,4-dihydrofuro[2,3-d]pyrimidin-6-yl)phenyl)cyclobutylcarbamate (0.09 g, 0.176 mmol) in DCM (3 ml) at 0° C. was added TFA (1 ml). The reaction mixture was stirred at 0° C. for 1 min before concentration to dryness. The reaction mixture was concentrated in vacuo, diluted with DCM (15 ml) and sat. Na₂CO₃ (aq) (10 ml) and extracted. The DCM extracts were dried (Na₂SO₄), concentrated and the residue purified by silica gel chromatography (0 to 10% MeOH/DCM) affording 6-(4-(1-aminocyclobutyl)phenyl)-3-(cyclopropylmethyl)-5-phenylfuro[2,3-d]pyrimidin-4(3H)-one, HCl (0.035 g, 44%) as a gum. The gum was dissolved in 1,4-dioxane, cooled to 0° C. under N₂ and HCl (2 eq) in MeOH (0.25 ml) added (prepared from 2 eq acetyl chloride in MeOH). The resultant precipitate was collected by filtration, washed with Et₂O and dried under high vacuum affording the title compound as a white solid (23 mg, 32%). LCMS (Method E) R_{T}=0.901 min, M−NH₃+H⁺=395.2. ¹H NMR (500 MHz, methanol-d₄): δ 8.44 (s, 1H), 7.63 (d, 2H), 7.4-7.5 (m, 7H), 3.92 (d, 2H), 2.79 (m, 2H), 2.6 (m, 2H), 2.25 (m, 1H), 1.99 (m, 1H), 1.34 (m, 1H), 0.6 (m, 2H), 0.45 (m, 2H).

### Example 101

### 2-(6-(4-(1-Aminocyclobutyl)phenyl)-4-oxo-5-phenylfuro[2,3-d]pyrimidin-3(4H)-yl)acetonitrile

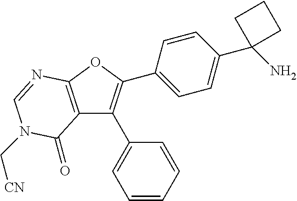

### Step 1: tert-Butyl 1-(4-(3-(cyanomethyl)-4-oxo-5-phenyl-3,4-dihydrofuro[2,3-d]pyrimidin-6-yl)phenyl)cyclobutylcarbamate

To a solution of tert-butyl 1-(4-(4-oxo-5-phenyl-3,4-dihydrofuro[2,3-d]pyrimidin-6-yl)phenyl)cyclobutylcarbamate (0.1 g, 0.219 mmol) in DMF (1 ml) was added K₂CO₃ (66.5 mg, 0.481 mmol), NaI (3.3 mg, 0.022 mmol) and 2-bromoacetonitrile (0.017 ml, 0.240 mmol). The reaction mixture was stirred at 60° C. for 2 h. The reaction mixture was diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford the title compound (0.015 g, 14%) as a gum. LCMS (Method E) R_{T}=1.521 min, M+H⁺=497.2.

### Step 2: 2-(6-(4-(1-Aminocyclobutyl)phenyl)-4-oxo-5-phenylfuro[2,3-d]pyrimidin-3(4H)-yl)acetonitrile

A solution of tert-butyl 1-(4-(3-(cyanomethyl)-4-oxo-5-phenyl-3,4-dihydrofuro[2,3-d]pyrimidin-6-yl)phenyl)cyclobutylcarbamate (0.015 g, 0.030 mmol) in DCM (0.5 ml) was loaded directly onto a 1 g SCX-2 column and allowed to standed for 1 h. MeOH (5 ml) was passed through the column, followed by 2N NH₃/MeOH (5 ml). The NH₃/MeOH fraction was concentrated in vacuo and the residue was purified by silica gel chromatography (0 to 10% 2N NH₃/MeOH/DCM) to afford the title compound (0.007 g, 59%) as an off-white solid after drying. LCMS (Method E) R_{T}=0.723 min, M−NH₃+H⁺=380.2. ¹H NMR (500 MHz, methanol-d₄): δ 8.4 (s, 1H), 7.4-7.5 (m, 9H), 5.08 (s, 2H), 2.55 (m, 2H), 2.25 (m, 2H), 2.1 (m, 1H), 1.75 (m, 1H).

### Example 102

### 6-(4-(1-Aminocyclobutyl)phenyl)-3-(2-hydroxyethyl)-5-phenylfuro[2,3-d]pyrimidin-4(3H)-one, HCl

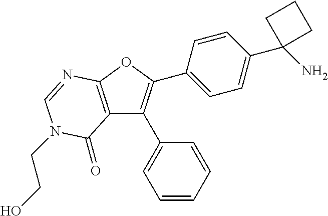

### Step 1: tert-Butyl 1-(4-(3-(2-hydroxyethyl)-4-oxo-5-phenyl-3,4-dihydrofuro[2,3-d]pyrimidin-6-yl)phenyl)cyclobutylcarbamate

To a solution of tert-butyl 1-(4-(4-oxo-5-phenyl-3,4-dihydrofuro[2,3-d]pyrimidin-6-yl)phenyl)cyclobutylcarbamate (0.15 g, 0.328 mmol) in DMF (1 ml) under N₂ was added K₂CO₃ (0.0997 g, 0.721 mmol), NaI (4.9 mg, 0.033 mmol) followed by 2-bromoethanol (0.025 ml, 0.361 mmol). The reaction mixture was stirred at 50° C. for 2 h. The reaction mixture was diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford tert-butyl 1-(4-(3-(2-hydroxyethyl)-4-oxo-5-phenyl-3,4-dihydrofuro[2,3-d]pyrimidin-6-yl)phenyl)cyclobutylcarbamate (0.113 g, 69%) as a white solid. LCMS (Method E) R_{T}=1.414 min, M+H⁺=502.2.

### Step 2: 6-(4-(1-Aminocyclobutyl)phenyl)-3-(2-hydroxyethyl)-5-phenylfuro[2,3-d]pyrimidin-4(3H)-one, HCl

A solution of tert-butyl 1-(4-(3-(2-hydroxyethyl)-4-oxo-5-phenyl-3,4-dihydrofuro[2,3-d]pyrimidin-6-yl)phenyl)cyclobutylcarbamate (0.113 g, 0.225 mmol) in DCM (2 ml) was loaded directly onto a 5 g SCX-2 column and allowed to stand for 5 h. The column was washed with MeOH (20 ml) followed by 2N NH₃/MeOH (20 ml). The NH₃/MeOH fraction was concentrated in vacuo affording a gum that was dissolved in 1,4-dioxane (1 ml), cooled to 0° C. under N₂ and HCl (2 eq) in MeOH (0.15 ml) added (prepared from 2 eq. acetyl chloride in MeOH). The resultant the title compound (0.07 g, 71%) as a white solid. LCMS (Method E) R_{T}=0.703 min, M−NH₃+H⁺=385. ¹H NMR (500 MHz, methanol-d₄): δ 8.34 (s, 1H), 7.63 (d, 2H), 7.4-7.5 (m, 7H), 4.18 (t, 2H), 3.8 (t, 2H), 2.75 (m, 2H), 2.6 (m, 2H), 2.25 (m, 1H), 2.0 (m, 1H).

### Example 103

### 6-(4-(1-Aminocyclobutyl)phenyl)-3-(2-fluoroethyl)-5-phenylfuro[2,3-d]pyrimidin-4(3H)-one, HCl

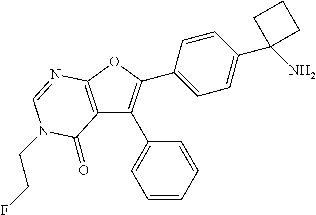

### Step 1: tert-Butyl 1-(4-(3-(2-fluoroethyl)-4-oxo-5-phenyl-3,4-dihydrofuro[2,3-d]pyrimidin-6-yl)phenyl)cyclobutylcarbamate

To a solution of tert-butyl 1-(4-(4-oxo-5-phenyl-3,4-dihydrofuro[2,3-d]pyrimidin-6-yl)phenyl)cyclobutylcarbamate (0.15 g, 0.328 mmol) in DMF (1.5 ml) under N₂ was added K₂CO₃ (0.100 g, 0.721 mmol) followed by 1-fluoro-2-iodoethane (0.035 ml, 0.426 mmol). The reaction mixture was stirred at 50° C. for 2 h and was then diluted with EtOAc (15 ml) and brine (15 ml) and extracted. The organic extracts were then washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford the title compound (0.121 g, 73%) as a clear gum. LCMS (Method E) R_{T}=1.581 min, M+H⁺=504.2.

### Step 2: 6-(4-(1-Aminocyclobutyl)phenyl)-3-(2-fluoroethyl)-5-phenylfuro[2,3-d]pyrimidin-4(3H)-one, HCl

A solution of tert-butyl 1-(4-(3-(2-fluoroethyl)-4-oxo-5-phenyl-3,4-dihydrofuro[2,3-d]pyrimidin-6-yl)phenyl)cyclobutylcarbamate (0.121 g, 0.240 mmol) in DCM (2 ml) was loaded directly onto a 5 g SCX-2 column and allowed to stand for 5 h. The column was washed with MeOH (20 ml) followed by 2N NH₃/MeOH (20 ml). The NH₃/MeOH fraction was concentrated in vacuo affording a gum that was dissolved in 1,4-dioxane (1 ml), cooled to 0° C. under N₂ and HCl (2 eq) in MeOH (0.15 ml) added (prepared from 2 eq. acetyl chloride in MeOH). The resultant precipitate was collected by filtration, washed with Et₂O and dried under high vacuum affording the title compound (0.092 g, 87%) as a white solid. LCMS (Method E) R_{T}=0.784 min, M−NH₃+H⁺=387. ¹H NMR (500 MHz, methanol-d₄): δ 8.37 (s, 1H), 7.63 (d, 2H), 7.4-7.5 (m, 7H), 4.75 (m, 1H), 4.67 (m, 1H), 4.43 (m, 1H), 4.37 (m, 1H), 2.76 (m, 2H), 2.6 (m, 2H), 2.25 (m, 1H), 2.0 (m, 1H).

### Example 104

### 6-(4-(1-Aminocyclobutyl)phenyl)-3-ethyl-5-phenylfuro[2,3-d]pyrimidin-4(3H)-one, HCl

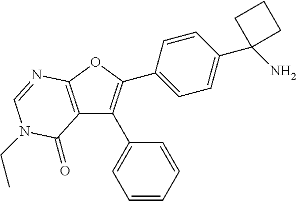

### Step 1: tert-Butyl 1-(4-(3-ethyl-4-oxo-5-phenyl-3,4-dihydrofuro[2,3-d]pyrimidin-6-yl)phenyl)cyclobutylcarbamate

To a solution of tert-butyl 1-(4-(4-oxo-5-phenyl-3,4-dihydrofuro[2,3-d]pyrimidin-6-yl)phenyl)cyclobutylcarbamate (0.15 g, 0.328 mmol) in DMF (1 ml) under N₂ was added K₂CO₃ (0.1 g, 0.721 mmol) followed by iodoethane (0.034 ml, 0.426 mmol). The reaction mixture was stirred at 50° C. for 2 h. The reaction mixture was then diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford the title compound (0.097 g, 61%) as a clear gum. LCMS (Method E) R_{T}=1.611 min, M+H⁺=486.2.

### Step 2: 6-(4-(1-Aminocyclobutyl)phenyl)-3-ethyl-5-phenylfuro[2,3-d]pyrimidin-4(3H)-one, HCl

A solution of tert-butyl 1-(4-(3-ethyl-4-oxo-5-phenyl-3,4-dihydrofuro[2,3-d]pyrimidin-6-yl)phenyl)cyclobutylcarbamate (0.097 g, 0.2 mmol) in DCM (2 ml) was loaded directly onto a 5 g SCX-2 column and allowed to stand for 5 h. The column was washed with MeOH (20 ml) followed by 2N NH₃/MeOH (20 ml). The NH₃/MeOH fraction was concentrated in vacuo affording a gum that was dissolved in 1,4-dioxane (1 ml), cooled to 0° C. under N₂ and HCl (2 eq) in MeOH (0.15 ml) added (prepared from 2 eq acetyl chloride in MeOH). The resultant precipitate was collected by filtration, washed with Et₂O and dried under high vacuum to afford the title compound (70 mg, 83%) as a white solid. LCMS (Method E) R_{T}=0.796 min, M−NH₃+H⁺=369.2. ¹H NMR (500 MHz, methanol-d₄): δ 8.31 (s, 1H), 7.51 (d, 2H), 7.3-7.4 (m, 7H), 4.0 (q, 2H), 2.7 (m, 2H), 2.5 (m, 2H), 2.15 (m, 1H), 1.85 (m, 1H), 1.25 (t, 3H).

### Example 105

### 6-(4-(1-Aminocyclobutyl)phenyl)-3-(2-methoxyethyl)-5-phenylfuro[2,3-d]pyrimidin-4(3H)-one, HCl

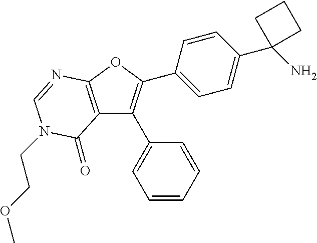

### Step 1: tert-Butyl 1-(4-(3-(2-methoxyethyl)-4-oxo-5-phenyl-3,4-dihydrofuro[2,3-d]pyrimidin-6-yl)phenyl)cyclobutylcarbamate

To a solution of tert-butyl 1-(4-(4-oxo-5-phenyl-3,4-dihydrofuro[2,3-d]pyrimidin-6-yl)phenyl)cyclobutylcarbamate (0.15 g, 0.328 mmol) in DMF (1.5 ml) under N₂ was added K₂CO₃ (0.1 g, 0.721 mmol) followed by NaI (4.9 mg, 0.033 mmol) and 1-bromo-2-methoxyethane (0.037 ml, 0.393 mmol). The reaction mixture was stirred at 50° C. for 2 h. The reaction mixture was then diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford the title compound (0.112 g, 66%) as a gum. LCMS (Method E) R_{T}=1.602 min, M+H⁺=516.2.

### Step 2: 6-(4-(1-Aminocyclobutyl)phenyl)-3-(2-methoxyethyl)-5-phenylfuro[2,3-d]pyrimidin-4(3H)-one, HCl

A solution of tert-butyl 1-(4-(3-(2-methoxyethyl)-4-oxo-5-phenyl-3,4-dihydrofuro[2,3-d]pyrimidin-6-yl)phenyl)cyclobutylcarbamate (0.112 g, 0.217 mmol) in DCM (2 ml) was loaded directly onto a 5 g SCX-2 column and allowed to stand for 5 h. The column was washed with MeOH (20 ml) followed by 2N NH₃/MeOH (20 ml). The NH₃/MeOH fraction was concentrated in vacuo affording a gum that was dissolved in 1,4-dioxane (1 ml), cooled to 0° C. under N₂ and HCl (2 eq) in MeOH (0.15 ml) added (prepared from 2 eq acetyl chloride in MeOH). The resultant precipitate was collected by filtration, washed with Et₂O and dried under high vacuum affording the title compound (0.077 g, 78%) as a white solid. LCMS (Method E) R_{T}=0.845 min, M−NH₃+H⁺=369.2.

¹H NMR (500 MHz, methanol-d₄): a 8.32 (s, 1H), 7.63 (d, 2H), 7.4-7.5 (m, 2H), 4.25 (t, 2H), 3.65 (m, 5H), 2.8 (m, 2H), 2.6 (m, 2H), 2.25 (m, 1H), 2.0 (m, 1H).

### Example 106

### 6-(4-(1-Aminocyclobutyl)phenyl)-5-phenyl-3-(2,12-trifluoroethyl)furo[2,3-d]pyrimidin-4(3H)-one, HCl

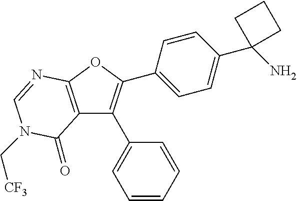

### Step 1: tert-Butyl 1-(4-(4-oxo-5-phenyl-3-(2,2,2-trifluoroethyl)-3,4-dihydrofuro[2,3-d]pyrimidin-6-yl)phenyl)cyclobutylcarbamate

To a solution of tert-butyl 1-(4-(4-oxo-5-phenyl-3,4-dihydrofuro[2,3-d]pyrimidin-6-yl)phenyl)cyclobutylcarbamate (0.15 g, 0.328 mmol) in DMF (1 ml) under N₂ was added K₂CO₃ (0.0997 g, 0.721 mmol) followed by 1,1,1-trifluoro-2-iodoethane (0.036 ml, 0.361 mmol). The reaction mixture was heated under microwave conditions at 100° C. for 1 h. The reaction mixture was then diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford the title compound (0.107 g, 61%) as an oil. LCMS (Method E) R_{T}=1.676 min, M+H⁺=540.2.

### Step 2: 6-(4-(1-Aminocyclobutyl)phenyl)-5-phenyl-3-(2,2,2-trifluoroethyl)furo[2,3-d]pyrimidin-4(3H)-one, HCl

A solution of tert-butyl 1-(4-(4-oxo-5-phenyl-3-(2,2,2-trifluoroethyl)-3,4-dihydrofuro[2,3-d]pyrimidin-6-yl)phenyl)cyclobutylcarbamate (0.107 g, 0.198 mmol) in DCM (2 ml) was loaded directly onto a 5 g SCX-2 column and allowed to stand for 5 h. The column was then washed with MeOH (20 ml) followed by 2N NH₃/MeOH (20 ml). The NH₃/MeOH fraction was concentrated in vacuo affording a gum. The gum was dissolved in 1,4-dioxane (1 ml), cooled to 0° C. under N₂ and HCl (2 eq) in MeOH (0.15 ml) added (prepared from 2 eq acetyl chloride in MeOH). The resultant precipitate was collected by filtration, washed with Et₂O and dried under high vacuum affording the title compound (0.066 g, 70%) as a white solid. LCMS (Method E) R_{T}=0.924 min, M−NH₃+H⁺=423.0. ¹H NMR (500 MHz, methanol-d₄): δ 8.44 (s, 1H), 7.64 (d, 2H), 7.4-7.5 (m, 7H), 4.9 (m, 2H), 2.8 (m, 2H), 2.6 (m, 2H), 2.25 (m, 2H), 2.0 (m, 2H).

### Example 107

### 6-(4-(1-Aminocyclobutyl)phenyl)-3-isopropyl-5-phenylfuro[2,3-d]pyrimidin-4(3H)-one, HCl

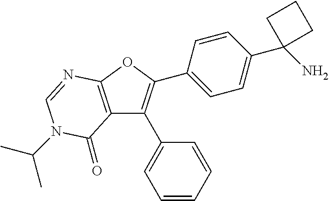

### Step 1: tert-Butyl 1-(4-(3-isopropyl-4-oxo-5-phenyl-3,4-dihydrofuro[2,3-d]pyrimidin-6-yl)phenyl)cyclobutylcarbamate

To a solution of tert-butyl 1-(4-(4-oxo-5-phenyl-3,4-dihydrofuro[2,3-d]pyrimidin-6-yl)phenyl)cyclobutylcarbamate (0.075 g, 0.164 mmol) in DMF (1.5 ml) under N₂ was added K₂CO₃ (0.050 g, 0.361 mmol), followed by 2-iodopropane (0.020 ml, 0.197 mmol). The reaction mixture was stirred at 50° C. for 2 h. The reaction mixture was then diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford the title compound (0.034 g, 42%) as a gum. LCMS (Method E) R_{T}=1.682 min, M+H⁺=500.2.

### Step 2: 6-(4-(1-Aminocyclobutyl)phenyl)-3-isopropyl-5-phenylfuro[2,3-d]pyrimidin-4(3H)-one, HCl

A solution of tert-butyl 1-(4-(3-isopropyl-4-oxo-5-phenyl-3,4-dihydrofuro[2,3-d]pyrimidin-6-yl)phenyl)cyclobutylcarbamate (0.034 g, 0.068 mmol) in DCM (1 ml) was loaded directly onto a 2 g SCX-2 column and allowed to stand for 5 h. The column was then washed with MeOH (20 ml) followed by 2N NH₃/MeOH (20 ml). The NH₃/MeOH fraction was concentrated in vacuo affording a gum that was dissolved in 1,4-dioxane (1 ml), cooled to 0° C. under N₂ and HCl (2 eq) in MeOH (0.15 ml) added (prepared from 2 eq acetyl chloride in MeOH). The resultant precipitate was collected by filtration, washed with Et₂O and dried under high vacuum to afford the title compound (0.015 g, 51%) as a white solid. LCMS (Method E) R_{T}=0.914 min, M−NH₃+H⁺=383.0. ¹H NMR (500 MHz, methanol-d₄): δ 8.45 (s, 1H), 7.63 (d, 2H), 7.4-7.5 (m, 7H), 5.1 (m, 1H), 2.8 (m, 2H), 2.6 (m, 2H), 2.25 (m, 1H), 2.0 (m, 1H), 1.5 (d, 6H).

### Example 108

### 6-(4-(1-Aminocyclobutyl)phenyl)-3-(2,2-difluoroethyl)-5-phenylfuro[2,3-d]pyrimidin-4(3H)-one

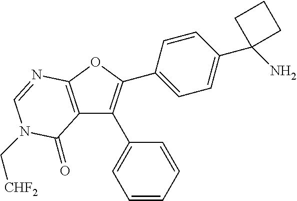

### Step 1: tert-Butyl (1-(4-(3-(2,2-difluoroethyl)-4-oxo-5-phenyl-3,4-dihydrofuro[2,3-d]pyrimidin-6-yl)phenyl)cyclobutyl)carbamate

To a solution of tert-butyl (1-(4-(4-oxo-5-phenyl-3,4-dihydrofuro[2,3-d]pyrimidin-6-yl)phenyl)cyclobutyl)carbamate (0.075 g, 0.164 mmol) in DMF (1 ml) under N₂ was added Cs₂CO₃ (0.118 g, 0.361 mmol) followed by 1,1-difluoro-2-iodoethane (0.019 ml, 0.213 mmol). The reaction mixture was heated in the under microwave conditions at 100° C. for 1 h. The reaction mixture was then diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford the title compound (0.046 g, 54%) as an off-white solid. LCMS (Method E) R_{T}=1.635 min, M+H⁺=522.2.

### Step 2: 6-(4-(1-Aminocyclobutyl)phenyl)-3-(2,2-difluoroethyl)-5-phenylfuro[2,3-d]pyrimidin-4(3H)-one

A solution of tert-butyl (1-(4-(3-(2,2-difluoroethyl)-4-oxo-5-phenyl-3,4-dihydrofuro[2,3-d]pyrimidin-6-yl)phenyl)cyclobutyl)carbamate (0.046 g, 0.088 mmol) in DCM (2 ml) and methanol (0.5 ml) was loaded onto a pre-washed (DCM) 5 g SCX-2 column and allowed to stand for 18 h at RT. The column was washed with 5% MeOH/DCM (25 ml) followed by 15% 2N NH₃/MeOH in DCM (50 ml). The NH₃ wash fraction was concentrated in vacuo affording a gum that was purified by silica gel chromatography (0 to 10% 2N NH₃/MeOH/DCM) to afford the title compound as a beige solid (17 mg, 46%). LCMS (Method E) R_{T}=0.836 min, M−NH₃+H⁺=405.0. ¹H NMR (500 MHz, methanol-d₄): δ 8.27 (s, 1H), 7.52 (d, 2H), 7.3-7.45 (m, 7H), 6.0-6.2 (m, 1H), 4.4 (m, 2H), 2.65 (m, 2H), 2.5 (m, 2H), 2.15 (m, 1H), 1.85 (m, 1H).

### Example 109

### 2-(4-(1-Aminocyclobutyl)phenyl)-3-Phenyl-5-(2,2,2-trifluoroethyl)furo[3,2-c]pyridin-4(5H)-one, HCl

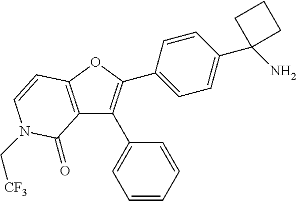

### Step 1: tert-Butyl 1-(4-(4-oxo-3-phenyl-5-(2,2,2-trifluoroethyl)-4,5-dihydrofuro[3,2-c]pyridin-2-yl)phenyl)cyclobutylcarbamate

To a solution of tert-butyl 1-(4-(4-oxo-3-phenyl-4,5-dihydrofuro[3,2-c]pyridin-2-yl)phenyl)cyclobutylcarbamate (0.09 g, 0.197 mmol) in DMF (1 ml) under N₂ was added K₂CO₃ (0.060 g, 0.434 mmol) followed by 1,1,1-trifluoro-2-iodoethane (0.021 ml, 0.217 mmol). The reaction mixture was heated under microwave conditions at 100° C. for 1 h. Analysis by LCMS showed incomplete reaction. A further 1 eq 1,1,1-trifluoro-2-iodoethane was added and heating continued for 1 h. The reaction mixture was diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford tert-butyl 1-(4-(4-oxo-3-phenyl-5-(2,2,2-trifluoroethyl)-4,5-dihydrofuro[3,2-c]pyridin-2-yl)phenyl)cyclobutylcarbamate (0.069 g, 65%) as an off-white solid. LCMS (Method E) R_{T}=1.724 min, M+H⁺=539.0.

### Step 2: 2-(4-(1-Aminocyclobutyl)phenyl)-3-phenyl-5-(2,2,2-trifluoroethyl)furo[3,2-c]pyridin-4(5H)-one, HCl

A solution of tert-butyl (1-(4-(4-oxo-3-phenyl-5-(2,2,2-trifluoroethyl)-4,5-dihydrofuro[3,2-c]pyridin-2-yl)phenyl)cyclobutyl)carbamate (0.069 g, 0.128 mmol) in DCM (2 ml) was loaded onto a prewashed (DCM) 5 g SCX-2 column and allowed to stand for 18 h at RT. The column was washed with 5% MeOH/DCM (20 ml) followed by 7N NH₃/MeOH:DCM 1:5 (25 ml). NH₃/MeOH washes were concentrated in vacuo and the resultant solid recrystallised from MeOH, collected by filtration and vacuum dried to afford the title compound as a white solid (27 mg, 44%). LCMS (Method E) R_{T}=0.915 min, M−NH₃+H⁺=422.0. ¹H NMR (500 MHz, methanol-d₄): δ 7.65 (d, 2H), 7.6 (d, 2H), 7.49 (m, 7H), 6.9 (d, 1H), 4.85 (m, 2H), 2.75 (m, 2H), 2.6 (m, 2H), 2.25 (m, 1H), 2.0 (m, 1H).

### Example 110

### 6-(4-(1-Aminocyclobutyl)phenyl)-3-(2-fluoroethyl)-2-(2-hydroxyethylamino)-5-phenylfuro[2,3-d]pyrimidin-4(3H)-one

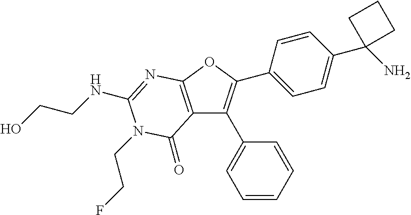

### Step 1: 6-Bromo-2-(methylthio)-5-phenylfuro[2,3-d]pyrimidin-4(3H)-one

To a solution of 2-(methylthio)-5-phenylfuro[2,3-d]pyrimidin-4(3M-one (0.25 g, 0.484 mmol) in DMF (1 ml) at 0° C. was added bromine (0.027 ml, 0.532 mmol). The reaction mixture was stirred at 0° C. until the reaction was complete. The reaction mixture was diluted with EtOAc (30 ml) and brine (25 ml) and extracted. The organic extracts were then washed with brine (3×10 ml), dried (Na₂SO₄) and concentrated in vacuo affording crude product as a beige solid. The solid was slurried in Et₂O, filtered and dried affording the title compound (0.105 g, 64%) as a beige solid. LCMS (Method E) R_{T}=1.33 min, M+H⁺=336.9

### Step 2: tert-Butyl 1-(4-(2-(methylthio)-4-oxo-5-phenyl-3,4-dihydrofuro[2,3-d]pyrimidin-6-yl)phenyl)cyclobutylcarbamate

To a solution of 6-bromo-2-(methylthio)-5-phenylfuro[2,3-d]pyrimidin-4(3H)-one (0.09 g, 0.267 mmol) in DME (2 ml) was added tert-butyl 1-(4-(4,4,5,5-tetramethyl-1,3,2-dioxaborolan-2-yl)phenyl)cyclobutylcarbamate (0.1196 g, 0.320 mmol, prepared as described in WO2008/070016) and K₂CO₃ (0.3689 g, 2.67 mmol) in water (0.5 ml). The mixture was then degassed with N₂ before the addition of Pd(PPh₃)₄ (0.0308 g, 0.027 mmol). The reaction mixture was then heated under microwave conditions at 120° C. for 30 min. The reaction mixture was diluted with EtOAc (15 ml) and brine (15 ml) and extracted. The organic extracts were washed with brine (3×10 ml), dried (Na₂SO₄) and concentrated in vacuo affording crude product as a beige solid. The solid was slurried in Et₂O, filtered and dried to afford the title compound (0.075 g, 56%) as a beige solid. LCMS (Method E) R_{T}=1.604 min, M+H⁺=504.2.

### Step 3: tert-Butyl 1-(4-(3-(2-fluoroethyl)-2-(methylthio)-4-oxo-5-phenyl-3,4-dihydrofuro[2,3-d]pyrimidin-6-yl)phenyl)cyclobutylcarbamate

To a solution of tert-butyl 1-(4-(2-(methylthio)-4-oxo-5-phenyl-3,4-dihydrofuro[2,3-d]pyrimidin-6-yl)phenyl)cyclobutylcarbamate (0.6 g, 1.191 mmol) in DMF (1 ml) was added Cs₂CO₃ (0.854 g, 2.62 mmol) followed by 1-fluoro-2-iodoethane (0.116 ml, 1.430 mmol). The reaction mixture was stirred at 70° C. for 1 h. The reaction mixture was then diluted with EtOAc (15 ml) and brine (15 ml) and extracted. The organic extracts were washed with brine (3×10 ml), dried (Na₂SO₄) and concentrated in vacuo affording crude product as an oil. The oil was purified by silica gel chromatography (0 to 50% EtOAc/cyclohexane) and the lower R_{f} fractions combined and concentrated to afford the title compound (0.17 g, 26%) as an off-white solid. LCMS (Method E) R_{T}=1.787 min, M+H⁺=550.1

### Step 4: tert-Butyl (1-(4-(3-(2-fluoroethyl)-2-(methylsulfinyl)-4-oxo-5-phenyl-3,4-dihydrofuro[2,3-d]pyrimidin-6-yl)phenyl)cyclobutyl)carbamate

To a solution of tert-butyl 1-(4-(3-(2-fluoroethyl)-2-(methylthio)-4-oxo-5-phenyl-3,4-dihydrofuro[2,3-d]pyrimidin-6-yl)phenyl)cyclobutylcarbamate (0.172 g, 0.313 mmol) in THF (5 ml) and methanol (1.25 ml) at 0° C. was added Oxone® (0.769 g, 1.252 mmol) in water (1.25 ml). The reaction mixture was stirred at 50° C. for 2 h. The reaction mixture was then diluted with EtOAc and water, extracted, dried (Na₂SO₄) and concentrated affording the title compound as an oil (0.15 g, 30%). LCMS (Method E) R_{T}=1.509 min, M+H⁺=566.0.

### Step 5: tert-Butyl (1-(4-(3-(2-fluoroethyl)-2-((2-hydroxyethyl)amino)-4-oxo-5-phenyl-3,4-dihydrofuro[2,3-d]pyrimidin-6-yl)phenyl)cyclobutyl)carbamate

To a solution of tert-butyl (1-(4-(3-(2-fluoroethyl)-2-(methylsulfinyl)-4-oxo-5-phenyl-3,4-dihydrofuro[2,3-d]pyrimidin-6-yl)phenyl)cyclobutyl)carbamate (0.15 g, 0.080 mmol) in tetrahydrofuran (2 ml) was added 2-aminoethanol (0.0486 g, 0.796 mmol). The reaction mixture was stirred at 50° C. for 1 h before being concentrated in vacuo, diluted with DCM (15 ml) and water (10 ml), and extracted. The DCM extracts were dried (Na₂SO₄), concentrated and the residue purified by silica gel chromatography (0 to 100% EtOAc/cyclohexane) to afford the title compound (0.015 g, 20%) as a gum. LCMS (Method E) R_{T}=1.478 min, M+H⁺=563.2.

### Step 6: 6-(4-(1-Aminocyclobutyl)phenyl)-3-(2-fluoroethyl)-2-(2-hydroxyethylamino)-5-phenylfuro[2,3-d]pyrimidin-4(3H)-one

A solution of tert-butyl (1-(4-(3-(2-fluoroethyl)-2-((2-hydroxyethyl)amino)-4-oxo-5-phenyl-3,4-dihydrofuro[2,3-d]pyrimidin-6-yl)phenyl)cyclobutyl)carbamate (15 mg, 0.027 mmol) in DCM (2 ml) was loaded directly onto a 5 g SCX-2 column and allowed to stand for 5 h. The column was then washed with MeOH (20 ml) followed by 2N NH₃/MeOH (20 ml). The NH₃/MeOH fraction was concentrated in vacuo affording a gum that was purified by silica gel chromatography (0 to 10% 1N NH₃/MeOH in DCM) to afford the title compound as an off-white solid (2 mg, 17%). LCMS (Method E) R_{T}=0.799 min, M−NH₃+H⁺=446.2.

### Example 111

### 2-((6-(4-(1-Aminocyclobutyl)phenyl)-4-(2-fluoroethoxy)-5-phenylfuro[2,3-d]pyrimidin-2-yl)amino)ethanol

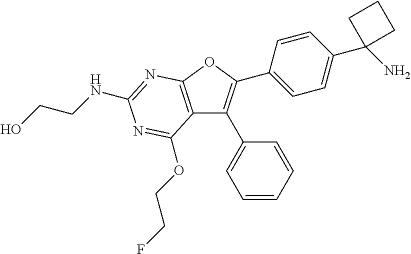

### Step 1: tert-Butyl (1-(4-(4-(2-fluoroethoxy)-2-(methylsulfonyl)-5-phenylfuro[2,3-d]pyrimidin-6-yl)phenyl)cyclobutyl)carbamate

To a solution of tert-butyl (1-(4-(4-(2-fluoroethoxy)-2-(methylthio)-5-phenylfuro[2,3-d]pyrimidin-6-yl)phenyl)cyclobutyl)carbamate (0.37 g, 0.673 mmol) (isolated as major product in Example 110, Step 3) in tetrahydrofuran (5 ml) and MeOH (1.7 ml) at 0° C. was added Oxone® (1.66 g, 2.69 mmol) in water (1.7 ml). The reaction mixture was stirred at 50° C. for 2 h. Analysis by LCMS showed that the reaction was complete. The reaction mixture was cooled to RT and diluted with EtOAc/brine. The EtOAc extracts were dried (Na₂SO₄) and concentrated to afford the title compound as an oil (0.31 g, 79%) that was used immediately in the next step. LCMS (Method E) R_{T}=1.478 min, M+H⁺=563.2.

### Step 2: tert-Butyl 1-(4-(4-(2-fluoroethoxy)-2-(2-hydroxyethylamino)-5-phenylfuro[2,3-d]pyrimidin-6-yl)phenyl)cyclobutylcarbamate

To a solution of tert-butyl 1-(4-(4-(2-fluoroethoxy)-2-(methylsulfonyl)-5-phenylfuro[2,3-d]pyrimidin-6-yl)phenyl)cyclobutylcarbamate (0.15 g, 0.258 mmol) in tetrahydrofuran (3 ml) was added 2-aminoethanol (0.158 g, 2.58 mmol). The reaction mixture was stirred at 50° C. for 1 h before being concentrated in vacuo, diluted with DCM (20 ml) and water (10 ml) and extracted. The DCM extracts were dried (Na₂SO₄), concentrated and the residue purified by silica gel chromatography (0 to 100% EtOAc/cyclohexane) to afford the title compound (0.104 g, 72%) as a gum. LCMS (Method E) R_{T}=1.589 min, M+H⁺=563.2.

### Step 3: 2-((6-(4-(1-Aminocyclobutyl)phenyl)-4-(2-fluoroethoxy)-5-phenylfuro[2,3-d]pyrimidin-2-yl)amino)ethanol

A solution of tert-butyl (1-(4-(4-(2-fluoroethoxy)-2-((2-hydroxyethyl)amino)-5-phenylfuro[2,3-d]pyrimidin-6-yl)phenyl)cyclobutyl)carbamate (15 mg, 0.027 mmol) in DCM (2 ml) was loaded directly onto a 5 g SCX-2 column and allowed to stand for 5 h. The column was washed with MeOH (20 ml) followed by 2N NH₃/MeOH (20 ml). The NH₃/MeOH fraction was concentrated in vacuo affording a gum that was purified by silica gel chromatography (0 to 10% 1N NH₃/MeOH in DCM) to give the title compound (7 mg, 57%) as an off-white solid. LCMS (Method E) R_{T}=0.89 min, M+H⁺=463.2.

### Example 112

### 2-((6-(4-(1-Aminocyclobutyl)phenyl)-7-bromo-5-(2-fluoroethyl)-3-phenylfuro[3,2-c]pyridin-4(5H)-one

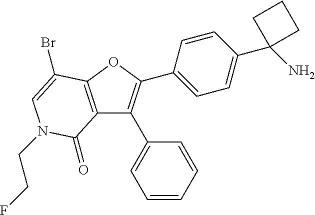

### Step 1: tert-Butyl (1-(4-(7-bromo-5-(2-fluoroethyl)-4-oxo-3-phenyl-4,5-dihydrofuro[3,2-c]pyridin-2-yl)phenyl)cyclobutyl)carbamate

To a solution of tert-butyl (1-(4-(7-bromo-4-oxo-3-phenyl-4,5-dihydrofuro[3,2-c]pyridin-2-yl)phenyl)cyclobutyl)carbamate (0.05 g, 0.093 mmol) (isolated in Example 114, Step 1) in DMF (0.5 ml) was added Cs₂CO₃ (0.067 g, 0.205 mmol) followed by 1-fluoro-2-iodoethane (8.37 μl, 0.103 mmol). The reaction mixture was heated to 50° C. for 1 h. The reaction mixture was then diluted with EtOAc and water and extracted. The organic extracts were washed with brine (3×15 ml), dried and concentrated in vacuo. The resultant residue was purified by silica gel chromatography (0 to 50% EtOAc/hexane) to afford the title compound (0.03 g, 55%) as a beige solid. LCMS (Method E) R_{T}=1.754 min, M+H⁺=583 & 584.

### Step 2: 2-(4-(1-Aminocyclobutyl)phenyl)-7-bromo-5-(2-fluoroethyl)-3-phenylfuro[3,2-c]pyridin-4(5H)-one

A solution of tert-butyl (1-(4-(7-bromo-5-(2-fluoroethyl)-4-oxo-3-phenyl-4,5-dihydrofuro[3,2-c]pyridin-2-yl)phenyl)cyclobutyl)carbamate (10 mg, 0.027 mmol) in DCM (2 ml) was loaded directly onto a 5 g SCX-2 column and allowed to stand for 5 h. The column was washed with MeOH (20 ml) followed by 2N NH₃/MeOH (20 ml). The NH₃/MeOH fraction was concentrated in vacuo affording a gum that was purified by silica gel chromatography (0 to 10% 1N NH₃/MeOH in DCM) affording the title compound as an off-white solid (4 mg, 48%). LCMS (Method E) R_{T}=0.943 min, M+H⁺=483, 484. ¹H NMR (500 MHz, methanol-d₄): δ 7.74 (s, 1H), 7.3-7.4 (m, 9H), 4.63 (m, 1H), 4.51 (m, 1H), 4.22 (m, 1H), 4.18 (m, 1H), 2.42 (m, 2H), 2.15 (m, 2H), 2.0 (m, 1H), 1.64 (m, 1H).

### Example 113

### 2-(4-(1-Aminocyclobutyl)phenyl)-5-(2-fluoroethyl)-7-(1-methyl-1H-pyrazol-4-yl)-3-phenylfuro[3,2-d]pyridin-4(5H)-one

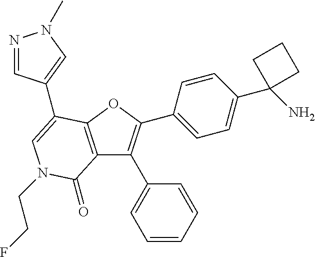

### Step 1: tert-Butyl (1-(4-(5-(2-fluoroethyl)-7-(1-methyl-1H-pyrazol-4-yl)-4-oxo-3-phenyl-4,5-dihydrofuro[3,2-c]pyridin-2-yl)phenyl)cyclobutyl)carbamate

To a solution of tert-butyl (1-(4-(7-bromo-5-(2-fluoroethyl)-4-oxo-3-phenyl-4,5-dihydrofuro[3,2-c]pyridin-2-yl)phenyl)cyclobutyl)carbamate (38 mg, 0.065 mmol) in DMF (1 ml) was added potassium phosphate tribasic (41.6 mg, 0.196 mmol) in water (0.250 ml) followed by 1-methyl-4-(4,4,5,5-tetramethyl-1,3,2-dioxaborolan-2-yl)-1H-pyrazole (20.4 mg, 0.098 mmol). The mixture was then degassed with N₂ before the addition of Pd(PPh₃)₄ (3.78 mg, 3.27 μmol). The reaction mixture was heated at 110° C. for 30 minutes under microwave heating. The reaction mixture was then cooled to room temperature, diluted with EtOAc and water and extracted. The organic extracts were washed with brine (3×15 ml), dried and concentrated in vacuo. The resultant residue was purified by silica gel chromatography (0 to 100% EtOAc/cyclohexane) to afford the title compound (29.7 mg, 78%) as an off-white solid. LCMS (Method E) R_{T}=1.575 min, M+H⁺=583.2.

### Step 2: 2-(4-(1-Aminocyclobutyl)phenyl)-5-(2-fluoroethyl)-7-(1-methyl-1H-pyrazol-4-yl)-3-phenylfuro[3,2-c]pyridin-4(5H)-one

A solution of tert-butyl (1-(4-(5-(2-fluoroethyl)-7-(1-methyl-1H-pyrazol-4-yl)-4-oxo-3-phenyl-4,5-dihydrofuro[3,2-c]pyridin-2-yl)phenyl)cyclobutyl)carbamate (29 mg, 0.050 mmol) in DCM (2 ml) & methanol (0.5 ml) was loaded onto a pre-washed (DCM) 5 g SCX-2 column and allowed to stand for 18 h at RT. The column was then washed with 5% MeOH/DCM (25 ml) followed by 15% 2N NH₃/MeOH in DCM (50 ml). The NH₃/MeOH/DCM wash was concentrated in vacuo affording a residue that was freeze-dried from water/MeOH yielding the title compound (19 mg, 79%) as an off-white solid. LCMS (Method E) R_{T}=0.83 min, M+H⁺=483.2. ¹H NMR (500 MHz, methanol-d₄): δ 8.07 (s, 1H), 7.9 (s, 1H), 7.8 (s, 1H), 7.47 (d, 2H), 7.3-7.4 (m, 7H), 4.69 (m, 1H), 4.58 (m, 1H), 4.2-4.3 (m, 2H), 3.9 (s, 3H), 2.5 (m, 2H), 2.25 (m, 2H), 2.0 (m, 1H), 1.73 (m, 1H).

### Example 114

### 2-(4-(1-Aminocyclobutyl)phenyl)-5-methyl-7-(1-methyl-1H-pyrazol-4-yl)-3-phenylfuro[3,2-c]pyridin-4(5H)-one

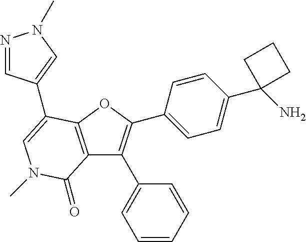

### Step 1: tert-Butyl (1-(4-(7-bromo-4-methoxy-3-phenylfuro[3,2-c]pyridin-2-yl)phenyl)cyclobutyl)carbamate

Bromine (0.36 ml, 7.010 mmol) was added to a stirred solution of tert-butyl (1-(4-(4-methoxy-3-phenylfuro[3,2-c]pyridin-2-yl)phenyl)cyclobutyl)carbamate (1.0 g, 2.125 mmol) in carbon tetrachloride (10 ml) and DMF (3.3 ml) at 0° C. After 1.5 h, the solution was poured into an aqueous mixture of sodium hydrogen carbonate, water and 20% sodium thiosulfate solution (1:3:4 v/v, 40 ml) which was extracted with ethyl acetate (4×25 ml). The combined organic phases were washed with brine/water (1:1 v/v, 4×25 ml), dried (Na₂SO₄), filtered and concentrated in vacuo to afford crude material of the title compound (1.39 g) which was used without further purification.

### Step 2: 2-(4-(1-Aminocyclobutyl)phenyl)-7-bromo-3-phenylfuro[3,2-c]pyridin-4(5H)-one

To a stirred solution of tert-butyl (1-(4-(7-bromo-4-methoxy-3-phenylfuro[3,2-c]pyridin-2-yl)phenyl)cyclobutyl)carbamate (1.39 g) in 1,4-dioxane (10 ml) was added 2M hydrochloric acid (10 ml) and the solution stirred for 16 h at 110° C. After cooling, the 1,4-dioxane was removed in vacuo and the resulting aqueous solution neutralised by addition of saturated sodium hydrogen carbonate (aq) solution. A solid precipitated which was collected by vacuum filtration, washed with water and dried under suction for 16 h to afford crude material of the title compound (0.90 g) which was used without further purification.

### Step 3: tert-Butyl (1-(4-(7-bromo-4-oxo-3-phenyl-4,5-dihydrofuro[3,2-c]pyridin-2-yl)phenyl)cyclobutyl)carbamate

To a stirred suspension of 2-(4-(1-aminocyclobutyl)phenyl)-7-bromo-3-phenylfuro[3,2-c]pyridin-4(5H)-one (0.90 g) in THF (20 ml) was added N,N-dimethylaminopyridine (52.0 mg, 0.425 mmol) followed by di-tert-butyl dicarbonate (0.487 g, 2.231 mmol). The suspension was heated to reflux for 16 h. Di-tert-butyl dicarbonate (0.487 g, 2.231 mmol) was added. After 2.5 h, N,N-dimethylaminopyridine (52.0 mg, 0.425 mmol) was added followed 1 h later by di-tert-butyl dicarbonate (0.487 g, 2.231 mmol). After 2 h, N,N′-dimethylaminopyridine (156.0 mg, 1.275 mmol) were added followed 2 h later by di-tert-butyl dicarbonate (0.974 g, 4.462 mmol). After 0.5 h, the reaction was cooled to 50° C., DMF (10 ml) was added followed by potassium carbonate (0.646 g, 4.68 mmol) and methyl iodide (0.15 ml, 2.338 mmol) and the mixture stirred for 16 h. After cooling the THF was removed in vacuo and water/brine (1:1 v/v, 100 ml) was added and the resulting aqueous suspension extracted with ethyl acetate (4×25 ml). The combined organic phases were washed with brine/water (1:1 v/v, 4×25 ml), dried (Na₂SO₄), filtered and concentrated in vacuo. The residue was purified by silica gel chromatography (gradient 0 to 50% EtOAc in cyclohexane) to afford the title compound (552.1 mg, 45%*). LCMS (Method E) R_{T}=1.73 min, M+H(⁷⁹Br)⁺=549.0, M+H(⁸¹Br)⁺=551.0. ¹H NMR (500 MHz, methanol-d₄): δ 7.86 (s, 1H), 7.42-7.46 (m, 7H), 7.38 (d, 2H), 3.57 (s, 3H), 2.44-2.50 (m, 4H), 2.03-2.15 (m, 1H), 1.82-1.95 (m, 1H), 1.31 (br s, 9H).

* Yield is overall yield of steps 1-3

### Step 4: tert-Butyl (1-(4-(5-methyl-7-(1-methyl-1H-pyrazol-4-yl)-4-oxo-3-phenyl-4,5-dihydrofuro[3,2-c]pyridin-2-yl)phenyl)cyclobutyl)carbamate

A stirring suspension of tert-butyl (1-(4-(7-bromo-5-methyl-4-oxo-3-phenyl-4,5-dihydrofuro[3,2-c]pyridin-2-yl)phenyl)cyclobutyl)carbamate (50 mg, 0.091 mmol), 1-methyl-4-(4,4,5,5-tetramethyl-1,3,2-dioxaborolan-2-yl)-1H-pyrazole (28.4 mg, 0.136 mmol) and potassium phosphate (57.9 mg, 0.273 mmol) in DMF (1 ml) and water (0.25 ml) in a 10 ml microwave vial was degassed with N₂ for 5 min. Tetrakis(triphenylphosphine)palladium(0) (5.3 mg, 0.005 mmol) was added and the suspension degassed with N₂ for 5 mins. The vial was sealed and irradiated at 100° C. for 30 min (CEM Explorer/Discover). The mixture was diluted with water/brine (1:1 v/v, 10 ml) and extracted with ethyl acetate (4×25 ml). The combined organic phases were washed with brine/water (1:1 v/v, 4×25 ml), dried (Na₂SO₄), filtered and concentrated in vacuo. The residue was purified by silica gel chromatography (gradient 0 to 100% EtOAc in cyclohexane) to afford the title compound (50.6 mg, 94%). LCMS (Method E) R_{T}=1.52 min, M+H⁺=551.2.

### Step 5: 2-(4-(1-Aminocyclobutyl)phenyl)-5-methyl-7-(1-methyl-1H-pyrazol-4-yl)-3-phenylfuro[3,2-c]pyridin-4(5H)-one

A Biotage® SCX-2 silica cartridge was flushed with a solution of methanol in DCM (20% v/v, 20 ml). tert-Butyl (1-(4-(5-methyl-7-(1-methyl-1H-pyrazol-4-yl)-4-oxo-3-phenyl-4,5-dihydrofuro[3,2-c]pyridin-2-yl)phenyl)cyclobutyl)carbamate (50.6 mg, 0.092 mmol) was dissolved in a minimum of DCM and placed on the cartridge. After 16 h, the cartridge was flushed with methanol in DCM (20% v/v, 20 ml) then 7M ammonia in methanol in DCM (20% v/v, 20 ml). The ammonia/methanol/DCM phase was concentrated in vacuo. The residue was purified by silica gel chromatography (gradient 0 to 10% methanol in DCM) to afford the title compound (9.9 mg, 24%). LCMS (Method E) R_{T}⁻=0.82 min, M+H⁺=451.2. ¹H NMR (500 MHz, methanol-d₄): δ 7.96 (s, 1H), 7.82 (s, 1H), 7.71 (s, 1H), 7.31 (d, 2H), 7.22-7.27 (m, 7H), 3.82 (s, 3H), 3.41 (s, 3H), 2.32-2.41 (m, 2H), 2.04-2.11 (m, 2H), 1.85-1.94 (m, 1H), 1.53-1.62 (m, 1H).

### Example 115

### 2-(4-(1-Aminocyclobutyl)phenyl)-5-methyl-3-phenyl-7-(pyrimidin-5-yl)furo[3,2-c]pyridin-4(5H)-one

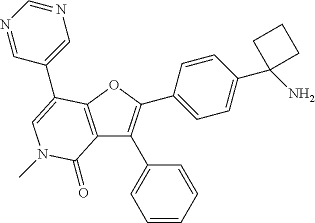

### Step 1: tert-Butyl (1-(4-(5-methyl-4-oxo-3-phenyl-7-(pyrimidin-5-yl)-4,5-dihydrofuro[3,2-c]pyridin-2-yl)phenyl)cyclobutyl)carbamate

A stirred suspension of tert-butyl (1-(4-(7-bromo-5-methyl-4-oxo-3-phenyl-4,5-dihydrofuro[3,2-c]pyridin-2-yl)phenyl)cyclobutyl)carbamate (50 mg, 0.091 mmol), pyrimidin-5-ylboronic acid (16.9 mg, 0.136 mmol) and potassium phosphate (57.9 mg, 0.273 mmol) in DMF (1 ml) and water (0.25 ml) in a 10 ml microwave vial was degassed with N₂ for 5 min. Tetrakis(triphenylphosphine)palladium(0) (5.3 mg, 0.005 mmol) was added and the suspension degassed with N₂ for 5 min. The vial was sealed and irradiated at 100° C. for 30 min (CEM Explorer/Discover). The mixture was diluted with water/brine (1:1 v/v, 10 ml) and extracted with ethyl acetate (4×25 ml). The combined organic phases were washed with brine/water (1:1 v/v, 4×25 ml), dried (Na₂SO₄), filtered and concentrated in vacuo. The residue was purified by silica gel chromatography (gradient 0 to 100% EtOAc in cyclohexane) to afford the title compound (28.9 mg, 58%). LCMS (Method E) R_{T}=1.51 min, M+H⁺=549.2.

### Step 2: 2-(4-(1-Aminocyclobutyl)-phenyl)-5-methyl-3-phenyl-7-(pyrimidin-5-yl)furo[3,2-c]pyridin-4(5H)-one

To a stirred suspension of tert-butyl (1-(4-(5-methyl-4-oxo-3-phenyl-7-(pyrimidin-5-yl)-4,5-dihydrofuro[3,2-c]pyridin-2-yl)phenyl)cyclobutyl)carbamate (28.9 mg, 0.052 mmol) in DCM (2 ml) was added TFA (0.25 ml) and the suspension stirred for 1 h. A Biotage® SCX-2 silica cartridge was flushed with a solution of methanol in DCM (20% v/v, 50 ml). The reaction mixture was placed on the cartridge for 30 min. The cartridge was flushed with methanol in DCM (20% v/v, 50 ml) then 7M ammonia in methanol in DCM (20% v/v, 50 ml). The ammonia/methanol/DCM phase was concentrated in vacuo. The residue was purified by silica gel chromatography (gradient 0 to 100%, (10% methanol in DCM) in DCM) to afford the title compound (9.5 mg, 49%).

LCMS (Method E) R_{T}=0.78 min, M+H⁺=449.2. ¹H NMR (500 MHz, methanol-d₄): δ 9.20 (d, 2H), 9.08 (s, 1H), 8.02 (d, 1H), 7.28-7.38 (m, 9H), 3.56 (s, 3H), 2.42-2.46 (m, 2H), 2.13-2.18 (m, 2H), 1.96-2.02 (m, 1H), 1.61-1.70 (m, 1H).

### Example 116

### 2-(4-(1-Aminocyclobutyl)phenyl)-5-methyl-3-phenyl-7-(1H-pyrazol-5-yl)furo[3,2-c]pyridin-4(5H)-one

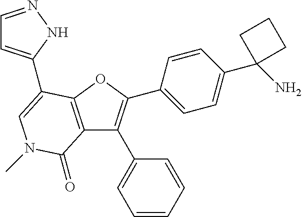

### Step 1: tert-Butyl (1-(4-(5-methyl-4-oxo-3-phenyl-7-(1-((2-(trimethylsilyl)ethoxy)methyl)-1H-pyrazol-5-yl)-4,5-dihydrofuro[3,2-c]pyridin-2-yl)phenyl)cyclobutyl)carbamate

To a solution of tert-butyl (1-(4-(7-bromo-5-methyl-4-oxo-3-phenyl-4,5-dihydrofuro[3,2-c]pyridin-2-yl)phenyl)cyclobutyl)carbamate (50 mg, 0.091 mmol) in DMF (1 ml) was added potassium phosphate tribasic (58.0 mg, 0.273 mmol) in water (0.250 ml) followed by 5-(4,4,5,5-tetramethyl-1,3,2-dioxaborolan-2-yl)-1-((2-(trimethylsilyl)ethoxy)methyl)-1,4-pyrazole (44.3 mg, 0.136 mmol). The mixture was then degassed with N₂ before the addition of Pd(PPh₃)₄ (5.26 mg, 4.55 μmol). The reaction mixture was heated at 110° C. for 30 minutes under microwave heating. The reaction mixture was then cooled to room temperature, diluted with EtOAc and water and extracted. The organic extracts were washed with brine (3×15 ml), dried and concentrated in vacuo. The resultant residue was purified by silica gel chromatography (0 to 100% EtOAc/cyclohexane) affording tert-butyl (1-(4-(5-methyl-4-oxo-3-phenyl-7-(14(2-(trimethylsilyl)ethoxy)methyl)-1H-pyrazol-5-yl)-4,5-dihydrofuro[3,2-c]pyridin-2-yl)phenyl)cyclobutyl)carbamate (47.3 mg, 78%) as an off-white solid. LCMS (Method E) R_{T}=1.882 min, M+H⁺=667.2.

### Step 2: 2-(4-(1-Aminocyclobutyl)phenyl)-5-methyl-3-phenyl-7-(1H-pyrazol-5-yl)furo[3,2-c]pyridin-4(5H)-one

A solution of tert-butyl (1-(4-(5-methyl-4-oxo-3-phenyl-7-(1-((2-(trimethylsilyl)ethoxy)methyl)-1H-pyrazol-5-yl)-4,5-dihydrofuro[3,2-c]pyridin-2-yl)phenyl)cyclobutyl)carbamate (65 mg, 0.097 mmol) in HCl (4M in 1,4-dioxane) (1 ml, 32.9 mmol) and water (1 ml) was heated under microwave conditions at 100° C. for 30 minutes. The reaction mixture was then carefully neutralized with sat. Na₂CO₃ (aq) before extraction with DCM (3×15 ml). The combined DCM extracts were concentrated in vacuo and purified by silica gel chromatography (0 to 10% 1N NH₃/MeOH in DCM) to afford the title compound as an off-white solid (18 mg, 42%). LCMS (Method E) R_{T}=0.773 min, M+H⁺=437.2. ¹H NMR (500 MHz, DMSO-d₆): δ 8.2 (s, 1H), 7.9 (d, 1H), 7.43 (m, 9H), 3.54 (s, 3H), 2.35 (m, 2H), 2.09 (m, 2H), 1.95 (m, 1H), 1.6 (m, 1H).

### Example 117

### 2-(4-(1-Aminocyclobutyl)phenyl)-5-methyl-4-oxo-3-phenyl-4,5-dihydrofuro[3,2-c]pyridine-7-carboxamide

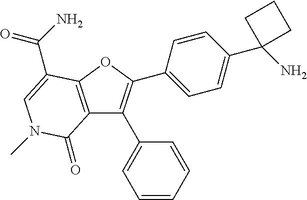

### Step 1: Methyl 2-(4-(1-((tert-butoxycarbonyl)amino)cyclobutyl)phenyl)-5-methyl-4-oxo-3-phenyl-4,5-dihydrofuro[3,2-c]pyridine-7-carboxylate

To a solution of tert-butyl (1-(4-(7-bromo-5-methyl-4-oxo-3-phenyl-4,5-dihydrofuro[3,2-c]pyridin-2-yl)phenyl)cyclobutyl)carbamate (200 mg, 0.364 mmol) in MeOH (15 ml) was added PdCl₂(dppf)(66.6 mg, 0.091 mmol) and triethylamine (0.254 ml, 1.820 mmol). The mixture was then degassed with N₂ before stirring at 50° C. under an atmosphere of carbon monoxide for 72 h. The reaction mixture was then cooled to RT, filtered through a pad of Celite and concentrated in vacuo. The residue was then suspended in DCM (25 ml) and water (25 ml) and extracted. The DCM extracts were dried (Na₂SO₄), filtered and concentrated and the residue purified by silica gel chromatography (0 to 50% EtOAc/cyclohexane) to afford the title compound as a white solid (135 mg, 70%). LCMS (Method E) R_{T}=1.628 min, M+H⁺=529.2.

### Step 2: tert-Butyl (1-(4-(7-carbamoyl-5-methyl-4-oxo-3-phenyl-4,5-dihydrofuro[3,2-c]pyridin-2-yl)phenyl)cyclobutyl)carbamate

To a solution of methyl 2-(4-(1-((tert-butoxycarbonyl)amino)cyclobutyl)phenyl)-5-methyl-4-oxo-3-phenyl-4,5-dihydrofuro[3,2-c]pyridine-7-carboxylate (50 mg, 0.095 mmol) in MeOH (0.5 ml) and DMF (0.250 ml) was added magnesium nitride (33.4 mg, 0.331 mmol). The reaction mixture was then heated at 80° C. for 24 h. After this time the reaction mixture was cooled, diluted with DCM (25 ml) and filtered. The filtrate was concentrated in vacuo and found to be a 1:1 mixture of tert-butyl (1-(4-(7-carbamoyl-5-methyl-4-oxo-3-phenyl-4,5-dihydrofuro[3,2-c]pyridin-2-yl)phenyl)cyclobutyl)carbamate and methyl 2-(4-(1-((tert-butoxycarbonyl)amino)cyclobutyl)phenyl)-5-methyl-4-oxo-3-phenyl-4,5-dihydrofuro[3,2-c]pyridine-7-carboxylate by LCMS. This material was used directly in the subsequent step without further purification.

### Step 3: 2-(4-(1-Aminocyclobutyl)phenyl)-5-methyl-4-oxo-3-phenyl-4,5-dihydrofuro[3,2-c]pyridine-7-carboxamide

To a solution of a crude mixture of tert-butyl (1-(4-(7-carbamoyl-5-methyl-4-oxo-3-phenyl-4,5-dihydrofuro[3,2-c]pyridin-2-yl)phenyl)cyclobutyl)carbamate (0.02 g, 0.039 mmol) and methyl 2-(4-(1-((tert-butoxycarbonyl)amino)cyclobutyl)phenyl)-5-methyl-4-oxo-3-phenyl-4,5-dihydrofuro[3,2-c]pyridine-7-carboxylate (0.02 g, 0.038 mmol) in DCM (2 ml) at 0° C. was added TFA (1 ml). The reaction mixture was stirred at 0° C. for 2 min before concentration in vacuo. It was then diluted with DCM (15 ml) and sat. Na₂CO₃ (aq) (10 ml) and extracted. The DCM extracts were dried (Na₂SO₄), concentrated and the residue purified by silica gel chromatography (Biotage KP-Sil—eluent 0 to 2% MeOH in EtOAc). This afforded the title compound (5 mg, 31%) as a white solid. LCMS (Method E) R_{T}=0.683 min, M−NH₃+H⁺=397.0. ¹H NMR (500 MHz, methanol-d₄): δ 8.2 (s, 1H), 7.41 (d, 2H), 7.31 (m, 7H), 3.54 (s, 3H), 2.45 (m, 2H), 2.15 (m, 2H), 2.0 (m, 1H), 1.65 (m, 1H).

### Example 118

### Methyl 2-(4-(1-aminocyclobutyl)phenyl)-5-methyl-4-oxo-3-phenyl-4,5-dihydrofuro[3,2-c]pyridine-7-carboxylate

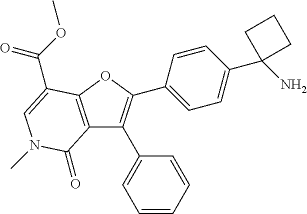

Isolated from Example 118, Step 3 as an off-white solid. LCMS (Method E) R_{T}=0.838 min, M+H⁺=429.2. ¹H NMR (500 MHz, methanol-d₄): δ 8.33 (s, 1H), 7.42 (d, 2H), 7.3 (m, 7H), 3.89 (s, 3H), 3.52 (s, 3H), 2.45 (m, 2H), 2.16 (m, 2H), 2.0 (m, 1H), 1.68 (m, 1H).

### Example 119

### 2-(4-(1-Aminocyclobutyl)phenyl)-N,5-dimethyl-4-oxo-3-phenyl-4,5-dihydrofuro[3,2-c]pyridine-7-carboxamide

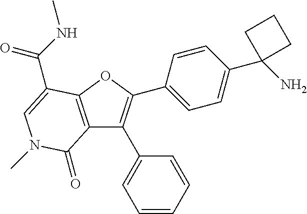

Step 1: tert-Butyl (1-(4-(5-methyl-7-(methylcarbamoyl)-4-oxo-3-phenyl-4,5-dihydrofuro[3,2-c]pyridin-2-yl)phenyl)cyclobutyl)carbamate

To a sealed tube was charged methyl 2-(4-(1-((tert-butoxycarbonyl)amino)cyclobutyl)phenyl)-5-methyl-4-oxo-3-phenyl-4,5-dihydrofuro[3,2-c]pyridine-7-carboxylate (70 mg, 0.132 mmol) and a 33% solution of methylamine in ethanol (2 ml). The vessel was sealed and heated to 60° C. overnight. The reaction was concentrated in vacuo and the residue purified by flash chromatography (SiO₂, gradient 20-80% ethyl acetate in cyclohexane) to afford the title compound (45 mg, 64%) as an off white solid. LCMS (Method E) R_{T}=1.44, M+H⁺=528.

### Step 2: 2-(4-(1-Aminocyclobutyl)phenyl)-N,5-dimethyl-4-oxo-3-phenyl-4,5-dihydrofuro[3,2-c]pyridine-7-carboxamide

A solution of tert-butyl (1-(4-(5-methyl-7-(methylcarbamoyl)-4-oxo-3-phenyl-4,5-dihydrofuro[3,2-c]pyridin-2-yl)phenyl)cyclobutyl)carbamate (45 mg, 0.08 mmol) in DCM (3 ml) and TFA (1 ml) was stirred at RT for 5 min. The reaction mixture was concentrated in vacuo and the residue neutralised with aq. NaHCO₃ and extracted into DCM. The biphasic solution was separated using a phase separator (Isolute® SPE). The organic layer was concentrated in vacuo and the crude material purified by flash chromatography (SiO₂, gradient 0-10% methanol in DCM) to afford the title compound as a white solid (16 mg, 44%). LCMS (Method E) R_{T}=0.73 min, M+H⁺=428. ¹FI NMR (500 MHz, methanol-d₄): δ 8.11 (s, 1H), 7.29-7.43 (m, 9H), 3.52 (s, 3H), 2.94 (s, 3H), 2.41-2.46 (m, 2H), 2.08-2.17 (m, 2H) 1.92-2.01 (m, 1H), 1.60-1.68 (m, 1H).

### Example 120

### 2-(4-(1-Aminocyclobutyl)phenyl)-N-(2-hydroxyethyl)-5-methyl-4-oxo-3-phenyl-4,5-dihydrofuro[3,2-c]pyridine-7-carboxamide

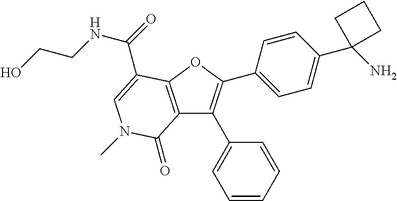

### Step 1: 2-(4-(1-((tert-Butoxycarbonyl)amino)cyclobutyl)phenyl)-5-methyl-4-oxo-3-phenyl-4,5-dihydrofuro[3,2-c]pyridine-7-carboxylic acid

To a solution of methyl 2-(4-(1-((tert-butoxycarbonyl)amino)cyclobutyl)phenyl)-5-methyl-4-oxo-3-phenyl-4,5-dihydrofuro[3,2-c]pyridine-7-carboxylate (50 mg, 0.095 mmol) in 1,4-dioxane (3 ml) was added 2N NaOH (aq) (2 ml, 4.00 mmol). The reaction mixture was heated to 50° C. for 2 h. The reaction mixture was then diluted with aq. 2M HCl (5 ml) followed by extraction with DCM (×2). The combined DCM extracts were dried (phase separator) and concentrated in vacuo to afford the title compound (43.8 mg, 90%) as a clear gum. LCMS (Method E) R_{T}=1.41 min, M+H⁺=515.2.

### Step 2: tert-Butyl (1-(4-(74(2-hydroxyethyl)carbamoyl)-5-methyl-4-oxo-3-phenyl-4,5-dihydrofuro[3,2-c]pyridin-2-yl)phenyl)cyclobutyl)carbamate

To a solution of 2-(4-(1-((tert-butoxycarbonyl)amino)cyclobutyl)phenyl)-5-methyl-4-oxo-3-phenyl-4,5-dihydrofuro[3,2-c]pyridine-7-carboxylic acid (50 mg, 0.097 mmol) in THF (2 ml) was added Hunig's base (0.051 ml, 0.292 mmol), 2-aminoethanol (8.80 μl, 0.146 mmol) followed by HATU (55.4 mg, 0.146 mmol). The reaction mixture was stirred at room temperature for 1 h. The reaction mixture was then diluted with DCM (20 ml) and brine (15 ml) and extracted. The DCM portion was dried (phase separator) and concentrated in vacuo before purification by silica gel chromatography (0 to 5% MeOH in DCM) to afford the title compound as a gum (48 mg, 89%). LCMS (Method E) R_{T}=1.338 min, M+H⁺=558.2.

### Step 3: 2-(4-(1-Aminocyclobutyl)phenyl)-N-(2-hydroxyethyl)-5-methyl-4-oxo-3-phenyl-4,5-dihydrofuro[3,2-c]pyridine-7-carboxamide

To a solution of tert-butyl (1-(4-(74(2-hydroxyethyl)carbamoyl)-5-methyl-4-oxo-3-phenyl-4,5-dihydrofuro[3,2-c]pyridin-2-yl)phenyl)cyclobutyl)carbamate (48 mg, 0.086 mmol) in DCM (2 ml) at 0° C. was added TFA (1 ml). The reaction mixture was stirred at 0° C. for 2 minutes and then concentrated in vacuo. The residue was dissolved in DCM and washed with sat. Na₂HCO₃ (aq) and the DCM extracts concentrated in vacuo. The residue was purified by silica gel chromatography (0 to 10% 1N NH₃/MeOH in DCM) to afford an oil that was subsequently freeze-dried from water/ACN to give the title compound (14 mg, 0.031 mmol, 36%) as a white solid. LCMS (Method E) R_{T}=0.702 min, M+H⁺=458.2. ¹H NMR (500 MHz, methanol-d₄): δ 8.19 (s, 1H), 7.45 (d, 2H), 7.3 (m, 7H), 3.72 (t, 2H), 3.56 (m, 5H), 2.42 (m, 2H), 2.14 (m, 2H), 1.98 (m, 1H), 1.63 (m, 1H).

### Example 121

### 6-(4-(1-Aminocyclobutyl)phenyl)-2-((2-hydroxyethyl)amino)-5-phenyl-3-(2,2,2-trifluoroethyl)furo[2,3-d]pyrimidin-4(3H)-one

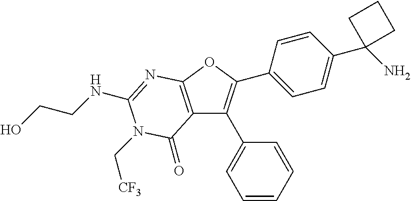

### Step 1: 6-Bromo-2-(methylthio)-5-phenyl-3-(2,2,2-trifluoroethyl)furo[2,3-d]pyrimidin-4(3H)-one

To a solution of 6-bromo-2-(methylthio)-5-phenylfuro[2,3-d]pyrimidin-4(3H)-one (2 g, 5.93 mmol) in DMF (10 ml) was added 2,2,2-trifluoroethanol (0.859 ml, 11.9 mmol) and PPh₃ (3.11 g, 11.9 mmol). The reaction mixture was cooled to 0° C. under N₂ followed by the addition of DEAD (1.88 ml, 11.9 mmol) dropwise. The reaction was allowed to stir at 50° C. for 1 h. Analysis by LCMS showed mostly O-alkylated product as well as a small amount of N-alkylated product. The reaction mixture was diluted with EtOAc (100 ml) and brine (75 ml) and extracted. The organic extracts were washed with brine (3×50 ml), dried (Na₂SO₄) and concentrated in vacuo. The resultant residue was purified by silica gel chromatography (0 to 50% EtOAc/hexane) to afford the title compound (0.1 g, 0.239 mmol, 4%) as a gum. LCMS (Method C) R_{T}=1.657 min, M+H⁺=418.9, 419.9. [N.B. O-Alkylated material, 6-bromo-2-(methylthio)-5-phenyl-4-(2,2,2-trifluoroethoxy)furo[2,3-d]pyrimidine (1.74 g, 70%), was isolated as a white solid. LCMS (Method E) R_{T}=1.776 min, M+H⁺=418.9, 419.9].

### Step 2: tert-Butyl 1-(4-(2-(methylthio)-4-oxo-5-phenyl-3-(2,2,2-trifluoroethyl)-3,4-dihydrofuro[2,3-d]pyrimidin-6-yl)phenyl)cyclobutylcarbamate

To a solution of 6-bromo-2-(methylthio)-5-phenyl-3-(2,2,2-trifluoroethyl)furo[2,3-d]pyrimidin-4(3H)-one (0.1 g, 0.239 mmol) in DME (3 ml) was added K₂CO₃ (0.165 g, 1.193 mmol) in water (0.750 ml) followed by tert-butyl 1-(4-(4,4,5,5-tetramethyl-1,3,2-dioxaborolan-2-yl)phenyl)cyclobutylcarbamate (0.107 g, 0.286 mmol, prepared as described in WO2008/070016). The mixture was then degassed with N₂ before the addition of Pd(PPh₃)₄ (0.028 g, 0.024 mmol). The reaction mixture was then heated at 80° C. for 18 h. Analysis by LCMS showed a mixture of starting material and product and therefore, further equivalents of Pd(PPh₃)₄ (0.05 eq) and boronate ester (0.5 eq) were added and heating continued for a further 18 h. The reaction mixture was cooled to room temperature, diluted with EtOAc and water and extracted. The organic extracts were washed with brine (3×25 ml), dried (Na₂SO₄) and concentrated in vacuo. The resultant residue was slurried in Et₂O, filtered and dried under vacuum to afford the title compound (0.102 g, 73%) as a white solid. LCMS (Method E) R_{T}=1.85 min, M+H⁺=586.

### Step 3: tert-Butyl 1-(4-(2-(methylsulfinyl)-4-oxo-5-phenyl-3-(2,2,2-trifluoroethyl)-3,4-dihydrofuro[2,3-d]pyrimidin-6-yl)phenyl)cyclobutylcarbamate

To a solution of tert-butyl 1-(4-(2-(methylthio)-4-oxo-5-phenyl-3-(2,2,2-trifluoroethyl)-3,4-dihydrofuro[2,3-d]pyrimidin-6-yl)phenyl)cyclobutylcarbamate (0.05 g, 0.085 mmol) in THF (3 ml) and methanol (1.5 ml) at 0° C. was added Oxone® (0.210 g, 0.342 mmol) in water (1.5 ml). The reaction mixture was heated to 50° C. and maintained at this temperature for 3 h. The reaction mixture was then cooled to room temperature, diluted with EtOAc and water and extracted. The organic extracts were washed with brine (2×15 ml), dried (Na₂SO₄) and concentrated in vacuo affording the crude title compound as an oil (49 mg, 95%). LCMS (Method E) R_{T}=1.626 min, M+H⁺=602.

### Step 4: tert-Butyl (1-(4-(2-((2-hydroxyethyl)amino)-4-oxo-5-phenyl-3-(2,2,2-trifluoroethyl)-3,4-dihydrofuro[2,3-d]pyrimidin-6-yl)phenyl)cyclobutyl)carbamate

To a solution of tert-butyl (1-(4-(2-(methylsulfinyl)-4-oxo-5-phenyl-3-(2,2,2-trifluoroethyl)-3,4-dihydrofuro[2,3-d]pyrimidin-6-yl)phenyl)cyclobutyl)carbamate (0.05 g, 0.083 mmol) in tetrahydrofuran (1 ml) was added 2-aminoethanol (0.051 g, 0.831 mmol). The reaction mixture was heated to 50° C. and maintained at this temperature for 2 h. The reaction mixture was then concentrated in vacuo, diluted with DCM (15 ml) and brine (10 ml) and extracted. The DCM extracts were dried (Na₂SO₄), concentrated and the residue purified by silica gel chromatography (0 to 100% EtOAc/cyclohexane) to afford the title compound (0.030 g, 60%) as a white solid. LCMS (Method E) R_{T}=1.579 min, M+H⁺=599.2.

### Step 5: 6-(4-(1-Aminocyclobutyl)phenyl)-2-((2-hydroxyethyl)amino)-5-phenyl-3-(2,2,2-trifluoroethyl)furo[2,3-d]pyrimidin-4(3H)-one

A solution of tert-butyl (1-(4-(2-((2-hydroxyethyl)amino)-4-oxo-5-phenyl-3-(2,2,2-trifluoroethyl)-3,4-dihydrofuro[2,3-d]pyrimidin-6-yl)phenyl)cyclobutyl)carbamate (0.030 g, 0.050 mmol) in DCM (2 ml) was loaded onto a pre-washed (DCM) 5 g SCX-2 column and allowed to stand for 6 h at RT. The column was washed with 5% MeOH/DCM (25 ml) followed by 15% 2N NH₃/MeOH in DCM (50 ml). The NH₃ wash fraction was concentrated in vacuo affording a gum that was purified by silica gel chromatography (0 to 10% MeOH/DCM) affording a gum. The gum was dissolved in MeOH (˜2 ml) and allowed to stand overnight. The resultant precipitate was collected by filtration, washed with cold MeOH, Et₂O and dried to afford the title compound as a white solid (0.006 g, 24%). LCMS (Method E) R_{T}=0.891 min, M−NH₃+H⁺=482.2. ¹H NMR (500 MHz, methanol-d₄): δ 7.3-7.5 (m, 9H), 4.9 (m, 2H), 3.82 (m, 2H), 3.7 (m, 2H), 2.55 (m, 2H), 2.25 (m, 2H), 2.1 (m, 1H), 1.75 (m, 1H).

### Example 122

### 6-(4-(1-Aminocyclobutyl)phenyl)-2-(methylamino)-5-phenyl-3-(2,2,2-trifluoroethyl)furo[2,3-d]pyrimidin-4(3H)-one

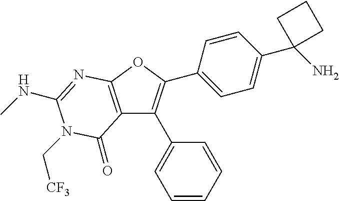

### Step 1: tert-Butyl (1-(4-(2-(methylamino)-4-oxo-5-phenyl-3-(2,2,2-trifluoroethyl)-3,4-dihydrofuro[2,3-d]pyrimidin-6-yl)phenyl)cyclobutyl)carbamate

To a solution of tert-butyl

(1-(4-(2-(methylsulfinyl)-4-oxo-5-phenyl-3-(2,2,2-trifluoroethyl)-3,4-dihydrofuro[2,3-d]pyrimidin-6-yl)phenyl)cyclobutyl)carbamate (25 mg, 0.042 mmol) in tetrahydrofuran (1 ml) was added methanamine (2M in THF) (1 ml, 0.042 mmol). The reaction mixture was heated to 40° C. until deemed complete by LCMS analysis. The reaction mixture was then concentrated in vacuo, diluted with DCM and water and extracted. The combined DCM extracts were dried and concentrated to afford the title compound (18 mg, 76%) as a gum. LCMS (Method E) R_{T}=1.704 min, M+H⁺=569.2.

### Step 2: 6-(4-(1-Aminocyclobutyl)phenyl)-2-(methylamino)-5-phenyl-3-(2,2,2-trifluoroethyl)furo[2,3-d]pyrimidin-4(3H)-one

A solution of tert-butyl (1-(4-(2-(methylamino)-4-oxo-5-phenyl-3-(2,2,2-trifluoroethyl)-3,4-dihydrofuro[2,3-d]pyrimidin-6-yl)phenyl)cyclobutyl)carbamate (0.03 g, 0.053 mmol) in DCM (2 ml) was loaded onto a 5 g SCX-2 column and allowed to stand for 18 h at RT. The column was then washed with 5% MeOH/DCM (25 ml) followed by 15% 2N NH₃/MeOH in DCM (50 ml). The NH₃ wash fraction was concentrated in vacuo down to −2 ml volume and allowed to stand for 60 h. The resultant crystalline solid that had formed was collected, washed with Et₂O and dried under vacuum. This afforded the title compound (0.011 g, 45%) as a white solid. LCMS (Method E) R_{T}=0.951 min, M−NH₃+H⁺=452.0. ¹H NMR (500 MHz, methanol-d₄): δ 7.3-7.6 (m, 9H), 4.85 (m, 2H), 3.06 (s, 3H), 2.75 (m, 2H), 2.58 (m, 2H), 2.25 (m, 1H), 1.95 (m, 1H).

### Example 123

### 6-(4-(1-Aminocyclobutyl)phenyl)-2-methyl-5-phenyl-3-(2,2,2-trifluoroethyl)furo[2,3-d]pyrimidin-4(3H)-one

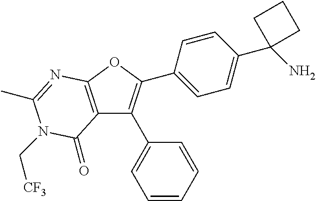

### Step 1: tert-Butyl (1-(4-(4-methoxy-2-methyl-5-phenylfuro[2,3-d]pyrimidin-6-yl)phenyl)cyclobutyl)carbamate

To a stirred solution of tert-butyl (1-(4-(4-methoxy-2-(methylsulfonyl)-5-phenylfuro[2,3-d]pyrimidin-6-yl)phenyl)cyclobutyl)carbamate (0.05 g, 0.091 mmol) in THF (0.5 ml) at 0° C. was added methylmagnesium iodide (3M in THF) (0.091 ml, 0.273 mmol) dropwise. The reaction mixture was allowed to warm to room temperature over 1 h. The reaction mixture was quenched with the addition of sat. NH₄Cl (aq) (15 ml) and extracted with DCM (2×15 ml). The combined DCM extracts were dried (Na₂SO₄) and concentrated to afford the title compound as a gum (42 mg, 86%). LCMS (Method E) R_{T}=1.858 min, M+H⁺=486.2.

### Step 2: tert-Butyl (1-(4-(2-methyl-4-oxo-5-phenyl-3,4-dihydrofuro[2,3-d]pyrimidin-6-yl)phenyl)cyclobutyl)carbamate

To a solution of tert-butyl (1-(4-(4-methoxy-2-methyl-5-phenylfuro[2,3-d]pyrimidin-6-yl)phenyl)cyclobutyl)carbamate (177 mg, 0.365 mmol) in dioxane (3 ml) was added 2N NaOH (2N) (3 ml, 0.365 mmol). The reaction mixture was heated to reflux until deemed complete by LCMS. The reaction mixture was then cooled to room temperature, diluted with DCM and water and extracted. The organic extracts were washed with brine (3×15 ml), dried (Na₂SO₄), filtered and concentrated in vacuo. The resultant residue was purified by silica gel chromatography (0 to 30% EtOAc/hexane) to afford the title compound (122 mg, 71%) as an off-white solid. LCMS (Method E) R_{T}=1.448 min, M+H⁺=472.2.

### Step 3: tert-Butyl (1-(4-(2-methyl-4-oxo-5-phenyl-3-(2,2,2-trifluoroethyl)-3,4-dihydrofuro[2,3-d]pyrimidin-6-yl)phenyl)cyclobutyl)carbamate

To a solution of tert-butyl (1-(4-(2-methyl-4-oxo-5-phenyl-3,4-dihydrofuro[2,3-d]pyrimidin-6-yl)phenyl)cyclobutyl)carbamate (25 mg, 0.053 mmol) in DMF (0.5 ml) was added Cs₂CO₃ (38.0 mg, 0.117 mmol) followed by 1,1,1-trifluoro-2-iodoethane (6.27 μl, 0.064 mmol). The reaction mixture was heated to 100° C. under microwave conditions until deemed complete by LCMS analysis. The reaction mixture was then diluted with EtOAc and water and extracted. The organic extracts were washed with brine (3×15 ml), dried (Na₂SO₄), filtered and concentrated in vacuo. The resultant residue was purified by silica gel chromatography (0 to 50% EtOAc/hexane) to afford the title compound (8 mg, 27.3%) as a gum. LCMS (Method E) R_{T}=1.733 min, M+H⁺=554.2.

### Step 4: 6-(4-(1-Aminocyclobutyl)phenyl)-2-methyl-5-phenyl-3-(2,2,2-trifluoroethyl)furo[2,3-d]pyrimidin-4(3H)-one

A solution of tert-butyl (1-(4-(2-methyl-4-oxo-5-phenyl-3-(2,2,2-trifluoroethyl)-3,4-dihydrofuro[2,3-d]pyrimidin-6-yl)phenyl)cyclobutyl)carbamate (0.023 g, 0.042 mmol) in DCM (2 ml) was loaded onto a pre-washed (DCM) 5 g SCX-2 column and allowed to stand for 48 h at RT. The column was then washed with 5% MeOH/DCM (25 ml) followed by 15% 2N NH₃/MeOH in DCM (50 ml). The NH₃ wash fraction was concentrated in vacuo affording a gum that was purified by silica gel chromatography (0 to 5% 2N NH₃/MeOH in DCM) to afford the title compound (13 mg, 69%) as an off-white solid. LCMS (Method E) R_{T}=0.923 min, M−NH₃+H⁺=437.0. ¹H NMR (500 MHz, CDCl₃): δ 7.54 (m, 4H), 7.4 (m, 3H), 7.3 (d, 2H), 4.8 (br s, 2H), 2.73 (s, 3H), 2.51 (m, 2H), 2.0-2.2 (m, 3H), 1.75 (m, 1]-1).

### Example 124

### 6-(4-(1-Aminocyclobutyl)phenyl)-3-methyl-5-phenylfuro[2,3-d]pyrimidine-2,4(1H,3H)-dione hydrochloride

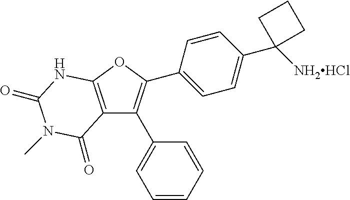

### Step 1: tert-Butyl (1-(4-(3-methyl-2,4-dioxo-5-phenyl-1,2,3,4-tetrahydrofuro[2,3-d]pyrimidin-6-yl)phenyl)cyclobutyl)carbamate

A solution of tert-butyl 1-(4-(3-methyl-2-(methylsulfonyl)-4-oxo-5-phenyl-3,4-dihydrofuro[2,3-d]pyrimidin-6-yl)phenyl)cyclobutylcarbamate (100 mg, 0.18 mmol) in a 2M aq. sodium hydroxide (2 ml), THF (2 ml) and MeOH (2 ml) was heated at reflux for 3 h. The reaction was diluted with water and extracted with a mixture of THF/EtOAc 1:1. The organic layer was dried (MgSO₄), filtered and concentrated in vacuo to afford the title compound (70 mg, 79%) as an off-white solid. LCMS (Method E) R_{T}=1.46 min, M+H⁺=488.

### Step 2: 6-(4-(1-Aminocyclobutyl)phenyl)-3-methyl-5-phenylfuro[2,3-d]pyrimidine-2,4(1H,3H)-dione

Following the procedure for 2-(4-(1-aminocyclobutyl)phenyl)-6-methyl-3-phenylfuro[2,3-c]pyridin-7(61-1)-one, tert-butyl (1-(4-(3-methyl-2,4-dioxo-5-phenyl-1,2,3,4-tetrahydrofuro[2,3-d]pyrimidin-6-yl)phenyl)cyclobutyl)carbamate (50 mg, 0.06 mmol) was deprotected to afford the desired compound (10 mg, 42%). The HCl salt was prepared by dissolving the material in DMF (2 ml) and adding 2 eq of HCl in methanol (freshly prepared from acetyl chloride/MeOH). A white solid precipitated that was collected by filtration and dried to afford the title compound (21 mg, 48%). LCMS (Method E) R_{T}=0.76 min M+H⁺=388, M−NH₃⁺=371, ¹H NMR (500 MHz, DMSO-d₆): δ 8.60 (br s, 3H), 7.35-7.50 (m, 9H), 3.16 (s, 3H), 2.09-2.18 (m, 2H), 1.73-1.82 (m, 2H), 1.13-1.43 (m, 2H).

### Example 125

### 6-(4-(1-Aminocyclobutyl)phenyl)-1,3-dimethyl-5-phenylfuro[2,3-d]pyrimidine-2,4(1H,3H)-dione

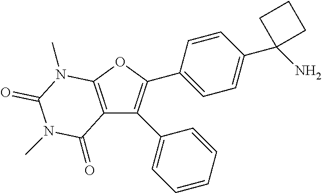

### Step 1: tert-Butyl (1-(4-(1,3-dimethyl-2,4-dioxo-5-phenyl-1,2,3,4-tetrahydrofuro[2,3-d]pyrimidin-6-yl)phenyl)cyclobutyl)carbamate

To a solution of tert-butyl 1-(4-(3-methyl-2,4-dioxo-5-phenyl-1,2,3,4-tetrahydrofuro[2,3-d]pyrimidin-6-yl)phenyl)cyclobutylcarbamate (100 mg, 0.21 mmol) in DMF (3 ml) was charged iodomethane (0.014 ml, 0.23 mmol) and potassium carbonate (85 mg, 0.62 mmol). The reaction was stirred at RT for 3 h. The reaction mixture was diluted with water and extracted twice with EtOAc. The combined organic extracts were washed 4 times with 1:1, water/brine, dried (MgSO₄), filtered, and concentrated in vacuo to afford the title compound (60 mg, 58%) as an off white solid. LCMS (Method E) R_{T}=1.61 min, M+H⁺=502.

### Step 2: 6-(4-(1-Aminocyclobutyl)phenyl)-1,3-dimethyl-5-phenylfuro[2,3-d]pyrimidine-2,4(1H,3H)-dione

Following the procedure for 2-(4-(1-aminocyclobutyl)phenyl)-6-methyl-3-phenylfuro[2,3-c]pyridin-7(6H)-one, tert-butyl 1-(4-(1,3-dimethyl-2,4-dioxo-5-phenyl-1,2,3,4-tetrahydrofuro[2,3-d]pyrimidin-6-yl)phenyl)cyclobutylcarbamate (60 mg, 0.12 mmol) was reacted to yield the title compound (23 mg, 48%) as a white solid. LCMS (Method E) R_{T}=0.81 min, M+H⁺=402. ¹H NMR (500 MHz, methanol-d₄): δ 7.35-7.45 (m, 9H), 3.66 (s, 3H), 3.33 (s, 3H), 2.51-2.57 (m, 2H), 2.21-2.27 (m, 2H), 2.02-2.10 (m, 1H), 1.70-1.79 (m, 1H).

### Example 126

### 1-(4-(7-Methoxy-3-phenylfuro[2,3-c]pyridin-2-yl)phenyl)cyclobutanamine

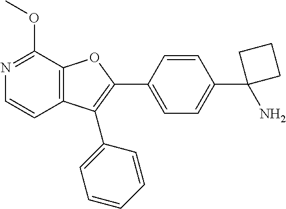

### Step 1: 4-Bromo-2,3-dimethoxypyridine

To a solution of 2,3-dimethoxypyridine (2.5 g, 18 mmol), in THF (100 mL) at −75° C. was added n-BuLi (15.8 ml, 39.5 mmol), slowly dropwise. The reaction mixture was stirred at 0° C. for 1 h and cooled again to −70° C. before the addition of bromine (0.93 ml, 18 mmol). After addition, the reaction was stirred at −70° C. for 2 h and then allowed to warm to RT. The reaction was quenched with water, and diluted with ethyl acetate. The layers were separated and the organic layer washed with aq. sodium thiosulphate (0.05M) and water. The organic layer was dried (MgSO₄), filtered and concentrated in vacuo to afford a brown oil. The resultant residue was subjected to flash chromatography (SiO₂, gradient 0 to 5% ethyl acetate in cyclohexane) to afford the title compound as a colorless oil (1.5 g, 29%). LCMS (Method A) R_{T}=5.20 min, M+H⁺=218/220.

### Step 2: tert-Butyl (1-(4-((2,3-dimethoxypyridin-4-yl)ethynyl)phenyl)cyclobutyl)carbamate

A solution of 4-bromo-2,3-dimethoxypyridine (1.5 g, 6.88 mmol), in triethylamine (15 mL) was degassed by bubbling N₂ through the reaction mixture for 10 min. Pd(t-Bu₃P)₂ (0.21 g, 0.41 mmol) was then charged to the reaction and the reaction cooled to 0° C. under a nitrogen atmosphere. After 10 min, copper(I) iodide (0.026 g, 0.14 mmol) was charged followed by dropwise addition of tert-butyl 1-(4-ethynylphenyl)cyclobutylcarbamate (2.24 g, 8.26 mmol) as a solution in triethylamine (30 mL, 215 mmol). The reaction was stirred for 2 h at 0° C. then at RT overnight. The reaction mixture was concentrated in vacuo and the residue was co-evaporated with DCM. The residue was dissolved in DCM and filtered through Celite®. The filtrate was concentrated in vacuo and the residue purified by flash chromatography (SiO₂, gradient 5 to 35% ethyl acetate in cyclohexane), to afford the title compound as a light brown solid (2.4 g, 85%). LCMS (Method A) R_{T}=7.55 min, M+H⁺=409.

### Step 3: tert-Butyl (1-(4-(3-iodo-7-methoxyfuro[2,3-c]pyridin-2-yl)phenyl)cyclobutyl)carbamate

To a solution of tert-butyl 1-(4-((2,3-dimethoxypyridin-4-yl)ethynyl)phenyl)cyclobutylcarbamate (530 mg, 1.30 mmol), in DCM (12 ml) at 0° C. was charged iodine monochloride 1M in DCM (1.95 ml, 1.95 mmol). After stirring at RT for 1 h, analysis by LCMS showed complete reaction. The reaction mixture was diluted with DCM and washed with satd. aq. sodium thiosulphate solution. The resulting biphasic solution was separated using a phase separator (Isolute® SPE), washing twice with DCM. The solvent was removed in vacuo to afford the title compound (590 mg, 87%) as a brown foamy solid. LCMS (Method A) R_{T}=8.37 min, M+H=521.

### Step 4: tert-Butyl (1-(4-(7-methoxy-3-phenylfuro[2,3-c]pyridin-2-yl)phenyl)cyclobutyl)carbamate

A solution of tert-butyl 1-(4-(3-iodo-7-methoxyfuro[2,3-c]pyridin-2-yl)phenyl)cyclobutylcarbamate (400 mg, 0.769 mmol), phenylboronic acid (141 mg, 1.15 mmol) and sodium carbonate, 2M in H₂O (1.92 ml, 3.84 mmol) in DME was degassed with N₂ for 10 min. Palladium tetrakis triphenylphosphine (44.4 mg, 0.038 mmol) was then charged to the reaction mixture and was heated for 20 min at 120° C. under microwave conditions. The reaction was diluted with water, extracted with ethyl acetate, dried (MgSO₄), filtered and concentrated in vacuo. The resultant residue was subjected to flash chromatography (SiO₂, gradient 0 to 20% ethyl acetate in cyclohexane) to afford the title compound as a white solid (136 mg, 29%) [contained 15% unreacted starting material]. LCMS (Method A) R_{T}=8.58 min, M+H⁺=471.

### Step 5: 1-(4-(7-Methoxy-3-phenylfuro[2,3-c]pyridin-2-yl)phenyl)cyclobutanamine

To a solution of tert-butyl 1-(4-(7-methoxy-3-phenylfuro[2,3-c]pyridin-2-yl)phenyl)cyclobutylcarbamate[Contained ˜15% of tert-butyl (1-(4-(3-iodo-7-methoxyfuro[2,3-c]pyridin-2-yl)phenyl)cyclobutyl)carbamate] (135 mg, 0.215 mmol) in DCM (2 ml) was charged TFA (0.25 ml, 3.24 mmol). The reaction was stirred at RT under a nitrogen atmosphere for 2 h. The solvent was removed in vacuo and the residue neutralised using aq.NaHCO₃ and extracted twice with ethyl acetate. The organic layers were combined, dried (MgSO₄), filtered, and concentrated in vacuo. The crude material was purified by preparative HPLC (Method F) to afford the title compound (54 mg, 68%) as a white solid. LCMS (Method A) R_{T}=4.45 min, M−NH₃⁺=354, M+2H⁺=372. ¹H NMR (500 MHz, methanol-d₄): δ 7.93 (d, 1H), 7.74-7.76 (m, 2H), 7.48-7.55 (m, 7H), 7.10 (d, 1H), 4.17 (s, 3H), 2.68-2.73 (m, 2H), 2.43-2.49 (m, 2H), 2.15-2.23 (m, 1H), 1.86-1.95 (m, 1H).

### Example 127

### 1-(4-(3-Iodo-7-methoxyfuro[2,3-c]pyridin-2-yl)phenyl)cyclobutanamine

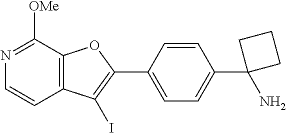

The Iodide was isolated from the preparation of 2-(4-(1-aminocyclobutyl)phenyl)-3-iodofuro[2,3-c]pyridin-7(6H)-one by preparative HPLC (Method F). LCMS (Method A) R_{T}=4.18 min, M+2H⁺=422, ¹H NMR (500 MHz, methanol-d₄): δ 8.25 (d, 2H), 8.00 (d, 1H), 7.68 (d, 2H), 7.09 (d, 1H), 4.15 (s, 3H), 2.67-2.72 (m, 2H), 2.38-2.43 (m, 2H), 2.14-2.22 (m, 1H), 1.84-1.92 (m, 1H).

### Example 128

### 2-(4-(1-Aminocyclobutyl)phenyl)-3-phenylfuro[2,3-d]pyridin-7(6H)-one

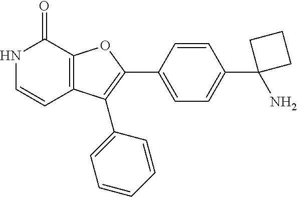

### Step 1: 2-(4-(1-Aminocyclobutyl)phenyl)-3-phenylfuro[2,3-c]pyridin-7(6H)-one

To a solution of 1-(4-(7-methoxy-3-phenylfuro[2,3-c]pyridin-2-yl)phenyl)cyclobutanamine in 1,4-dioxane (2 mL) was charged 2M HCl (2 mL). The reaction was heated at 80° C. for 3 h. The reaction mixture was concentrated in vacuo, the residue was neutralised using aq. NaHCO₃, and extracted with ethyl acetate (×3). The organic layers were combined, dried (MgSO₄), filtered, and concentrated in vacuo. The crude material was purified by preparative HPLC (Method F) to afford the title compound as a white solid (5 mg, 15%).

LCMS (Method A) R_{T}=3.72 min, M+2H⁺=358. ¹H NMR (500 MHz, DMSO-d₆) 8.39 (br s 3H) 7.44-7.55 (m, 9H), 7.22 (d, 1H), 6.28 (d, 1H), 2.35-2.40 (m, 2H), 2.06-2.12 (m, 2H), 1.95-2.03 (m, 1H), 1.61-1.70 (m, 1H).

### Example 129

### 2-(4-(1-Aminocyclobutyl)phenyl)-6-methyl-3-phenylfuro[2,3-c]pyridin-7(6H)-one

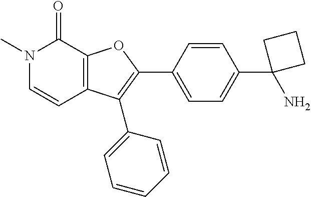

### Step 1: 2-(4-(1-Aminocyclobutyl)phenyl)-3-phenylfuro[2,3-c]pyridin-7(6H)-one

To a mixture of tert-butyl (1-(4-(7-methoxy-3-phenylfuro[2,3-c]pyridin-2-yl)phenyl)cyclobutyl)carbamate (400 mg, 0.85 mmol) and sodium iodide (382 mg, 2.55 mmol) in acetonitrile (10 ml) at RT under an atmosphere of nitrogen was charged chlorotrimethylsilane (0.11 ml, 0.85 mmol) dropwise over 5 min. The reaction was then stirred for 4 h at RT. Analysis by LCMS showed starting material remaining and therefore a further 0.5 eq of chlorotrimethylsilane was added and the reaction was stirred at RT over the weekend. The solvent was removed in vacuo and the residue partitioned between aq. NaHCO₃ and DCM, extracting with further DCM. The combined DCM extracts were dried (MgSO₄), filtered and concentrated in vacuo to afford the title compound (286 mg, 94%) as an orange foamy solid. LCMS (Method E) R_{T}=0.78 min, M-NH₃+H⁺340.

### Step 2: tert-Butyl (1-(4-(7-oxo-3-phenyl-6,7-dihydrofuro[2,3-c]pyridin-2-yl)phenyl)cyclobutyl)carbamate

To a suspension of 2-(4-(1-aminocyclobutyl)phenyl)-3-phenylfuro[2,3-c]pyridin-7(6H)-one (118 mg, 0.33 mmol) in THF (10 ml) was charged DMAP (2.0 mg, 0.017 mmol) followed by di-tert-butyl-dicarbonate (0.085 ml, 0.36 mmol). The resulting reaction mixture was stirred at 60° C. for 4 h. Analysis by LCMS showed that the reaction was almost complete. After 16 h, the reaction was diluted with ethyl acetate and water and the layers were separated. The organic layer was dried (MgSO₄), filtered and concentrated in vacuo to afford the title compound (114 mg, 75%). LCMS (Method E) R_{T}=1.45 min, M+H⁺=457.

### Step 3: tert-Butyl (1-(4-(6-methyl-7-oxo-3-phenyl-6,7-dihydrofuro[2,3-c]pyridin-2-yl)phenyl)cyclobutyl)carbamate

A solution of tert-butyl (1-(4-(7-oxo-3-phenyl-6,7-dihydrofuro[2,3-c]pyridin-2-yl)phenyl)cyclobutyl)carbamate (40 mg, 0.088 mmol), potassium carbonate (12.1 mg, 0.088 mmol) and iodomethane (10.9 μl, 0.175 mmol) in DMF (1.5 ml) was stirred at RT overnight. The reaction was diluted with ethyl acetate and water, separated, and the aqueous was further extracted with ethyl acetate. The ethyl acetate layers were combined and washed with 1:1, water/brine (×4), dried (MgSO₄), filtered, and concentrated in vacuo. The crude material was purified by column (SiO₂, 20 to 60% ethyl acetate in cyclohexane) to afford the title compound (20 mg, 49%) as an off-white solid. LCMS (Method E) R_{T}=1.56 min, M+H⁺=471.

### Step 4: 2-(4-(1-Aminocyclobutyl)phenyl)-6-methyl-3-phenylfuro[2,3-c]pyridin-7(6H)-one

A solution of tert-butyl (1-(4-(6-methyl-7-oxo-3-phenyl-6,7-dihydrofuro[2,3-c]pyridin-2-yl)phenyl)cyclobutyl)carbamate (20 mg, 0.043 mmol) in DCM (1 ml) and MeOH (0.5 ml) was charged to a pre-wetted (DCM: MeOH 8:2) Biotage SCX-2 column. The column was sealed and the material let stand on the column overnight. The column was washed with DCM:MeOH 8:2 followed by 2M NH₃ in MeOH. The ammonia eluent was concentrated in vacuo to afford a white solid. The material was dissolved in DCM:MeOH 95:5 and filtered. The solvent was concentrated in vacuo and dried under high vacuum overnight to yield the title compound (12 mg, 76%) as a white solid. LCMS (Method E) R_{T}=0.81 min, M+H⁺=371. ¹H NMR (500 MHz, DMSO-d₆): δ 7.44-7.57 (m, 10H), 6.33 (d, 1H), 3.58 (s, 3H), 2.37-2.43 (m, 2H), 2.12-2.18 (m, 2H), 1.97-2.05 (m, 1H), 1.63-1.73 (m, 1H).

### Example 130

### 2-(4-(1-Aminocyclobutyl)phenyl)-6-(2-fluoroethyl)-3-phenylfuro[2,3-c]pyridin-7(6H)-one

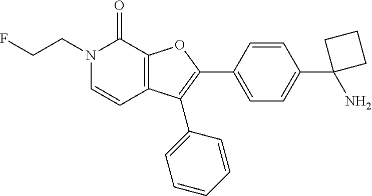

### Step 1: tert-Butyl 1-(4-(6-(2-fluoroethyl)-7-oxo-3-phenyl-6,7-dihydrofuro[2,3-c]pyridin-2-yl)phenyl)cyclobutylcarbamate

A solution of tert-butyl 1-(4-(7-oxo-3-phenyl-6,7-dihydrofuro[2,3-c]pyridin-2-yl)phenyl)cyclobutylcarbamate (84 mg, 0.184 mmol), potassium carbonate (25.4 mg, 0.184 mmol) and 1-fluoro-2-iodoethane (64.0 mg, 0.368 mmol) in DMF (3 ml) was stirred at RT over the weekend. The reaction was diluted with ethyl acetate and water and the layers separated the aqueous was further extracted with ethyl acetate. The ethyl acetate layers were combined and washed four times with water/brine 1:1, dried (MgSO₄), filtered, and concentrated in vacuo. The residue was purified by column (SiO₂, 15-55% ethyl acetate in cyclohexane) to afford the title compound (43 mg, 47%) as an off white solid. LCMS (Method E) R_{T}=1.60 min, M+H⁺=503.

### Step 2: 2-(4-(1-Aminocyclobutyl)phenyl)-6-(2-fluoroethyl)-3-phenylfuro[2,3-c]pyridin-7(6H)-one

Following the procedure for 2-(4-(1-aminocyclobutyl)phenyl)-6-methyl-3-phenylfuro[2,3-c]pyridin-7(6H)-one, tert-butyl 1-(4-(6-(2-fluoroethyl)-7-oxo-3-phenyl-6,7-dihydrofuro[2,3-c]pyridin-2-yl)phenyl)cyclobutylcarbamate (43 mg, 0.086 mmol) was reacted to afford the title compound (10 mg, 29%) as a white solid. LCMS R_{T}=0.87 min, 386 M-NH₃+H⁺. ¹H NMR (DMSO-d₆): δ 7.43-7.57 (m, 10H), 6.35 (d, 1H), 4.68-4.78 (m, 2H), 4.35-4.44 (m, 2H), 2.35-2.48 (m, 2H), 2.16-2.31 (m, 2H), 1.97-2.11 (m, 1H), 1.63-1.76 (m, 1H).

### Example 131

### 2-(4-(1-Aminocyclobutyl)phenyl)-6-(3-hydroxypropyl)-3-phenylfuro[2,3-c]pyridin-7(6H)-one

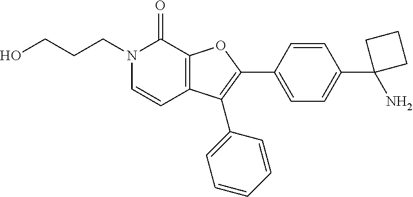

### Step 1: tert-Butyl 1-(4-(6-(3-hydroxypropyl)-7-oxo-3-phenyl-6,7-dihydrofuro[2,3-c]pyridin-2-yl)phenyl)cyclobutylcarbamate

To a solution of tert-butyl 1-(4-(7-oxo-3-phenyl-6,7-dihydrofuro[2,3-c]pyridin-2-yl)phenyl)cyclobutylcarbamate (75 mg, 0.164 mmol) in DMF (1 ml) was charged propane-1,3-diol (0.024 ml, 0.329 mmol) and triphenyl phosphine (86 mg, 0.329 mmol). The reaction mixture was cooled to 0° C. and diethyl azodicarboxylate (0.052 ml, 0.329 mmol) was charged to the reaction. LCMS after 1 h at 0° C. showed mainly starting material. The reaction was stirred at RT overnight. LCMS showed that the was reaction incomplete and therefore, a further batch of each of the reagents was charged to the reaction. After a further 24 h, the reaction was diluted with water and ethyl acetate, separated, the ethyl acetate was washed with water:brine 1:1 four times, dried (MgSO₄), filtered, and concentrated in vacuo. The crude material was purified by flash chromatography (SiO₂, 40 to 90% ethyl acetate in cyclohexane) to afford the title compound (16 mg, 19%) as an off white solid. LCMS (Method E) R_{T}=1.47 min, M+H⁺=515.

### Step 2: 2-(4-(1-Aminocyclobutyl)phenyl)-6-(3-hydroxypropyl)-3-phenylfuro[2,3-c]pyridin-7(6H)-one

Following the procedure for 2-(4-(1-aminocyclobutyl)phenyl)-6-methyl-3-phenylfuro[2,3-c]pyridin-7(6H)-one, tert-butyl (1-(4-(6-(3-hydroxypropyl)-7-oxo-3-phenyl-6,7-dihydrofuro[2,3-c]pyridin-2-yl)phenyl)cyclobutyl)carbamate (16 mg, 0.031 mmol) was reacted and purified by preparative HPLC (Method F) to afford the title compound (5.5 mg, 43%) as a white solid. LCMS (Method E) R_{T}=0.80 min, M+H⁺=415. ¹H NMR (500 MHz, methanol-d₄): δ 7.71 (d, 2H), 7.48-7.57 (m, 8H), 6.56 (d, 1H), 4.29 (t, 2H), 3.67 (t, 2H), 2.57-2.63 (m, 2H), 2.27-2.33 (m, 2H), 2.04-2.17 (m, 3H), 1.77-1.85 (m, 1H).

### Example 132

### 1-(4-(4-Bromo-7-methoxy-3-phenylfuro[2,3-c]pyridin-2-yl)phenyl)cyclobutanamine

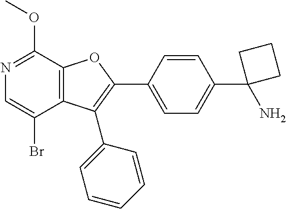

### Step 1: tert-Butyl 1-(4-(4-bromo-7-methoxy-3-phenylfuro[2,3-c]pyridin-2-yl)phenyl)cyclobutylcarbamate

To a suspension of tert-butyl 1-(4-(7-methoxy-3-phenylfuro[2,3-c]pyridin-2-yl)phenyl)cyclobutylcarbamate (100 mg, 0.213 mmol) in acetonitrile was charged N-bromosuccinimde (41.6 mg, 0.234 mmol) and the reaction heated to 90° C. overnight. The reaction was concentrated in vacuo and the residue dissolved in DCM and washed with water. The resulting biphasic solution was separated using a phase separator (Isolute® SPE) and the solvent removed in vacuo. The resultant residue was subjected to flash chromatography (SiO₂, gradient 0 to 10% ethyl acetate in cyclohexane) to afford the title compound as a white solid (80 mg, 68%). LCMS (Method E) R_{T}=1.99 min, M+H⁺=549/551.

### Step 2: 1-(4-(4-Bromo-7-methoxy-3-phenylfuro[2,3-c]pyridin-2-yl)phenyl)cyclobutanamine

Following the procedure for 2-(4-(1-aminocyclobutyl)phenyl)-6-methyl-3-phenylfuro[2,3-c]pyridin-7(6H)-one, tert-butyl 1-(4-(4-bromo-7-methoxy-3-phenylfuro[2,3-c]pyridin-2-yl)phenyl)cyclobutylcarbamate (30 mg, 0.05 mmol) was reacted and purified by flash chromatography (SiO₂, gradient 0 to 10% 2M NH₃ MeOH in DCM) to afford the title compound (12 mg, 50%) as a white solid LCMS (Method E) R_{T}=1.10 min, M+H⁺=449, 451. ¹H NMR (500 MHz, DMSO-d₆): δ 8.03 (s, 1H), 7.52-7.54 (m, 3H), 7.44-7.47 (m, 6H), 4.10 (s, 3H), 2.30-2.35 (m, 2H), 1.92-2.13 (m, 3H), 1.58-1.66 (m, 1H).

### Example 133

### 1-(4-(3-Phenyl-4-(1H-pyrazol-4-yl)furo[3,2-c]pyridin-2-yl)phenyl)cyclobutanamine

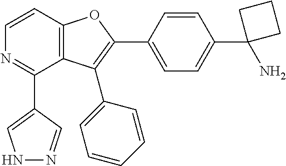

### Step 1: 2-(4-(1-((tert-Butoxycarbonyl)amino)cyclobutyl)phenyl)-3-phenylfuro[3,2-c]pyridin-4-yl trifluoromethanesulfonate

To a suspension of tert-butyl (1-(4-(4-oxo-3-phenyl-4,5-dihydrofuro[3,2-c]pyridin-2-yl)phenyl)cyclobutyl)carbamate (150 mg, 0.33 mmol) in DMF (5 ml) at 0° C. under an atmosphere of nitrogen was charged NaH (29 mg, 0.72 mmol). The reaction was stirred at 0° C. for 45 min. 1,1,1-trifluoro-N-phenyl-N-(trifluoromethylsulfonyl)methanesulfonamide (176 mg, 0.49 mmol) was charged to the reaction and the reaction stirred at RT for 2.5 h. The reaction was quenched by the addition of ice, diluted with water and extracted twice with ethyl acetate. The combined organic extracts were washed with H₂O:brine 1:1 four times, dried (MgSO₄), filtered, and concentrated in vacuo to afford the title compound as an off white solid (150 mg, 78%).

LCMS (Method A) R_{T}=8.75 min, M+H⁺=589.

### Step 2: tert-Butyl (1-(4-(3-phenyl-4-(1H-pyrazol-4-yl)furo[3,2-c]pyridin-2-yl)phenyl)cyclobutyl)carbamate

To a microwave vial were charged 2-(4-(1-((tert-butoxycarbonyl)amino)cyclobutyl)phenyl)-3-phenylfuro[3,2-c]pyridin-4-yl trifluoromethanesulfonate (20 mg, 0.03 mmol), 4-(4,4,5,5-tetramethyl-1,3,2-dioxaborolan-2-yl)-1H-pyrazole (10 mg, 0.05 mmol) and aqueous sodium carbonate (250 μl, 2M) in DME (500 μL). The mixture was degassed by bubbling N₂ through the reaction mixture for 5 min. Palladium tetrakis triphenylphosphine (2 mg, 1.7 μmol) was charged to the reaction and heated in a microwave reactor at 120° C. for 20 min. The mixture was partitioned between water and ethyl acetate. The layers were separated and the aqueous phase was extracted once more with ethyl acetate. The organic phase was dried (MgSO₄), filtered and concentrated in vacuo. The resultant residue was subjected to flash chromatography (SiO₂, gradient 20 to 100% ethyl acetate in hexane) to afford the title compound as a white solid (9.5 mg, 55%). LCMS (Method A) R_{T}=4.91 min, M+H^{#}=507.

### Step 3: 1-(4-(3-Phenyl-4-(1H-pyrazol-4-yl)furo[3,2-c]pyridin-2-yl)phenyl)cyclobutanamine

Following the procedure for 6-(4-(1-aminocyclobutyl)phenyl)-4-methoxy-N,N-dimethyl-5-phenylfuro[2,3-d]pyrimidin-2-amine, tert-butyl (1-(4-(3-phenyl-4-(1H-pyrazol-4-yl)furo[3,2-c]pyridin-2-yl)phenyl)cyclobutyl)carbamate was reacted to afford the title compound (14 mg, 54%) as a white solid. LCMS (Method A) R_{T}=2.87 min, M+H^{#}=407. ¹H NMR (500 MHz, methanol-d₄): δ 8.56 (d, 1H), 7.82 (d, 1H), 7.70 (d, 2H), 7.47-7.52 (m, 3H), 7.41 (t, 2H), 7.25-7.29 (m, 4H), 2.75-2.86 (m, 2H), 2.58-2.64 (m, 2H), 2.20-2.29 (m, 1H), 1.93-1.98 (m, 1H).

### Example 134

### 1-(4-(2-Chloro-7-phenylfuro[2,3-b]pyrazin-6-yl)phenyl)cyclobutanamine

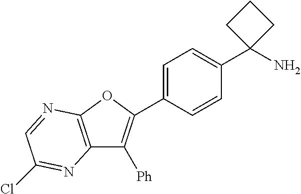

### Step 1: 2-(4-Methoxybenzylamino)acetonitrile

[Adapted from: Eur. J. Org. Chem., 2004, 20, 4158-4166 for preparation of the same compound]. 2-Chloroacetonitrile (17.30 ml, 273 mmol) and potassium carbonate (50.4 g, 364 mmol) were added to a stirred solution of (4-methoxyphenyl)methanamine (25.0 g, 182 mmol) in acetonitrile (2.0 l). The temperature was increased to 60° C. After 16 hours, the reaction was cooled to RT and filtered (Phase Separator) and the solvents were removed in vacuo. The remaining residue was passed through a silica plug and the solvents were removed in vacuo to give crude material of the title compound (ca. 32 g) that was carried through to the next step without further purification.

### Step 2: 3,5-Dichloro-1-(4-methoxybenzyl)pyrazin-2(1H)-one

Oxalyl chloride (39.9 ml, 455 mmol) was added to a stirred solution of the crude 2-(4-methoxybenzylamino)acetonitrile (assumed 32.1 g, 182 mmol) in chlorobenzene (250 ml) at RT under an atmosphere of nitrogen. After 30 minutes, triethylamine hydrochloride (100 g, 729 mmol) was added. After 16 hours, the solvents were removed in vacuo and the remaining residue was dissolved in DCM and washed successively with water (2×250 ml) and brine (2×200 ml). The combined organic phase was dried (Na2CO3), solvent removed in vacuo and the remaining reside was purified by flash chromatography (SiO₂, 0→20% EtOAc in n-hexane) to give the title compound (15.9 g, 31%) as a viscous orange oil. LCMS (Method E): R_{T}=1.217 min, M+Cl⁻=319.2.

### Step 3: tert-Butyl 1-(4-((6-chloro-4-(4-methoxybenzyl)-3-oxo-3,4-dihydropyrazin-2-yl)ethynyl)phenyl)cyclobutylcarbamate

tert-Butyl 1-(4-ethynylphenyl)cyclobutylcarbamate (2.00 g, 7.37 mmol) in DMF (20 ml) was added dropwise (over 4 hours via syringe pump) to a pre-degassed (bubbling nitrogen) stirred solution of 3,5-dichloro-1-(4-methoxybenzyl)pyrazin-2(1H)-one (1.617 g, 5.67 mmol), bis(triphenylphosphine)palladium(II) chloride (0.119 g, 0.170 mmol) and copper(I) iodide (10.80 mg, 0.057 mmol) in triethylamine (30 ml, 215 mmol)/DMF (10 ml) at 80° C. under an atmosphere of nitrogen. After 16 hours, the reaction mixture was cooled to RT and partitioned between EtOAc and water, separated, further washed with water and brine, dried (Phase Separator). The solvents were removed in vacuo and the remaining residue was purified by flash chromatography (SiO₂, 0-30% EtOAc/cyclohexane) to give the title compound (2.19 g, 74%) as a yellow foam. LCMS (Method A): R_{T}=7.49 min, M+Na⁺=542.1.

### Step 4: tert-Butyl 1-(4-(2-chloro-7-iodofuro[2,3-b]pyrazin-6-yl)phenyl)cyclobutylcarbamate

Iodine (2.43 g, 9.57 mmol) was added to a stirred solution of tert-butyl 1-(4-((6-chloro-4-(4-methoxybenzyl)-3-oxo-3,4-dihydropyrazin-2-yl)ethynyl)phenyl)cyclobutylcarbamate (2.49 g, 4.79 mmol) in DCM (30 ml) at RT under an atmosphere of nitrogen. After 15 minutes, analysis by LCMS showed complete reaction. The reaction mixture was partitioned between DCM and saturated aqueous sodium thiosulfate solution, separated, extracted (DCM×3), dried (Phase Separator), the solvents were removed in vacuo and the remaining residue was purified by flash chromatography (SiO₂, 0→20% EtOAc in cyclohexane) to give the title compound (1.54 g, 61%) as a yellow solid. LCMS (Method A): R_{T}=8.01 min, M-Cl⁺=489.9, 491.9.

### Step 5: tert-Butyl 1-(4-(2-chloro-7-phenylfuro[2,3-b]pyrazin-6-yl)phenyl)cyclobutylcarbamate and tert-butyl (1-(4-(2,7-diphenylfuro[2,3-b]pyrazin-6-yl)phenyl)cyclobutyl)carbamate

Tetrakis(triphenylphosphine)palladium(0) (0.162 g, 0.140 mmol) was added to a pre-degassed (bubbling nitrogen) solution of tert-butyl 1-(4-(2-chloro-7-iodofuro[2,3-b]pyrazin-6-yl)phenyl)cyclobutylcarbamate (1.47 g, 2.80 mmol), phenylboronic acid (0.376 g, 3.08 mmol) and potassium phosphate, tribasic (1.78 g, 8.40 mmol) in DMF (60 ml)/water (15 ml) and the reaction mixture was heated to 80° C. under an atmosphere of nitrogen. After 16 hours, the reaction mixture was partitioned between EtOAc and water, separated and washed again with water and brine, dried (Phase Separator), solvents removed in vacuo and the remaining residue purified by flash chromatography (SiO₂, 0-10% EtOAc in cyclohexane) to give the title compound (703 mg, 53%) as a white solid, LCMS (Method A): t=8.44 min, M+H⁺=475.8; and some of the over-reacted product, tert-butyl (1-(4-(2,7-diphenylfuro[2,3-b]pyrazin-6-yl)phenyl)cyclobutyl)carbamate (76.5 mg, 5%) was also isolated, LCMS (Method A): R_{T}=8.97 min, M+H⁺=518.2.

### Step 6: 1-(4-(2-Chloro-7-phenylfuro[2,3-b]pyrazin-6-yl)phenyl)cyclobutanamine, 2HCl

4M HCl in 1,4-dioxane (2.0 ml, 8.00 mmol) was added to a stirred solution of tert-butyl (1-(4-(2-chloro-7-phenylfuro[2,3-b]pyrazin-6-yl)phenyl)cyclobutyl)carbamate (8.5 mg, 0.018 mmol) in tetrahydrofuran (0.5 ml) at RT under nitrogen. After 16 hours, the precipitate that formed was purified by trituration with diethyl ether (3×2.0 ml), the solvents were removed in vacuo and the remaining residue was freeze dried to give the title compound (7.4 mg, 92%) as a white solid. ¹H NMR (500 MHz, methanol-d₄): δ 8.38 (s, 1H), 7.87 (d, 2H), 7.63-7.47 (m, 7H), 2.84-2.76 (m, 2H), 2.64-2.56 (m, 2H), 2.30-2.20 (m, 1H), 2.04-1.94 (m, 1H). LCMS (Method A): R_{T}=4.55 min, M+2⁺=377.0.

### Example 135

### 1-(4-(7-Phenyl-2-(1H-pyrazol-4-yl)furo[2,3-b]pyrazin-6-yl)phenyl)cyclobutanamine

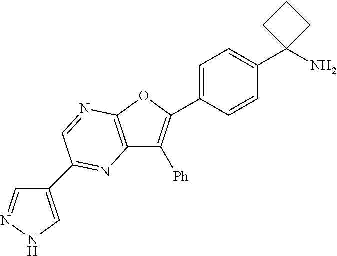

### Step 1: tert-Butyl (1-(4-(7-phenyl-2-(1H-pyrazol-4-yl)furo[2,3-b]pyrazin-6-yl)phenyl)cyclobutyl)carbamate

Tetrakis(triphenylphosphine)palladium(0) (6.0 mg, 0.005 mmol) was added to a pre-degassed (bubbling N₂) solution of tert-butyl 1-(4-(2-chloro-7-phenylfuro[2,3-b]pyrazin-6-yl)phenyl)cyclobutylcarbamate (50.0 mg, 0.105 mmol), 1H-pyrazole-4-boronic acid pinacol ester (30.6 mg, 0.158 mmol) and potassium phosphate, tribasic (66.9 mg, 0.315 mmol) in DMF (1.0 ml)/water (0.25 ml) at RT in a microwave vial. The reaction vessel was sealed and heated under microwave conditions at 120° C. for 20 minutes with stirring. LCMS showed some residual starting material and therefore, the reaction was further heated under microwave conditions at 120° C. for 10 minutes. The reaction mixture was partitioned between EtOAc and water, separated, extracted further with water and brine, dried (Phase Separator) and the solvents were removed in vacuo. The remaining residue was purified using flash chromatography (SiO₂, 0-50%, EtOAc in cyclohexane) to give the title compound (43.3 mg, 81%) as a white solid. LCMS (Method A): R_{T}=7.17 min; M+H⁺=508.2.

### Step 2: 1-(4-(7-Phenyl-2-(1H-pyrazol-4-yl)furo[2,3-b]pyrazin-6-yl)phenyl)cyclobutanamine, 2HCl

4 M HCl in 1,4-dioxane (1.5 ml, 6.00 mmol) was added dropwise to a stirred solution of tert-butyl 1-(4-(7-phenyl-2-(1H-pyrazol-4-yl)furo[2,3-b]pyrazin-6-yl)phenyl)cyclobutylcarbamate (43.3 mg, 0.085 mmol) in tetrahydrofuran (0.5 ml) at RT under nitrogen. After 2 hours LCMS appeared to show residual starting material and therefore the reaction was continued over the weekend. After 2.5 days, diethyl ether (1.5 ml) was added and the precipitate that formed was triturated using further diethyl ether (4×2.0 mL) to remove impurities. The residual solvents were removed and the residue was freeze dried to give the title compound (25.7 mg, 63%) as a pale yellow solid. ¹H NMR (500 MHz, methanol-d₄): δ 8.70 (s, 1H), 8.31 (bs, 2H), 7.89-7.84 (m, 2H), 7.67-7.63 (m, 2H), 7.60-7.47 (m, 5H), 2.85-2.77 (m, 2H), 2.66-2.58 (m, 2H), 2.31-2.21 (m, 1H), 2.05-1.95 (m, 1H). LCMS (Method A): R_{T}=3.90 min, M+2⁺=409.2.

### Example 136

### 1-(4-(2-(1-Methyl-1H-pyrazol-4-yl)-7-phenylfuro[2,3-b]pyrazin-6-yl)phenyl)cyclobutanamine

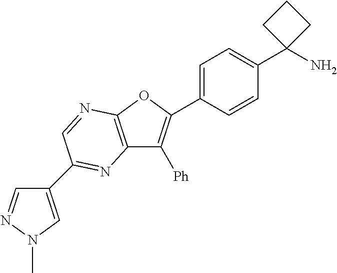

### Step 1: tert-Butyl 1-(4-(2-(1-methyl-1H-pyrazol-4-yl)-7-phenylfuro[2,3-b]pyrazin-6-yl)phenyl)cyclobutylcarbamate

Tetrakis(triphenylphosphine)palladium(0) (6.1 mg, 0.005 mmol) was added to a pre-degassed mixture of tert-butyl 1-(4-(2-chloro-7-phenylfuro[2,3-b]pyrazin-6-yl)phenyl)cyclobutylcarbamate (50.0 mg, 0.105 mmol), 1-methyl-1H-pyrazole-4-boronic acid pinacol ester (32.8 mg, 0.158 mmol) and potassium phosphate, tribasic (66.9 mg, 0.315 mmol) in DMF (1.0 ml)/water (0.25 ml) in a microwave vial. The reaction vessel was sealed and was subjected to microwave irradiation (CEM Explorer/Discover) at 120° C. for 20 minutes. Analysis by LCMS showed complete conversion to product. The reaction mixture was partitioned between EtOAc and water, separated and washed further with water and brine. The combined organic phase was dried (Phase Separator) and the solvents were removed in vacuo. The remaining residue was purified by flash chromatography (SiO₂, 0-50%, EtOAc in cyclohexane) to give the title compound (33.1 mg, 60%) as white solid. LCMS (Method A): R_{T}=7.71 min, M+H⁺=522.2.

### Step 2: 1-(4-(2-(1-Methyl-1H-pyrazol-4-yl)-7-phenylfuro[2,3-b]pyrazin-6-yl)phenyl)cyclobutanamine, 2HCl

4 M HCl in 1,4-dioxane (4.00 ml, 16.0 mmol) was added dropwise to a stirred solution of tert-butyl 1-(4-(2-(1-methyl-1H-pyrazol-4-yl)-7-25 phenylfuro[2,3-b]pyrazin-6-yl)phenyl)cyclobutylcarbamate (33.1 mg, 0.063 mmol) in tetrahydrofuran (1.0 ml) at RT under nitrogen. After 16 hours, analysis by LCMS showed complete conversion to product. Diethyl ether (2.0 ml) was added and the precipitate that formed was triturated using further diethyl ether (4×2.0 mL) to remove impurities. The residual solvents were removed in vacuo to give the title compound (27.2 mg, 87%) as a pale yellow solid, ¹H NMR (500 MHz, methanol-d₄): δ 8.63 (s, 1H), 8.24 (s, 1H), 8.08 (s, 1H), 7.85 (d, 2H), 7.66-7.63 (m, 2H), 7.58-7.46 (m, 5H), 3.95 (s, 3H), 2.81-2.73 (m, 2H), 2.60-2.52 (m, 2H), 2.28-2.18 (m, 1H), 2.02-1.92 (m, 1H). LCMS (Method A): R_{T}=4.24 min, M+2⁴=423.2.

### Example 137

### 1-(4-(2-(1-methyl-1H-pyrazol-5-yl)-7-phenylfuro[2,3-b]pyrazin-6-yl)phenyl)cyclobutanamine

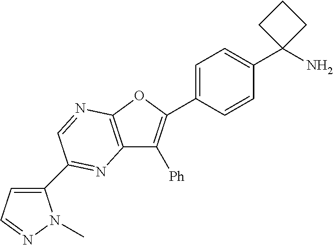

### Step 1: tert-Butyl 1-(4-(2-(1-methyl-1H-pyrazol-5-yl)-7-phenylfuro[2,3-b]pyrazin-6-yl)phenyl)cyclobutylcarbamate

Tetrakis(triphenylphosphine)palladium(0) (6.1 mg, 0.005 mmol) was added to a pre-degassed solution of tert-butyl 1-(4-(2-chloro-7-phenylfuro[2,3-b]pyrazin-6-yl)phenyl)cyclobutylcarbamate (50.0 mg, 0.105 mmol), 1-methyl-1H-pyrazole-5-boronic acid pinacol ester (32.8 mg, 0.158 mmol) and potassium phosphate, tribasic (66.9 mg, 0.315 mmol) in DMF (1.0 ml)/water (0.25 ml) in a microwave vial. The reaction vessel was sealed was subjected to microwave irradiation (CEM Explorer/Discover) at 120° C. for 20 minutes. Analysis by LCMS showed complete conversion to product. The reaction mixture was partitioned between EtOAc and brine, separated, extracted (EtOAc×2), combined organics were dried (Phase Separator), solvents were removed in vacuo and the remaining residue was purified by flash chromatography (SiO₂, 0→20% EtOAc in cyclohexane) to give the title compound (53.7 mg, 98%). LCMS (Method A): R_{T}=7.98 min, M+H⁺=522.2.

### Step 2: 1-(4-(2-(1-Methyl-1H-pyrazol-5-yl)-7-phenylfuro[2,3-b]pyrazin-6-yl)phenyl)cyclobutanamine, 2HCl

4 M HCl in 1,4-dioxane (4.0 ml, 16.00 mmol) was added to a stirred solution of tert-butyl 1-(4-(2-(1-methyl-1H-pyrazol-5-yl)-7-phenylfuro[2,3-b]pyrazin-6-yl)phenyl)cyclobutylcarbamate (53.7 mg, 0.103 mmol) in tetrahydrofuran (1.0 ml) at RT under nitrogen. After 2.5 days, LCMS showed complete reaction. Diethyl ether (2.0 ml) was added and the precipitate that formed was triturated using further diethyl ether (4×2.0 mL) to remove impurities. The residual solvents were removed in vacuo and the residue was freeze-dried to give the title compound (42.9 mg, 84%) as a pale yellow solid. LCMS (Method A): R_{T}=4.36 min, M+2⁺=423.2. ¹H NMR (500 MHz, methanol-d₄): δ 8.77 (s, 1H), 7.94-7.90 (d, 2H), 7.70-7.66 (m, 2H), 7.62-7.59 (m, 2H), 7.56 (d, 1H), 7.54-7.47 (m, 3H), 6.90 (d, 1H), 4.17 (s, 3H), 2.86-2.78 (m, 2H), 2.67-2.59 (m, 2H), 2.32-2.22 (m, 1H), 2.06-1.96 (m, 1H).

### Example 138

### 1-(4-(2-Cyclopropyl-7-phenylfuro[2,3-b]pyrazin-6-yl)phenyl)cyclobutanamine

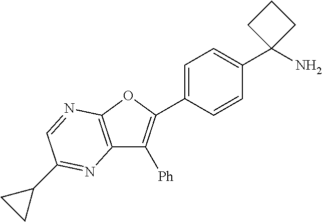

### Step 1: tert-Butyl 1-(4-(2-cyclopropyl-7-phenylfuro[2,3-b]pyrazin-6-yl)phenyl)cyclobutylcarbamate

Palladium(II)acetate (0.5 mg, 0.002 mmol) was added to a pre-degassed solution of tert-butyl 1-(4-(2-chloro-7-phenylfuro[2,3-t]pyrazin-6-yl)phenyl)cyclobutylcarbamate (50.0 mg, 0.105 mmol), di(1-adamantyl)-n-butylphosphine (1.1 mg, 0.003 mmol), potassium cyclopropyltrifluoroborate (17.1 mg, 0.116 mmol), and cesium carbonate (103 mg, 0.315 mmol) in α,α,α-trifluorotoluene (1.0 ml)/water (0.1 ml) in a microwave vial. The vessel was sealed and irradiated at 120° C. for 20 minutes. Analysis by LCMS showed incomplete reaction and therefore, the reaction was further heated under microwave conditions at 120° C. for 40 minutes. LCMS indicated complete reaction. The reaction mixture was partitioned between EtOAc and 1:1 water/brine, separated, dried (Phase Separator), solvents removed in vacuo and remaining residue purified by flash chromatography (SiO₂, 0→20%, EtOAc in cyclohexane) to give the title compound (50.6 mg, 0.118 mmol, quantitative) as a yellow oil. LCMS (Method A): R_{T}=8.73 min, M+H⁺=482.2.

### Step 2: 1-(4-(2-Cyclopropyl-7-phenylfuro[2,3-b]pyrazin-6-yl)phenyl)cyclobutanamine, 2HCl

4 M HCl in 1,4-dioxane (4.0 ml, 16.0 mmol) was added to a stirred solution of tert-butyl 1-(4-(2-cyclopropyl-7-phenylfuro[2,3-b]pyrazin-6-yl)phenyl)cyclobutylcarbamate (50.6 mg, 0.105 mmol) in tetrahydrofuran (1.0 ml) at RT under nitrogen. After leaving over the weekend, LCMS showed complete reaction. Diethyl ether was added (10 ml) to encourage precipitation and the material was purified by trituration using diethyl ether (5×2.0 ml). The residual solvents were removed in vacuo and the residue was freeze-dried to give the title compound (35.6 mg, 75%) as a pale yellow solid. ¹H NMR (500 MHz, methanol-d₄): δ 8.25 (s, 1H), 7.84 (d, 2H), 7.62-7.42 (m, 7H), 2.86-2.75 (m, 2H), 2.68-2.57 (m, 2H), 2.33-2.20 (m, 2H), 2.04-1.94 (m, 1H), 1.10-1.00 (m, 4H). LCMS (Method A): R_{T}=4.80 min, M+2⁺=383.1.

### Example 139

### 1-(4-(2,7-Diphenylfuro[2,3-b]pyrazin-6-yl)phenyl)cyclobutanamine, HCl

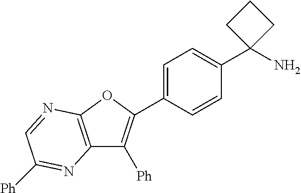

tert-Butyl 1-(4-(2,7-diphenylfuro[2,3-b]pyrazin-6-yl)phenyl)cyclobutylcarbamate (76.5 mg, 0.148 mmol) was dissolved in THF (0.5 ml) and 4 M HCl in 1,4-dioxane (1.5 ml, 43.2 mmol) was added dropwise at RT under nitrogen. After 16 hours, the reaction mixture was partitioned between DCM and saturated NaHCO₃ (aq) solution, separated, extracted, dried (Phase Separator). The solvents were removed in vacuo and the residue was purified by preparative HPLC (Method F) to give the product material as the free base. The material was dissolved in THF (1.0 ml) and 2M HCl in diethyl ether added. The precipitate that formed was washed successively with n-hexane (3×2.0 ml) and diethyl ether (3×2.0 ml). The residual solvents were removed in vacuo and the residue was freeze-dried to give the title compound (4.8 mg, 7%) as a pale yellow solid. LCMS (Method A): R_{T}=5.08 min, M+2⁺=419.1. ¹H NMR (500 MHz, methanol-d₄): δ 8.85 (s, 1H), 8.11-8.08 (m, 2H), 7.90-7.86 (m, 2H), 7.71-7.68 (m, 2H), 7.60-7.56 (d, 2H), 7.55-7.44 (m, 6H), 2.81-2.73 (m, 2H), 2.59-2.51 (m, 2H), 2.28-2.18 (m, 1H), 2.02-1.93 (m, 1H).

### Example 140

### 1-(4-(7-Phenyl-2-(pyrimidin-5-yl)furo[2,3-b]pyrazin-6-yl)phenyl)cyclobutanamine

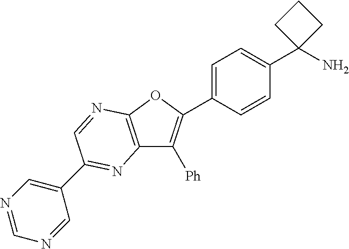

### Step 1: tert-Butyl 1-(4-(7-phenyl-2-(pyrimidin-5-yl)furo[2,3-b]pyrazin-6-yl)phenyl)cyclobutylcarbamate

Tetrakis(triphenylphosphine)palladium(0) (4.1 mg, 0.004 mmol) was added to a pre-degassed (bubbling nitrogen) solution of tert-butyl 1-(4-(2-chloro-7-phenylfuro[2,3-b]pyrazin-6-yl)phenyl)cyclobutylcarbamate (33.6 mg, 0.071 mmol), pyrimidine-5-boronic acid (13.1 mg, 0.106 mmol) and potassium phosphate, tribasic (45.0 mg, 0.212 mmol) in DMF (1.0 ml)/water (0.25 ml) at RT in a microwave vial. The reaction vessel was sealed and subjected to microwave irradiation at 120° C. for 20 minutes with stirring. LCMS showed complete conversion to product. The reaction mixture was partitioned between EtOAc and 1:1 brine/water, separated, extracted (EtOAc×2), dried (Phase Separator), solvents removed in vacuo and purified by flash chromatography (SiO₂, 0-50%, EtOAc in cyclohexane) to give the title compound (28.5 mg, 78%) as a pale yellow solid. LCMS (Method A): R_{T}=7.61 min, M+H⁺=520.3.

### Step 2: 1-(4-(7-Phenyl-2-(pyrimidin-5-yl)furo[2,3-b]pyrazin-6-yl)phenyl)cyclobutanamine, 2HCl

4 M HCl in 1,4-dioxane (4.0 ml, 16.0 mmol) was added to a stirred solution of tert-butyl (1-(4-(7-phenyl-2-(pyrimidin-5-yl)furo[2,3-b]pyrazin-6-yl)phenyl)cyclobutyl)carbamate (28.5 mg, 0.055 mmol) in tetrahydrofuran (1.0 ml) at RT under nitrogen. After 2 hours, analysis by LCMS showed complete reaction. Diethyl ether (2.0 ml) was added and the precipitate that formed was purified by trituration with diethyl ether (4×2.0 ml). The residual solvents were removed and the residue was freeze-dried to give the title compound (16.2 mg, 60%) as a pale yellow fluffy solid. ¹H NMR (500 MHz, methanol-d₄): δ 9.48 (s, 2H), 9.24 (d, 1H), 9.01 (d, 1H), 7.94-7.90 (m, 2H), 7.71-7.68 (m, 2H), 7.62-7.58 (d, 2H), 7.57-7.49 (m, 3H), 2.86-2.78 (m, 2H), 2.65-2.57 (m, 2H), 2.31-2.21 (m, 1H), 2.05-1.95 (m, 1H). LCMS (Method A): R_{T}=4.06 min, M+2⁺=421.2.

### Example 141

### 1-(4-(7-Phenyl-2-(pyridin-2-yl)furo[2,3-b]pyrazin-6-yl)phenyl)cyclobutanamine

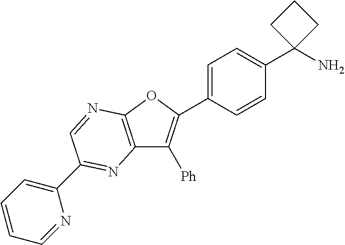

### Step 1: tert-Butyl 1-(4-(7-phenyl-2-(pyridin-2-yl)furo[2,3-b]pyrazin-6-yl)phenyl)cyclobutylcarbamate

Tetrakis(triphenylphosphine)palladium(0) (5.0 mg, 0.004 mmol), copper(I) iodide (0.8 mg, 0.004 mmol) and 2-(tributylstannyl)pyridine (0.038 ml, 0.105 mmol) were added to a pre-degassed (bubbling nitrogen) solution of tert-butyl 1-(4-(2-chloro-7-phenylfuro[2,3-b]pyrazin-6-yl)phenyl)cyclobutylcarbamate (50 mg, 0.087 mmol) and cesium fluoride (39.7 mg, 0.262 mmol) in DME (1.0 ml) in a 10 ml microwave vial. The vessel was sealed and irradiated at 120° C. for 20 minutes (CEM Explorer/Discover). LCMS indicated almost complete conversion. The solvents were removed in vacuo and the material was purified by flash chromatography (SiO₂, 0-50%, EtOAc in cyclohexane) to give the title compound (7.2 mg, 16%) as a yellow solid. LCMS (Method A): R_{T}=8.55 min, M+H⁺=519.2.

### Step 2: 1-(4-(7-Phenyl-2-(pyridin-2-yl)furo[2,3-b]pyrazin-6-yl)phenyl)cyclobutanamine, 2HCl

4 M HCl in 1,4-dioxane (1.0 ml, 28.8 mmol) was added to a stirred solution of tert-butyl 1-(4-(7-phenyl-2-(pyridin-2-yl)furo[2,3-b]pyrazin-6-yl)phenyl)cyclobutylcarbamate (7.2 mg, 0.014 mmol) in tetrahydrofuran (0.5 ml) at RT under nitrogen. After 16 hours, the precipitate that formed was purified by trituration with diethyl ether (3×2.0 ml). The residual solvents were removed in vacuo and the residue was freeze dried to give the title compound (1.5 mg, 22%). ¹H NMR (500 MHz, methanol-d₄): δ 9.32 (s, 1H), 8.70-8.66 (m, 1H), 8.41-8.38 (m, 1H), 7.96-7.92 (m, 1H), 7.87-7.84 (d, 2H), 7.72-7.69 (d, 2H), 7.59-7.44 (m, 6H), 2.75-2.65 (m, 2H), 2.51-2.41 (m, 2H), 2.23-2.13 (m, 1H), 1.95-1.85 (m, 1H)._LCMS (Method E): R_{T}=1.040 min, M+H⁺=419.1.

### Example 142

### 1-(4-(2-(Benzyloxymethyl)-7-phenylfuro[2,3-b]pyrazin-6-yl)phenyl)cyclobutanamine

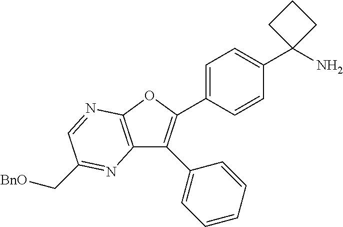

### Step 1: tert-Butyl 1-(4-(2-(benzyloxymethyl)-7-phenylfuro[2,3-b]pyrazin-6-yl)phenyl)cyclobutylcarbamate

Palladium(II)acetate (0.7 mg, 0.003 mmol) was added to a pre-degassed solution of tert-butyl 1-(4-(2-chloro-7-phenylfuro[2,3-b]pyrazin-6-yl)phenyl)cyclobutylcarbamate (75.0 mg, 0.158 mmol), potassium benzyloxymethyltrifluoroborate (43.1 mg, 0.189 mmol), di(1-adamantyl)-n-butylphosphine (1.7 mg, 0.005 mmol) and cesium carbonate (154 mg, 0.473 mmol) in 1,4-dioxane (1.8 ml)/water (0.2 ml) under nitrogen in a microwave vial. The vessel was sealed and subjected heating under the following microwave conditions: 120° C. for 20 minutes, 120° C. for 60 minutes, 120° C. for 40 minutes, 125° C. for 30 minutes, and 135° C. for 30 minutes, until almost complete consumption of starting material. The reaction mixture was partitioned between DCM and water, extracted (DCM×3), dried (Phase Separator), the solvents were removed in vacuo, and the remaining residue was purified by flash chromatography (SiO₂, 0-10% EtOAc in cyclohexane) to give the title compound (76.3 mg, 86%) as a pale yellow oil. LCMS (Method E): R_{T}=1.926 min, M+H⁺=562.1.

### Step 2: 1-(4-(2-(Benzyloxymethyl)-7-phenylfuro[2,3-b]pyrazin-6-yl)phenyl)cyclobutanamine, 2HCl

4 M HCl in 1,4-dioxane (2.0 ml, 8.00 mmol) was added to a stirred solution of tert-butyl 1-(4-(2-(benzyloxymethyl)-7-phenylfuro[2,3-b]pyrazin-6-yl)phenyl)cyclobutylcarbamate (25.4 mg, 0.045 mmol) in tetrahydrofuran (1.0 ml) at RT under an atmosphere of nitrogen. After 30 minutes, diethyl ether (2.0 ml) was added and the precipitate that formed was triturated using diethyl ether (3×2.0 ml). The residual solvents were removed in vacuo and the residue was freeze dried to give the title compound (14.5 mg, 60%) [Note: contains 7.5% de-chlorinated compound (i.e. 5,6-dihydro compound) by LCMS]. ¹H NMR (500 MHz, methanol-d₄): δ 8.47 (s, 1H), 7.88-7.84 (m, 2H), 7.62-7.55 (m, 4H), 7.54-7.46 (m, 3H), 7.42-7.38 (m, 2H), 7.37-7.32 (m, 2H), 7.31-7.27 (m, 1H), 4.80 (s, 2H), 4.70 (s, 2H), 2.84-2.76 (m, 2H), 2.64-2.56 (m, 2H), 2.30-2.20 (m, 1H), 2.04-1.94 (m, 1H). LCMS (Method E): R_{T}=1.16 min, M+H⁺=463.2.

### Example 143

### 1-(4-(2-(3,5-Dimethyl-1H-pyrazol-4-yl)-7-phenylfuro[2,3-b]pyrazin-6-yl)phenyl)cyclobutanamine

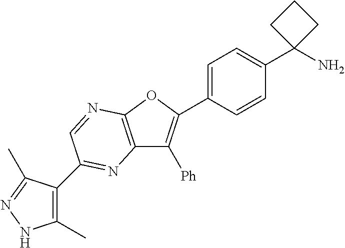

### Step 1: tert-Butyl 1-(4-(2-(3,5-dimethyl-1H-pyrazol-4-yl)-7-phenylfuro[2,3-b]pyrazin-6-yl)phenyl)cyclobutylcarbamate

Tetrakis(triphenylphosphine)palladium(0) (9.1 mg, 0.008 mmol) was added to a pre-degassed (bubbling nitrogen) solution of tert-butyl 1-(4-(2-chloro-7-phenylfuro[2,3-b]pyrazin-6-yl)phenyl)cyclobutylcarbamate (75 mg, 0.158 mmol), 3,5-dimethylpyrazole-4-boronic acid pinacol ester (45.5 mg, 0.205 mmol) and potassium phosphate, tribasic (100 mg, 0.473 mmol) in DMF (1.0 ml)/water (0.25 ml) in a 10 ml microwave vial. The vessel was sealed and irradiated at 120° C. for 20 minutes (CEM Explorer/Discover). Analysis by LCMS (2.3 min run) showed residual starting material and the reaction mixture was twice further heated under same conditions, until analysis by LCMS showed no residual starting material. The reaction mixture was partitioned between DCM and water, separated, dried (Phase Separator), solvents were removed in vacuo, and the remaining residue purified by flash chromatography (SiO₂, 0-50%, cyclohexane/EtOAc) to give the title compound (42.5 mg, 50%) as a yellow oil. LCMS (Method E): R_{T}=1.62 min, M+H⁺=536.2.

### Step 2: 1-(4-(2-(3,5-Dimethyl-1H-pyrazol-4-yl)-7-phenylfuro[2,3-b]pyrazin-6-yl)phenyl)cyclobutanamine, 2HCl

4 M HCl in 1,4-dioxane (4.0 ml, 16.0 mmol) was added to a stirred solution of tert-butyl 1-(4-(2-(3,5-dimethyl-1H-pyrazol-4-yl)-7-phenylfuro[2,3-b]pyrazin-6-yl)phenyl)cyclobutylcarbamate (42.5 mg, 0.079 mmol) in tetrahydrofuran (1.0 ml) at RT under an atmosphere of nitrogen. After 16 hours, the solvents were removed in vacuo and the remaining residue was freeze-dried to give the title compound (25.9 mg, 64%) as a fluffy pale yellow solid. ¹H NMR (500 MHz, methanol-d₄): δ 8.46 (s, 1H), 7.93-7.89 (m, 2H), 7.69-7.65 (m, 2H), 7.61-7.57 (m, 2H), 7.53-7.45 (m, 3H), 2.86-2.78 (m, 2H), 2.66-2.58 (m, 2H), 2.48 (s, 6H), 2.31-2.21 (m, 1H), 2.06-1.96 (m, 1H). LCMS (Method E): R_{T}=0.899 min, M+H⁺=436.0.

### Example 144

### 1-(4-(7-Phenyl-2-(1H-pyrazol-5-yl)furo[2,3-b]pyrazin-6-yl)phenyl)cyclobutanamine

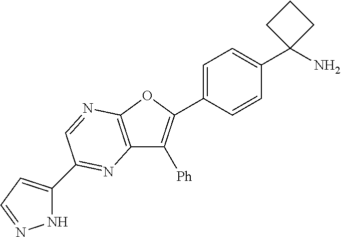

### Step 1: tert-Butyl 1-(4-(7-phenyl-2-(14(2-(trimethylsilyl)ethoxy)methyl)-1H-pyrazol-5-yl)furo[2,3-b]pyrazin-6-yl)phenyl)cyclobutylcarbamate

Tetrakis(triphenylphosphine)palladium(0) (5.0 mg, 0.004 mmol) was added to a pre-degassed mixture of tert-butyl 1-(4-(2-chloro-7-phenylfuro[2,3-b]pyrazin-6-yl)phenyl)cyclobutylcarbamate (50.0 mg, 0.087 mmol), 1-SEM-1H-pyrazole-5-boronic acid pinacol ester (42.4 mg, 0.131 mmol) and potassium phosphate, tribasic (55.5 mg, 0.262 mmol) in DMF (1.0 ml)/water (0.25 ml) in a microwave vial. The reaction vessel was sealed and was subjected to microwave irradiation (CEM Explorer/Discover) at 120° C. for 20 minutes. LCMS showed complete conversion to the product. The reaction mixture was partitioned between EtOAc and 1:1 brine/water, extracted, dried (Phase Separator), and the solvents were removed in vacuo. The remaining residue was purified by flash chromatography (SiO₂, 0-10% EtOAc in cyclohexane) to give the title compound (crude: ca. 58 mg) that was taken through to the next step without further purification. LCMS (Method E): R_{T}=9.32 min, M+H⁺=638.0.

### Step 2: 1-(4-(7-Phenyl-2-(1H-pyrazol-5-yl)furo[2,3-b]pyrazin-6-yl)phenyl)cyclobutanamine, 2HCl

tert-Butyl 1-(4-(7-phenyl-2-(1-((2-(trimethylsilyl)ethoxy)methyl)-1H-pyrazol-5-yl)furo[2,3-b]pyrazin-6-yl)phenyl)cyclobutylcarbamate (crude, assumed 0.087 mmol) was heated to 100° C. with stirring in 4 M HCl in 1,4-dioxane (1.0 ml, 4.00 mmol)/water (1.0 ml) under nitrogen. After 1 hour, analysis by LCMS showed complete conversion to product. The solvents were removed in vacuo and the remaining residue was partitioned between DCM and saturated NaHCO3 (aq) solution, separated, extracted (DCM×3), dried (Phase Separator), and the solvents were removed in vacuo. The residue was dissolved in THF (2.0 ml) and treated with 4M HCl in dioxane (1.0 ml) to form the HCl salt that crashed out of solution. The solvents were removed in vacuo and the residue was recrystallised in methanol and triturated using diethyl ether. The residual solvents were removed in vacuo and the resultant material was freeze-dried. ¹H NMR analysis of the material obtained (22.2 mg) showed the presence of a minor impurity. The material was purified by preparative HPLC (Method F) to give the free base that was treated with 2 M HCl in diethyl ether to generate the HCl salt. The solvents were removed in vacuo and the remaining residue was freeze-dried to give the title compound (5.4 mg, 13%) as a pale yellow solid. ¹H NMR (500 MHz, methanol-d₄): δ 8.93 (s, 1H), 7.90-7.86 (m, 2H), 7.75 (d, 1H), 7.69-7.65 (m, 2H), 7.60-7.48 (m, 5H), 6.98 (s, 1H), 2.87-2.77 (m, 2H), 2.67-2.57 (m, 2H), 2.31-2.21 (m, 1H), 2.05-1.95 (m, 1H). LCMS (Method A): R_{T}=4.12 min, M+2⁺=409.2.

### Example 145

### (6-(4-(1-Aminocyclobutyl)phenyl)-7-phenylfuro[2,3-b]pyrazin-2-yl)methanol

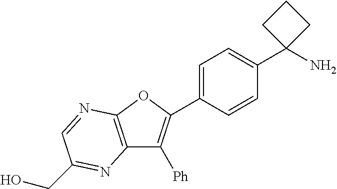

### Step 1: tert-Butyl 1-(4-(2-(hydroxymethyl)-7-phenylfuro[2,3-b]pyrazin-6-yl)phenyl)cyclobutylcarbamate

Palladium on carbon, 10% wt. Degussa type E101 NE/W (4.2 mg, 0.039 mmol) was added to a pre-degassed solution of tert-butyl 1-(4-(2-(benzyloxymethyl)-7-phenylfuro[2,3-b]pyrazin-6-yl)phenyl)cyclobutylcarbamate (42.2 mg, 0.075 mmol) in methanol (1.0 ml) at RT. The reaction mixture was degassed and backfilled with H2, three times. After 16 hours, analysis by LCMS showed ca. 20% conversion to the product. The temperature was increased to 40° C., however, after a further 24 hours, analysis by LCMS showed almost no change. Palladium(II) hydroxide on carbon, 20% wt. Degussa type (4.2 mg, 0.030 mmol) was added and the reaction left over the weekend. LCMS showed ca. 60% conversion to the product. Further palladium (II) hydroxide on carbon, 20% wt. Degussa type (4.2 mg, 0.030 mmol) was added and acetic acid (0.1 ml). After a further 16 hours, LCMS indicated that the reaction was complete. The reaction mixture was filtered over Celite, dried (Phase Separator), the solvents were removed in vacuo, and the remaining residue purified by flash chromatography (SiO₂, 0-30%, EtOAc in cyclohexane) to give the title compound (24.8 mg, 70%) as a white gum. LCMS (Method E): R_{T}=1.61 min, M+H⁺=472.2.

### Step 2: (6-(4-(1-Aminocyclobutyl)phenyl)-7-phenylfuro[2,3-b]pyrazin-2-yl)methanol, HCl

4 M HCl in 1,4-dioxane (1.5 ml, 6.00 mmol) was added to a stirred solution of tert-butyl 1-(4-(2-(hydroxymethyl)-7-phenylfuro[2,3-t]pyrazin-6-yl)phenyl)cyclobutylcarbamate (24.8 mg, 0.053 mmol) in tetrahydrofuran (0.5 ml) at RT under an atmosphere of nitrogen. After 16 hours, analysis by LCMS showed complete reaction. The precipitate that formed was purified by trituration (diethyl ether×3), the solvents were removed in vacuo, and the residue was freeze dried to give the title compound (14.1 mg, 66%) as an off-white solid. ¹H NMR (500 MHz, methanol-d₄): δ 8.47 (s, 1H), 7.88-7.84 (m, 2H), 7.62-7.46 (m, 7H), 4.84 (s, 2H), 2.83-2.74 (m, 2H), 2.63-2.53 (m, 2H), 2.29-2.19 (m, 1H), 2.03-1.93 (m, 1H)._LCMS (Method E): R_{T}=0.768 min, M+H⁺=372.0.

### Example 146

### 1-(4-(2-Ethynyl-7-phenylfuro[2,3-b]pyrazin-6-yl)phenyl)cyclobutanamine

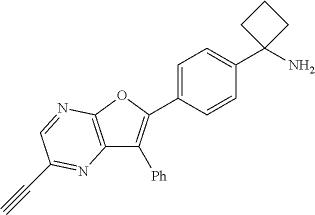

### Step 1: tert-Butyl 1-(4-(7-phenyl-2-((trimethylsilyl)ethynyl)furo[2,3-b]pyrazin-6-yl)phenyl)cyclobutylcarbamate

Copper(I) iodide (5.0 mg, 0.026 mmol) and bis(triphenylphosphine)palladium(II) chloride (36.9 mg, 0.053 mmol) were added to a pre-degassed solution of tert-butyl 1-(4-(2-chloro-7-phenylfuro[2,3-b]pyrazin-6-yl)phenyl)cyclobutylcarbamate (500 mg, 1.05 mmol), TMS-acetylene (0.30 ml, 2.10 mmol) and tetrabutylammonium iodide (776 mg, 2.10 mmol) in triethylamine (1.0 ml)/DMF (5.0 ml) in a microwave vial. The reaction vessel was sealed and heated under microwave conditions (CEM Explorer/Discover) at 100° C. (80 W ceiling) for 15 minutes. Analysis by LCMS showed complete conversion to product. The reaction mixture was concentrated in vacuo and partitioned between EtOAc and water, separated, extracted (3×EtOAc), dried (Phase Separator), and the solvents were removed in vacuo and remaining residue purified by flash chromatography (SiO₂, 0-5-10% EtOAc in cyclohexane) to give the title compound (536 mg, 95%). LCMS (Method E): R_{T}=2.025 min, M+H⁺=538.2.

### Step 2: tert-Butyl 1-(4-(2-ethynyl-7-phenylfuro[2,3-b]pyrazin-6-yl)phenyl)cyclobutylcarbamate

tert-Butyl 1-(4-(7-phenyl-2-((trimethylsilyl)ethynyl)furo[2,3-b]pyrazin-6-yl)phenyl)cyclobutylcarbamate (532.2 mg, 0.990 mmol) was dissolved in MeOH (5.0 ml) and potassium carbonate (205 mg, 1.49 mmol) added. After 30 minutes, analysis by LCMS showed complete conversion to product. The material was filtered using a phase separator, washing with methanol. The solvents were removed in vacuo and the remaining residue was purified by flash chromatography (SiO₂, 0→20%, EtOAc in cyclohexane) to give the title compound (414 mg, 90%) as a pale yellow solid. LCMS (Method E): R_{T}=1.769 min, M+H⁺=466.2.

### Step 3: 1-(4-(2-Ethynyl-7-phenylfuro[2,3-b]pyrazin-6-yl)phenyl)cyclobutanamine, HCl

TFA (1.0 ml, 13.0 mmol) was added to a stirred solution of tert-butyl 1-(4-(2-ethynyl-7-phenylfuro[2,3-b]pyrazin-6-yl)phenyl)cyclobutylcarbamate (50.0 mg, 0.107 mmol) in DCM (1.0 ml) at RT under an atmosphere of nitrogen. After 2 minutes, analysis by LCMS showed almost no residual starting material. The solvents were removed in vacuo and the remaining residue was partitioned between DCM and saturated NaHCO₃ (aq) solution, stirred for 30 minutes, separated, extracted (DCM×3), and the solvents were removed in vacuo to give the product as the free base. The residue was treated with 2M HCl in Et₂O to form the HCl salt and freeze-dried to give the title compound (38.0 mg, 88%) as a pale brown solid. LCMS (Method E): R_{T}=0.968 min, M+H⁺=366.0. ¹H NMR (500 MHz, methanol-d₄): δ 8.50 (s, 1H), 7.87 (d, 2H), 7.62-7.56 (m, 4H), 7.54-7.47 (m, 3H), 3.89 (s, 1H), 2.84-2.75 (m, 2H), 2.65-2.55 (m, 2H), 2.30-2.20 (m, 1H), 2.04-1.94 (m, 1H).

### Example 147

### 1-(4-(7-Phenyl-2-(1H-1,2,3-triazol-5-yl)furo[2,3-b]pyrazin-6-yl)phenyl)cyclobutanamine

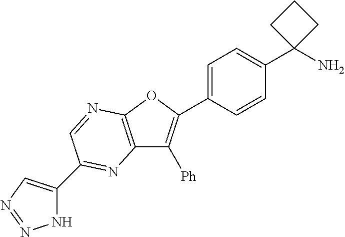

### Step 1: tert-Butyl 1-(4-(7-phenyl-2-(1H-1,2,3-triazol-5-yl)furo[2,3-b]pyrazin-6-yl)phenyl)cyclobutylcarbamate

Copper(I) iodide (1.0 mg, 0.005 mmol) was added to a pre-degassed solution of tert-butyl 1-(4-(2-ethynyl-7-phenylfuro[2,3-b]pyrazin-6-yl)phenyl)cyclobutylcarbamate (50.0 mg, 0.107 mmol) and TMS-azide (0.021 ml, 0.161 mmol) in DMF (0.9 ml)/MeOH (0.1 ml) in a microwave vial. The vessel was sealed and irradiated at 100° C. for 20 minutes (CEM Explorer/Discover). Analysis by LCMS showed complete conversion to product. The reaction mixture was concentrated (to remove MeOH) and partitioned between EtOAc and 1:1 water/brine, separated, extracted (EtOAc×2), dried (Phase Separator), and the solvents were removed in vacuo. The remaining residue purified by flash chromatography (SiO₂, 0→20% EtOAc in cyclohexane) to give the title compound (43.8 mg, 80%). LCMS (Method E): R_{T}=1.572 min, M+H⁺=509.2.

### Step 2: 1-(4-(7-Phenyl-2-(1H-1,2,3-triazol-5-yl)furo[2,3-b]pyrazin-6-yl)phenyl)cyclobutanamine, 2HCl

4 M HCl in 1,4-dioxane (3.0 ml, 12.0 mmol) was added to a stirred solution of tert-butyl 1-(4-(7-phenyl-2-(1H-1,2,3-triazol-5-yl)furo[2,3-b]pyrazin-6-yl)phenyl)cyclobutylcarbamate (43.8 mg, 0.086 mmol) in THF (1.0 ml) at RT under an atmosphere of nitrogen. After 16 hours, analysis by LCMS indicated complete reaction. The resultant precipitation was purified by trituration with diethyl ether (×3), the residual solvents were removed in vacuo and the residue was freeze-dried to give the title compound (30.5 mg, 74%) as a pale yellow solid. ¹H NMR (500 MHz, methanol-d₄): δ 9.02 (s, 1H), 8.36 (s, 1H), 7.90-7.86 (m, 2H), 7.68-7.64 (s, 2H), 7.60-7.48 (m, 5H), 2.84-2.76 (m, 2H), 2.65-2.56 (m, 2H), 2.30-2.20 (m, 1H), 2.04-1.94 (m, 1H). LCMS (Method E): R_{T}=0.807 min, M+H⁺=409.2.

### Example 148

### 2-(6-(4-(1-Aminocyclobutyl)phenyl)-7-phenylfuro[2,3-b]pyrazin-2-ylamino)ethanol

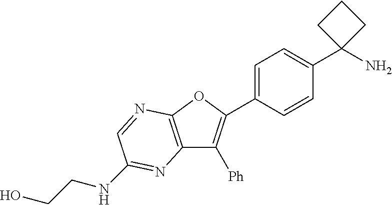

### Step 1: tert-Butyl 1-(4-(2-(2-hydroxyethylamino)-7-phenylfuro[2,3-b]pyrazin-6-yl)phenyl)cyclobutylcarbamate

Palladium(II)acetate (1.2 mg, 0.005 mmol) and (R)—(S)-Josiphos (3.3 mg, 0.005 mmol) were added to a pre-degassed solution of tert-butyl 1-(4-(2-chloro-7-phenylfuro[2,3-b]pyrazin-6-yl)phenyl)cyclobutylcarbamate (50 mg, 0.105 mmol), ethanolamine (7.6 μl, 0.126 mmol), TBAI (38.8 mg, 0.105 mmol) and cesium carbonate (103 mg, 0.315 mmol) in toluene (1.0 ml) in a 10 ml microwave vial. The reaction vessel was sealed and heated under conventional methods at 130° C. After 16 hours, analysis by LCMS showed the correct product as the major peak, along with de-chlorinated starting material and some Boc-removed product as by-products. The reaction mixture was partitioned between DCM and water, separated, extracted (3×DCM), dried (Phase Separator), and the solvents were removed in vacuo. The remaining residue was purified by flash chromatography (SiO₂, 0-10-20-100%, EtOAc in cyclohexane) to give the title compound (8.2 mg, 16%). LCMS (Method E): R_{T}=1.526 min, M+H⁺=501.2.

### Step 2: 2-(6-(4-(1-Aminocyclobutyl)phenyl)-7-phenylfuro[2,3-b]pyrazin-2-ylamino)ethanol, HCl

4 M HCl in 1,4-dioxane (4.0 ml, 16.0 mmol) was added to a stirred solution of tert-butyl 1-(4-(2-(2-hydroxyethylamino)-7-phenylfuro[2,3-b]pyrazin-6-yl)phenyl)cyclobutylcarbamate (6.8 mg, 0.014 mmol) in THF (1.0 ml) at RT. After 16 hours, diethyl ether (2.0 ml) was added and the precipitate that formed was triturated (×3) using diethyl ether. The residual solvents were removed in vacuo and the residue was freeze-dried to give the title compound (4.0 mg, 67%) as yellow/orange solid. ¹H NMR (500 MHz, methanol-d₄): δ 7.79-7.76 (m, 2H), 7.66 (s, 1H), 7.58-7.55 (m, 2H), 7.53-7.50 (m, 2H), 7.48-7.42 (m, 3H), 3.72 (t, 2H), 3.50 (t, 2H), 2.83-2.75 (m, 2H), 2.64-2.54 (m, 2H), 2.29-2.19 (m, 1H), 2.03-1.93 (m, 1H). LCMS (Method E): R_{T}=0.792 min, M+H⁺=401.2.

### Example 149

### 1-(4-(2-Methyl-7-phenylfuro[2,3-b]pyrazin-6-yl)phenyl)cyclobutanamine

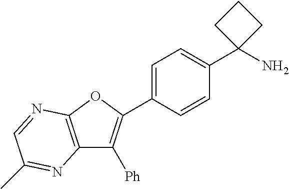

### Step 1: tert-Butyl (1-(4-(7-phenylfuro[2,3-b]pyrazin-6-yl)phenyl)cyclobutyl)carbamate and tert-butyl (1-(4-(2-methyl-7-phenylfuro[2,3-b]pyrazin-6-yl)phenyl)cyclobutyl)carbamate

Tetrakis(triphenylphosphine)palladium(0) (9.1 mg, 0.008 mmol) was added to a pre-degassed (bubbling nitrogen) solution of tert-butyl 1-(4-(2-chloro-7-phenylfuro[2,3-b]pyrazin-6-yl)phenyl)cyclobutylcarbamate (75 mg, 0.158 mmol), methylboronic acid (14.2 mg, 0.236 mmol) and potassium phosphate, tribasic (100 mg, 0.473 mmol) in DMF (1.0 ml)/water (0.25 ml) in a 10 ml microwave vial. The vessel was sealed and irradiated at 120° C. for 20 minutes (CEM Explorer/Discover). Analysis by LCMS showed a mixture of the de-chlorinated starting material and desired product. Separation of this mixture on silica looked very difficult by TLC. The reaction mixture was partitioned between EtOAc and 1:1, water/brine, extracted (EtOAc×3), dried (Phase Separator), and the solvents were removed in vacuo to give a ca. 3:2 mixture of tert-butyl (1-(4-(7-phenylfuro[2,3-b]pyrazin-6-yl)phenyl)cyclobutyl)carbamate (LCMS (Method E): R_{T}=1.72 min, M+H⁺=422.2) and tert-butyl (1-(4-(2-methyl-7-phenylfuro[2,3-b]pyrazin-6-yl)phenyl)cyclobutyl)carbamate (LCMS (Method E): R_{T}=1.80 min, M+H⁺=456.2) that was carried through to the next step without further purification.

### Step 2: 1-(4-(2-Methyl-7-phenylfuro[2,3-b]pyrazin-6-yl)phenyl)cyclobutanamine, HCl

TFA (2.0 ml) was added to a stirred solution of the ca. 3:2 mixture of tert-butyl (1-(4-(7-phenylfuro[2,3-b]pyrazin-6-yl)phenyl)cyclobutyl)carbamate/tert-butyl (1-(4-(2-methyl-7-phenylfuro[2,3-b]pyrazin-6-yl)phenyl)cyclobutyl)carbamate in DCM (2.0 ml) at RT under nitrogen. After 30 minutes, analysis by LCMS showed complete conversion to the mixture of amines. The mixture was separated using preparative HPLC (Method I) and the solvents were removed in vacuo to give the free base of the product. The residue was stirred in 2 M HCl in diethyl ether for 15 minutes, the solvents were removed in vacuo and the remaining material was freeze-dried to give the title compound (9.5 mg, 15% yield over 2 steps) as a yellow solid. ¹H NMR (500 MHz, methanol-d₄): δ 8.25 (s, 1H), 7.84 (d, 2H), 7.60-7.47 (m, 7H), 2.84-2.76 (m, 2H), 2.66-2.56 (m, 2H) o/l with 2.64 (s, 3H), 2.30-2.20 (m, 1H), 2.04-1.94 (m, 1H). LCMS (Method E): R_{T}=1.04 min, M+H⁺=356.2.

### Example 150

### 1-(4-(7-Phenyl-2-(pyridin-3-yl)furo[2,3-b]pyrazin-6-yl)phenyl)cyclobutanamine, 2HCl

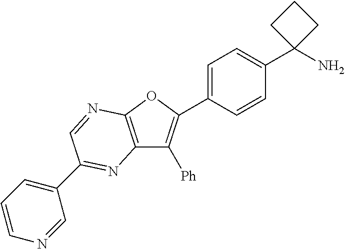

### Step 1: tert-Butyl 1-(4-(7-phenyl-2-(pyridin-3-yl)furo[2,3-b]pyrazin-6-yl)phenyl)cyclobutylcarbamate

To a stirred solution of tert-butyl 1-(4-(2-chloro-7-phenylfuro[2,3-b]pyrazin-6-yl)phenyl)cyclobutylcarbamate (66 mg, 0.139 mmol) and 3-pyridin-3-ylboronic acid (25.6 mg, 0.208 mmol) in 1,4-dioxane (1 ml) in a microwave vial was added a solution of cesium carbonate (136 mg, 0.416 mmol) in water (0.166 ml). The reaction mixture was then degassed with N₂ for 15 min. 1,1′-Bis(diphenylphosphino)ferrocenedichloro palladium(II)dichloromethane complex (5.66 mg, 0.007 mmol) was added and the reaction mixture degassed with N₂ for 3 min before heating in a microwave reactor at 120° C. for 20 min (CEM Explorer/Discover). The mixture was filtered (cotton wool) and concentrated in vacuo. The residue was partitioned between brine (2 ml) and DCM (2 ml). The layers were separated and the aqueous phase extracted with DCM (3×1 ml). The combined organic phases were concentrated in vacuo. The resulting residue was purified by silica gel chromatography (gradient 0 to 20% acetone in cyclohexane) to afford the title compound (46.8 mg, 63%). LCMS (Method A) R_{T}=7.44 min, M+H⁺=519.2. ¹H NMR (500 MHz, CDCl₃): δ 9.22 (br s, 1H), 8.65 (s, 1H), 8.61 (br s, 1H), 8.30 (d, 1H), 7.72 (d, 2H), 7.65 (d, 2H), 7.35-7.43 (m, 6H), 5.13 (s, 1H), 2.42-2.55 (m, 4H), 2.02-2.10 (m, 1H), 1.78-1.87 (m, 1H), 1.26 (br s, 9H).

### Step 2: 1-(4-(7-Phenyl-2-(pyridin-3-yl)furo[2,3-b]pyrazin-6-yl)phenyl)cyclobutanamine, 2HCl

To a stirring solution of tert-butyl 1-(4-(7-phenyl-2-(pyridin-3-yl)furo[2,3-b]pyrazin-6-yl)phenyl)cyclobutylcarbamate (45.8 mg, 0.088 mmol) in anhydrous THF (1 ml) was added 4M HCl in 1,4-dioxane (4 ml, 16.0 mmol) over 5 min and the resulting solution stirred for 16 h. Diethyl ether (5 ml) was added and the resulting suspension stirred for 40 min. The solid was isolated by centrifugation after pouring off the supernatant liquid. The centrifugation was repeated twice further with diethyl ether (2×5 ml). The solid was dissolved in water and freeze-dried to afford the title compound (33.2 mg, 77%). LCMS (Method A) R_{T}=3.85 min, M+2H⁺=420.2. ¹H NMR (500 MHz, methanol-d₄) δ 9.40 (br s, 1H), 9.01 (d, 1H), 8.98 (s, 1H), 8.78 (br s, 1H), 7.95 (br s, 1H), 7.82 (d, 2H), 7.59 (d, 2H), 7.46 (d, 2H), 7.40-7.43 (m, 3H), 2.69-2.74 (m, 2H), 2.51-2.57 (m, 2H), 2.15-2.20 (m, 1H), 1.88-1.93 (m, 1H).

### Example 151

### N-(3-(6-(4-(1-Aminocyclobutyl)phenyl)-7-phenylfuro[2,3-b]pyrazin-2-yl)phenyl)acetamide, HCl

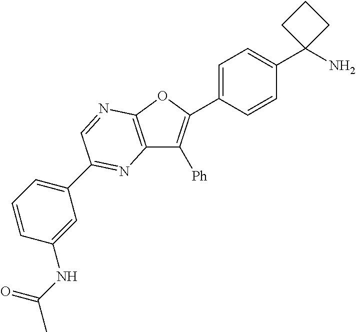

### Step 1: tert-butyl 1-(4-(2-chloro-7-phenylfuro[2,3-b]pyrazin-6-yl)phenyl)cyclobutylcarbamate

To a stirring solution of tert-butyl 1-(4-(2-chloro-7-iodofuro[2,3-b]pyrazin-6-yl)phenyl)cyclobutylcarbamate (2740 mg, 5.210 mmol) in anhydrous DMF (112 ml) was added phenylboronic acid (699 mg, 5.730 mmol) and potassium phosphate (3320 mg, 15.63 mmol). The reaction mixture was then de-gassed with N₂ for 30 mins. Tetrakis(triphenylphosphine)palladium(0) (301 mg, 0.261 mmol) was then added and the reaction heated to 80° C. for 42 h. The reaction mixture was cooled to RT and filtered through Celite®. The DMF was removed in vacuo. Water (200 ml) was added to the residue and the aqueous suspension extracted with ethyl acetate (3×100 ml). The combined organic extracts were washed with brine, dried (Na₂SO₄), filtered and concentrated in vacuo. The resulting residue was purified by silica gel chromatography (gradient 0 to 100% EtOAc in cyclohexane) to afford the title compound (1780 mg, 72%).

LCMS (Method D) R_{T}=6.34 min, M+H⁺=476.1.

### Step 2: tert-butyl 1-(4-(2-(3-acetamidophenyl)-7-phenylfuro[2,3-b]pyrazin-6-yl)phenyl)cyclobutylcarbamate

A solution of cesium carbonate (164 mg, 0.504 mmol) in water (0.2 ml) was added to a solution of tert-butyl 1-(4-(2-chloro-7-phenylfuro[2,3-b]pyrazin-6-yl)phenyl)cyclobutylcarbamate (80.0 mg, 0.168 mmol), 3-acetamidophenylboronic acid (45.1 mg, 0.252 mmol) in 1,4-dioxane (1.2 ml) in a 10 ml microwave vial. The suspension was then de-gassed with N₂ for 15 mins. 1,1′-Bis(diphenylphosphino)ferrocenedichloro palladium(II)dichloromethane complex (6.86 mg, 0.008 mmol) was added and the mixture de-gassed with N₂ for 3 mins. The vial was sealed and irradiated at 120° C. for 20 minutes (CEM Explorer/Discover). The mixture was filtered (cotton wool) washing through with DCM (3 ml). The solvent was removed in vacuo. The residue was partitioned between DCM (2 ml) and water (2 ml). The separated aqueous phase was further extracted with DCM (3×1 ml). The combined organic phases were concentrated in vacuo and the residue purified by silica gel chromatography (gradient 0 to 20% acetone in cyclohexane) to afford the title compound (89.9 mg, 87%).

LCMS (Method D) R_{T}=3.24 min, M+H⁺=575.2.

### Step 3: N-(3-(6-(4-(1-Aminocyclobutyl)phenyl)-7-phenylfuro[2,3-b]pyrazin-2-yl)phenyl)acetamide, HCl

To a stirred solution of tert-butyl 1-(4-(2-(3-acetamidophenyl)-7-phenylfuro[2,3-b]pyrazin-6-yl)phenyl)cyclobutylcarbamate (89.9 mg, 0.156 mmol) in anhydrous THF (1 ml) was added 4M HCl in 1,4-dioxane (4 ml, 16.0 mmol) over 5 min and the resulting solution stirred for 16 h. Diethyl ether (5 ml) was added and the resulting suspension stirred for 1 h. The solid was isolated by filtration washing with diethyl ether (5 ml). The solid was dried under vacuum to afford the title compound (49.5 mg, 62%). LCMS (Method D) R_{T}=2.93 min, M+H⁺=475.2. ¹11 NMR (500 MHz, DMSO-d₆) δ 10.1 (br s, 1H), 8.84 (s, 2H), 8.78 (br s, 2H), 8.13 (s, 1H), 7.68-7.75 (m, 4H), 7.59-7.61 (m, 4H), 7.45-7.53 (m, 3H), 7.38 (t, 1H), 2.52-2.55 (m, 4H), 2.11-2.16 (m, 1H), 2.00 (s, 3H), 1.72-1.78 (m, 1H).

### Example 152

### 3-(6-(4-(1-Aminocyclobutyl)phenyl)-7-phenylfuro[2,3-b]pyrazin-2-yl)benzamide, HCl

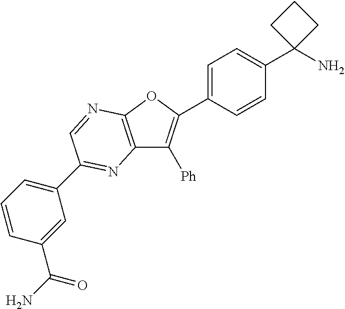

### Step 1: tert-Butyl 1-(4-(2-(3-carbamoylphenyl)-7-phenylfuro[2,3-b]pyrazin-6-yl)phenyl)cyclobutylcarbamate

A solution of cesium carbonate (164 mg, 0.504 mmol) in water (0.2 ml) was added to a solution of tert-butyl 1-(4-(2-chloro-7-phenylfuro[2,3-b]pyrazin-6-yl)phenyl)cyclobutylcarbamate (80.0 mg, 0.168 mmol) and 3-carbamoylphenylboronic acid (41.6 mg, 0.252 mmol) in 1,4-dioxane (1.2 ml) in a 10 ml microwave vial. The suspension was degassed with N₂ for 15 min. 1,1′-Bis(diphenylphosphino)ferrocenedichloro palladium(II)dichloromethane complex (6.86 mg, 0.008 mmol) was added and the mixture degassed with N₂ for 3 min. The vial was sealed and irradiated at 120° C. for 20 min (CEM Explorer/Discover). Irradiation was repeated at 120° C. for a further 20 min. 1,1′-Bis(diphenylphosphino)ferrocenedichloro palladium(II)dichloromethane complex (6.9 mg, 0.008 mmol) was added and the mixture irradiated at 120° C. for 10 min. The mixture was filtered (cotton wool) washing through with DCM (3 ml). The solvent was removed in vacuo. The residue was partitioned between DCM (2 ml) and water (2 ml). The separated aqueous phase was further extracted with DCM (3×1 ml). The combined organic phases were concentrated in vacuo and the residue purified by silica gel chromatography (gradient 0 to 25% acetone in cyclohexane) to afford the title compound (79.0 mg, 84%). LCMS (Method D) R_{T}=5.33 min, M+H⁺=561.2.

### Step 2: 3-(6-(4-(1-Aminocyclobutyl)phenyl)-7-phenylfuro[2,3-b]pyrazin-2-yl)benzamide, HCl

To a stirred solution of tert-butyl (1-(4-(2-(3-carbamoylphenyl)-7-phenylfuro[2,3-b]pyrazin-6-yl)phenyl)cyclobutyl)carbamate (79.0 mg, 0.141 mmol) in anhydrous THF (1 ml) was added 4M HCl in 1,4-dioxane (4 ml, 16.0 mmol) over 5 min and the resulting solution stirred for 16 h. Diethyl ether (5 ml) was added and the resulting suspension stirred for 1 h. The solid was isolated by filtration washing with diethyl ether (5 ml). The solid was dried under vacuum to afford the title compound (27.8 mg, 40%). LCMS (Method D) R_{T}=2.64 min, M+H⁺=461.2. ¹H NMR (500 MHz, DMSO-d₆): δ 9.10 (s, 1H), 8.79 (br s, 1H), 8.75 (br s, 2H), 8.59 (s, 1H), 8.26 (d, 1H), 8.15 (br s, 1H), 7.98 (d, 1H), 7.81 (d, 1H), 7.49-7.70 (m, 10H), 2.58-2.63 (m, 4H), 2.13-2.23 (m, 1H), 1.80-1.87 (m, 1H).

### Example 153

### 1-(4-(3-(Methylthio)-7-phenyl-5H-pyrrolo[3,2-b]pyrazin-6-yl)phenyl)cyclobutanamine

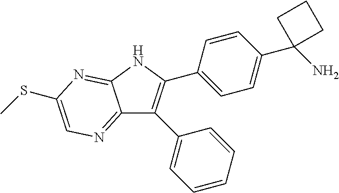

### Step 1: N-(3-Bromo-6-(methylthio)pyrazin-2-yl)-2,2,2-trifluoroacetamide

Trifluoroacetic anhydride (0.45 ml, 3.15 mmol) was added to a stirred solution of 3-bromo-6-(methylthio)pyrazin-2-amine (462 mg, 2.10 mmol) [prepared according to U.S. Pat. No. 6,147,078 A1] and triethylamine (0.878 ml, 6.30 mmol) in tetrahydrofuran (10 ml) at RT under an atmosphere of nitrogen. After 1 hour, analysis by LCMS (2.5 min run) showed conversion to product. The reaction mixture was partitioned between DCM and water, separated, extracted (DCM×3), dried (Na₂SO₄), and the solvents were removed in vacuo. The remaining residue was purified by flash chromatography (SiO₂, 0-5%, EtOAc in cyclohexane) to give the title compound (397 mg, 60%) as a pale yellow solid. LCMS (Method E): R_{T}=1.511 min, M−H⁻=314.0, 316.0.

### Step 2: tert-Butyl 1-(4-(3-(methylthio)-5H-pyrrolo[3,2-b]pyrazin-6-yl)phenyl)cyclobutylcarbamate

Bis(tri-t-butylphosphine)palladium(0) (8.1 mg, 0.016 mmol), copper(I) iodide (1.5 mg, 0.008 mmol) and tert-butyl 1-(4-ethynylphenyl)cyclobutylcarbamate (112 mg, 0.411 mmol) were added successively to a pre-degassed solution of N-(3-bromo-6-(methylthio)pyrazin-2-yl)-2,2,2-trifluoroacetamide (100 mg, 0.316 mmol) in diisopropylamine (1.0 ml, 7.02 mmol)/DMF (1.0 ml) in a 10 ml microwave vial. The vessel was sealed and irradiated under the following microwave conditions: twice at 120° C. for 15 minutes (80 W ceiling), 120° C. for 20 mins (100 W ceiling), twice at 120° C. for 60 mins (100 W ceiling), twice at 135° C. for 60 mins (100 W ceiling), until LCMS showed no residual starting material. The reaction mixture was partitioned between EtOAc and water/brine, separated, extracted (EtOAc×2), dried (Phase Separator), and the solvents were removed in vacuo. The remaining residue was purified by flash chromatography (SiO₂, 0→20%, EtOAc in cyclohexane) to give the title compound (52.7 mg, 41%). LCMS (Method E): R_{T}=1.469 min, M+H⁺=411.2.

### Step 3: tert-Butyl 1-(4-(7-bromo-3-(methylthio)-5H-pyrrolo[3,2-b]pyrazin-6-yl)phenyl)cyclobutylcarbamate

NBS (12.0 mg, 0.067 mmol) was added to a stirred solution of tert-butyl 1-(4-(3-(methylthio)-5H-pyrrolo[3,2-b]pyrazin-6-yl)phenyl)cyclobutylcarbamate (55.2 mg, 0.067 mmol) in acetonitrile (1.0 ml) at RT under an atmosphere of nitrogen. After 16 hours, LCMS showed complete conversion to product. The reaction mixture was concentrated and residue partitioned between DCM and 10% sodium thiosulfate (aq) solution, extracted (DCM×3), dried (Phase Separator), and the solvents were removed in vacuo. The remaining residue was purified by flash chromatography (SiO₂, 0-20%, EtOAc in cyclohexane) to give the title compound (30.6 mg, 93%) as a yellow oil. LCMS (Method E): R_{T}=1.603 min, M+H⁺=489.0, 491.0.

### Step 4: tert-Butyl 1-(4-(7-bromo-3-(methylthio)-5-((2-(trimethylsilyl)ethoxy)methyl)-5H-pyrrolo[3,2-b]pyrazin-6-yl)phenyl)cyclobutylcarbamate

SEM-Cl (0.060 ml, 0.337 mmol) was added to a stirred solution of tert-butyl 1-(4-(7-bromo-3-(methylthio)-5H-pyrrolo[3,2-b]pyrazin-6-yl)phenyl)cyclobutylcarbamate (138 mg, 0.281 mmol) and sodium hydride (33.7 mg, 0.843 mmol) in THF (10 ml) at RT under an atmosphere of nitrogen. After 1 hour, analysis by LCMS showed complete conversion to product. Methanol (2.0 ml) was added slowly to quench the excess hydride. After 1 hour, the solvents were removed in vacuo and the remaining residue was partitioned between DCM and water, separated, extracted (DCM×3), and dried (Phase Separator). The solvents were removed in vacuo and the remaining residue was purified by flash chromatography (SiO₂, 0-10%, EtOAc in cyclohexane) to give the title compound (121 mg, 70%) as a yellow oil. LCMS (Method E): R_{T}=2.045 min, M+H⁺=619.0, 621.0.

### Step 5: tert-Butyl 1-(4-(3-(methylthio)-7-phenyl-5-((2-(trimethylsilyl)ethoxy)methyl)-5H-pyrrolo[3,2-b]pyrazin-6-yl)phenyl)cyclobutylcarbamate

Palladium Tetrakis (11.3 mg, 0.010 mmol) was added to a pre-degassed solution of phenylboronic acid (35.8 mg, 0.293 mmol), potassium carbonate (81.1 mg, 0.587 mmol) and tert-butyl 1-(4-(7-bromo-3-(methylthio)-5-((2-(trimethylsilyl)ethoxy)methyl)-5H-pyrrolo[3,2-b]pyrazin-6-yl)phenyl)cyclobutylcarbamate (121 mg, 0.196 mmol) in DMF (1.5 ml) in a 10 ml microwave vial. The vessel was sealed and irradiated at 100° C. for 20 minutes (CEM Explorer/Discover). LCMS showed no reaction had occurred and therefore, the reaction was allowed to stir at 80° C. under conventional heating. Over two weeks, the reaction was monitored by LCMS and was found to progress very slowly and therefore, the temperature was increased from 80° C. to 100° C. during this period. It was decided to stop the reaction after 2 weeks (when analysis by LCMS showed the product peak was the slightly major peak over starting material). The reaction mixture was partitioned between EtOAc and water/brine, separated, extracted (3×EtOAc), dried (Phase Separator), and solvents were removed in vacuo. The remaining residue purified by flash chromatography (SiO₂, 0-6%, EtOAc in cyclohexane) to give the title compound (27.0 mg, 22%) as a pale yellow oil. LCMS (Method E): R_{T}=2.127 min, M+H⁺=617.2.

### Step 6: 1-(4-(3-(Methylthio)-7-phenyl-5H-pyrrolo[3,2-b]pyrazin-6-yl)phenyl)cyclobutanamine, HCl

4 M HCl in 1,4-dioxane (2.0 ml) was added to a stirred solution of tert-butyl 1-(4-(3-(methylthio)-7-phenyl-5-((2-(trimethylsilyl)ethoxy)methyl)-5H-pyrrolo[3,2-b]pyrazin-6-yl)phenyl)cyclobutylcarbamate (27.0 mg, 0.044 mmol) in water (2.0 ml) and heated to 100° C. under nitrogen. After 45 minutes, analysis by LCMS showed incomplete conversion to product. The reaction mixture was further heated under microwave irradiation at 100° C. for 20 minutes (CEM Explorer/Discover). Analysis by LCMS showed complete conversion to product. The solvents were removed in vacuo and the remaining residue was triturated using diethyl ether (×3). The residual solvents were removed in vacuo and the remaining residue was freeze-dried to give the title compound (13.1 mg, 71%) as an orange/red solid. ¹H NMR (500 MHz, methanol-d₄): δ 8.30 (s, 1H), 7.67 (d, 2H), 7.54 (d, 2H), 7.47-7.36 (m, 5H), 2.83-2.75 (m, 2H), 2.71 (s, 3H), 2.65-2.57 (m, 2H), 2.30-2.20 (m, 1H), 2.04-1.94 (m, 1H). LCMS (Method E): R_{T}=0.961 min, M+H⁺=387.0.

AKT Kinase Assay Testing

Testing of the compounds was performed using an AKT Kinase Assay:

Activated AKT isoforms 1, 2 and 3 were assayed utilising a 5′ FAM Crosstide (Seq. GRPRTSSFAEG-OH). The extent of kinase phosphorylation was determined by fluorescent polarisation using IMAP progressive binding reagent, which introduces binding beads which allow the reagent to specifically bind to phosphate residues via covalent co-ordination complex bonds.

iMAP binding solution stops Crosstide/kinase interaction and specifically binds phosphorylated substrates. The degree of phosphorylation is determined by fluorescent polarisation (excitation 485 nm; emission 528 nm) or the reduction in speed of rotation of the excited substrate.

The following materials were used in the assay:
a) Activated AKT isoforms (SignalChem.) dissolved in Complete Reaction buffer at a pre-determined concentration selected so that the assay was carried out in the linear range.
b) AKT substrate peptide: FAM Crosstide (R7110) Molecular Devices, diluted in complete reaction buffer.
c) iMAP Progressive Screening Express Kit (R8127) Molecular Devices
d) Complete Reaction Buffer containing 0.1% BSA, 10 mM Tris-HCl, 10 mM MgCl₂, 0.05% NaN₃ and 0.01% phosphate free BSA, 1 mM DTT
e) Progressive Binding Solution containing 75% Buffer A, 25% Buffer B and low volume Binding Reagent which contains the binding entity for the assay
f) ATP diluted in complete reaction buffer
g) Black polystyrene 384 well assay plates (Nunc).
h) Biotek Synergy 4 Hybrid Plate reader.

50 μl of test compound was dissolved in DMSO (Sigma Aldrich) and serially diluted in complete reaction buffer to give a fourteen point half log dose response and plated into 384 well black plates. The compound was incubated at room temperature with activated AKT isoform (50) at the pre-determined concentration, for 45 minutes.

2.50 of ATP solution mixed with 2.5 μl of AKT substrate peptide (FAM Crosstide (R7110) Molecular Devices) were dispensed into each well and the plate centrifuged at 1000 rpm for 20 seconds to ensure homogenous mixing of reagents. The reaction mix was incubated in the dark for one hour at room temperature.

The kinase reaction was stopped by the addition of Progressive Binding Solution and the mixture allowed to equilibrate for one hour in the dark, at room temperature.

The fluorescent polarisation generated in each well was determined using a Biomek Synergy 4 Hybrid plate reader. In brief, each reaction solution was excited at 485 nm with the emission measured at 528 nm in both the parallel and perpendicular pathway.

The polarisation value generated in each well was calculated by Gen5 software (Biotek) and the % inhibition of kinase activity compared to vehicle control was calculated via GraphPad Prism. IC₅₀ values for each compound were calculated by non-linear regression analysis using Prism software.

All plates were internally controlled by two methods. Firstly, by calculating the signal:noise ratio; based on kinase polarisation without inhibitor and polarisation generated by complete reaction buffer in the absence of activated kinase. Secondly by determining IC50 values generated by known inhibitors of the AKT isoforms.

Testing of the compounds was also performed using in vitro cell proliferation assays:

Cell Titre Glo (Promega) is a highly sensitive homogeneous reagent used to determine the viability of cells. The reagent uses a stable form of luciferase to measure ATP as an output of viability. The luminescent values generated in the assay are directly proportional to the number of viable cells in your assay.

The following materials were used: White, clear bottomed 96 well assay plates (Costar); Cell titre Glo reagents; LnCaP (ECACC) cells grown in RPMI medium (Invitrogen) supplemented with 10 mM HEPES (Invitrogen), 1 mM Sodium pyruvate (Invitrogen), 2 mM L-Glutamine (Invitrogen) and 10% Foetal calf serum (Invitrogen); PC3 (ECACC) cells grown in RPMI medium supplemented with 10% Foetal calf serum (Invitrogen); Trypsin (Invitrogen); PBS (Invitrogen); Biotek Synergy 4 Hybrid Plate reader; 96 well plate shaker (Stuart SSL5); Eppendorf 5414 desk-top centrifuge; Beckman Coulter cell counter Z1 single threshold system.

Prostate cell lines, PC3 and LnCaP, were washed, detached and re-suspended in their respective fresh media. The cells were pelleted by centrifugation (Eppendorf 5414) and spent supernatant discarded. The cells were re-suspended by vortex mixing, counted and seeded into clear bottom white 96 well plates at a density of 5000 cells per well. The cells were incubated (Sanyo) overnight at 37° C. (95% O₂/5% CO₂), and next day treated with increasing concentrations of test compound formulated in fresh medium. The plates were returned to the incubator for 72 hours.

Cell Titre Glo (Promega) was prepared by mixing the supplied reagents as per manufacturer's instructions and left to stand at room temperature. The cell plates were removed from the incubator and 80 μl of the Cell Titre Glo solution added to each well. The plate was shaken for five minutes to ensure homogenous mixing of reagents and cells, then left to stand for ten minutes at room temperature.

The cell viability post compound treatment was determined by the luminescent intensity emitted from the drug treated wells in the plate. In brief, the assay plate was placed in the Biotex Synergy 4 Hybrid plate reader and the luminescence read in each well. The compound treated wells were compared to vehicle treated wells and the % inhibition of cell viability calculated.

The data was analysed using GraphPad Prism, with IC₅₀ values generated using non-linear regression of the data set.

Analysis of Compound Effects on AKT Signalling Pathways

Phosphorylation status of various members of the AKT/PI3K pathway were investigated via western blotting.

Materials Required for this Assay

20× Running buffer (Invitrogen); Rainbow marker ladder (GE Healthcare); Reducing buffer (Invitrogen); 20× Transfer buffer (Invitrogen); 4-12% Bis-Tris Gels (Invitrogen); Filter paper (Whatman); Nitrocellulose (Amersham); ECL plus detection reagents (GE Healthcare); Radiographic film (Kodak); Biorad Protein determination reagent (Biorad); AKT pathway signalling antibodies (Cell Signalling)

LnCaP and PC3 cell lines were washed, detached and re-suspended in fresh medium. They were seeded in 90 mm² dishes and incubated overnight (95% O₂/5% CO₂) to allow adherence. When the cells had reached 60% confluence, the medium was removed and replaced with compound or vehicle supplemented medium. The plates were incubated for a range of time points.

The medium was removed and the cells placed on ice and washed in PBS. 300 μl of lysis buffer was added to the dish and left for a few minutes, before the cells were scraped into the solution and pipetted into a centrifuge tube. The tube was placed on ice for 10 minutes and then vortexed to aid cellular lysis. The sample was centrifuged (Eppendorf bench-top ultra-fuge) at 13.2 k rpm for 10 minutes at 4° C. The resultant supernatant was assayed for protein content using the Bradford method (Biorad) and equal quantities of protein calculated and heated in sample reducing buffer to 95° C. for ten minutes.

The samples were run on 4-12% Bis-Tris gels (Invitrogen), transferred onto nitrocellulose membrane (Amersham) and blocked with a 5% non-fat milk solution.

The membranes were used to determine difference in total AKT, pSer473 AKT, pGSK3β and total GSK3β (all sourced from Cell Signalling). The respective primary antibodies were diluted in 1% non-fat milk blocking solution and incubated on the membranes overnight at 4° C. The membranes were washed three times in PBS and incubated with the respective secondary antibodies (Sigma Aldrich) for two hours and the proteins were detected using ECL plus detection reagents (GE Healthcare).

1. A compound according to Formula (I): 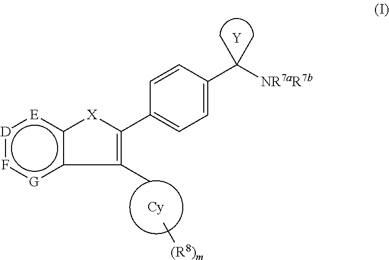
wherein:
0, 1 or 2 of D, E, F and G are independently selected from N, NH and NR¹ and the others are independently selected from CH and CR², wherein each R¹ is independently selected from aryl, C1-C10 alkyl, CONHR³, CONR^{3a}R^{3b}, COR³ and CO₂R³ and each R² is independently selected from aryl, C1-C10 alkyl, CN, CHO, CO₂H, CONH₂, CONHR³, CONR^{3a}R^{3b}, COR³, CO₂R³, NH₂, NHR³, NR^{3a}R^{3b}, NHCOR³, NHSO₂R³, NR^{3a}COR^{3b}, NR^{3a}SO₂R^{3b}, oxo, OH, OR³, SH, SR³, SOR³, SO₂R³, SO₂NHR³, SO₂NR^{3a}R^{3b}, F, Cl, Br and I, wherein each R³, R³⁸ and R^{3b} is independently selected from C1-C10 alkyl, including wherein R^{3a} and R^{3b} are joined to one another to form a heterocycle that includes the nitrogen to which they are attached;
wherein at least D or G is NH or NR¹ if E or F is CO and at least E or F is NH or NR¹ if D or G is CO;
wherein separate R¹ and/or R² groups may be joined to one another to form a heterocycle that includes the C and/or N atoms to which they are attached if the separate R¹ and/or R² groups are contained on D and E and/or F and G, or D and F and the separate R² groups are selected from OR³, SR³, SOR³, SO₂R³, SO₂NHR³, SO₂NR^{3a}R^{3b}, NHR³, NR^{3a}R^{3b}, CO₂R³, CONHR³ and CONR^{3a}R³, and/or wherein separate R¹ and/or R² groups on F and G may be joined to form the structure: 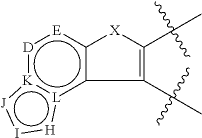
where 0 or 1 of K and L are N and the other(s) is/are C, and wherein H, I and J are independently selected from O, S, SO, SO₂, NH, NR⁴, N, CH and CR⁵, wherein each R⁴ is independently selected from aryl, C1-C10 alkyl, CONHR⁶, CONR^{6a}R^{6b}, COR⁶ and CO₂R⁶ and each R⁸ is independently selected from aryl, C1-C10 alkyl, CN, CHO, CO₂H, CONH₂, CONHR⁶, CONR^{6a}R^{6b}, COR⁶, CO₂R⁶, oxo, NH₂, NHR⁶, NR^{6a}R^{6b}, OH, OR⁶, SH, SR⁶, SOR⁶, SO₂R⁶, SO₂NHR⁶, SO₂NR^{6a}R^{6b}, F, Cl, Br and I, wherein each R⁶, R^{6a} and R^{6b} is independently selected from C1-C10 alkyl, including wherein R^{6a} and R^{6b} are joined to one another to form a heterocycle that includes the nitrogen to which they are attached,
X is selected from O, NR¹⁰, S, SO or SO₂.
R^{7a} and R^{7b} are independently selected from H and alkyl, including wherein R⁷ and R^{7b} are joined to one another to form a heterocycle that includes the nitrogen to which they are attached; and 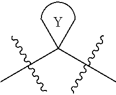
is selected from: 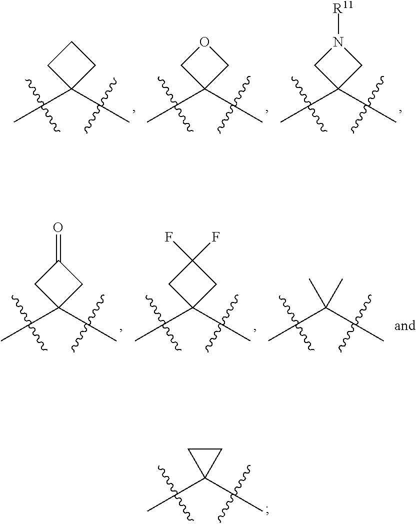
ring Cy is selected from (C₃ to C₈)cycloalkyl and aryl, wherein m is 0, 1, 2, 3, 4 or 5, and each R⁸ is independently selected from alkyl, CN, CHO CO₂H, CONH₂, CONHR⁹, CONHR^{9a}R^{9b}, COR⁹, CO₂R⁹, NH₂, NHR⁹, NR^{9a}R^{9b}, NHCOR⁹, NHSO₂R⁹, NR^{9a}COR^{9b}, NR^{9a}SO₂R^{9b}, OH, OR⁹, SH, SR⁹, F, Cl, Br and I, wherein each R⁹, R^{9a} and R^{9b} is independently selected from alkyl, including wherein R^{9a} and R^{9b} form a heterocycle that includes the nitrogen to which they are attached or Cy may be iodine;
R¹⁰ and R¹¹ are independently selected from hydrogen and C₁-C₁₀ alkyl; C₁-C₁₀ acyl, and C₁-C₁₀ sulfonyl.
and pharmaceutically acceptable salts, stereoisomers and tautomers thereof.

2. The compound of claim 1, wherein 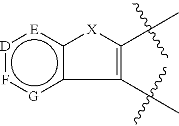
is selected from: 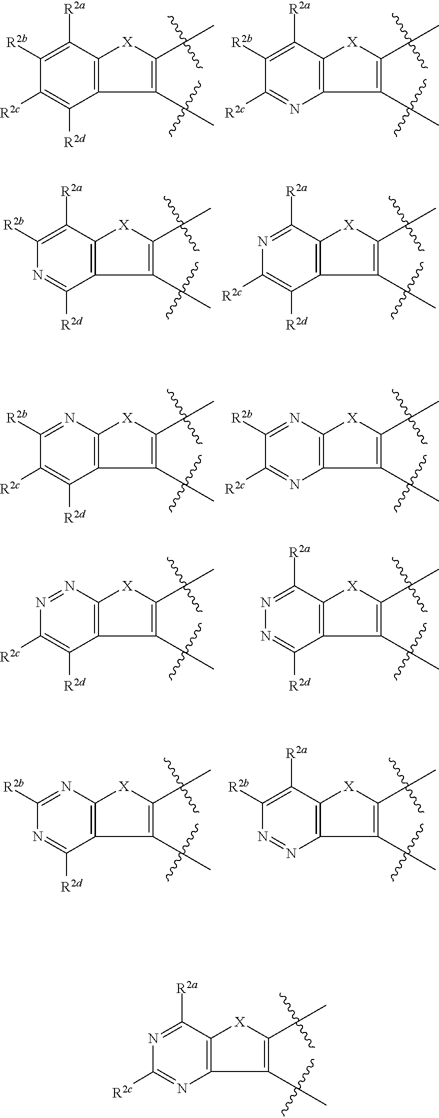
wherein R^{2a}, R^{2b}, R^{2C} and R^{2d} are independently selected from R² and H.

3. The compound of claim 1, wherein 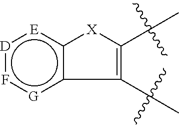
is selected from: 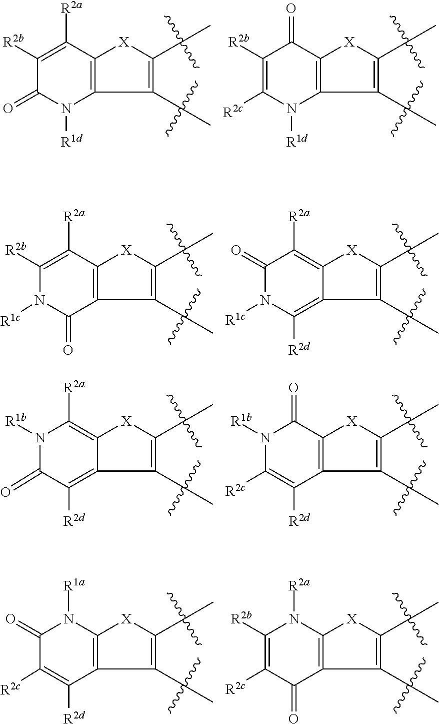
wherein R^{1a}, R^{1b}, R^{1c} and R^{1d} are independently selected from R¹ and H and R^{2a}, R^{2b}, R^{2c} and R^{2d} are independently selected from R² and H.

4. The compound of claim 1, wherein 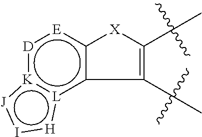
is selected from: 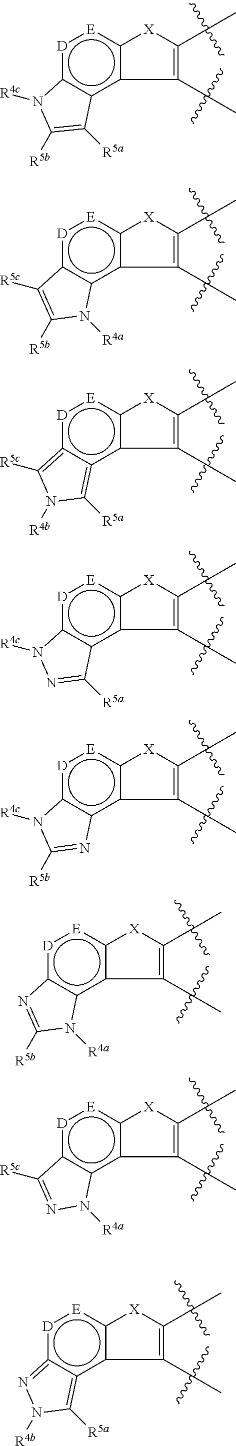 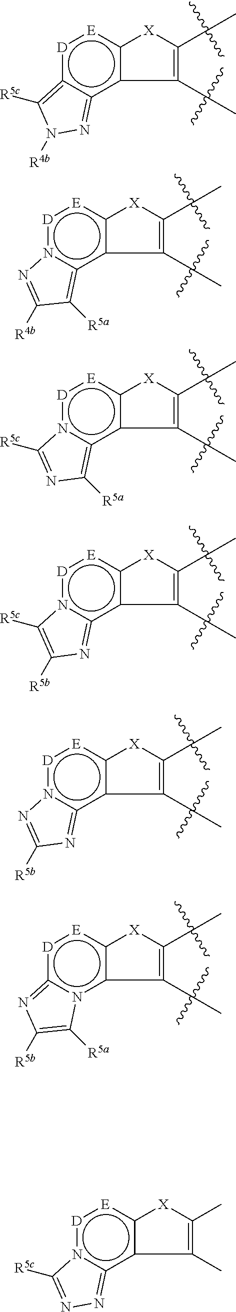 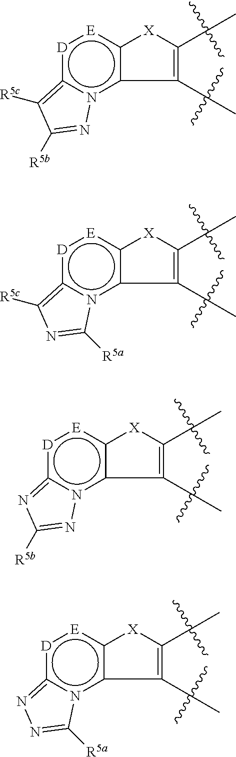
wherein R^{4a}, R^{4b} and R^{4c} are independently selected from R⁴ and H and R^{5a}, R^{5b} and R^{5c} are independently selected from R⁵ and H.

5. The compound of any one of the preceding claims, wherein D and E are independently selected from N, CH and CR².

6. The compound of claim 1 wherein D, E, F and G together with the two carbon atoms on the 5 membered ring together form a moiety having the structure: 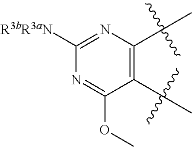
wherein R^{3a} and R^{3b} are as defined herein.

7. The compound of claim 6 wherein R^{3a} and R^{3b} are either both hydrogen or are selected so as to give one of the following moieties:

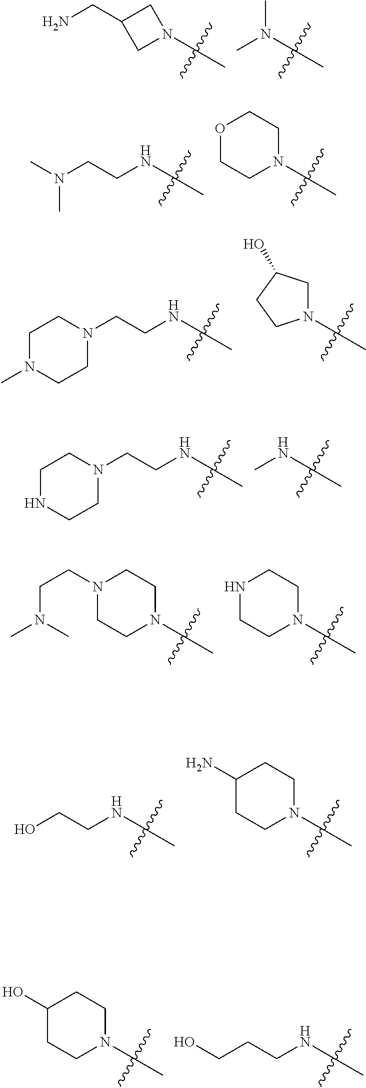

8. The compound of claim 7 wherein R^{3a} and R^{3b} are either both hydrogen or are selected so as to give one of the following moieties:

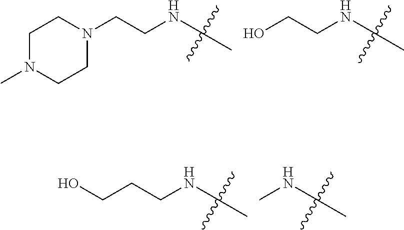

9. The compound of claim 1 wherein D, E, F and G together with the two carbon atoms on the 5 membered ring together form a moiety having either of the following structures:

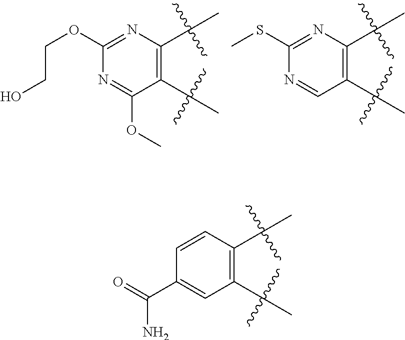

10. The compound of claim 1 wherein D, E, F and G together with the two carbon atoms on the 5 membered ring together form a moiety having either of the following structures:

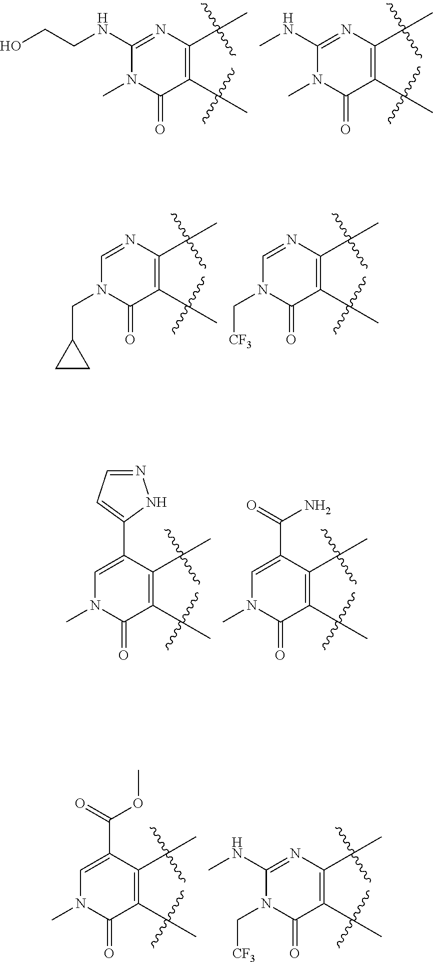

11. The compound of any one of the preceding claims, wherein Cy is phenyl and m is 0.

12. The compound of any one of the preceding claims, wherein X is O or S.

13. The compound of any one of the preceding claims, wherein X is O.

14. The compound of any one of the preceding claims, wherein 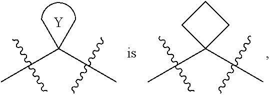
that is the ring Y is cyclobutane.

15. The compound of claim 14 wherein R^{7a} and R^{7b} are both H such that the group bound to the phenyl ring in structure I is 1-aminocyclobutyl.

16. The compound of claim 1 being:
1-(4-(3-phenylfuro[3,2-c]pyridin-2-yl)phenyl)cyclobutanamine
1-(4-(3-phenylfuro[2,3-c]pyridin-2-yl)phenyl)cyclobutanamine
1-(4-(3-iodofuro[2,3-c]pyridin-2-yl)phenyl)cyclobutanamine
2-(4-(1-aminocyclobutyl)phenyl)-5-methyl-3-phenylfuro[3,2-c]pyridin-4(5H)-one
2-(4-(1-aminocyclobutyl)phenyl)-3-phenylbenzofuran-6-carboxamide
2-(4-(1-aminocyclobutyl)phenyl)-3-phenylbenzofuran-5-carbonitrile
2-(4-(1-aminocyclobutyl)phenyl)-3-phenylbenzofuran-5-carboxamide
2-(4-(1-aminocyclobutyl)phenyl)-3-phenylbenzofuran-7-carbonitrile
2-(4-(1-aminocyclobutyl)phenyl)-3-phenylbenzofuran-7-carboxamide
1-(4-(7-phenylfuro[2,3-b]pyrazin-6-yl)phenyl)cyclobutanamine
1-(4-(5-bromo-3-phenylfuro[2,3-b]pyridin-2-yl)phenyl)cyclobutanamine
6-(4-(1-aminocyclobutyl)phenyl)-5-phenylfuro[2,3-d]pyrimidin-4(3H)-one
1-(4-(4-methoxy-2-morpholino-5-phenylfuro[2,3-d]pyrimidin-6-yl)phenyl)cyclobutanamine
N1-(6-(4-(1-aminocyclobutyl)phenyl)-4-methoxy-5-phenylfuro[2,3-d]pyrimidin-2-yl)-N2,N2-dimethylethane-1,2-diamine
6-(4-(1-aminocyclobutyl)phenyl)-2-(methylthio)-5-phenylfuro[2,3-d]pyrimidin-4(3H)-one
6-(4-(1-aminocyclobutyl)phenyl)-5-phenylthieno[2,3-d]pyrimidin-4(3H)-one
1-(4-(4-methoxy-3-phenylfuro[3,2-c]pyridin-2-yl)phenyl)cyclobutanamine
2-(4-(1-aminocyclobutyl)phenyl)-3-phenylfuro[3,2-c]pyridin-4(5H)-one
1-(4-(3-phenylfuro[2,3-b]pyridin-2-yl)phenyl)cyclobutanamine
1-(4-(5-Phenylfuro[2,3-d]pyrimidin-6-yl)phenyl)cyclobutanamine
1-(4-(5-Nitro-3-phenylfuro[2,3-b]pyridin-2-yl)phenyl)cyclobutanamine
2-(4-(1-Aminocyclobutyl)phenyl)-3-phenylfuro[2,3-b]pyridin-5-amine
1-(4-(2-Phenylfuro[3,2-b]pyridin-3-yl)phenyl)cyclobutanamine
1-(4-(2-(Methylthio)-5-phenylfuro[2,3-d]pyrimidin-6-yl)phenyl)cyclobutanamine
2-(4-(1-aminocyclobutyl)phenyl)-3-phenylbenzofuran-6-carbonitrile
6-(4-(1-aminocyclobutyl)phenyl)-4-methoxy-N,N-dimethyl-5-phenylfuro[2,3-d]pyrimidin-2-amine
1-(4-(2-(3-(aminomethyl)azetidin-1-yl)-4-methoxy-5-phenylfuro[2,3-d]pyrimidin-6-yl)phenyl)cyclobutanamine
1-(4-(5-morpholino-3-phenylfuro[2,3-b]pyridin-2-yl)phenyl)cyclobutanamine
2-(4-(3-aminoazetidin-3-yl)phenyl)-3-phenylbenzofuran-7-carbonitrile
6-(4-(1-aminocyclobutyl)phenyl)-4-methoxy-5-phenylfuro[2,3-d]pyrimidin-2-amine
6-(4-(1-aminocyclobutyl)phenyl)-4-methoxy-N-methyl-5-phenylfuro[2,3-d]pyrimidin-2-amine
1-(4-(2-(4-(2-(dimethylamino)ethyl)piperazin-1-yl)-4-methoxy-5-phenylfuro[2,3-d]pyrimidin-6-yl)phenyl)cyclobutanamine
6-(4-(1-aminocyclobutyl)phenyl)-4-methoxy-N-(2-(4-methylpiperazin-1-yl)ethyl)-5-phenylfuro[2,3-d]pyrimidin-2-amine
(8)-1-(6-(4-(1-aminocyclobutyl)phenyl)-4-methoxy-5-phenylfuro[2,3-d]pyrimidin-2-yl)pyrrolidin-3-ol
2-((6-(4-(1-aminocyclobutyl)phenyl)-4-methoxy-5-phenylfuro[2,3-d]pyrimidin-2-yl)oxy)ethanol
1-(4-(4-Methoxy-5-phenyl-2-(piperazin-1-yl)furo[2,3-d]pyrimidin-6-yl)phenyl)cyclobutanamine
2-((6-(4-(1-Aminocyclobutyl)phenyl)-4-methoxy-5-phenylfuro[2,3-d]pyrimidin-2-yl)amino)ethanol
1-(6-(4-(1-Aminocyclobutyl)phenyl)-4-methoxy-5-phenylfuro[2,3-d]pyrimidin-2-yl)piperidin-4-amine
1-(6-(4-(1-Aminocyclobutyl)phenyl)-4-methoxy-5-phenylfuro[2,3-d]pyrimidin-2-yl)piperidin-4-ol
3-((6-(4-(1-Aminocyclobutyl)phenyl)-4-methoxy-5-phenylfuro[2,3-d]pyrimidin-2-yl)amino)propan-1-ol
6-(4-(1-aminocyclobutyl)phenyl)-4-methoxy-N-(2-methoxyethyl)-5-phenylfuro[2,3-d]pyrimidin-2-amine
N1-(6-(4-(1-aminocyclobutyl)phenyl)-4-methoxy-5-phenylfuro[2,3-d]pyrimidin-2-yl)ethane-1,2-diamine
1-(4-(2-(1H-imidazol-1-yl)-4-methoxy-5-phenylfuro[2,3-d]pyrimidin-6-yl)phenyl)cyclobutanamine
1-(4-(6-(4-(1-aminocyclobutyl)phenyl)-4-methoxy-5-phenylfuro[2,3-d]pyrimidin-2-yl)piperazin-1-yl)ethanone
1-(4-(4-Ethoxy-5-phenyl-2-(piperazin-1-yl)furo[2,3-d]pyrimidin-6-yl)phenyl)cyclobutanamine
1-(4-(4-Methoxy-5-phenyl-2-(piperidin-1-yl)furo[2,3-d]pyrimidin-6-yl)phenyl)cyclobutanamine
N-(6-(4-(1-aminocyclobutyl)phenyl)-4-methoxy-5-phenylfuro[2,3-d]pyrimidin-2-yl)acetamide
1-(4-(2,4-dimethoxy-5-phenylfuro[2,3-d]pyrimidin-6-yl)phenyl)cyclobutanamine:
1-(6-(4-(1-Aminocyclobutyl)phenyl)-5-phenylfuro[2,3-d]pyrimidin-2-yl)piperidin-4-amine
2-((6-(4-(1-Aminocyclobutyl)phenyl)-5-phenylfuro[2,3-d]pyrimidin-2-yl)amino)ethanol
1-(4-(2-Morpholino-5-phenylfuro[2,3-d]pyrimidin-6-yl)phenyl)cyclobutanamine
1-(4-(5-Phenyl-2-(piperazin-1-yl)furo[2,3-d]pyrimidin-6-yl)phenyl)cyclobutanamine
2-((6-(4-(1-Aminocyclobutyl)phenyl)-5-phenylfuro[2,3-d]pyrimidin-2-yl)oxy)ethanol
6-(4-(1-Aminocyclobutyl)phenyl)-N-methyl-5-phenylfuro[2,3-d]pyrimidin-2-amine
(S)-6-(4-(1-aminocyclobutyl)phenyl)-2-(3-hydroxypyrrolidin-1-yl)-3-methyl-5-phenylfuro[2,3-d]pyrimidin-4(3H)-one
(R)-6-(4-(1-aminocyclobutyl)phenyl)-2-(3-hydroxypyrrolidin-1-yl)-3-methyl-5-phenylfuro[2,3-d]pyrimidin-4(3H)-one
6-(4-(1-aminocyclobutyl)phenyl)-3-methyl-5-phenyl-2-(pyrrolidin-1-yl)furo[2,3-d]pyrimidin-4(3H)-one
6-(4-(1-aminocyclobutyl)phenyl)-2-(4-hydroxypiperidin-1-yl)-3-methyl-5-phenylfuro[2,3-d]pyrimidin-4(3H)-one
2-((6-(4-(1-aminocyclobutyl)phenyl)-3-methyl-4-oxo-5-phenyl-3,4-dihydrofuro[2,3-d]pyrimidin-2-yl)amino)-N-methylacetamide
6-(4-(1-aminocyclobutyl)phenyl)-2-(3-hydroxyazetidin-1-yl)-3-methyl-5-phenylfuro[2,3-d]pyrimidin-4(3H)-one
(R)-6-(4-(1-aminocyclobutyl)phenyl)-2-((2-hydroxypropyl)amino)-3-methyl-5-phenylfuro[2,3-d]pyrimidin-4(3H)-one
2-((6-(4-(1-aminocyclobutyl)phenyl)-3-methyl-4-oxo-5-phenyl-3,4-dihydrofuro[2,3-d]pyrimidin-2-yl)amino)acetamide
6-(4-(1-aminocyclobutyl)phenyl)-2-(2-hydroxyethylamino)-3-methyl-5-phenylfuro[2,3-d]pyrimidin-4(3H)-one
6-(4-(1-aminocyclobutyl)phenyl)-3-methyl-2-(methylamino)-5-phenylfuro[2,3-d]pyrimidin-4(3H)-one
6-(4-(1-aminocyclobutyl)phenyl)-3-methyl-5-phenylfuro[2,3-d]pyrimidin-4(3H)-one
6-(4-(1-aminocyclobutyl)phenyl)-3-(2-methoxyethyl)-2-(methylthio)-5-phenylfuro[2,3-d]pyrimidin-4(3H)-one
6-(4-(1-aminocyclobutyl)phenyl)-N-methyl-2-(methylthio)-5-phenylfuro[2,3-d]pyrimidin-4-amine
6-(4-(1-aminocyclobutyl)phenyl)-3-(2-methoxyethyl)-2-(methylamino)-5-phenylfuro[2,3-d]pyrimidin-4(3H)-one
2-(6-(4-(1-aminocyclobutyl)phenyl)-4-(dimethylamino)-5-phenylfuro[2,3-d]pyrimidin-2-ylamino)ethanol
2-((6-(4-(1-aminocyclobutyl)phenyl)-4-morpholino-5-phenylfuro[2,3-d]pyrimidin-2-yl)amino)ethanol
2-((6-(4-(1-aminocyclobutyl)phenyl)-4-methyl-5-phenylfuro[2,3-d]pyrimidin-2-yl)amino)ethanol
2-((6-(4-(1-aminocyclobutyl)phenyl)-5-phenyl-4-(trifluoromethyl)furo[2,3-d]pyrimidin-2-yl)amino)ethanol
1-(4-(2-(methylthio)-5-phenyl-4-(trifluoromethyl)furo[2,3-d]pyrimidin-6-yl)phenyl)cyclobutanamine
1-(4-(4-iodo-2-(methylthio)-5-phenylfuro[2,3-d]pyrimidin-6-yl)phenyl)cyclobutanamine
6-(4-(1-aminocyclobutyl)phenyl)-2-((2-hydroxyethyl)amino)-5-phenylfuro[2,3-d]pyrimidin-4(3H)-one:
2,2′4(6-(4-(1-aminocyclobutyl)phenyl)-5-phenylfuro[2,3-d]pyrimidine-2,4-diyl)bis(azanediyl))diethanol
1-(4-(4-methoxy-5-phenylfuro[2,3-d]pyrimidin-6-yl)phenyl)cyclobutanamine
2-(6-(4-(1-aminocyclobutyl)phenyl)-4-(methylamino)-5-phenylfuro[2,3-d]pyrimidin-2-ylamino)ethanol
1-(4-(7-bromo-4-methoxy-3-phenylfuro[3,2-c]pyridin-2-yl)phenyl)cyclobutanamine
2-(6-(4-(1-aminocyclobutyl)phenyl)-4-(ethylamino)-5-phenylfuro[2,3-d]pyrimidin-2-ylamino)ethanol
6-(4-(1-aminocyclobutyl)phenyl)-3-methyl-2-morpholino-5-phenylfuro[2,3-d]pyrimidin-4(3H)-one
6-(4-(1-aminocyclobutyl)phenyl)-2-(2-methoxyethylamino)-3-methyl-5-phenylfuro[2,3-d]pyrimidin-4(3H)-one
6-(4-(1-aminocyclobutyl)phenyl)-2-((2-hydroxyethyl)(methyl)amino)-3-methyl-5-phenylfuro[2,3-d]pyrimidin-4(3H)-one
6-(4-(1-aminocyclobutyl)phenyl)-2-(2-hydroxyethoxy)-3-methyl-5-phenylfuro[2,3-d]pyrimidin-4(3H)-one
(S)-6-(4-(1-aminocyclobutyl)phenyl)-2-((2-hydroxypropyl)amino)-3-methyl-5-phenylfuro[2,3-d]pyrimidin-4(3H)-one
6-(4-(1-aminocyclobutyl)phenyl)-2,3-dimethyl-5-phenylfuro[2,3-d]pyrimidin-4(3H)-one.
6-(4-(1-aminocyclobutyl)phenyl)-2-(ethylamino)-3-methyl-5-phenylfuro[2,3-d]pyrimidin-4(3H)-one
6-(4-(1-aminocyclobutyl)phenyl)-4-(2-fluoroethoxy)-N-methyl-5-phenylfuro[2,3-d]pyrimidin-2-amine
6-(4-(1-aminocyclobutyl)phenyl)-2-cyclopropyl-3-methyl-5-phenylfuro[2,3-d]pyrimidin-4(3H)-one
2-(4-(1-aminocyclobutyl)phenyl)-5-(2-fluoroethyl)-3-phenylfuro[3,2-c]pyridin-4(5H)-one
6-(4-(1-aminocyclobutyl)phenyl)-3-ethyl-2-(methylamino)-5-phenylfuro[2,3-d]pyrimidin-4(3H)-one
6-(4-(1-aminocyclobutyl)phenyl)-3-ethyl-2-((2-hydroxyethyl)amino)-5-phenylfuro[2,3-d]pyrimidin-4(3H)-one
6-(4-(1-aminocyclobutyl)phenyl)-3-(cyclopropylmethyl)-2-(methylamino)-5-phenylfuro[2,3-d]pyrimidin-4(3H)-one
6-(4-(1-aminocyclobutyl)phenyl)-3-(cyclopropylmethyl)-2-((2-hydroxyethyl)amino)-5-phenylfuro[2,3-d]pyrimidin-4(3H)-one
2-(6-(4-(1-aminocyclobutyl)phenyl)-2-(methylthio)-4-oxo-5-phenylfuro[2,3-d]pyrimidin-3(4H)-yl)acetonitrile
2-(6-(4-(1-aminocyclobutyl)phenyl)-2-(methylthio)-4-oxo-5-phenylfuro[2,3-d]pyrimidin-3(4H)-yl)acetamide
(S)-6-(4-(1-aminocyclobutyl)phenyl)-2-(1-hydroxypropan-2-ylamino)-3-methyl-5-phenylfuro[2,3-d]pyrimidin-4(3H)-one
6-(4-(1-aminocyclobutyl)phenyl)-3-methyl-2-(3-oxopiperazin-1-yl)-5-phenylfuro[2,3-d]pyrimidin-4(3H)-one
6-(4-(1-aminocyclobutyl)phenyl)-3-(cyclopropylmethyl)-5-phenylfuro[2,3-d]pyrimidin-4(3H)-one
2-(6-(4-(1-aminocyclobutyl)phenyl)-4-oxo-5-phenylfuro[2,3-d]pyrimidin-3(4H)-yl)acetonitrile
6-(4-(1-aminocyclobutyl)phenyl)-3-(2-hydroxyethyl)-5-phenylfuro[2,3-d]pyrimidin-4(3H)-one
6-(4-(1-aminocyclobutyl)phenyl)-3-(2-fluoroethyl)-5-phenylfuro[2,3-d]pyrimidin-4(3H)-one
6-(4-(1-aminocyclobutyl)phenyl)-3-ethyl-5-phenylfuro[2,3-d]pyrimidin-4(3H)-one
6-(4-(1-aminocyclobutyl)phenyl)-3-(2-methoxyethyl)-5-phenylfuro[2,3-d]pyrimidin-4(3H)-one
6-(4-(1-aminocyclobutyl)phenyl)-5-phenyl-3-(2,2,2-trifluoroethyl)furo[2,3-d]pyrimidin-4(3H)-one
6-(4-(1-aminocyclobutyl)phenyl)-3-isopropyl-5-phenylfuro[2,3-d]pyrimidin-4(3H)-one
6-(4-(1-aminocyclobutyl)phenyl)-3-(2,2-difluoroethyl)-5-phenylfuro[2,3-d]pyrimidin-4(3H)-one
2-(4-(1-aminocyclobutyl)phenyl)-3-phenyl-5-(2,2,2-trifluoroethyl)furo[3,2-c]pyridin-4(5H)-one
6-(4-(1-aminocyclobutyl)phenyl)-3-(2-fluoroethyl)-2-(2-hydroxyethylamino)-5-phenylfuro[2,3-d]pyrimidin-4(3H)-one:
2-((6-(4-(1-aminocyclobutyl)phenyl)-4-(2-fluoroethoxy)-5-phenylfuro[2,3-d]pyrimidin-2-yl)amino)ethanol
2-(4-(1-aminocyclobutyl)phenyl)-7-bromo-5-(2-fluoroethyl)-3-phenylfuro[3,2-c]pyridin-4(5H)-one
2-(4-(1-aminocyclobutyl)phenyl)-5-(2-fluoroethyl)-7-(1-methyl-1H-pyrazol-4-yl)-3-phenylfuro[3,2-c]pyridin-4(5H)-one
2-(4-(1-aminocyclobutyl)phenyl)-5-methyl-7-(1-methyl-1H-pyrazol-4-yl)-3-phenylfuro[3,2-c]pyridin-4(5H)-one
2-(4-(1-aminocyclobutyl)phenyl)-5-methyl-3-phenyl-7-(pyrimidin-5-yl)furo[3,2-c]pyridin-4(5H)-one
2-(4-(1-aminocyclobutyl)phenyl)-5-methyl-3-phenyl-7-(1H-pyrazol-5-yl)furo[3,2-c]pyridin-4(5H)-one:
2-(4-(1-aminocyclobutyl)phenyl)-5-methyl-4-oxo-3-phenyl-4,5-dihydrofuro[3,2-c]pyridine-7-carboxamide methyl 2-(4-(1-aminocyclobutyl)phenyl)-5-methyl-4-oxo-3-phenyl-4,5-dihydrofuro[3,2-c]pyridine-7-carboxylate
2-(4-(1-aminocyclobutyl)phenyl)-N,5-dimethyl-4-oxo-3-phenyl-4,5-dihydrofuro[3,2-c]pyridine-7-carboxamide
2-(4-(1-aminocyclobutyl)phenyl)-N-(2-hydroxyethyl)-5-methyl-4-oxo-3-phenyl-4,5-dihydrofuro[3,2-c]pyridine-7-carboxamide
6-(4-(1-aminocyclobutyl)phenyl)-2-((2-hydroxyethyl)amino)-5-phenyl-3-(2,2,2-trifluoroethyl)furo[2,3-d]pyrimidin-4(3H)-one
6-(4-(1-aminocyclobutyl)phenyl)-2-(methylamino)-5-phenyl-3-(2,2,2-trifluoroethyl)furo[2,3-d]pyrimidin-4(3H)-one
6-(4-(1-aminocyclobutyl)phenyl)-2-methyl-5-phenyl-3-(2,2,2-trifluoroethyl)furo[2,3-d]pyrimidin-4(3H)-one
6-(4-(1-aminocyclobutyl)phenyl)-3-methyl-5-phenylfuro[2,3-d]pyrimidine-2,4(1H,3H)-dione
6-(4-(1-aminocyclobutyl)phenyl)-1,3-dimethyl-5-phenylfuro[2,3-d]pyrimidine-2,4(1H,3H)-dione
1-(4-(7-methoxy-3-phenylfuro[2,3-c]pyridin-2-yl)phenyl)cyclobutanamine
1-(4-(3-iodo-7-methoxyfuro[2,3-c]pyridin-2-yl)phenyl)cyclobutanamine
2-(4-(1-aminocyclobutyl)phenyl)-3-phenylfuro[2,3-c]pyridin-7(6H)-one
2-(4-(1-aminocyclobutyl)phenyl)-6-methyl-3-phenylfuro[2,3-c]pyridin-7(6H)-one
2-(4-(1-aminocyclobutyl)phenyl)-6-(2-fluoroethyl)-3-phenylfuro[2,3-c]pyridin-7(6H)-one
2-(4-(1-aminocyclobutyl)phenyl)-6-(3-hydroxypropyl)-3-phenylfuro[2,3-c]pyridin-7(6H)-one
1-(4-(4-bromo-7-methoxy-3-phenylfuro[2,3-c]pyridin-2-yl)phenyl)cyclobutanamine
1-(4-(3-phenyl-4-(1H-pyrazol-4-yl)furo[3,2-c]pyridin-2-yl)phenyl)cyclobutanamine
1-(4-(2-Chloro-7-phenylfuro[2,3-b]pyrazin-6-yl)phenyl)cyclobutanamine
1-(4-(7-Phenyl-2-(1H-pyrazol-4-yl)furo[2,3-b]pyrazin-6-yl)phenyl)cyclobutanamine
1-(4-(2-(1-Methyl-1H-pyrazol-4-yl)-7-phenylfuro[2,3-b]pyrazin-6-yl)phenyl)cyclobutanamine
1-(4-(2-(1-methyl-1H-pyrazol-5-yl)-7-phenylfuro[2,3-b]pyrazin-6-yl)phenyl)cyclobutanamine
1-(4-(2-Cyclopropyl-7-phenylfuro[2,3-b]pyrazin-6-yl)phenyl)cyclobutanamine
1-(4-(2,7-Diphenylfuro[2,3-b]pyrazin-6-yl)phenyl)cyclobutanamine
1-(4-(7-Phenyl-2-(pyrimidin-5-yl)furo[2,3-b]pyrazin-6-yl)phenyl)cyclobutanamine
1-(4-(7-Phenyl-2-(pyridin-2-yl)furo[2,3-b]pyrazin-6-yl)phenyl)cyclobutanamine
1-(4-(2-(Benzyloxymethyl)-7-phenylfuro[2,3-t]pyrazin-6-yl)phenyl)cyclobutanamine
1-(4-(2-(3,5-Dimethyl-1H-pyrazol-4-yl)-7-phenylfuro[2,3-b]pyrazin-6-yl)phenyl)cyclobutanamine:
1-(4-(7-Phenyl-2-(1H-pyrazol-5-yl)furo[2,3-b]pyrazin-6-yl)phenyl)cyclobutanamine
(6-(4-(1-Aminocyclobutyl)phenyl)-7-phenylfuro[2,3-b]pyrazin-2-yl)methanol
1-(4-(2-Ethynyl-7-phenylfuro[2,3-b]pyrazin-6-yl)phenyl)cyclobutanamine
1-(4-(7-Phenyl-2-(1H-1,2,3-triazol-5-yl)furo[2,3-b]pyrazin-6-yl)phenyl)cyclobutanamine
2-(6-(4-(1-Aminocyclobutyl)phenyl)-7-phenylfuro[2,3-b]pyrazin-2-ylamino)ethanol
1-(4-(2-Methyl-7-phenylfuro[2,3-b]pyrazin-6-yl)phenyl)cyclobutanamine
1-(4-(7-phenyl-2-(pyridin-3-yl)furo[2,3-b]pyrazin-6-yl)phenyl)cyclobutanamine
N-(3-(6-(4-(1-aminocyclobutyl)phenyl)-7-phenylfuro[2,3-b]pyrazin-2-yl)phenyl)acetamide
3-(6-(4-(1-aminocyclobutyl)phenyl)-7-phenylfuro[2,3-b]pyrazin-2-yl)benzamide
1-(4-(3-(Methylthio)-7-phenyl-5H-pyrrolo[3,2-b]pyrazin-6-yl)phenyl)cyclobutanamine and pharmaceutically acceptable salts, stereoisomers and tautomers thereof.

17. The compound of claim 1 being:
2-(4-(1-aminocyclobutyl)phenyl)-3-phenylbenzofuran-5-carboxamide
1-(4-(4-methoxy-2-morpholino-5-phenylfuro[2,3-d]pyrimidin-6-yl)phenyl)cyclobutanamine
N1-(6-(4-(1-aminocyclobutyl)phenyl)-4-methoxy-5-phenylfuro[2,3-d]pyrimidin-2-yl)-N2,N2-dimethylethane-1,2-diamine
6-(4-(1-aminocyclobutyl)phenyl)-2-(methylthio)-5-phenylfuro[2,3-d]pyrimidin-4(3H)-one
6-(4-(1-aminocyclobutyl)phenyl)-4-methoxy-N,N-dimethyl-5-phenylfuro[2,3-d]pyrimidin-2-amine
1-(4-(2-(3-(aminomethyl)azetidin-1-yl)-4-methoxy-5-phenylfuro[2,3-d]pyrimidin-6-yl)phenyl)cyclobutanamine
6-(4-(1-aminocyclobutyl)phenyl)-4-methoxy-5-phenylfuro[2,3-d]pyrimidin-2-amine
6-(4-(1-aminocyclobutyl)phenyl)-4-methoxy-N-methyl-5-phenylfuro[2,3-d]pyrimidin-2-amine
1-(4-(2-(4-(2-(dimethylamino)ethyl)piperazin-1-yl)-4-methoxy-5-phenylfuro[2,3-d]pyrimidin-6-yl)phenyl)cyclobutanamine
6-(4-(1-aminocyclobutyl)phenyl)-4-methoxy-N-(2-(4-methylpiperazin-1-yl)ethyl)-5-phenylfuro[2,3-d]pyrimidin-2-amine
(S)-1-(6-(4-(1-aminocyclobutyl)phenyl)-4-methoxy-5-phenylfuro[2,3-d]pyrimidin-2-yl)pyrrolidin-3-ol
2-((6-(4-(1-aminocyclobutyl)phenyl)-4-methoxy-5-phenylfuro[2,3-d]pyrimidin-2-yl)oxy)ethanol
1-(4-(4-Methoxy-5-phenyl-2-(piperazin-1-yl)furo[2,3-d]pyrimidin-6-yl)phenyl)cyclobutanamine
2-((6-(4-(1-Aminocyclobutyl)phenyl)-4-methoxy-5-phenylfuro[2,3-d]pyrimidin-2-yl)amino)ethanol
1-(6-(4-(1-Aminocyclobutyl)phenyl)-4-methoxy-5-phenylfuro[2,3-d]pyrimidin-2-yl)piperidin-4-amine
1-(6-(4-(1-Aminocyclobutyl)phenyl)-4-methoxy-5-phenylfuro[2,3-d]pyrimidin-2-yl)piperidin-4-ol
3-((6-(4-(1-Aminocyclobutyl)phenyl)-4-methoxy-5-phenylfuro[2,3-d]pyrimidin-2-yl)amino)propan-1-ol and pharmaceutically acceptable salts, stereoisomers and tautomers thereof.

18. The compound of claim 1 being:
6-(4-(1-aminocyclobutyl)phenyl)-4-methoxy-5-phenylfuro[2,3-d]pyrimidin-2-amine
6-(4-(1-aminocyclobutyl)phenyl)-4-methoxy-N-methyl-5-phenylfuro[2,3-d]pyrimidin-2-amine
6-(4-(1-aminocyclobutyl)phenyl)-4-methoxy-N-(2-(4-methylpiperazin-1-yl)ethyl)-5-phenylfuro[2,3-d]pyrimidin-2-amine
2-((6-(4-(1-aminocyclobutyl)phenyl)-4-methoxy-5-phenylfuro[2,3-d]pyrimidin-2-yl)oxy)ethanol
2-((6-(4-(1-Aminocyclobutyl)phenyl)-4-methoxy-5-phenylfuro[2,3-d]pyrimidin-2-yl)amino)ethanol
3-((6-(4-(1-Aminocyclobutyl)phenyl)-4-methoxy-5-phenylfuro[2,3-d]pyrimidin-2-yl)amino)propan-1-ol and pharmaceutically acceptable salts, stereoisomers and tautomers thereof.

19. The compound of claim 1 being:
6-(4-(1-aminocyclobutyl)phenyl)-2-(2-hydroxyethylamino)-3-methyl-5-phenylfuro[2,3-d]pyrimidin-4(3H)-one
6-(4-(1-aminocyclobutyl)phenyl)-3-methyl-2-(methylamino)-5-phenylfuro[2,3-d]pyrimidin-4(3H)-one
6-(4-(1-aminocyclobutyl)phenyl)-3-(cyclopropylmethyl)-5-phenylfuro[2,3-d]pyrimidin-4(3H)-one,
6-(4-(1-aminocyclobutyl)phenyl)-5-phenyl-3-(2,2,2-trifluoroethyl)furo[2,3-d]pyrimidin-4(3H)-one,
2-(4-(1-aminocyclobutyl)phenyl)-5-methyl-3-phenyl-7-(1H-pyrazol-5-yl)furo[3,2-c]pyridin-4(5H)-one:
2-(4-(1-aminocyclobutyl)phenyl)-5-methyl-4-oxo-3-phenyl-4,5-dihydrofuro[3,2-c]pyridine-7-carboxamide methyl 2-(4-(1-aminocyclobutyl)phenyl)-5-methyl-4-oxo-3-phenyl-4,5-dihydrofuro[3,2-c]pyridine-7-carboxylate
6-(4-(1-aminocyclobutyl)phenyl)-2-(methylamino)-5-phenyl-3-(2,2,2-trifluoroethyl)furo[2,3-d]pyrimidin-4(3H)-one and pharmaceutically acceptable salts, stereoisomers and tautomers thereof.

20. A pharmaceutical composition comprising a pharmaceutical carrier and, dispersed therein, a compound of any one of the previous claims.

21. The compound of any one of claims 1 to 19 for use in therapy.

22. The compound of any one of claims 1 to 19 for use in the treatment or prevention of cancer.

23. A method of treating or preventing cancer comprising administering a compound according to any one of claims 1 to 19 to a subject in need thereof.


SEQUENCE LISTING (1 sequence)

SEQ ID NO: 1 - protein, 11 aa, Artificial Sequence
  Synthetic peptide 5′ FAM Crosstide
GRPRTSSFAEG